(19) United States
(12) Patent Application Publication
(10) Pub. No.: US 2023/0348571 A1
(43) Pub. Date: Nov. 2, 2023

(54) CROSS-REACTIVE CORONAVIRUS ANTIBODIES AND USES THEREOF

(71) Applicants: VANDERBILT UNIVERSITY, Nashville, TN (US); BOARD OF REGENTS, THE UNIVERSITY OF TEXAS SYSTEM, Austin, TX (US); TRUSTEES OF DARTMOUTH COLLEGE, Hanover, NH (US)

(72) Inventors: Ivelin Stefanov Georgiev, Nashville, TN (US); Andrea R. Shiakolas, Nashville, TN (US); Kevin J. Kramer, Nashville, TN (US); James E. Crowe, Jr., Nashville, TN (US); Robert Carnahan, Nashville, TN (US); Nianshuang Wang, Austin, TX (US); Daniel Wrapp, Chandler, AZ (US); Jason McLellan, Austin, TX (US)

(21) Appl. No.: 17/917,174

(22) PCT Filed: Apr. 6, 2021
(86) PCT No.: PCT/US2021/025909

§ 371 Date: Oct. 5, 2022

Related U.S. Application Data
(60) Provisional application No. 63/005,788, filed on Apr. 6, 2020.
(60) Provisional application No. 63/018,637, filed on May 1, 2020.
(60) Provisional application No. 63/036,016, filed on Jun. 8, 2020.
(60) Provisional application No. 63/091,517, filed on Oct. 14, 2020.
(60) Provisional application No. 63/140,355, filed on Jan. 22, 2021.

(51) Int. Cl. C07K 16/10; A61P 31/14

(52) U.S. Cl. CPC C07K 16/1003; A61P 31/14; A61K 2039/505

(57) ABSTRACT

The present disclosure relates to antibodies and uses thereof for treating, preventing, and detecting coronavirus infection.

### CROSS REFERENCE TO RELATED APPLICATIONS

This application is a national stage application filed under 35 U.S.C. § 371 of PCT/US2021/025909, filed Apr. 6, 2021, which claims the benefit of U.S. Provisional Patent Application Ser. No. 63/005,788 filed Apr. 6, 2020, U.S. Provisional Patent Application Ser. No. 63/018,637 filed May 1, 2020, U.S. Provisional Patent Application Ser. No. 63/036,016 filed Jun. 8, 2020, U.S. Provisional Patent Application Ser. No. 63/091,517 filed Oct. 14, 2020, and U.S. Provisional Patent Application Ser. No. 63/140,355 filed Jan. 22, 2021, the disclosures of which are expressly incorporated herein by reference in their entireties.

### STATEMENT REGARDING FEDERALLY SPONSORED RESEARCH

This invention was made with government support under Grant No. RO1 AI131722ACV awarded by the National Institutes of Health. The government has certain rights in the invention.

### FIELD

The present disclosure relates to antibodies and uses thereof for treating, preventing, and detecting coronavirus infection.

### REFERENCE TO SEQUENCE LISTING

The Sequence Listing submitted Apr. 6, 2021 as a text file named “10644-115WO1_2021_04_06_Sequence_Listing.txt,” created on Apr. 6, 2021, and having a size of 9,784 bytes is hereby incorporated by reference pursuant to 37 C.F.R. § 1.52(e)(5).

### BACKGROUND

SARS-CoV-2, or the 2019 novel coronavirus (COVID-19), is a significant pandemic threat that has resulted in over 126,000,000 diagnosed cases including 2,760,000 deaths as of Mar. 26, 2021. Initially detected in Wuhan, China, human-human transmission has resulted in confirmed cases all over the world. On Jan. 30, 2020, the World Health Organization declared a Public Health of International Concern due to the COVID-19 outbreak and pronounced it a global pandemic on Mar. 12, 2020. The development of preventive and therapeutic measures that can counteract the ongoing, and any future, coronavirus pandemics is therefore of utmost significance for public health worldwide. What is needed are novel compositions and methods for treating and diagnosing SARS-CoV-2 infection.

### SUMMARY

Disclosed herein are recombinant antibodies and uses thereof for preventing, treating, and detecting coronavirus infection. Antibody sequences were obtained from an individual previously infected with a SARS-CoV-1 infection.

In some aspects, disclosed herein is a recombinant antibody, wherein the antibody comprises a light chain variable region (VL) that comprises a light chain complementarity determining region (CDRL)1, CDRL2, and CDRL3 and/or a heavy chain variable region (VH) that comprises a heavy chain complementarity determining region (CDRH)1, CDRH2, and CDRH3, wherein: CDRH3 comprises an amino acid sequence at least 60% identical to SEQ ID NOs: 109-126; and CDRL3 comprises an amino acid sequence at least 60% identical to SEQ ID NOs: 163-180.

In some embodiments, CDRH3 comprises at least one amino acid substitution when compared to SEQ ID NOs: 109-126. In some embodiments, CDRL3 comprises at least one amino acid substitution when compared to SEQ ID NOs: 163-180.

In some embodiments, CDRH1 comprises an amino acid sequence at least 60% identical to SEQ ID NOs: 73-90; and/or CDRL1 comprises an amino acid sequence at least 60% identical to SEQ ID NOs: 127-144.

In some embodiments, CDRH1 comprises at least one amino acid substitution when compared to SEQ ID NOs: 73-90. In some embodiments, CDRL1 comprises at least one amino acid substitution when compared to SEQ ID NOs: 127-144.

In some embodiments, CDRH2 comprises an amino acid sequence at least 60% identical to SEQ ID NOs: 91-108; and/or CDRL2 comprises an amino acid sequence at least 60% identical to SEQ ID NOs: 145-162.

In some embodiments, CDRH2 comprises at least one amino acid substitution when compared to SEQ ID NOs: 91-108. In some embodiments, CDRL2 comprises at least one amino acid substitution when compared to SEQ ID NOs: 145-162.

In some embodiments, VH comprises an amino acid sequence selected from the group consisting of SEQ ID NOs: 37-54. In some embodiments, VL comprises an amino acid sequence selected from the group consisting of SEQ ID NOs: 55-72.

In some embodiments, the recombinant antibody is selected from Table 1. In some embodiments, the recombinant antibody is selected from Table 2.

In one aspect, disclosed herein is a nucleic acid encoding a recombinant antibody as disclosed herein.

In one aspect, disclosed herein is a recombinant expression cassette or plasmid comprising a sequence to express a recombinant antibody as disclosed herein.

In one aspect, disclosed herein is a host cell comprising an expression cassette or a plasmid as disclosed herein.

In one aspect, disclosed herein is a method of producing an antibody, comprising cultivating or maintaining a host cell under conditions to produce the antibody.

In one aspect, disclosed herein is a method of treating a coronavirus infection in a subject, comprising administering to the subject a therapeutically effective amount of a recombinant antibody as disclosed herein. In some embodiments, the coronavirus is SARS-CoV-2.

In some aspects, disclosed herein is a method for detecting a coronavirus infection in a subject, comprising: providing a biological sample from the subject, and detecting a coronavirus antigen in the biological sample with an antibody that specifically binds to the coronavirus antigen, wherein the antibody is from any aspect as disclosed herein, and wherein the presence of the coronavirus antigen in the biological sample indicates the subject is infected with a coronavirus. In some embodiments, the coronavirus is SARS-CoV-2.

### DESCRIPTION OF DRAWINGS

The accompanying figures, which are incorporated in and constitute a part of this specification, illustrate aspects described below.

FIG. 1 shows schematic of the LIBRA-seq assay. Fluorescently-labelled, DNA-barcoded antigens are used to sort antigen-positive B cells before co-encapsulation of single B cells with bead-delivered oligos using droplet microfluidics. Bead-delivered oligos index both cellular BCR transcripts and antigen barcodes during reverse transcription, enabling direct mapping of BCR sequence to antigen specificity following sequencing. Note: schematic is not to scale, and the number and placement of oligonucleotides on each antigen can vary.

FIGS. 2A-2C show LIBRA-seq identification of cross-reactive HIV and influenza antibodies in HIV-infected samples from donors N90 (FIG. 2A) and NIAID 45 (FIG. 2B). ELISA binding (shown as area under the ELISA binding curve, AUC) and LIBRA-seq scores. Heatmap is tan(min)-white-blue(max). FIG. 2C shows neutralization of diverse HIV-1 strains by 3602-870 (N90) and published values for VRC38.01 for comparison (red-yellow-green-white heatmap).

FIG. 3 shows hepatitis C neutralization by mAbs 180 and 692, identified by LIBRA-seq. mAb 180 showed exceptional hepatitis C neutralization breadth against a panel of 19 HCV pseudoparticles, shown as % neutralized virus at 100 μg/ml ab; broadest known HEPC74 antibody from the Bailey group is used a control.

FIG. 4 shows LIBRA-seq identification of SARS-CoV-2 cross-reactive antibodies from a patient with a previous SARS-CoV-1 infection. Heatmap shows magnitude of LIBRA-seq scores for example SARS-CoV-2 cross-reactive IgG+ B cells (rows) against a set of CoV antigens (columns): blue (low)-white-red (high).

FIGS. 5A-5E show LIBRA-seq assay schematic. The assay consists of the following general steps: FIG. 5A. Antigens are recombinantly produced, biotinylated, and labeled with a DNA “barcode” oligonucleotide. The DNA-barcoded antigens are mixed with cells of interest and labeled with streptavidin fluorophores. FIG. 5B. Antigen positive B cells are bulk sorted and diluted to an appropriate concentration for single cell sequencing. FIG. 5C. Using the 10×Chromium controller, each cell (along with its bound antigens) is isolated in a single cell emulsion droplet along with a bead that has primers for downstream library preparation. FIG. 5D. Bead delivered oligos index both cellular BCR transcripts and antigen barcodes during reverse transcription. FIG. 5E. Library preparation results in amplification of transcripts for each cell that are indexed with the same cell barcode to enable direct mapping of BCR sequence to antigen specificity.

FIG. 6 shows LIBRA-seq applied to a SARS convalescent donor PBMC sample. An antigen screening library of oligonucleotide-labeled antigens was generated. This consisted of CoV antigens: SARS-CoV-2 spike, SARS-CoV-1 spike, MERS spike, MERS S1 trimer, OC43 spike, and HKU1 spike. There were also two HIV envelope trimer antigens included in the library, ZM197 and CZA97. The antigen screening library was mixed with the donor PBMCs, and the LIBRA-seq workflow was executed.

FIG. 7 shows antigen reactivity of LIBRA-seq identified antibodies. After next generation sequencing, 2526 B cells were recovered that had paired heavy/light chain sequencing information and antigen reactivity information. This heatmap shows the LIBRA-seq scores for each antigen for each cell recovered—each cell is one row in the matrix. The matrix is further divided by antibody isotype, shown to the right of the heatmap. LIBRA-seq scores are shown from −2 to 2, as dark blue to red respectively. Scores outside of this range were shown as the minimum and maximum values.

FIGS. 8A-8B show identification of SARS-CoV-2, SARS-CoV-1, and MERS mono-reactive and cross-reactive antibodies. FIG. 8A shows a Venn diagram depiction of the number of antibodies that had high LIBRA-seq scores (LIBRA-seq score >1) for SARS-CoV-2, SARS-CoV-1, and MERS. There were 10 antibodies that showed high scores to all three antigens: SARS-CoV-2, SARS-CoV-1, and MERS. FIG. 8B shows CDRH3 lengths and heavy chain variable (VH) region identity for different categories of antibodies with high LIBRA-seq scores (LIBRA-seq score >1) for CoV antigens shown, including both monoreactive and cross-reactive antibodies.

FIG. 9 shows identification of other CoV-specific antibodies. Genetic characteristics and antigen reactivity as determined by LIBRA-seq score of antibodies prioritized for expression and validation from the SARS-CoV-1 convalescent donor sample. Percent identity is calculated at the nucleotide level, and CDR length and sequences are noted at the amino acid level. LIBRA-seq scores are shown from −2 to 2, as dark blue to red respectively. Scores outside of this range were shown as the minimum and maximum values. FIG. 9 includes sequences

(SEQ ID NO: 120)
ARRPQYLLLSMTTGRRHHDFVMDV,
(SEQ ID NO: 16031)
ARDRVERTGNVGFGYYAMDV,
(SEQ ID NO: 16929)
VRGRTY,
(SEQ ID NO: 18090)
AREVNYYSAFDD,
(SEQ ID NO: 117)
ARDRSATYYGPFDY,
(SEQ ID NO: 123)
AKDLLSHSGTYSAGSTFDY,
(SEQ ID NO: 111)
ARLDYSKQT,
(SEQ ID NO: 114)
AAGPTGYDLLTGQYFPYFNY,
(SEQ ID NO: 122)
VKEDTPLVFDS,
(SEQ ID NO: 124)
VKMRTAVVGVTPL,
(SEQ ID NO: 17251)
ASLPTYGSGRWGIDS,
(SEQ ID NO: 113)
ARDFDLVVPSATYPPFYYHGMDV,
(SEQ ID NO: 16198)
ARVTIVSSFTNRFDP,
(SEQ ID NO: 110)
ALGRKDYGDYYR,
(SEQ ID NO: 118)
AGFLPVYNNGWSYFDS,
(SEQ ID NO: 174)
QQYYNTPRT,
(SEQ ID NO: 163)
QQTYSSPSYT,
(SEQ ID NO: 169)
QQYNRWLWT,
(SEQ ID NO: 175)
QQYNFWWT,
(SEQ ID NO: 25295)
NSRDNSGNHPVI,
(SEQ ID NO: 26363)
QESYSTNT,
(SEQ ID NO: 165)
MQALQTPLT,
(SEQ ID NO: 168)
QSYHGSDVV,
(SEQ ID NO: 176)
QQYDSYST,
(SEQ ID NO: 178)
LODYNYLFS,
(SEQ ID NO: 25135)
QQGNSFPLT,
(SEQ ID NO: 167)
NSRDSSGDQTFYV,
(SEQ ID NO: 166)
QHRVT,
(SEQ ID NO: 164)
QQYAPSPPWYI,
and
(SEQ ID NO: 172)
AVWDDSLNGPV

FIG. 10 shows antibody binding by ELISA. 15 prioritized antibodies were recombinantly expressed, purified, and tested for binding to antigens by ELISA. 46472-11 has a 15-nucleotide insertion in the heavy chain variable region and was expressed with (46472-1 lins) and without (46472-11) the insertion. 46472-1, 46472-2, 46472-3, 46472-4, 46472-6, and 46472-12 showed binding to SARSCoV-2 spike, SARS-CoV-1 spike, and MERS spike. 46472-6 and 46472-12 showed additional binding to OC43 and HKU1 by ELISA. None of the antibodies bound to irrelevant antigen H1 NC99, an influenza hemagglutinin protein. All antibodies tested are shown in the legend, including a negative IgG control. A positive control for H1 NC99, 3602-1707, is also shown, which was only tested in the H1 NC99 ELISA.

FIGS. 11A-11C show additional binding properties of antibodies. FIG. 11A shows that antibodies were tested at a single concentration (10 μg/ml) for binding to SARS-CoV-2 receptor binding domain, and 46472-12 showed binding to this subunit. FIG. 11B shows that antibodies were also tested for binding to SARS-CoV-2 spike in a cell surface display assay. Expi 293F cells were transfected with full length SARS-CoV-2 spike plasmid and then antibodies were tested for binding using flow cytometry. Binding was detected using an anti-human Fc antibody conjugated to FITC. 46472-1 data and gating is shown. A mock transfection control was also included. In FIG. 11C, antibody binding to cell surface displayed SARS-CoV-2 spike protein is shown as the percent of FITC+ cells at a single concentration (10 μg/ml). 46472-1, 46472-2, 46472-3, 46472-4, 46472-6, 46472-11ins and 46472-12 showed binding to cell surface displayed spike protein.

FIG. 12 shows recovery of virus specific antibodies suing LIBRA-seq.

FIG. 13 shows that antibodies were prioritized using sequence features and LIBRA-seq scores.

FIGS. 14A-14D show identification of coronavirus cross-reactive antibodies from SARS-CoV-1 convalescent PBMC sample using LIBRA-seq. FIG. 14A shows schematic of DNA-barcoded antigens used to probe a SARS-CoV-1 donor PBMC sample. The LIBRA-seq experiment setup consisted of nine oligo-labelled antigens in the screening library: SARS-CoV-2 S, SARS-CoV-1 S, MERS-CoV S, MERS-CoV S1, OC43 S, HKU1 S, and two HIV negative controls (ZM197, and CZA97). FIG. 14B shows LIBRA-seq scores for SARS-CoV-1 (x-axis) and SARS-CoV-2 (y-axis) for all IgG cells recovered from sequencing are shown as circles. The 15 selected antibodies are highlighted in purple. FIG. 14C shows that antibodies were tested for binding to SARS-CoV-2 S (S2P), SARS-CoV-1 S, MERS Spike OC43-CoV S, HKU1-CoV S, and SARS-CoV-2 S (HexaPro) by ELISA. HIV-specific antibody VRC01 is used as a negative control. Anti-SARS-CoV-1 mouse antibody 240CD was also used (BEI Resources). ELISAs were performed in technical duplicates with at least two biological duplicates. FIG. 14D shows that ELISA binding data against the antigens are displayed as a heatmap of the AUC analysis calculated from the data in FIG. 14C, with AUC of 0 displayed as white, and maximum AUC as purple. ELISAs were performed in technical duplicates with at least two biological duplicates.

FIGS. 15A-15F show epitope mapping of cross-reactive antibodies. FIG. 15A shows, for cross-reactive coronavirus antibodies, ELISA binding data against the antigens are displayed as a heatmap of the AUC analysis calculated from the data in FIG. 18A and FIG. 15B for SARS-CoV-2 S1 reactive antibodies, ELISA binding data against the RBD and NTD are displayed as a heatmap of the AUC analysis calculated from the data in FIG. 19B. AUC of 0 is displayed as white and maximum AUC as purple. ELISA data are representative of at least two independent experiments. ELISA AUC is displayed as a heat map. (Anti-HIV antibody VRCO1 and anti-VEGF antibody are shown as a negative control and anti-SARS-CoV-1 antibody 240CD is shown as positive control.) FIG. 15C shows surface plasmon resonance binding of 46472-12 Fab to SARS-CoV-2 RBD. Affinity measurements are shown to the right of the graph. FIG. 15D shows that cross-reactive antibodies were used in a competition ELISA to determine if binding of one antibody affected binding of another. Competitor antibodies were added at 10 μg/ml, and then detected antibodies were added at 0.1 μg/ml. The percent reduction in binding compared to binding without a competitor is shown. An anti-HIV antibody was also used as a negative control. ELISAs were performed in technical duplicates with at least two biological duplicates. FIG. 15E shows that antibodies were tested for autoreactivity against a variety of antigens in the Luminex AtheNA assay. Anti-HIV antibody 4E10 was used as a positive control and Ab82 was used as a negative control. FIG. 15F shows that cross-reactive coronavirus antibodies target a variety of epitopes on the SARS-CoV-2 S protein, including the RBD, NTD, and S2 domains, highlighted on the structure (pdb: 6VSB). Antibodies targeting each epitope are listed and color coded for each domain.

FIGS. 16A-16E show functional activity of cross-reactive coronavirus antibodies. FIG. 16A shows that cross-reactive coronavirus antibodies were tested for antibody-dependent cellular phagocytosis activity (ADCP) against SARS-CoV-2 S, compared to positive control antibody CR3022 and negative control Palivizumab, an anti-RSV antibody. Area under the curve of the phagocytosis score is shown, calculated from data in FIG. 20C. FIG. 16B shows that 46472-4 and 46472-12 were tested for antibody-dependent cellular phagocytosis activity against SARS-CoV-1 S, compared to CR3022 antibody and anti-RSV antibody Palivizumab. Area under the curve of the phagocytosis score is shown, calculated from data in FIG. 20D. FIG. 16C shows that cross-reactive coronavirus antibodies were tested for antibody-dependent trogocytosis (ADCT) activity against SARS-CoV-2 S coated on cells, compared to positive control CR3022 and anti-RSV antibody Palivizumab. Area under the curve of the trogocytosis score is shown, calculated from data in FIG. 20E. FIG. 16D shows that cross-reactive coronavirus antibodies were tested for antibody-dependent trogocytosis activity against SARS-CoV-2 S displayed on transfected cells, compared to positive control CR3022 and anti-RSV antibody Palivizumab. Area under the curve of the trogocytosis score is shown, calculated from data in FIG. 20F. FIG. 16E shows that cross-reactive coronavirus antibodies were tested for antibody-dependent complement deposition (ADCD) activity against SARS-CoV-2 S, compared to positive control CR3022 and anti-RSV antibody Palivizumab. Area under the curve of the C3b deposition score is shown, calculated from data in FIG. 20G.

FIGS. 17A-17D shows in vivo effects of cross-reactive antibodies. FIG. 17A shows timeline of the prophylactic antibody experiment in SARS-CoV-2 mouse adapted (MA) in vivo infection model. 200 μg antibody was given via intraperitoneal route to 12 month old female BALB/c mice 12 hours prior to virus inoculation (n=5 per group). 1×10³ or 1×10⁴ PFU infectious dose of SARS-CoV-2 MA was administered intranasally for the low dose and high dose experiments, respectively. Weights were measured daily, and on day 4 tissue was collected for histopathology and viral load quantification. FIG. 17B shows lung hemorrhage scores of gross pathology are shown for each low dose (1×10³ PFU of SARS-CoV-2 MA) treatment group. An ordinary one-way ANOVA test with multiple comparisons was performed. FIG. 17C shows, for the experiment utilizing 1×10⁴ PFU of SARS-CoV-2 MA, percent survival for each antibody group is shown. 2/5, 4/5, 3/5, and 2/5 mice survived to day 4 for antibodies 46472-4, 46472-12, CR3022 and isotype control DENV-2D22 respectively. FIG. 17D shows lung hemorrhage scores of gross pathology are shown for each high dose (1×10⁴ PFU of SARS-CoV-2 MA) treatment group. An ordinary one-way ANOVA test with multiple comparisons was performed.

FIG. 18A shows gating scheme for fluorescent-activated cell sorting of convalescent SARS-CoV-1 donor. Cells were initially stained with Ghost Red 780, CD14-APC-Cy7, CD3-FITC, CD19-BV711, and IgG-PE-Cy5 along with a DNA-barcoded antigen screening library. To detect antigen-positive B cells, cells were washed and treated with a streptavidin-PE secondary stain. Gates as drawn are based on gates used during the sort, and percentages from the sort are listed. FIG. 18B shows the categorization of processing of Cell ranger identified cells after sequencing. FIG. 18C shows genetic sequence characteristics and antigen specificity of the 15 highlighted antibodies of FIG. 14. Percent identity is calculated at the nucleotide level and CDRH3 and CDRL3 lengths and sequences are noted at the amino acid level. FIG. 18D shows ELISA binding data against coronavirus S antigens. HIV-specific antibody VRC01 was used as a negative control and anti-SARS-CoV-1 mouse antibody 240CD was used as a positive control (BEI Resources). ELISAs were performed in technical duplicates with at least two biological duplicates. FIG. 18E shows ELISA binding data to independent preparations of MERS-CoV S protein. An influenza HA-specific mAb 3502-1707 was used as a negative control along with positive control antibodies 1F8 (expressed and purified recombinantly) and MERS S1 mAb and MERS S2 mAbs (Sino Biological).

The sequences listed in FIG. 18C are AREVNYYSAFDD (SEQ ID NO: 121); AAGPTGYDLLTGQYFPYFNY (SEQ ID NO: 114); ARDRSATYYGPFDY (SEQ ID NO: 117); AKDLLSHSGTYSAGSTFDY (SEQ ID NO: 123); ARLDYSKQT (SEQ ID NO: 111); ARRPQYLLLSMTTGRRHHDFVMDV (SEQ ID NO: 120); VKEDTPLVFDS (SEQ ID NO: 122); ALGRKDYGDYYR (SEQ ID NO: 110); ASLPTYGSGRWGIDS (SEQ ID NO: 116); AGFLPVYNNGWSYFDS (SEQ ID NO: 118); VKMRTAVVGVTPL (SEQ ID NO: 119); ARDRVERTGNVGFGYYAMDV (SEQ ID NO: 109); ARVTIVSSFTNRFDP (SEQ ID NO: 112); VRGRTY (SEQ ID NO: 115); ARDFDLVVPSATYPPFYYHGMDV (SEQ ID NO: 113); QQYNFWWT (SEQ ID NO: 175); QSYHGSDVV (SEQ ID NO: 168); NSRDNSGNHPVI (SEQ ID NO: 171); QESYSTNT (SEQ ID NO: 177); MQALQTPLT (SEQ ID NO: 165); QQYYNTPRT (SEQ ID NO: 174); QQYDSYST (SEQ ID NO: 176); QQYAPSPPWYI (SEQ ID NO: 164); QQGNSFPLT (SEQ ID NO: 170); AVWDDSLNGPV (SEQ ID NO: 172); LQDYNYLFS (SEQ ID NO: 173); QQTYSSPSYT (SEQ ID NO: 163); QHRVT (SEQ ID NO: 166); QQYNRWLWT (SEQ ID NO: 169); and NSRDSSGDQTFYV (SEQ ID NO: 167).

FIG. 19A show that cross-reactive antibodies were tested for binding to SARS-CoV-2 S1 domain, SARS-CoV-2 S1 domain D614G, SARS-CoV-2 S2 domain, and SARS-CoV-2 S (HexaPro). Anti-HIV antibody VRC01 is shown as a negative control and anti-SARS-CoV-1 antibody 240CD is shown as positive control.) FIG. 19B shows that S1-directed antibodies 46472-6 and 46472-12 were tested for binding against SARS-CoV-2 RBD, SARS-CoV-1 RBD, SARS-CoV-2 NTD, and SARS-CoV-2 S (HexaPro). (Anti-HIV antibody VRC01 is shown as a negative control and anti-SARS-CoV-1 antibody 240CD is shown as positive control.) FIG. 19C shows that 46472-12 was tested for its ability to block ACE2 binding to SARS-CoV-2 S. Signal shown is anti-Flag tag detection of an ACE2-Flag tag protein construct. FIG. 19D shows that 46472-6 and 46472-12 were tested for binding to SARS-CoV-2 S (HexaPro) mutants, N165A and N709A by ELISA. FIG. 19E shows that mannose competition binding assays were performed to see if cross-reactive antibody binding to SARS-CoV-2 S can be modulated by mannose.

FIG. 20A shows that antibodies were tested for neutralization in a SARS-CoV-1 and SARS-CoV-2 nano-luciferase neutralization assay. FIG. 20B shows that antibodies were tested for neutralization in a SARS-CoV-2 RTCA assay. FIG. 20C shows that cross-reactive coronavirus antibodies were tested for ability to mediate antibody-dependent cellular phagocytosis against SARS-CoV-2 S. FIG. 20D shows that cross-reactive coronavirus antibodies were tested for ability to mediate antibody-dependent cellular trogocytosis against SARS-CoV-2 coated on cells. FIG. 20E shows that Cross-reactive coronavirus antibodies were tested for ability to mediate antibody-dependent cellular trogocytosis against transfected cells displaying SARS-CoV-2 S WT or SARS-CoV-2 S D614G. FIG. 20F shows that cross-reactive coronavirus antibodies were tested for ability to mediate antibody-dependent cellular phagocytosis against SARS-CoV-1 S. FIG. 20G shows that cross-reactive coronavirus antibodies were tested for ability to mediate antibody-dependent complement deposition against SARS-CoV-2 S.

FIG. 21A shows for each antibody treatment group for the experiment utilizing 1×10³ PFU of SARS-CoV-2 MA, a table showing the number of animals to survive per group, per day is shown. Body weights of each mouse in the four treatment groups were measured daily. FIG. 21B shows RT-qPCR quantification of lung viral burden. FIG. 21C shows for each antibody treatment group for the experiment utilizing 1×10⁴ PFU of SARS-CoV-2 MA, a table showing the number of animals to survive per group, per day is shown (survival curves shown in FIG. 17C). 2/5, 4/5, 3/5, and 2/5 mice survived to day 4 for antibodies 46472-4, 46472-12, CR3022 and isotype control DENV-2D22 respectively. Body weights of each mouse in the four treatment groups in both experiments were measured daily. FIG. 21D shows RT-qPCR quantification of lung viral burden.

### DETAILED DESCRIPTION

Therefore, in some aspects, disclosed herein are recombinant antibodies that specifically bind a viral protein of a coronavirus and uses thereof for treating, preventing, inhibiting, reducing, and detecting coronavirus infection, wherein the coronavirus is SARS-CoV-2.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

The term “about” as used herein when referring to a measurable value such as an amount, a percentage, and the like, is meant to encompass variations of ±20%, ±10%, ±5%, or ±1% from the measurable value.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

As used herein, the term “antigen” refers to a molecule that is capable of binding to an antibody. In some embodiments, the antigen stimulates an immune response such as by production of antibodies specific for the antigen.

In the present invention, “specific for” and “specificity” means a condition where one of the molecules is involved in selective binding. Accordingly, an antibody that is specific for one antigen selectively binds that antigen and not other antigens.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof. The antibodies can be tested for their desired activity using the in vitro assays described herein, or by analogous methods, after which their in vivo therapeutic and/or prophylactic activities are tested according to known clinical testing methods. Native antibodies are usually heterotetrameric glycoproteins of about 150,000 daltons, composed of two identical light (L) chains and two identical heavy (H) chains. Each heavy chain has at one end a variable domain (VH) followed by a number of constant domains. Each light chain has a variable domain at one end (VL) and a constant domain at its other end. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

Each antibody molecule is made up of the protein products of two genes: heavy-chain gene and light-chain gene. The heavy-chain gene is constructed through somatic recombination of V, D, and J gene segments. In human, there are 51 VH, 27 DH, 6 RI, 9 CH gene segments on human chromosome 14. The light-chain gene is constructed through somatic recombination of V and J gene segments. There are 40 Vκ, 31 Vλ, 5 Jκ, 4 Jλ× gene segments on human chromosome 14 (80 VJ). The heavy-chain constant domains that correspond to the different classes of immunoglobulins are called α, δ, ε, γ, and μ, respectively. The “light chains” of antibodies from any vertebrate species can be assigned to one of two clearly distinct types, called kappa (κ) and lambda (λ), based on the amino acid sequences of their constant domains.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or antigen binding fragment thereof” or “antibody or fragments thereof” encompasses chimeric antibodies and hybrid antibodies, with dual or multiple antigen or epitope specificities, and fragments, such as F(ab′)2, Fab′, Fab, Fv, sFv, scFv, nanoantibody and the like, including hybrid fragments. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

The terms “antigen binding site”, “binding site” and “binding domain” refer to the specific elements, parts or amino acid residues of a polypeptide, such as an antibody, that bind the antigenic determinant or epitope.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations, κ and λ light chains refer to the two major antibody light chain isotypes.

The term “CDR” as used herein refers to the “complementarity determining regions” of the antibody which consist of the antigen binding loops. (Kabat E. A. et al., (1991) Sequences of proteins of immunological interest. NIH Publication 91-3242). Each of the two variable domains of an antibody Fv fragment contain, for example, three CDRs.

The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen-binding regions. The amino acid sequence boundaries of a CDR can be determined by one of skill in the art using any of a number of known numbering schemes, including those described by Kabat et al., supra (“Kabat” numbering scheme): Al-Lazikani et al., 1997. J. Mol. Biol., 273:927-948 (“Chothia” numbering scheme); MacCallum et al., 1996, J. Mol. Biol, 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme); each of which is incorporated by reference in its entirety.

“Effective amount” encompasses, without limitation, an amount that can ameliorate, reverse, mitigate, prevent, or diagnose a symptom or sign of a medical condition or disorder. Unless dictated otherwise, explicitly or by context, an “effective amount” is not limited to a minimal amount sufficient to ameliorate a condition. The severity of a disease or disorder, as well as the ability of a treatment to prevent, treat, or mitigate, the disease or disorder can be measured, without implying any limitation, by a biomarker or by a clinical parameter. In some embodiments, the term “effective amount of a recombinant antibody” refers to an amount of a recombinant antibody sufficient to prevent, treat, or mitigate a coronavirus infection (e.g., SARS-CoV-2 infection).

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the functional fragment must possess a bioactive property, such as binding to a coronavirus antigen (e.g., SARS-CoV-2 antigen), and/or ameliorating the viral infection.

The term “identity” or “homology” shall be construed to mean the percentage of nucleotide bases or amino acid residues in the candidate sequence that are identical with the bases or residues of a corresponding sequence to which it is compared, after aligning the sequences and introducing gaps, if necessary to achieve the maximum percent identity for the entire sequence, and not considering any conservative substitutions as part of the sequence identity. A polynucleotide or polynucleotide region (or a polypeptide or polypeptide region) that has a certain percentage (for example, 80%, 85%, 90%, or 95%) of “sequence identity” to another sequence means that, when aligned, that percentage of bases (or amino acids) are the same in comparing the two sequences. This alignment and the percent homology or sequence identity can be determined using software programs known in the art. Such alignment can be provided using, for instance, the method of Needleman et al. (1970) J. Mol. Biol. 48: 443-453, implemented conveniently by computer programs such as the Align program (DNAstar, Inc.).

The term “increased” or “increase” as used herein generally means an increase by a statically significant amount; for example, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the terms “nanobody”, “V_{H}H”, “V_{H}H antibody fragment” and “single domain antibody” are used indifferently and designate a variable domain of a single heavy chain of an antibody of the type found in Camelidae, which are without any light chains, such as those derived from Camelids as described in PCT Publication No. WO 94/04678, which is incorporated by reference in its entirety.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

“Nucleotide,” “nucleoside,” “nucleotide residue,” and “nucleoside residue,” as used herein, can mean a deoxyribonucleotide, ribonucleotide residue, or another similar nucleoside analogue. A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

The method and the system disclosed here including the use of primers, which are capable of interacting with the disclosed nucleic acids, such as the antigen barcode as disclosed herein. In certain embodiments the primers are used to support DNA amplification reactions. Typically, the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically, the disclosed primers hybridize with the disclosed nucleic acids or region of the nucleic acids or they hybridize with the complement of the nucleic acids or complement of a region of the nucleic acids.

The term “amplification” refers to the production of one or more copies of a genetic fragment or target sequence, specifically the “amplicon”. As it refers to the product of an amplification reaction, amplicon is used interchangeably with common laboratory terms, such as “PCR product.”

The term “polypeptide” refers to a compound made up of a single chain of D- or L-amino acids or a mixture of D- and L-amino acids joined by peptide bonds.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA.

An “expression cassette” refers to a DNA coding sequence or segment of DNA that code for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct”.

Expression vectors comprise the expression cassette and additionally usually comprise an origin for autonomous replication in the host cells or a genome integration site, one or more selectable markers (e.g. an amino acid synthesis gene or a gene conferring resistance to antibiotics such as zeocin, kanamycin, G418 or hygromycin), a number of restriction enzyme cleavage sites, a suitable promoter sequence and a transcription terminator, which components are operably linked together. The term “vector” as used herein includes autonomously replicating nucleotide sequences as well as genome integrating nucleotide sequences. A common type of vector is a “plasmid”, which generally is a self-contained molecule of double-stranded DNA that can readily accept additional (foreign) DNA and which can readily be introduced into a suitable host cell. A plasmid vector often contains coding DNA and promoter DNA and has one or more restriction sites suitable for inserting foreign DNA. Specifically, the term “vector” or “plasmid” refers to a vehicle by which a DNA or RNA sequence (e.g. a foreign gene) can be introduced into a host cell, so as to transform the host and promote expression (e.g. transcription and translation) of the introduced sequence.

The term “host cell” as used herein shall refer to primary subject cells trans-formed to produce a particular recombinant protein, such as an antibody as described herein, and any progeny thereof. It should be understood that not all progeny are exactly identical to the parental cell (due to deliberate or inadvertent mutations or differences in environment), however, such altered progeny are included in these terms, so long as the progeny retain the same functionality as that of the originally transformed cell. The term “host cell line” refers to a cell line of host cells as used for expressing a recombinant gene to produce recombinant polypeptides such as recombinant antibodies. The term “cell line” as used herein refers to an established clone of a particular cell type that has acquired the ability to proliferate over a prolonged period of time. Such host cell or host cell line may be maintained in cell culture and/or cultivated to produce a recombinant polypeptide.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “specificity” refers to the number of different types of antigens or antigenic determinants to which a particular antigen-binding molecule (such as the recombinant antibody of the invention) can bind. As used herein, the term “specifically binds,” as used herein with respect to a recombinant antibody refers to the recombinant antibody's preferential binding to one or more epitopes as compared with other epitopes. Specific binding can depend upon binding affinity and the stringency of the conditions under which the binding is conducted. In one example, an antibody specifically binds an epitope when there is high affinity binding under stringent conditions.

It should be understood that the specificity of an antigen-binding molecule (e.g., the recombinant antibodies of the present invention) can be determined based on affinity and/or avidity. The affinity, represented by the equilibrium constant for the dissociation of an antigen with an antigen-binding molecule (K_{D}), is a measure for the binding strength between an antigenic determinant and an antigen-binding site on the antigen-binding molecule: the lesser the value of the K_{D}, the stronger the binding strength between an antigenic determinant and the antigen-binding molecule (alternatively, the affinity can also be expressed as the affinity constant (KA), which is 1/K_{D}). As will be clear to the skilled person (for example on the basis of the further disclosure herein), affinity can be determined in a manner known per se, depending on the specific antigen of interest. Avidity is the measure of the strength of binding between an antigen-binding molecule (such as the recombinant antibodies of the present invention) and the pertinent antigen. Avidity is related to both the affinity between an antigenic determinant and its antigen binding site on the antigen-binding molecule and the number of pertinent binding sites present on the antigen-binding molecule. Typically, antigen-binding proteins (such as the recombinant antibodies of the invention) will bind to their antigen with a dissociation constant (K_{D}) of 10⁻⁵ to 10⁻¹² moles/liter or less, and preferably 10⁻⁷ to 10⁻¹² moles/liter or less, and more preferably 10⁻⁸ to 10⁻¹² moles/liter.

“Therapeutically effective amount” refers to the amount of a composition such as recombinant antibody that will elicit the biological or medical response of a tissue, system, animal, or human that is being sought by the researcher, veterinarian, medical doctor or other clinician over a generalized period of time. In some embodiments, a desired response is reduction of coronaviral titers in a subject. In some embodiments, the desired response is mitigation of coronavirus infection and/or related symptoms. In some instances, a desired biological or medical response is achieved following administration of multiple dosages of the composition to the subject over a period of days, weeks, or years. The therapeutically effective amount will vary depending on the composition, the disorder or conditions and its severity, the route of administration, time of administration, rate of excretion, drug combination, judgment of the treating physician, dosage form, and the age, weight, general health, sex and/or diet of the subject to be treated. The therapeutically effective amount of recombinant antibodies as described herein can be determined by one of ordinary skill in the art.

A therapeutically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a biological marker, such as decreased viral titers, decreased viral RNA levels, increase in CD4 T lymphocyte counts, and/or prolonged survival of a subject. It will be understood, that the total daily usage of the compositions and formulations as disclosed herein will be decided by the attending physician within the scope of sound medical judgment. The exact amount required will vary depending on factors such as the type of disease being treated.

The terms “treat,” “treating,” “treatment,” and grammatical variations thereof as used herein, include partially or completely delaying, alleviating, mitigating or reducing the intensity of one or more attendant symptoms. Treatments according to the invention may be applied preventively, prophylactically, palliatively or remedially. Prophylactic treatments are administered to a subject prior to onset (e.g., before obvious signs of an infection), during early onset (e.g., upon initial signs and symptoms of an infection), after an established development of an infection, or during chronic infection. Prophylactic administration can occur for several minutes to months prior to the manifestation of an infection.

As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

### Antibodies and Compositions

In some aspects, disclosed herein is a recombinant antibody, said antibody comprising a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 109-126; and
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 163-180.

In some aspects, disclosed herein is a recombinant antibody, said antibody comprising a light chain variable region (VL) that comprises a light chain complementarity determining region (CDRL)1, CDRL2, and CDRL3 or a heavy chain variable region (VH) that comprises a heavy chain complementarity determining region (CDRH)1, CDRH2, and CDRH3, wherein
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 109-126; and
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 163-180.

In some embodiments, the CDRL3 comprises at least one amino acid substitution when compared to SEQ ID NOs: 163-180. In some embodiments, the CDRL3 comprises at least 1, 2, 3, 4, 5, or 6 substitutions when compared to SEQ ID NO: 163. In some embodiments, the CDRL3 comprises at least 1, 2, 3, 4, 5, or 6 substitutions when compared to SEQ ID NO: 164. In some embodiments, the CDRL3 comprises at least 1, 2, 3, 4, 5, or 6 substitutions when compared to SEQ ID NO: 165. In some embodiments, the CDRL3 comprises at least 1, 2, 3, 4, 5, or 6 substitutions when compared to SEQ ID NO: 166. In some embodiments, the CDRL3 comprises at least 1, 2, 3, 4, 5, or 6 substitutions when compared to SEQ ID NO: 167. In some embodiments, the CDRL3 comprises at least 1, 2, 3, 4, 5, or 6 substitutions when compared to SEQ ID NO: 168. In some embodiments, the CDRL3 comprises at least 1, 2, 3, 4, 5, or 6 substitutions when compared to SEQ ID NO: 169. In some embodiments, the CDRL3 comprises at least 1, 2, 3, 4, 5, or 6 substitutions when compared to SEQ ID NO: 170. In some embodiments, the CDRL3 comprises at least 1, 2, 3, 4, 5, or 6 substitutions when compared to SEQ ID NO: 171. In some embodiments, the CDRL3 comprises at least 1, 2, 3, 4, 5, or 6 substitutions when compared to SEQ ID NO: 172. In some embodiments, the CDRL3 comprises at least 1, 2, 3, 4, 5, or 6 substitutions when compared to SEQ ID NO: 173. In some embodiments, the CDRL3 comprises at least 1, 2, 3, 4, 5, or 6 substitutions when compared to SEQ ID NO: 174. In some embodiments, the CDRL3 comprises at least 1, 2, 3, 4, 5, or 6 substitutions when compared to SEQ ID NO: 175. In some embodiments, the CDRL3 comprises at least 1, 2, 3, 4, 5, or 6 substitutions when compared to SEQ ID NO: 176. In some embodiments, the CDRL3 comprises at least 1, 2, 3, 4, 5, or 6 substitutions when compared to SEQ ID NO: 177. In some embodiments, the CDRL3 comprises at least 1, 2, 3, 4, 5, or 6 substitutions when compared to SEQ ID NO: 178. In some embodiments, the CDRL3 comprises at least 1, 2, 3, 4, 5, or 6 substitutions when compared to SEQ ID NO: 179. In some embodiments, the CDRL3 comprises at least 1, 2, 3, 4, 5, or 6 substitutions when compared to SEQ ID NO: 180.

In some embodiments, the CDRH3 comprises at least one amino acid substitution when compared to SEQ ID NOs: 109-126. In some embodiments, the CDRH3 comprises at least 1, 2, 3, 4, 5, or 6 substitutions when compared to SEQ ID NO: 109. In some embodiments, the CDRH3 comprises at least 1, 2, 3, 4, 5, or 6 substitutions when compared to SEQ ID NO: 110. In some embodiments, the CDRH3 comprises at least 1, 2, 3, 4, 5, or 6 substitutions when compared to SEQ ID NO: 111. In some embodiments, the CDRH3 comprises at least 1, 2, 3, 4, 5, or 6 substitutions when compared to SEQ ID NO: 112. In some embodiments, the CDRH3 comprises at least 1, 2, 3, 4, 5, or 6 substitutions when compared to SEQ ID NO: 113. In some embodiments, the CDRH3 comprises at least 1, 2, 3, 4, 5, or 6 substitutions when compared to SEQ ID NO: 114. In some embodiments, the CDRH3 comprises at least 1, 2, 3, 4, 5, or 6 substitutions when compared to SEQ ID NO: 115. In some embodiments, the CDRH3 comprises at least 1, 2, 3, 4, 5, or 6 substitutions when compared to SEQ ID NO: 116. In some embodiments, the CDRH3 comprises at least 1, 2, 3, 4, 5, or 6 substitutions when compared to SEQ ID NO: 117. In some embodiments, the CDRH3 comprises at least 1, 2, 3, 4, 5, or 6 substitutions when compared to SEQ ID NO: 118. In some embodiments, the CDRH3 comprises at least 1, 2, 3, 4, 5, or 6 substitutions when compared to SEQ ID NO: 119. In some embodiments, the CDRH3 comprises at least 1, 2, 3, 4, 5, or 6 substitutions when compared to SEQ ID NO: 120. In some embodiments, the CDRH3 comprises at least 1, 2, 3, 4, 5, or 6 substitutions when compared to SEQ ID NO: 121. In some embodiments, the CDRH3 comprises at least 1, 2, 3, 4, 5, or 6 substitutions when compared to SEQ ID NO: 122. In some embodiments, the CDRH3 comprises at least 1, 2, 3, 4, 5, or 6 substitutions when compared to SEQ ID NO: 123. In some embodiments, the CDRH3 comprises at least 1, 2, 3, 4, 5, or 6 substitutions when compared to SEQ ID NO: 124. In some embodiments, the CDRH3 comprises at least 1, 2, 3, 4, 5, or 6 substitutions when compared to SEQ ID NO: 125. In some embodiments, the CDRH3 comprises at least 1, 2, 3, 4, 5, or 6 substitutions when compared to SEQ ID NO: 126.

In some embodiments, the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 73-90; and CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 127-144.

In some embodiments, the CDRH1 comprises at least one amino acid substitution when compared to SEQ ID NOs: 73-90. In some embodiments, the CDRH1 comprises at least 1, 2, 3, 4, 5, or 6 substitutions when compared to SEQ ID NOs: 73-90.

In some embodiments, the CDRL1 comprises at least one amino acid substitution when compared to SEQ ID NOs: 127-144. In some embodiments, the CDRL1 comprises at least 1, 2, 3, 4, 5, or 6 substitutions when compared to SEQ ID NOs: 127-144.

In some embodiments, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 91-108; and CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 145-162.

In some embodiments, the CDRH2 comprises at least one amino acid substitution when compared to SEQ ID NOs: 91-108. In some embodiments, the CDRH2 comprises at least 1, 2, 3, 4, 5, or 6 substitutions when compared to SEQ ID NOs: 91-108.

In some embodiments, the CDRL2 comprises at least one amino acid substitution when compared to SEQ ID NOs: 145-162. In some embodiments, the CDRL2 comprises at least 1, 2, 3, 4, 5, or 6 substitutions when compared to SEQ ID NOs: 145-162.

In some embodiments, VH comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 37-54. In some embodiments, VH comprises an amino acid sequence selected from the group consisting of SEQ ID NOs: 37-54.

In some embodiments, VL comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 55-72. In some embodiments, VL comprises an amino acid sequence selected from the group consisting of SEQ ID NOs: 55-72.

In some embodiments, a CDR sequence (for example CDRL1, CDRL2, CDRL3, CDRH1, CDRH2, or CDRH3) comprises one amino acid mutation, two amino acid mutations, three amino acid mutations, four amino acid mutations, five amino acid mutations, etc. when compared to a CDR sequence as disclosed herein.

In some embodiments, the recombinant antibody is a monoclonal antibody. In some embodiments, the recombinant antibody is an isolated antibody. In some embodiments, the recombinant antibody is a non-naturally occurring antibody. In some embodiments, the recombinant antibody is an antibody or antigen binding fragment thereof. In some embodiments, combinations of antibodies or antigen binding fragments thereof disclosed herein are used for treating coronavirus infection.

In some embodiments, combinations of antibodies or antigen binding fragments thereof disclosed herein are used for treating SARS-CoV-2 infection.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:73,
CDRH2 is SEQ ID NO:91,
CDRH3 is SEQ ID NO:109,
CDRL1 is SEQ ID NO:127,
CDRL2 is SEQ ID NO:145, and
CDRL3 is SEQ ID NO:163.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:74,
CDRH2 is SEQ ID NO:92,
CDRH3 is SEQ ID NO:110,
CDRL1 is SEQ ID NO:128,
CDRL2 is SEQ ID NO:146, and
CDRL3 is SEQ ID NO:164.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:75,
CDRH2 is SEQ ID NO:93,
CDRH3 is SEQ ID NO:111,
CDRL1 is SEQ ID NO:129,
CDRL2 is SEQ ID NO:147, and
CDRL3 is SEQ ID NO:165.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:76,
CDRH2 is SEQ ID NO:94,
CDRH3 is SEQ ID NO:112,
CDRL1 is SEQ ID NO:130,
CDRL2 is SEQ ID NO:148, and
CDRL3 is SEQ ID NO:166.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:77,
CDRH2 is SEQ ID NO:95,
CDRH3 is SEQ ID NO:113,
CDRL1 is SEQ ID NO:131,
CDRL2 is SEQ ID NO:149, and
CDRL3 is SEQ ID NO:167.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:78,
CDRH2 is SEQ ID NO:96,
CDRH3 is SEQ ID NO:114,
CDRL1 is SEQ ID NO:132,
CDRL2 is SEQ ID NO:150, and
CDRL3 is SEQ ID NO:168.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:79,
CDRH2 is SEQ ID NO:97,
CDRH3 is SEQ ID NO:115,
CDRL1 is SEQ ID NO:133,
CDRL2 is SEQ ID NO:151, and
CDRL3 is SEQ ID NO:169.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:80,
CDRH2 is SEQ ID NO:98,
CDRH3 is SEQ ID NO:116,
CDRL1 is SEQ ID NO:134,
CDRL2 is SEQ ID NO:152, and
CDRL3 is SEQ ID NO:170.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:81,
CDRH2 is SEQ ID NO:99,
CDRH3 is SEQ ID NO:117,
CDRL1 is SEQ ID NO:135,
CDRL2 is SEQ ID NO:153, and
CDRL3 is SEQ ID NO:171.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:82,
CDRH2 is SEQ ID NO:100,
CDRH3 is SEQ ID NO:118,
CDRL1 is SEQ ID NO:136,
CDRL2 is SEQ ID NO:154, and
CDRL3 is SEQ ID NO:172.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:83,
CDRH2 is SEQ ID NO:101,
CDRH3 is SEQ ID NO:119,
CDRL1 is SEQ ID NO:137,
CDRL2 is SEQ ID NO:155, and
CDRL3 is SEQ ID NO:173.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:84,
CDRH2 is SEQ ID NO:102,
CDRH3 is SEQ ID NO:120,
CDRL1 is SEQ ID NO:138,
CDRL2 is SEQ ID NO:156, and
CDRL3 is SEQ ID NO:174.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:85,
CDRH2 is SEQ ID NO:103,
CDRH3 is SEQ ID NO:121,
CDRL1 is SEQ ID NO:139,
CDRL2 is SEQ ID NO:157, and
CDRL3 is SEQ ID NO:175.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:86,
CDRH2 is SEQ ID NO:104,
CDRH3 is SEQ ID NO:122,
CDRL1 is SEQ ID NO:140,
CDRL2 is SEQ ID NO:158, and
CDRL3 is SEQ ID NO:176.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:87,
CDRH2 is SEQ ID NO:105,
CDRH3 is SEQ ID NO:123,
CDRL1 is SEQ ID NO:141,
CDRL2 is SEQ ID NO:159, and
CDRL3 is SEQ ID NO:177.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:88,
CDRH2 is SEQ ID NO:106,
CDRH3 is SEQ ID NO:124,
CDRL1 is SEQ ID NO:142,
CDRL2 is SEQ ID NO:160, and
CDRL3 is SEQ ID NO:178.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:89,
CDRH2 is SEQ ID NO:107,
CDRH3 is SEQ ID NO:125,
CDRL1 is SEQ ID NO:143,
CDRL2 is SEQ ID NO:161, and
CDRL3 is SEQ ID NO:179.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:90,
CDRH2 is SEQ ID NO:108,
CDRH3 is SEQ ID NO:126,
CDRL1 is SEQ ID NO:144,
CDRL2 is SEQ ID NO:162, and
CDRL3 is SEQ ID NO:180.

In some embodiments, the antibody or antigen binding fragment thereof comprises a light chain variable region (VL) and/or a heavy chain variable region (VH), wherein:
VH is SEQ ID NO: 37, and
VL is SEQ ID NO: 55.

In some embodiments, the antibody or antigen binding fragment thereof comprises a light chain variable region (VL) and/or a heavy chain variable region (VH), wherein:
VH is SEQ ID NO: 38, and
VL is SEQ ID NO: 56.

In some embodiments, the antibody or antigen binding fragment thereof comprises a light chain variable region (VL) and/or a heavy chain variable region (VH), wherein:
VH is SEQ ID NO: 39, and
VL is SEQ ID NO: 57.

In some embodiments, the antibody or antigen binding fragment thereof comprises a light chain variable region (VL) and/or a heavy chain variable region (VH), wherein:
VH is SEQ ID NO: 40, and
VL is SEQ ID NO: 58.

In some embodiments, the antibody or antigen binding fragment thereof comprises a light chain variable region (VL) and/or a heavy chain variable region (VH), wherein:
VH is SEQ ID NO: 41, and
VL is SEQ ID NO: 59.

In some embodiments, the antibody or antigen binding fragment thereof comprises a light chain variable region (VL) and/or a heavy chain variable region (VH), wherein:
VH is SEQ ID NO: 42, and
VL is SEQ ID NO: 60.

In some embodiments, the antibody or antigen binding fragment thereof comprises a light chain variable region (VL) and/or a heavy chain variable region (VH), wherein:
VH is SEQ ID NO: 43, and
VL is SEQ ID NO: 61.

In some embodiments, the antibody or antigen binding fragment thereof comprises a light chain variable region (VL) and/or a heavy chain variable region (VH), wherein:
VH is SEQ ID NO: 44, and
VL is SEQ ID NO: 62.

In some embodiments, the antibody or antigen binding fragment thereof comprises a light chain variable region (VL) and/or a heavy chain variable region (VH), wherein:
VH is SEQ ID NO: 45, and
VL is SEQ ID NO: 63.

In some embodiments, the antibody or antigen binding fragment thereof comprises a light chain variable region (VL) and/or a heavy chain variable region (VH), wherein:
VH is SEQ ID NO: 46, and
VL is SEQ ID NO: 64.

In some embodiments, the antibody or antigen binding fragment thereof comprises a light chain variable region (VL) and/or a heavy chain variable region (VH), wherein:
VH is SEQ ID NO: 47, and
VL is SEQ ID NO: 65.

In some embodiments, the antibody or antigen binding fragment thereof comprises a light chain variable region (VL) and/or a heavy chain variable region (VH), wherein:
VH is SEQ ID NO: 48, and
VL is SEQ ID NO: 66.

In some embodiments, the antibody or antigen binding fragment thereof comprises a light chain variable region (VL) and/or a heavy chain variable region (VH), wherein:
VH is SEQ ID NO: 49, and
VL is SEQ ID NO: 67.

In some embodiments, the antibody or antigen binding fragment thereof comprises a light chain variable region (VL) and/or a heavy chain variable region (VH), wherein:
VH is SEQ ID NO: 50, and
VL is SEQ ID NO: 68.

In some embodiments, the antibody or antigen binding fragment thereof comprises a light chain variable region (VL) and/or a heavy chain variable region (VH), wherein:
VH is SEQ ID NO: 51, and
VL is SEQ ID NO: 69.

In some embodiments, the antibody or antigen binding fragment thereof comprises a light chain variable region (VL) and/or a heavy chain variable region (VH), wherein:
VH is SEQ ID NO: 52, and
VL is SEQ ID NO: 70.

In some embodiments, the antibody or antigen binding fragment thereof comprises a light chain variable region (VL) and/or a heavy chain variable region (VH), wherein:
VH is SEQ ID NO: 53, and
VL is SEQ ID NO: 71.

In some embodiments, the antibody or antigen binding fragment thereof comprises a light chain variable region (VL) and/or a heavy chain variable region (VH), wherein:
VH is SEQ ID NO: 54, and
VL is SEQ ID NO: 72.

In some embodiments, the recombinant antibody or antigen binding fragment thereof of any preceding aspect comprises a VH comprising an amino acid sequence selected from SEQ ID NOs: 5437-8064.

In some embodiments, the recombinant antibody or antigen binding fragment thereof of any preceding aspect comprises a VL comprising an amino acid sequence selected from SEQ ID NOs: 8065-10692.

In some embodiments, the recombinant antibody or antigen binding fragment thereof of any preceding aspect comprises a CDRH1 comprising an amino acid sequence selected from SEQ ID NOs: 10693-13311.

In some embodiments, the recombinant antibody or antigen binding fragment thereof of any preceding aspect comprises a CDRH2 comprising an amino acid sequence selected from SEQ ID NOs: 13312-15934.

In some embodiments, the recombinant antibody or antigen binding fragment thereof of any preceding aspect comprises a CDRH3 comprising an amino acid sequence selected from SEQ ID NOs: 15935-18562.

In some embodiments, the recombinant antibody or antigen binding fragment thereof of any preceding aspect comprises a CDRL1 comprising an amino acid sequence selected from SEQ ID NOs: 18563-21190.

In some embodiments, the recombinant antibody or antigen binding fragment thereof of any preceding aspect comprises a CDRL2 comprising an amino acid sequence selected from SEQ ID NOs: 21191-23818.

In some embodiments, the recombinant antibody or antigen binding fragment thereof of any preceding aspect comprises a CDRL3 comprising an amino acid sequence selected from SEQ ID NOs: 23819-26446.

### Methods

Disclosed herein are methods for preventing, treating, inhibiting, reducing, or detecting coronavirus infection.

In some aspects, disclosed herein is a method of producing a recombinant antibody comprising cultivating or maintaining the host cell of any preceding aspect under conditions to produce a recombinant antibody as described herein.

In some aspects, disclosed herein is a method of treating, preventing, reducing, and/or inhibiting coronavirus infection, comprising administering to a subject a therapeutically effective amount of a recombinant antibody, wherein the recombinant antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 109-126; and
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 163-180.

In some aspects, disclosed herein is a method of treating, preventing, reducing, and/or inhibiting coronavirus infection, comprising administering to a subject a therapeutically effective amount of a recombinant antibody, wherein the recombinant antibody comprises a light chain variable region (VL) that comprises a light chain complementarity determining region (CDRL)1, CDRL2, and CDRL3 or a heavy chain variable region (VH) that comprises a heavy chain complementarity determining region (CDRH)1, CDRH2, and CDRH3, wherein
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 109-126; and
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 163-180.

In some embodiments, the CDRL3 comprises at least one amino acid substitution when compared to SEQ ID NOs: 163-180. In some embodiments, the CDRL3 comprises at least 1, 2, 3, 4, 5, or 6 substitutions when compared to SEQ ID NO: 163. In some embodiments, the CDRL3 comprises at least 1, 2, 3, 4, 5, or 6 substitutions when compared to SEQ ID NO: 164. In some embodiments, the CDRL3 comprises at least 1, 2, 3, 4, 5, or 6 substitutions when compared to SEQ ID NO: 165. In some embodiments, the CDRL3 comprises at least 1, 2, 3, 4, 5, or 6 substitutions when compared to SEQ ID NO: 166. In some embodiments, the CDRL3 comprises at least 1, 2, 3, 4, 5, or 6 substitutions when compared to SEQ ID NO: 167. In some embodiments, the CDRL3 comprises at least 1, 2, 3, 4, 5, or 6 substitutions when compared to SEQ ID NO: 168. In some embodiments, the CDRL3 comprises at least 1, 2, 3, 4, 5, or 6 substitutions when compared to SEQ ID NO: 169. In some embodiments, the CDRL3 comprises at least 1, 2, 3, 4, 5, or 6 substitutions when compared to SEQ ID NO: 170. In some embodiments, the CDRL3 comprises at least 1, 2, 3, 4, 5, or 6 substitutions when compared to SEQ ID NO: 171. In some embodiments, the CDRL3 comprises at least 1, 2, 3, 4, 5, or 6 substitutions when compared to SEQ ID NO: 172. In some embodiments, the CDRL3 comprises at least 1, 2, 3, 4, 5, or 6 substitutions when compared to SEQ ID NO: 173. In some embodiments, the CDRL3 comprises at least 1, 2, 3, 4, 5, or 6 substitutions when compared to SEQ ID NO: 174. In some embodiments, the CDRL3 comprises at least 1, 2, 3, 4, 5, or 6 substitutions when compared to SEQ ID NO: 175. In some embodiments, the CDRL3 comprises at least 1, 2, 3, 4, 5, or 6 substitutions when compared to SEQ ID NO: 176. In some embodiments, the CDRL3 comprises at least 1, 2, 3, 4, 5, or 6 substitutions when compared to SEQ ID NO: 177. In some embodiments, the CDRL3 comprises at least 1, 2, 3, 4, 5, or 6 substitutions when compared to SEQ ID NO: 178. In some embodiments, the CDRL3 comprises at least 1, 2, 3, 4, 5, or 6 substitutions when compared to SEQ ID NO: 179. In some embodiments, the CDRL3 comprises at least 1, 2, 3, 4, 5, or 6 substitutions when compared to SEQ ID NO: 180.

In some embodiments, the CDRH3 comprises at least one amino acid substitution when compared to SEQ ID NOs: 109-126. In some embodiments, the CDRH3 comprises at least 1, 2, 3, 4, 5, or 6 substitutions when compared to SEQ ID NO: 109. In some embodiments, the CDRH3 comprises at least 1, 2, 3, 4, 5, or 6 substitutions when compared to SEQ ID NO: 110. In some embodiments, the CDRH3 comprises at least 1, 2, 3, 4, 5, or 6 substitutions when compared to SEQ ID NO: 111. In some embodiments, the CDRH3 comprises at least 1, 2, 3, 4, 5, or 6 substitutions when compared to SEQ ID NO: 112. In some embodiments, the CDRH3 comprises at least 1, 2, 3, 4, 5, or 6 substitutions when compared to SEQ ID NO: 113. In some embodiments, the CDRH3 comprises at least 1, 2, 3, 4, 5, or 6 substitutions when compared to SEQ ID NO: 114. In some embodiments, the CDRH3 comprises at least 1, 2, 3, 4, 5, or 6 substitutions when compared to SEQ ID NO: 115. In some embodiments, the CDRH3 comprises at least 1, 2, 3, 4, 5, or 6 substitutions when compared to SEQ ID NO: 116. In some embodiments, the CDRH3 comprises at least 1, 2, 3, 4, 5, or 6 substitutions when compared to SEQ ID NO: 117. In some embodiments, the CDRH3 comprises at least 1, 2, 3, 4, 5, or 6 substitutions when compared to SEQ ID NO: 118. In some embodiments, the CDRH3 comprises at least 1, 2, 3, 4, 5, or 6 substitutions when compared to SEQ ID NO: 119. In some embodiments, the CDRH3 comprises at least 1, 2, 3, 4, 5, or 6 substitutions when compared to SEQ ID NO: 120. In some embodiments, the CDRH3 comprises at least 1, 2, 3, 4, 5, or 6 substitutions when compared to SEQ ID NO: 121. In some embodiments, the CDRH3 comprises at least 1, 2, 3, 4, 5, or 6 substitutions when compared to SEQ ID NO: 122. In some embodiments, the CDRH3 comprises at least 1, 2, 3, 4, 5, or 6 substitutions when compared to SEQ ID NO: 123. In some embodiments, the CDRH3 comprises at least 1, 2, 3, 4, 5, or 6 substitutions when compared to SEQ ID NO: 124. In some embodiments, the CDRH3 comprises at least 1, 2, 3, 4, 5, or 6 substitutions when compared to SEQ ID NO: 125. In some embodiments, the CDRH3 comprises at least 1, 2, 3, 4, 5, or 6 substitutions when compared to SEQ ID NO: 126.

In some embodiments, the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 97-120; and CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 121-144.

In some embodiments, the CDRH1 comprises at least one amino acid substitution when compared to SEQ ID NOs: 73-90. In some embodiments, the CDRH1 comprises at least 1, 2, 3, 4, 5, or 6 substitutions when compared to SEQ ID NOs: 73-90.

In some embodiments, the CDRL1 comprises at least one amino acid substitution when compared to SEQ ID NOs: 127-144. In some embodiments, the CDRL1 comprises at least 1, 2, 3, 4, 5, or 6 substitutions when compared to SEQ ID NOs: 127-144.

In some embodiments, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 91-108; and CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 145-162.

In some embodiments, the CDRH2 comprises at least one amino acid substitution when compared to SEQ ID NOs: 91-108. In some embodiments, the CDRH2 comprises at least 1, 2, 3, 4, 5, or 6 substitutions when compared to SEQ ID NOs: 91-108.

In some embodiments, the CDRL2 comprises at least one amino acid substitution when compared to SEQ ID NOs: 145-162. In some embodiments, the CDRL2 comprises at least 1, 2, 3, 4, 5, or 6 substitutions when compared to SEQ ID NOs: 145-162.

In some embodiments, VH comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 37-54. In some embodiments, VH comprises an amino acid sequence selected from the group consisting of SEQ ID NOs: 37-54.

In some embodiments, VL comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 55-72. In some embodiments, VL comprises an amino acid sequence selected from the group consisting of SEQ ID NOs: 55-72.

In some embodiments, a CDR sequence (for example CDRL1, CDRL2, CDRL3, CDRH1, CDRH2, or CDRH3) comprises one amino acid mutation, two amino acid mutations, three amino acid mutations, four amino acid mutations, five amino acid mutations, etc. when compared to a CDR sequence as disclosed herein.

In some embodiments, the recombinant antibody is a monoclonal antibody. In some embodiments, the recombinant antibody is an isolated antibody. In some embodiments, the recombinant antibody is an antibody or antigen binding fragment thereof. In some embodiments, combinations of antibodies or antigen binding fragments thereof disclosed herein are used for treating coronavirus infection.

In some embodiments, combinations of antibodies or antigen binding fragments thereof disclosed herein are used for treating SARS-CoV-2 infection.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:73,
CDRH2 is SEQ ID NO:91,
CDRH3 is SEQ ID NO:109,
CDRL1 is SEQ ID NO:127,
CDRL2 is SEQ ID NO:145, and
CDRL3 is SEQ ID NO:163.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:74,
CDRH2 is SEQ ID NO:92,
CDRH3 is SEQ ID NO:110,
CDRL1 is SEQ ID NO:128,
CDRL2 is SEQ ID NO:146, and
CDRL3 is SEQ ID NO:164.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:75,
CDRH2 is SEQ ID NO:93,
CDRH3 is SEQ ID NO:111,
CDRL1 is SEQ ID NO:129,
CDRL2 is SEQ ID NO:147, and
CDRL3 is SEQ ID NO:165.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:76,
CDRH2 is SEQ ID NO:94,
CDRH3 is SEQ ID NO:112,
CDRL1 is SEQ ID NO:130,
CDRL2 is SEQ ID NO:148, and
CDRL3 is SEQ ID NO:166.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:77,
CDRH2 is SEQ ID NO:95,
CDRH3 is SEQ ID NO:113,
CDRL1 is SEQ ID NO:131,
CDRL2 is SEQ ID NO:149, and
CDRL3 is SEQ ID NO:167.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:78,
CDRH2 is SEQ ID NO:96,
CDRH3 is SEQ ID NO:114,
CDRL1 is SEQ ID NO:132,
CDRL2 is SEQ ID NO:150, and
CDRL3 is SEQ ID NO:168.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:79,
CDRH2 is SEQ ID NO:97,
CDRH3 is SEQ ID NO:115,
CDRL1 is SEQ ID NO:133,
CDRL2 is SEQ ID NO:151, and
CDRL3 is SEQ ID NO:169.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:80,
CDRH2 is SEQ ID NO:98,
CDRH3 is SEQ ID NO:116,
CDRL1 is SEQ ID NO:134,
CDRL2 is SEQ ID NO:152, and
CDRL3 is SEQ ID NO:170.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:81,
CDRH2 is SEQ ID NO:99,
CDRH3 is SEQ ID NO:117,
CDRL1 is SEQ ID NO:135,
CDRL2 is SEQ ID NO:153, and
CDRL3 is SEQ ID NO:171.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:82,
CDRH2 is SEQ ID NO:100,
CDRH3 is SEQ ID NO:118,
CDRL1 is SEQ ID NO:136,
CDRL2 is SEQ ID NO:154, and
CDRL3 is SEQ ID NO:172.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:83,
CDRH2 is SEQ ID NO:101,
CDRH3 is SEQ ID NO:119,
CDRL1 is SEQ ID NO:137,
CDRL2 is SEQ ID NO:155, and
CDRL3 is SEQ ID NO:173.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:84,
CDRH2 is SEQ ID NO:102,
CDRH3 is SEQ ID NO:120,
CDRL1 is SEQ ID NO:138,
CDRL2 is SEQ ID NO:156, and
CDRL3 is SEQ ID NO:174.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:85,
CDRH2 is SEQ ID NO:103,
CDRH3 is SEQ ID NO:121,
CDRL1 is SEQ ID NO:139,
CDRL2 is SEQ ID NO:157, and
CDRL3 is SEQ ID NO:175.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:86,
CDRH2 is SEQ ID NO:104,
CDRH3 is SEQ ID NO:122,
CDRL1 is SEQ ID NO:140,
CDRL2 is SEQ ID NO:158, and
CDRL3 is SEQ ID NO:176.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:87,
CDRH2 is SEQ ID NO:105,
CDRH3 is SEQ ID NO:123,
CDRL1 is SEQ ID NO:141,
CDRL2 is SEQ ID NO:159, and
CDRL3 is SEQ ID NO:177.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:88,
CDRH2 is SEQ ID NO:106,
CDRH3 is SEQ ID NO:124,
CDRL1 is SEQ ID NO:142,
CDRL2 is SEQ ID NO:160, and
CDRL3 is SEQ ID NO:178.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:89,
CDRH2 is SEQ ID NO:107,
CDRH3 is SEQ ID NO:125,
CDRL1 is SEQ ID NO:143,
CDRL2 is SEQ ID NO:161, and
CDRL3 is SEQ ID NO:179.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRH1 is SEQ ID NO:90,
CDRH2 is SEQ ID NO:108,
CDRH3 is SEQ ID NO:126,
CDRL1 is SEQ ID NO:144,
CDRL2 is SEQ ID NO:162, and
CDRL3 is SEQ ID NO:180.

In some embodiments, the antibody or antigen binding fragment thereof comprises a light chain variable region (VL) and/or a heavy chain variable region (VH), wherein:
VH is SEQ ID NO: 37, and
VL is SEQ ID NO: 55.

In some embodiments, the antibody or antigen binding fragment thereof comprises a light chain variable region (VL) and/or a heavy chain variable region (VH), wherein:
VH is SEQ ID NO: 38, and
VL is SEQ ID NO: 56.

In some embodiments, the antibody or antigen binding fragment thereof comprises a light chain variable region (VL) and/or a heavy chain variable region (VH), wherein:
VH is SEQ ID NO: 39, and
VL is SEQ ID NO: 57.

In some embodiments, the antibody or antigen binding fragment thereof comprises a light chain variable region (VL) and/or a heavy chain variable region (VH), wherein:
VH is SEQ ID NO: 40, and
VL is SEQ ID NO: 58.

In some embodiments, the antibody or antigen binding fragment thereof comprises a light chain variable region (VL) and/or a heavy chain variable region (VH), wherein:
VH is SEQ ID NO: 41, and
VL is SEQ ID NO: 59.

In some embodiments, the antibody or antigen binding fragment thereof comprises a light chain variable region (VL) and/or a heavy chain variable region (VH), wherein:
VH is SEQ ID NO: 42, and
VL is SEQ ID NO: 60.

In some embodiments, the antibody or antigen binding fragment thereof comprises a light chain variable region (VL) and/or a heavy chain variable region (VH), wherein:
VH is SEQ ID NO: 43, and
VL is SEQ ID NO: 61.

In some embodiments, the antibody or antigen binding fragment thereof comprises a light chain variable region (VL) and/or a heavy chain variable region (VH), wherein:
VH is SEQ ID NO: 44, and
VL is SEQ ID NO: 62.

In some embodiments, the antibody or antigen binding fragment thereof comprises a light chain variable region (VL) and/or a heavy chain variable region (VH), wherein:
VH is SEQ ID NO: 45, and
VL is SEQ ID NO: 63.

In some embodiments, the antibody or antigen binding fragment thereof comprises a light chain variable region (VL) and/or a heavy chain variable region (VH), wherein:
VH is SEQ ID NO: 46, and
VL is SEQ ID NO: 64.

In some embodiments, the antibody or antigen binding fragment thereof comprises a light chain variable region (VL) and/or a heavy chain variable region (VH), wherein:
VH is SEQ ID NO: 47, and
VL is SEQ ID NO: 65.

In some embodiments, the antibody or antigen binding fragment thereof comprises a light chain variable region (VL) and/or a heavy chain variable region (VH), wherein:
VH is SEQ ID NO: 48, and
VL is SEQ ID NO: 66.

In some embodiments, the antibody or antigen binding fragment thereof comprises a light chain variable region (VL) and/or a heavy chain variable region (VH), wherein:
VH is SEQ ID NO: 49, and
VL is SEQ ID NO: 67.

In some embodiments, the antibody or antigen binding fragment thereof comprises a light chain variable region (VL) and/or a heavy chain variable region (VH), wherein:
VH is SEQ ID NO: 50, and
VL is SEQ ID NO: 68.

In some embodiments, the antibody or antigen binding fragment thereof comprises a light chain variable region (VL) and/or a heavy chain variable region (VH), wherein:
VH is SEQ ID NO: 51, and
VL is SEQ ID NO: 69.

In some embodiments, the antibody or antigen binding fragment thereof comprises a light chain variable region (VL) and/or a heavy chain variable region (VH), wherein:
VH is SEQ ID NO: 52, and
VL is SEQ ID NO: 70.

In some embodiments, the antibody or antigen binding fragment thereof comprises a light chain variable region (VL) and/or a heavy chain variable region (VH), wherein:
VH is SEQ ID NO: 53, and
VL is SEQ ID NO: 71.

In some embodiments, the antibody or antigen binding fragment thereof comprises a light chain variable region (VL) and/or a heavy chain variable region (VH), wherein:
VH is SEQ ID NO: 54, and
VL is SEQ ID NO: 72.

In some embodiments, the recombinant antibody or antigen binding fragment thereof of any preceding aspect comprises a VH comprising an amino acid sequence selected from SEQ ID NOs: 5437-8064.

In some embodiments, the recombinant antibody or antigen binding fragment thereof of any preceding aspect comprises a VL comprising an amino acid sequence selected from SEQ ID NOs: 8065-10692.

In some embodiments, the recombinant antibody or antigen binding fragment thereof of any preceding aspect comprises a CDRH1 comprising an amino acid sequence selected from SEQ ID NOs: 10693-13311.

In some embodiments, the recombinant antibody or antigen binding fragment thereof of any preceding aspect comprises a CDRH2 comprising an amino acid sequence selected from SEQ ID NOs: 13312-15934.

In some embodiments, the recombinant antibody or antigen binding fragment thereof of any preceding aspect comprises a CDRH3 comprising an amino acid sequence selected from SEQ ID NOs: 15935-18562.

In some embodiments, the recombinant antibody or antigen binding fragment thereof of any preceding aspect comprises a CDRL1 comprising an amino acid sequence selected from SEQ ID NOs: 18563-21190.

In some embodiments, the recombinant antibody or antigen binding fragment thereof of any preceding aspect comprises a CDRL2 comprising an amino acid sequence selected from SEQ ID NOs: 21191-23818.

In some embodiments, the recombinant antibody or antigen binding fragment thereof of any preceding aspect comprises a CDRL3 comprising an amino acid sequence selected from SEQ ID NOs: 23819-26446.

In some embodiments, the recombinant antibody binds to at least one coronavirus antigen. In some embodiments, the recombinant antibody binds to at least one SARS-CoV-2 antigen.

In some embodiments, the target protein comprises a viral protein. In some embodiments, the viral protein is a coronavirus protein. Coronaviruses constitute the subfamily Orthocoronavirinae, in the family Coronaviridae, order Nidovirales, and realm Riboviria. They are enveloped viruses with a positive-sense single-stranded RNA genome and a nucleocapsid of helical symmetry. The genome size of coronaviruses ranges from approximately 27 to 34 kilobases. The structure of coronavirus generally consists of the following: spike protein, hemagglutinin-esterease dimer (HE), a membrane glycoprotein (M), an envelope protein (E) a nucleoclapid protein (N) and RNA. The coronavirus family comprises genera including, for example, alphacoronavius (e.g., Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512), betacoronavirus (e.g., SARS-CoV-2, Betacoronavirus 1, Human coronavirus HKU1, Murine coronavirus, Pipistrellus bat coronavirus HKU5, Rousettus bat coronavirus HKU9, Severe acute respiratory syndrome-related coronavirus, Tylonycteris bat coronavirus HKU4, Middle East respiratory syndrome-related coronavirus (MERS), Human coronavirus OC43, Hedgehog coronavirus 1 (EriCoV)), gammacoronavirus (e.g., Beluga whale coronavirus SW1, Infectious bronchitis virus), and deltacoronavirus (e.g., Bulbul coronavirus HKU11, Porcine coronavirus HKU15). In some embodiments, the viral protein is a protein of Severe acute respiratory syndrome-related coronavirus. In some embodiments, the viral protein is a protein of MERS coronavirus.

In some embodiments, the viral protein is a SARS-CoV-2 protein, including, for example, SARS-CoV-2 spike protein, SARS-CoV-2 envelope protein, SARS-CoV-2 membrane protein, or SARS-CoV-2 nucleocapsid protein, or a fragment thereof. In some embodiments, the viral protein is a receptor binding domain of a SARS-CoV-2 spike protein.

In some aspects, disclosed herein is a method of producing a recombinant antibody comprising cultivating or maintaining the host cell of any preceding aspect under conditions to produce said recombinant antibody.

In some aspects, disclosed herein is a method of treating, preventing, reducing, and/or inhibiting coronavirus infection comprising administering to a subject a therapeutically effective amount of the recombinant antibody of any preceding aspect.

The antibody repertoire characterization done herein is also readily generalizable to other pathogens, and as such, have a broad and lasting impact on the development of countermeasures for established and emerging infectious diseases.

Methods for determining antibody sequences and antigen-antibody specificities are known in the art. See, e.g., International Publication Number: WO 2020/033164, incorporated by reference.

In some aspects, disclosed herein is a method for detecting a coronavirus infection in a subject, comprising: providing a biological sample from the subject, and detecting a coronavirus antigen in the biological sample with an antibody that specifically binds to the coronavirus antigen, wherein the antibody is from any aspect as disclosed herein, and wherein the presence of the coronavirus antigen in the biological sample indicates the subject is infected with a coronavirus.

The biological sample can be from, for example, a throat swab, a nasal swab, a nasopharyngeal swab, an oropharyngeal swab, cells, blood, serum, plasma, saliva, urine, stool, sputum, or nasopharyngeal aspirates.

In some embodiments, the coronavirus infection is caused by SARS-CoV-2. In some embodiments, the method comprises contacting the biological sample with a SARS-CoV-2 antigen. In some embodiments, the SARS-CoV-2 antigen is directly immobilized on a substrate and is detected by an antibody disclosed herein directly or indirectly by a labeled heterologous anti-isotype antibody, wherein the bound antibody can be detected by a detection assay. The SARS-CoV-2 antigen can be selected from the spike (S), envelope (E), membrane (M), and nucleocapsid (N) proteins, or a fragment thereof.

The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a secondary antibody that is labeled a fluorescent probe or with biotin for detection. In vitro techniques for detection of the antibodies of SARS-CoV-2 include enzyme linked immunosorbent assays (ELISAs), Western blots, immunoprecipitations and immunofluorescence, IgM antibody capture enzyme immunoassay (MAC-ELISA), indirect IgG ELISA, indirect fluorescent antibody assay (IFAT), hemagglutination inhibition (HIT), and serum dilution cross-species plaque reduction neutralization tests (PRNTs).

In some embodiments, in vitro techniques for detection of an antigen of SARS-CoV-2 include enzyme linked immunosorbent assays (ELISAs), Western blots, immunoprecipitations and immunofluorescence. Furthermore, in vivo techniques for detection of SARS-CoV-2 include introducing into a subject a labeled antibody directed against the polypeptide. For example, the antibody can be labeled with a radioactive marker whose presence and location can be detected by standard imaging techniques, including autoradiography.

In some embodiments, the levels of the antibodies are determined by immunoassay comprising Enzyme linked immunospot (ELISPOT), Enzyme-linked immunosorbent assay (ELISA), western blot, or a multiplex ELISA assay. In some embodiments, the multiplex ELISA assay is selected from the group consisting of Luminex, Veriplex, LEGENDplex, Bio-Plex, Milliplex MAP, and FirePlex.

The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

The invention also encompasses kits for detecting the presence of SARS-CoV-2 or a polypeptide/antigen thereof in a biological sample. For antibody-based kits, the kit can comprise, for example: (1) a first antibody (e.g., attached to a solid support) which binds to a coronavirus antigen; and, optionally, (2) a second, different antibody which binds to either the coronavirus antigen or the first antibody and is conjugated to a detectable agent.

### EXAMPLES

The following examples are set forth below to illustrate the antibodies, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Antibodies for SARS-CoV-2

The Coronavirus (CoV) genus consists of positive sense RNA, zoonotic pathogens. The CoV genome is largely comprised of RNA-synthesis proteins, but approximately a third of the genetic payload encodes four structural proteins including the spike (S), envelope (E), membrane (M), and nucleocapsid (N). The S protein is the immunodominant region of the CoV recognized by the immune system and serves as the target for a number of neutralizing antibodies. Passive transfer of neutralizing antibodies can prevent coronavirus infection in animal models. Further, engineered prefusion-stabilized S protein immunogens have been shown to elicit high titers of coronavirus-neutralizing antibodies in animal models, in the context of MERS. More generally, a better understanding of the human antibody response to the S protein of SARS-CoV-2 as well as other related CoV members can help inform therapeutic antibody optimization and accelerate vaccine design efforts.

The LIBRA-seq technology (LInking B-cell Receptor to Antigen specificity through sequencing) is used for antibody discovery and characterization of antigen-specific antibody repertoires. Unlike other B cell approaches, LIBRA-seq is the first to enable the simultaneous determination of BCR sequence and antigen specificity for a large number of B cells against a theoretically unlimited number of diverse antigens, at the single-cell level. LIBRA-seq therefore provides a unique opportunity for characterizing the types and specificities of antibodies that can recognize the S protein from SARS-CoV-2, as well as other CoV viruses.

Here, the LIBRA-seq technology is used for identifying SARS-CoV-2-specific antibodies. Disclosed herein are cross-reactive antibodies that recognize multiple antigen variants associated with human coronavirus infection, including SARS-CoV-2, SARS-CoV-1, and MERS-CoV. The identification of cross-reactive coronavirus antibodies provides therapeutic and preventive measures against these highly pathogenic and lethal coronaviruses. Antibodies that cross-react with multiple known viruses target broadly conserved CoV S epitopes, and therefore become a first line of defense against new outbreaks with previously unencountered CoV viruses.

The types of antibody repertoire characterization done herein is also readily generalizable to other pathogens, and as such, have a broad and lasting impact on the development of countermeasures for established and emerging infectious diseases.

### Example 2. Broadly Neutralizing Antibodies for Coronavirus

Little is known about the ability of human antibodies to recognize multiple diverse CoV viruses, especially in the context of the newly identified SARS-CoV-2. The present disclosure identifies and characterizes such antibodies, providing novel insights into the frequencies, sequence characteristics, and epitope specificities of CoV cross-reactive antibodies. Further, given that such antibodies can be potent and broadly neutralizing, they are of exceptional value for the development of effective pan-CoV antibody-based therapeutics that can target novel CoV viruses that emerge in the future. Additionally, identifying antibodies that exhibit mono-specificity for SARS-CoV-2 are be of high significance for the development of immediate countermeasures.

The LIBRA-seq antigen screening library includes lead vaccine candidates for SARS-CoV-2 and other CoV. The protein version of the SARS-CoV-2 prefusion S vaccine that is now in clinical trials (ClinicalTrials.gov id: NCT04283461) is included herein.

### Example 3. Methods

The example shown here combines both experimental and computational efforts. In particular, a variety of techniques are utilized, bringing together microfluidics, next-generation sequencing, and protein science technologies, combined with computational analysis of the experimentally generated datasets.

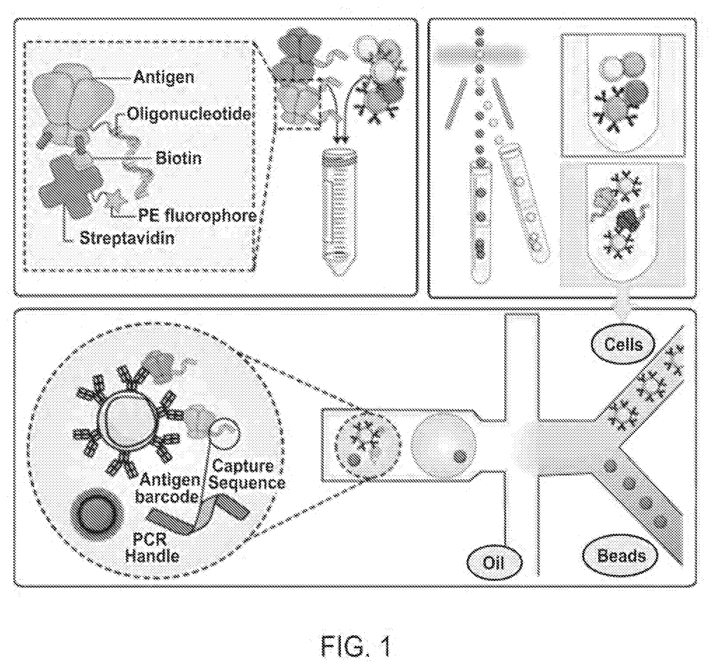

Summary of experimental framework. The LIBRA-seq technology is based on the following framework (FIG. 1). Each of a set of target antigens is “barcoded” with a unique DNA sequence that is sufficiently different from any other barcode (thus allowing for robust barcode identification even with sequencing read errors). A given sample of B cells is then mixed with the set of antigens, such that if a B cell recognizes a specific antigen, that antigen is able to bind to the BCRs of that B cell. The cell-antigen mixture is FACS-sorted and run through a microfluidics system (such as the 10× Chromium system) for single-cell processing and high-throughput NGS (such as through an Illumina MiSeq or NovaSeq). The barcode for any antigen bound to a given single B cell is sequenced, thus simultaneously delineating the BCR sequences and the antigen specificity of the cell. Finally, the sequencing data is analyzed to identify B cell-antigen complexes by searching for B cells for which both BCR sequence and antigen barcode data are available.

Antigen production. Recombinant soluble antigens are expressed in 293F cells using polyethylenimine (PEI) transfection reagent. Protein antigens are purified over StrepTactin resin (IBA). Concentrated protein is run on a Superdex 200 Increase 10/300 GL or Superose 6 Increase 10/300 GL sizing column on an AKTA FPLC system. AviTagged antigens are biotinylated using BirA biotin ligase and conjugated to streptavidin-APC or streptavidin-PE for use in flow cytometry. Non-AviTagged proteins are biotinylated non-specifically which targets secondary amines on the protein's N terminus as well as solvent exposed lysine residues. For flow cytometry, cells were stained with the biotinylated, oligo labeled antigens. Then, after washing, cells were stained with streptavidin-PE, so antigen-reactive cells could be identified during FACS sorting. Unique oligonucleotide barcodes are directly conjugated to each antigen using the Solulink Protein-Oligonucleotide Conjugation Kit (TriLink cat no. S-9011) according to manufacturer's instructions. Antigen concentrations are estimated by BCA assay. Barcoded antigens are analyzed by SDS-PAGE, and antigenicity by ELISA is characterized with known mAbs specific for that antigen.

Antigen-specific B-cell sorting. PBMCs from the donors are stained with a panel of cell markers, and with fluorescent antigen to identify antigen-specific B cells, e.g., live CD3⁻ CD14⁻ CD19⁺ IgG⁺ and antigen⁺.

LIBRA-seq experiments. Cells are bulk sorted for loading onto the Chromium microfluidics device (10× Genomics) and processed using the B-cell VDJ solution according to manufacturer's suggestions for a target capture of 10,000 B cells per ⅛ 10× cassette. NGS is performed using NovaSeq 6000. Output fastq files are processed using Cell Ranger (10× Genomics) to assemble quantify, and annotate paired V(D)J transcript sequences and antigen barcode counts on a cell-by-cell basis using 10× Chromium cellular barcodes. The number of antigen barcodes for each cell is counted and then transformed and scaled. The final output is a matrix containing information on a cell-by-cell basis, including normalized UMI counts (referred to as LIBRA-seq scores), heavy and light chain sequences, and annotated sequence features including V gene, J gene, CDR3 sequence, identity to germline, isotype, and others.

### Example 4. Results

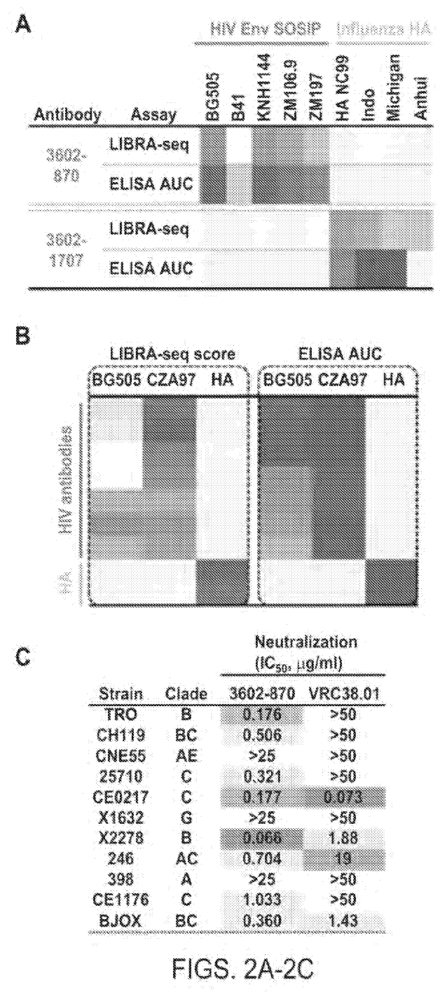

It has been established that LIBRA-seq can successfully identify broadly neutralizing antibodies (bNAbs) in the context of HIV-1. In particular, LIBRA-seq was used to analyze the antibody repertoires of two HIV-infected donors, N90 and NIAID 45, from whom HIV-1 bNAbs had been previously identified. In both cases, novel members were identified from the known bNAb lineages. Importantly, LIBRA-seq also successfully identified novel lineages of antigen-specific antibodies from both donors, with high concordance between LIBRA-seq scores and ELISA binding for tested antibodies (FIGS. 2A-2B). Further, the HIV-specific antibody 3602-870, a member of a new lineage in donor N90 utilizing different germline genes compared to VRC38, was able to neutralize more viruses and with greater potency compared to the broadest member of the VRC38 lineage (FIG. 2C).

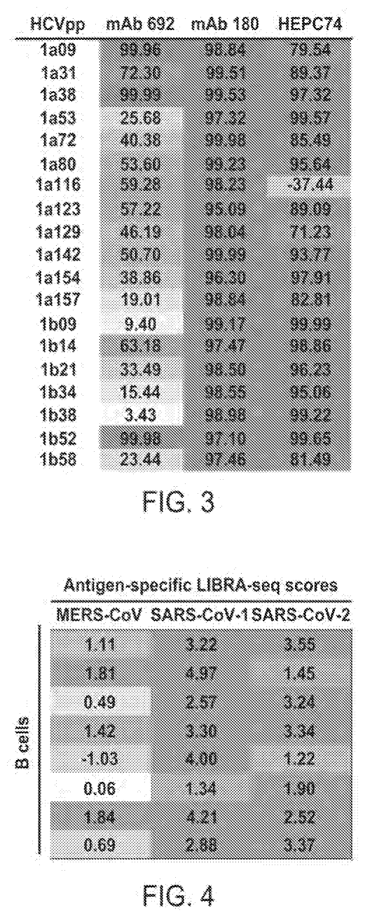

Further disclosed herein are data showing the application of the technology in the setting of another virus, hepatitis C. In particular, LIBRA-seq is applied to characterize the antibody repertoire in a sample co-infected with HIV-1 and hepatitis C. A number of antibodies were identified that showed reactivity against hepatitis C antigens that were part of the LIBRA-seq screening library. The HCV neutralization ability of these antibodies were tested (FIG. 3). Notably, mAb 180 exhibited exceptional neutralization breadth that was superior to the best known HCV broadly neutralizing antibodies.

Taken together, these data indicate that LIBRA-seq is successfully applied to the characterization of antibody repertoires in the context of diverse pathogens (for example, SARS-CoV-2).

### Example 5. Cross-Reactive Antibodies that Recognize Multiple Antigen Variants Associated with Human Coronavirus Infection, Including SARS-CoV-2, SARS-CoV-1, and MERS-CoV

An antigen screening library that includes antigens from multiple diverse CoV viruses is used for screening for B cells that exhibit CoV cross-reactivity.

Study samples. A sample (20M PBMCs) from an individual who had prior SARS-CoV-1 infection. The subject had been infected during the SARS-CoV-1 outbreak in 2004, whereas the sample was collected ˜14 years post infection. Recent work has indicated that there are indeed antibodies that can cross-react between SARS-CoV-1 and 2. Therefore, this sample represents an appropriate target for LIBRA-seq screening for cross-reactive antibodies using diverse CoV antigens.

Antigen screening library. The library includes S protein antigens associated with common CoV (HKU1 and OC43), as well as SARS-CoV-2, SARS-CoV-1, and MERS. All of these antigens are in their prefusion-stabilized conformation that represent neutralization-sensitive epitopes better than other forms of this antigen. In addition, two HIV-1 antigens are used as a negative control.

Monoclonal antibody selection and validation. Select monoclonal antibodies corresponding to the BCRs from LIBRA-seq-identified antigen-specific B cells are synthesized, produced, and characterized for binding to the target antigens. Antibody selection criteria. Antibodies are selected for validation using several criteria, including: (a) high level of overall CoV cross-reactivity (i.e., antibodies that are reactive with multiple antigens from the screening library); (b) high LIBRA-seq scores for individual antigens (e.g., a high score for SARS-CoV-2 prefusion S); (c) BCR sequence characteristics (e.g., high level of somatic hypermutation, long CDRH3, etc.); (d) size of B cell clonotype (i.e., a large number of unique B cells that belong to the respective B cell lineage); (e) source donor (i.e., antibody selection to multiple donors is distributed). These criteria allow for selecting antibodies that have target antigen specificity profiles. Antibody production and characterization. Antibody heavy and light chains are synthesized by Twist Bioscience. A two-tier system is utilized for antibody production and validation. As a first step, the Crowe laboratory performs high-throughput antibody expression, purification, and ELISA binding against the same antigens as used in the LIBRA-seq screening library. For high-throughput production of recombinant antibodies, designated as “microscale”, antibody expression plasmids are transiently transfected into ˜1 mL Chinese hamster ovary (CHO) cell cultures per antibody in deep 96-well blocks. After 7-10 days, individual cultures are pelleted by centrifugation of the deep-well plate, and supernatants individually harvested using 96-channel automated pipetting. High-throughput micro-scale antibody purification of clarified supernatants is carried out using deep well plates containing protein G resin (GE Healthcare Life Sciences). A total of up to 100 antibodies are tested in the high-throughput step. Up to 20 antibodies that show an antigenicity profile from the high-throughput expression step are selected for large-scale expression. 293T cells are co-transfected with plasmids expressing Ig heavy and light chain genes. Recombinant mAbs are purified with protein-A agarose columns. The antibodies are tested in a number of assays: (i) Antigen binding is confirmed through ELISA, as well as surface plasmon resonance and biolayer interferometry by the McLellan group; (ii) Antibody epitopes are mapped using standard techniques, such as antigen epitope-knockouts and antibody binding competition; (iii) Antibody-virus neutralization is also tested; (iv) For antibodies, high-resolution cryo-EM structures are obtained by the McLellan group, who is a leader in the field of CoV structural biology and recently published the first ever cryo-EM structure of SARS-CoV-2 prefusion S.

Data. LIBRA-seq was applied to a sample from an individual who had prior SARS-CoV-1 infection using an antigen screening library that included antigens from diverse CoV, including prefusion-stabilized S from SARS-CoV-2, SARS-CoV-1, and MERS-CoV. LIBRA-seq discovered >2,500 B cells with antigen specificity for at least one CoV antigen from the screening library. FIG. 4 shows examples of a number of cross-reactive IgG⁺ B cells that had high LIBRA-seq scores for SARS-CoV-2 in addition to SARS-CoV-1, MERS, or both.

The present disclosure identifies cross-reactive and mono-reactive coronavirus antibodies. (i) Given that there are conserved regions on the CoV S protein, and reports of CoV antibody cross-reactivity between certain viruses, such cross-reactive antibodies are identified. The availability of the newly developed LIBRA-seq technology provides unique advantage compared to previous technologies, providing an unprecedented understanding of cross-reactive antibody characteristics, including sequence, functional, and epitope properties.

### Example 6. Ability of Current Lead CoV Vaccine Candidates to Engage with Antibody Repertoires from Healthy Individuals

SARS-CoV-2 vaccine candidate based on a stabilized prefusion S immunogen is already in clinical trials (ClinicalTrials.gov id: NCT04283461). Similar constructs for SARS-CoV-1 and MERS are also viable targets for clinical development. Ultimately, a vaccine that broadly protects against multiple highly pathogenic CoV can prove of significant value both against known and potential future CoV pathogens. The traditional model of vaccine development involves clinical trials to test the safety, immunogenicity, and efficacy of a candidate vaccine, frequently being subjected to the risk of failing to meet objectives and not receiving approval, even after investment of immense resources. In contrast, platforms like the one disclosed here, that enable ex vivo evaluation of vaccine candidates, can help efficiently prioritize the selection of candidates for clinical studies, thereby saving substantial effort and resources, and optimizing the likelihood of selecting a vaccine candidate with a greater potential for success.

There are a multitude of factors that can impact the performance of a vaccine candidate. The study herein focuses on the ability of vaccine candidates to engage with the antibody repertoires of healthy individuals. Specifically, by screening the antibody repertoires of healthy individuals, the capacity of current SARS-CoV-2, MERS, and SARS-CoV-1 lead vaccine candidates to engage with naïve antibody repertoires is evaluated. In essence, these experiments address the question of whether B cells that can recognize a given vaccine candidate readily exist in healthy individuals, and what the sequence and phenotypic characteristics of these B cells are. The application of the LIBRA-seq technology to address this question enables an unparalleled, high-throughput, characterization of the antigen specificity and BCR sequence characteristics of B cells that are capable of recognizing the CoV vaccine candidates studied here, from healthy individuals. These assessments can determine how well a given vaccine candidate can be able to engage with the repertoires of healthy individuals upon vaccination.

LIBRA-seq data and analysis. The healthy-donor LIBRA-seq data generated above are utilized here. However, unlike the characterization above, where the focus is to identify potently neutralizing CoV-specific antibodies, this study defines the overall reactivity of healthy antibody repertoires against the SARS-CoV-2, SARS-CoV-1, and MERS prefusion S vaccine candidates. For a given vaccine candidate and each healthy donor sample, a number of variables are analyzed, including: (i) frequency of reactive B cells; (ii) frequency of virus-specific vs. cross-reactive B cells; (iii) BCR sequence profiles of the reactive B cells (e.g., V-gene usage frequency, distribution of somatic hypermutation levels, CDRH3 length distribution, clonality, etc.). For each vaccine candidate, the results are analyzed across all donor samples. These experiments determine whether the given vaccine candidate can readily engage with healthy antibody repertoires, and if so, what the common characteristics of the reactive B cells are.

Monoclonal antibody selection and validation. Select monoclonal antibodies are produced and tested recombinantly to develop a general understanding of the fraction of B cell repertoire in healthy individuals that can engage with lead vaccine candidates. The mAb production and characterization focus on SARS-CoV-2 and characterizing B cells reactive with the SARS-CoV-1 and MERS vaccine candidates. The antibody selection criteria targets B cells with diverse properties, including: (a) a diverse spread of SARS-CoV-2 LIBRAseq scores; (b) diverse profiles of BCR sequence characteristics (e.g., diverse levels of somatic hypermutation, different CDRH3 lengths, diverse isotypes, etc.); (c) representatives from multiple source donors; and others.

If SARS-CoV-2-specific B cells are observed infrequently, then the antibody characterization analysis is expanded with B cells that are reactive with the SARS-CoV-1 and MERS vaccine candidates, or further expanded to explore B cells that are reactive with the antigens from the common CoV that are included in the LIBRA-seq screening library. Given the commonality of these infections, a large number of antigen-specific B cells reactive with these common CoV antigens are identified, resulting in novel insights into antibody repertoire reactivity against these viruses.

### Example 7. Identification and Characterization of Coronavirus Cross-Reactive Antibodies Using LIBRA-Seq

The spike protein on the CoV virion engages with host cell receptors to mediate viral entry and is the main antigenic target of neutralizing antibodies. Here, the study shows the antibodies that can cross-react with spike proteins from multiple CoVs. To identify these antibodies, an assay called LIBRA-seq (Linking B cell receptor to antigen specificity through sequencing) was performed, which enables the recovery of paired heavy/light chain antibody sequences along with antigen reactivity information for thousands of single B cells simultaneously. LIBRA-seq was used to screen B cells from a SARS-CoV-1 convalescent donor using an antigen screening library composed of stabilized prefusion spike proteins from pandemic strains (SARS-CoV-2, SARS-CoV-1, MERS-CoV) and endemic strains (HKU1, OC43), resulting in paired antibody sequence-antigen specificity information for 2526 B cells. A number of antibodies that were cross-reactive against SARS-CoV-2, SARS-CoV-1, and MERS-CoV, and in some cases additionally against HKU1 and OC43, were identified and validated in ELISA binding assays. Antibodies are evaluated in various functional assays.

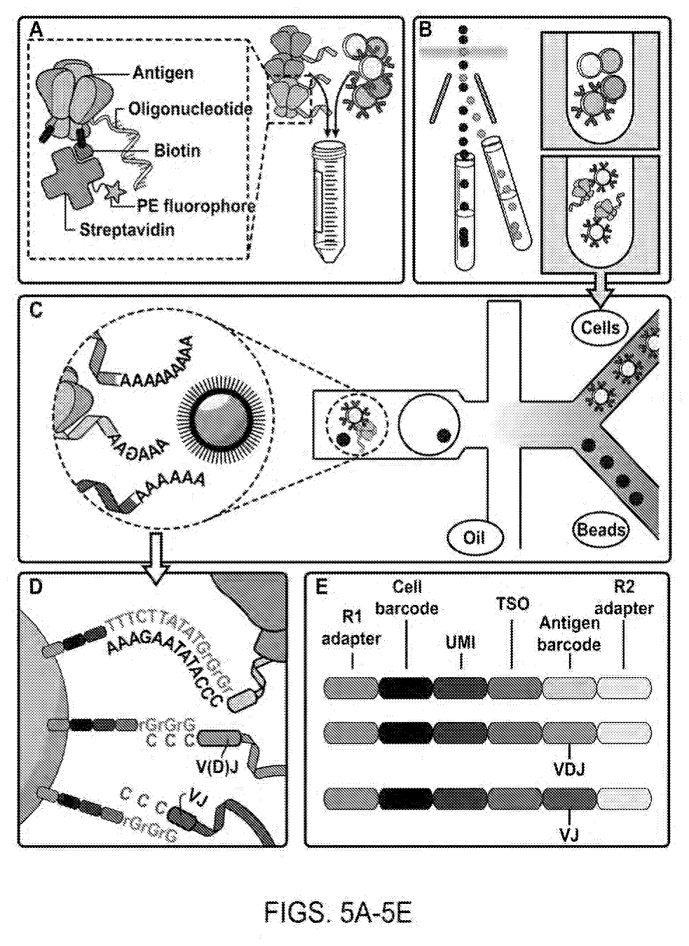

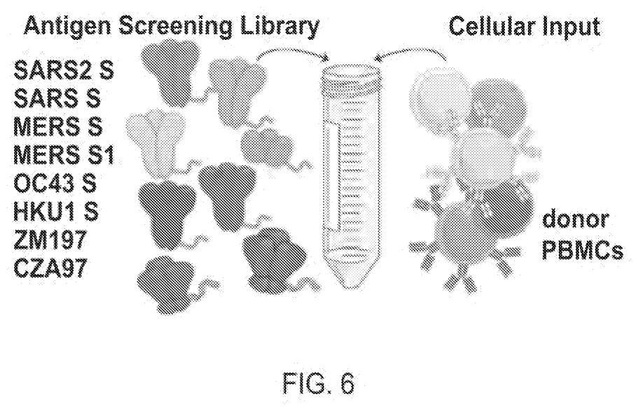

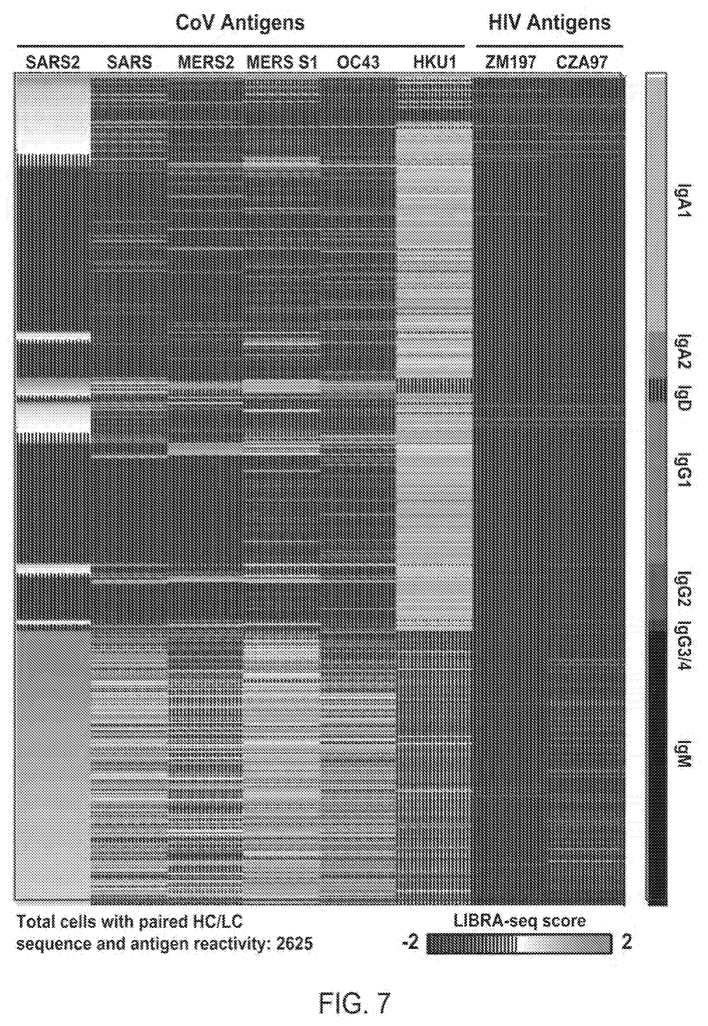

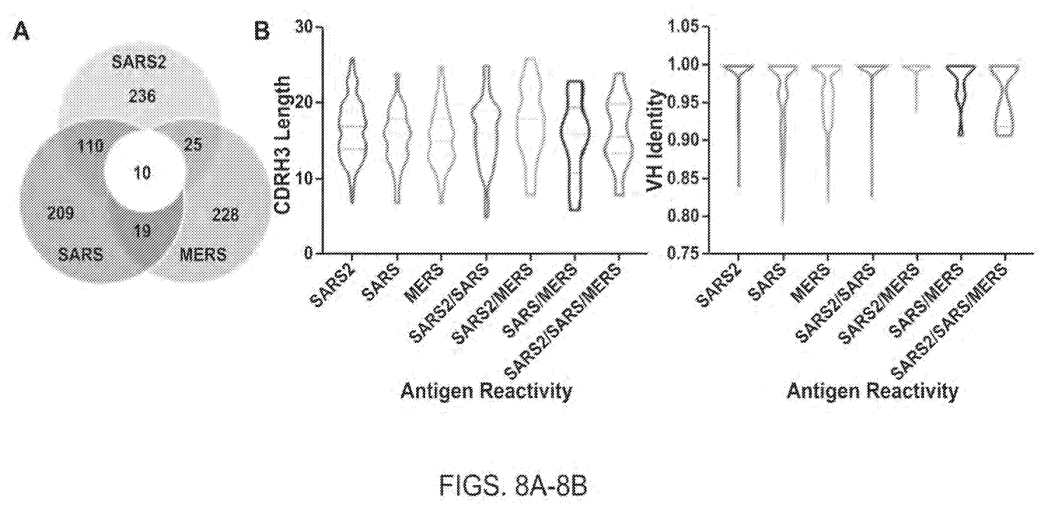

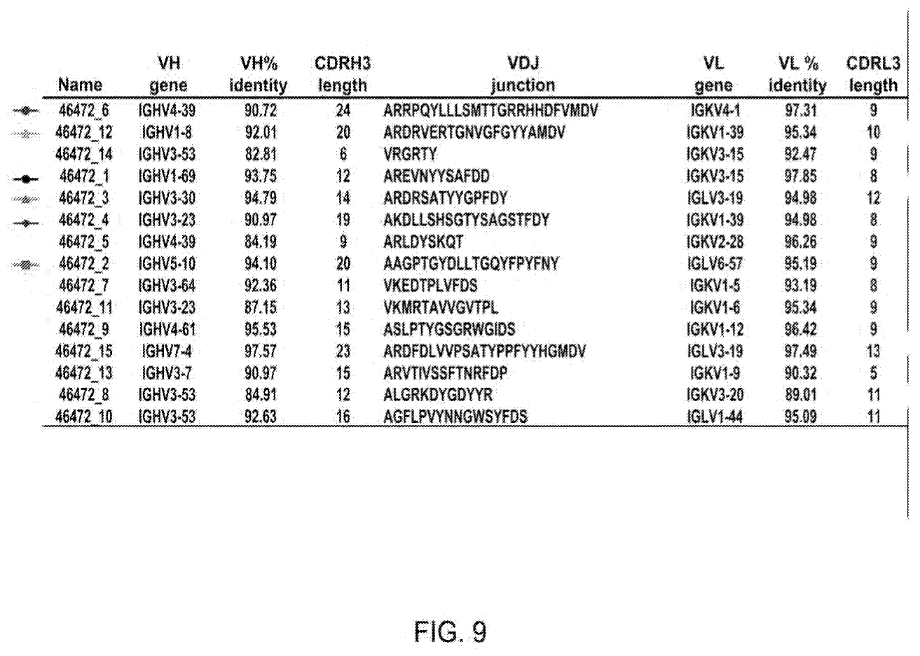

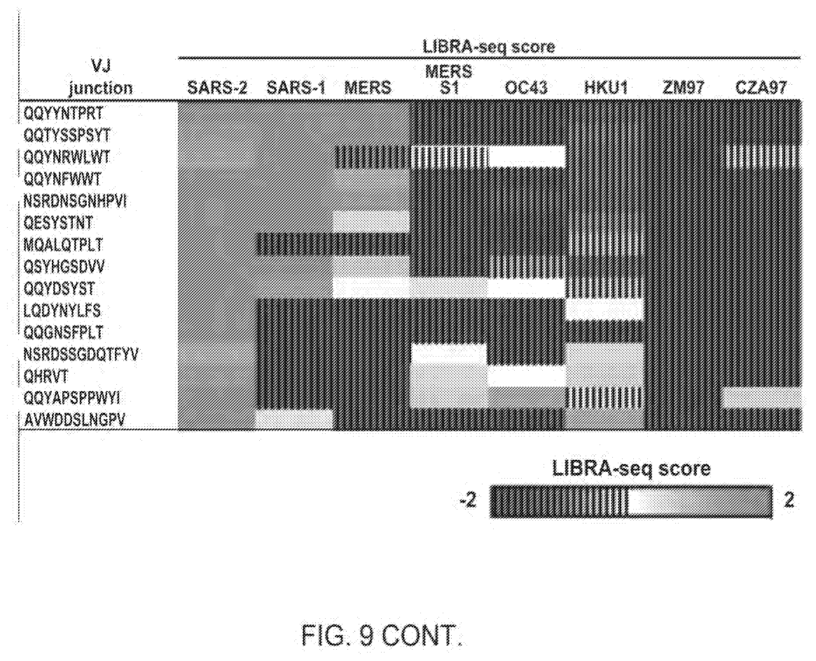

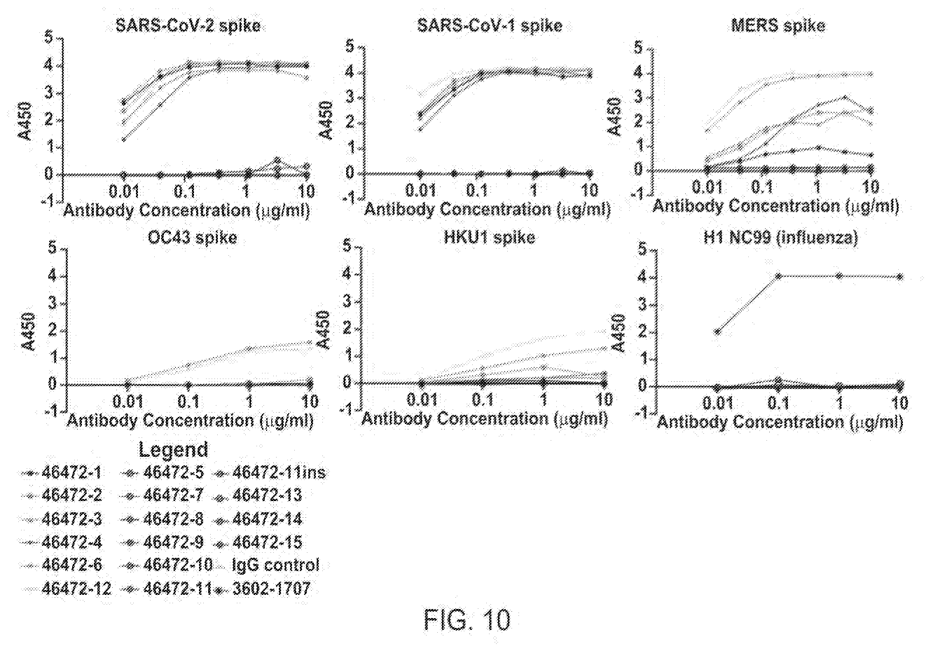

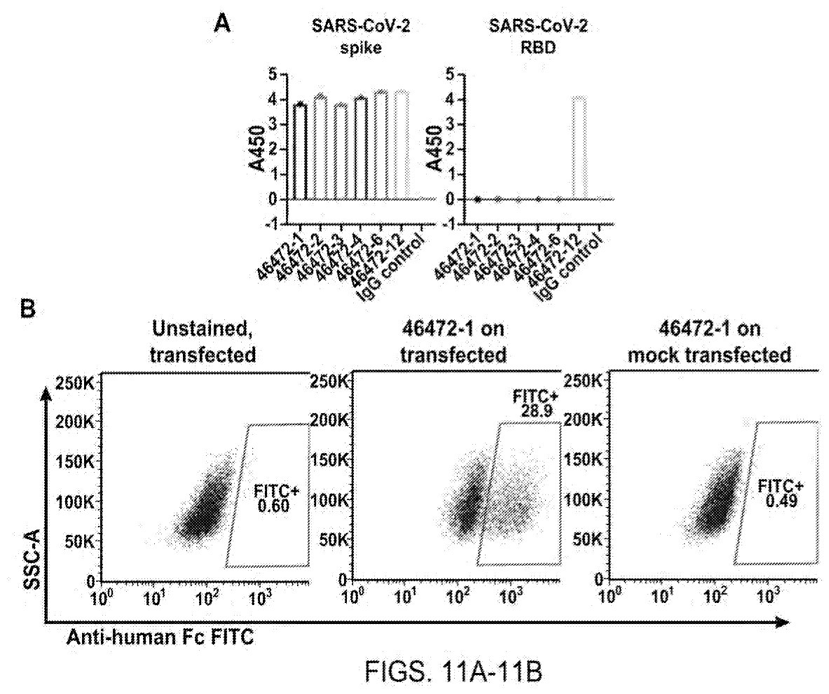

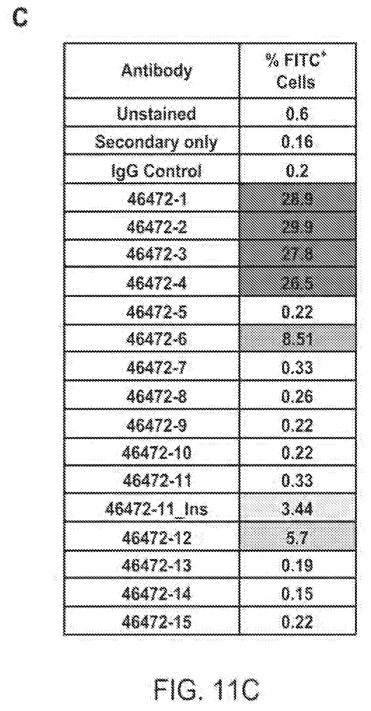

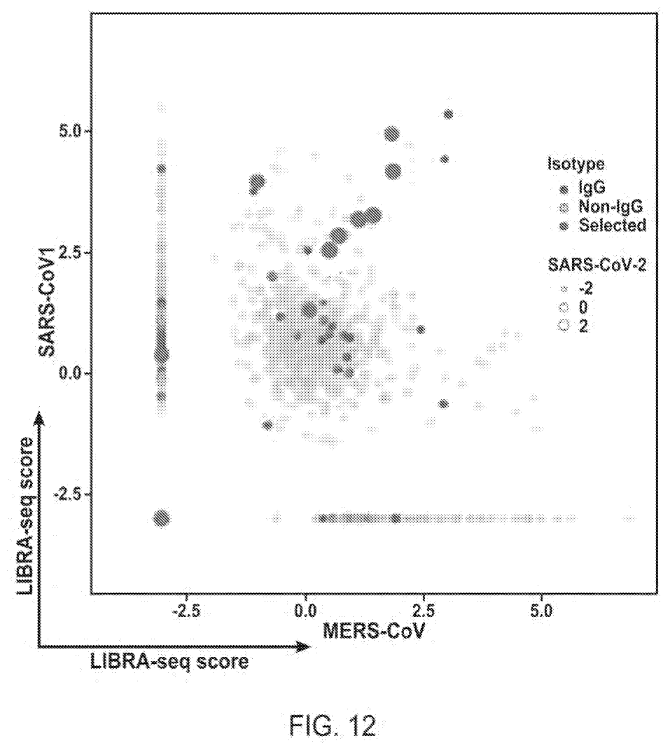

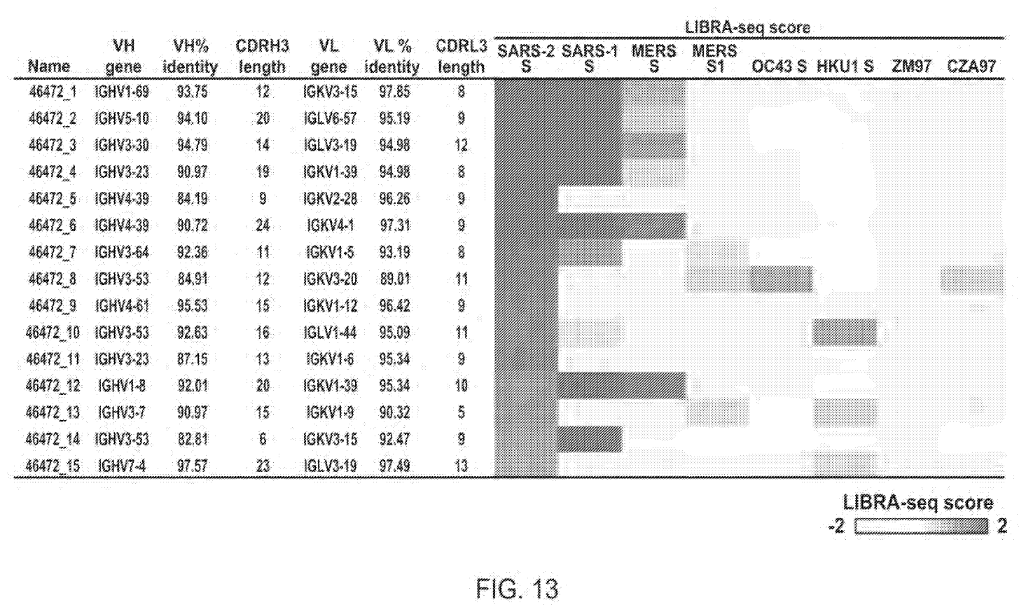

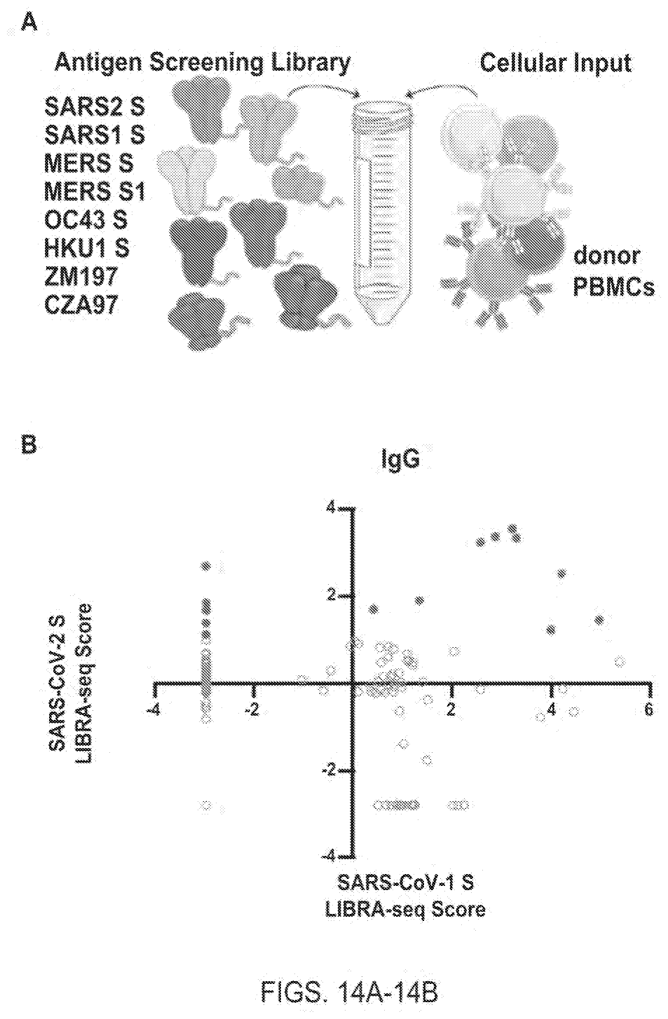

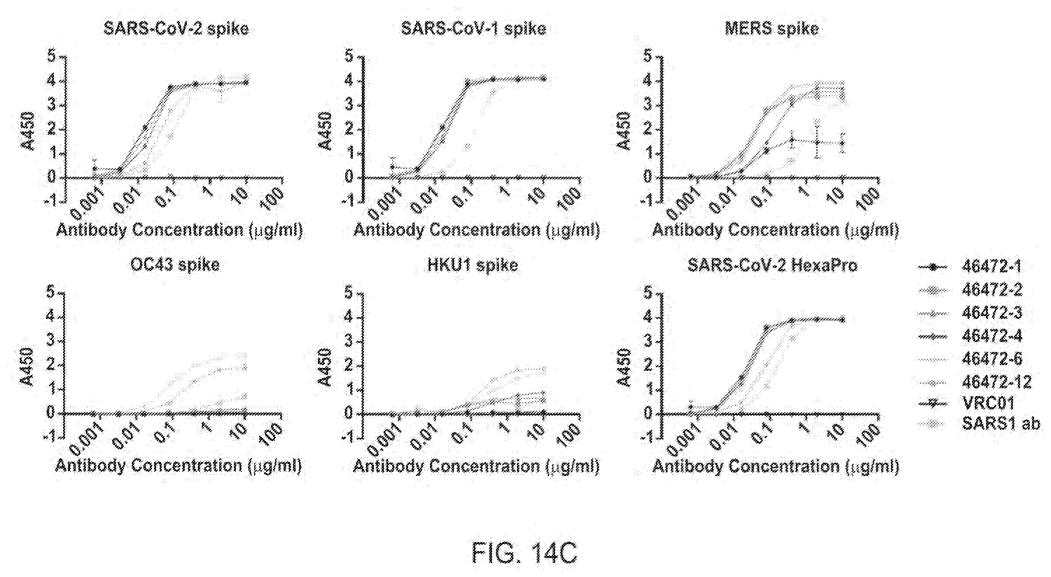

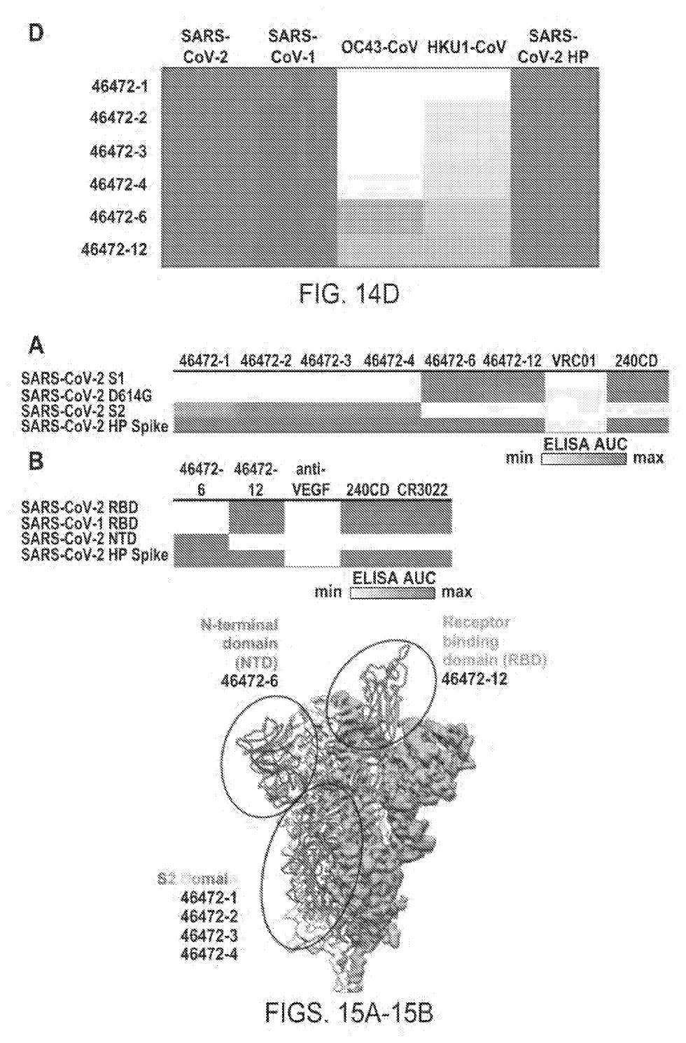

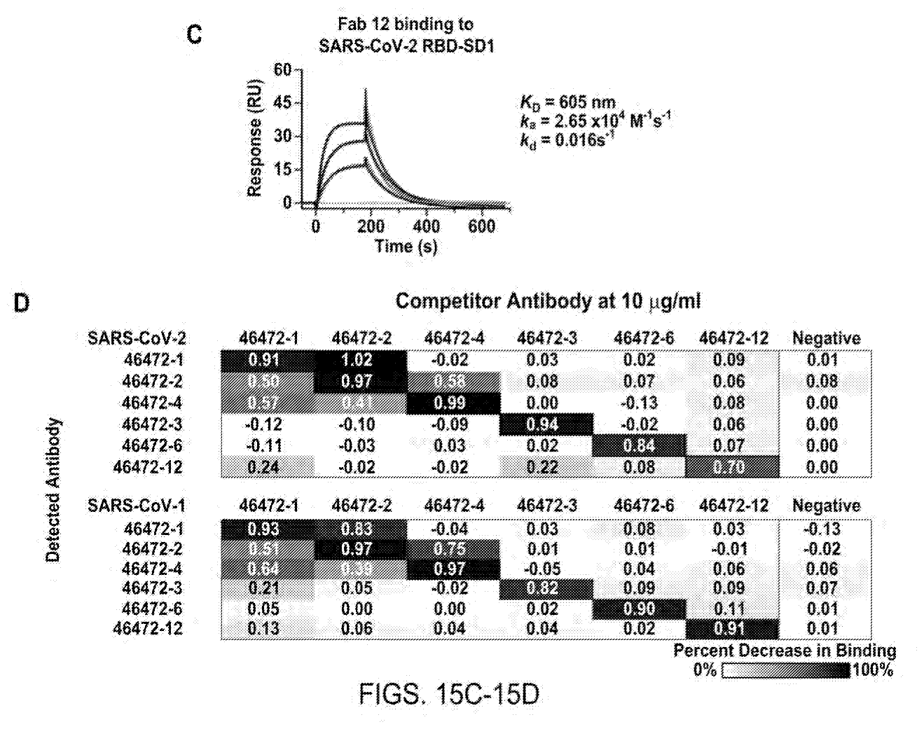

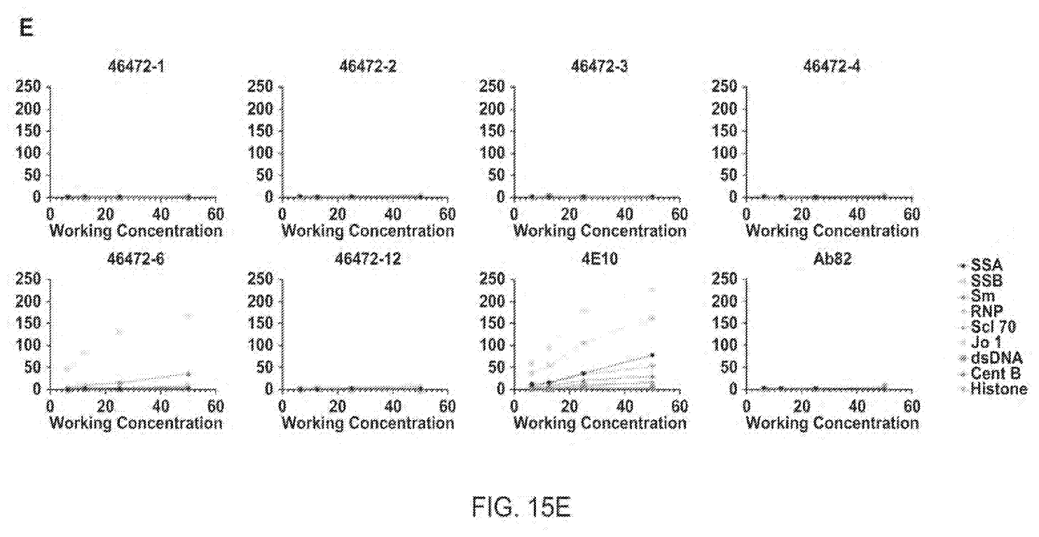

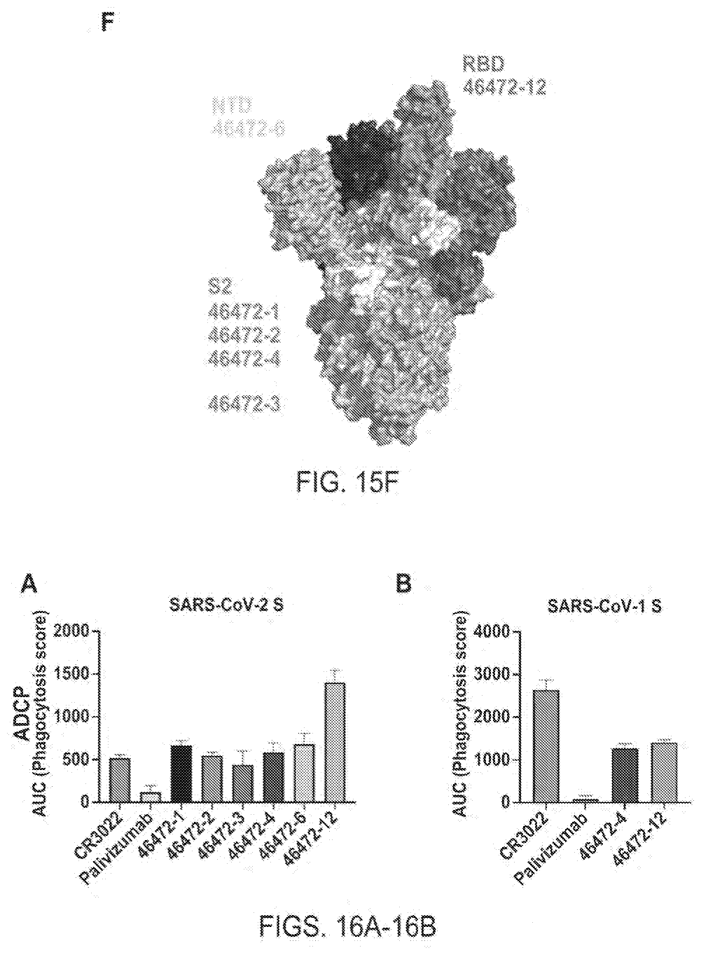

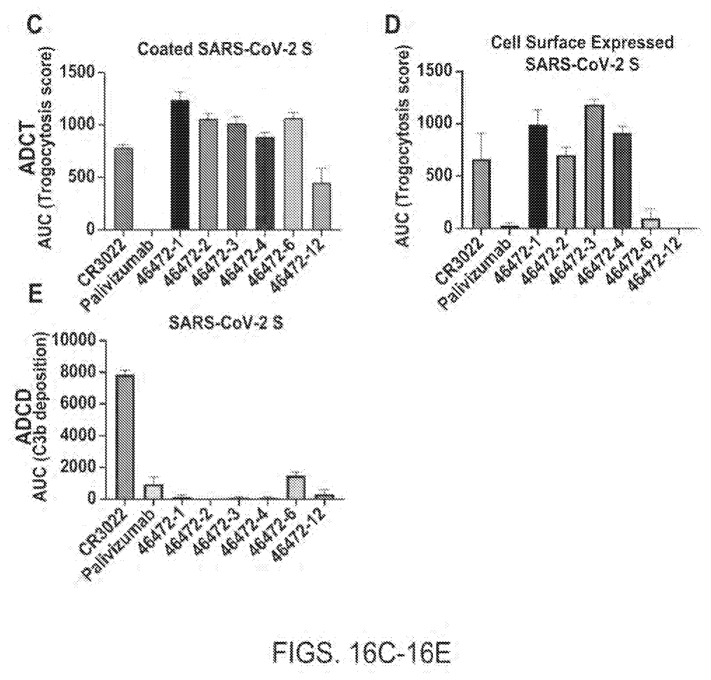

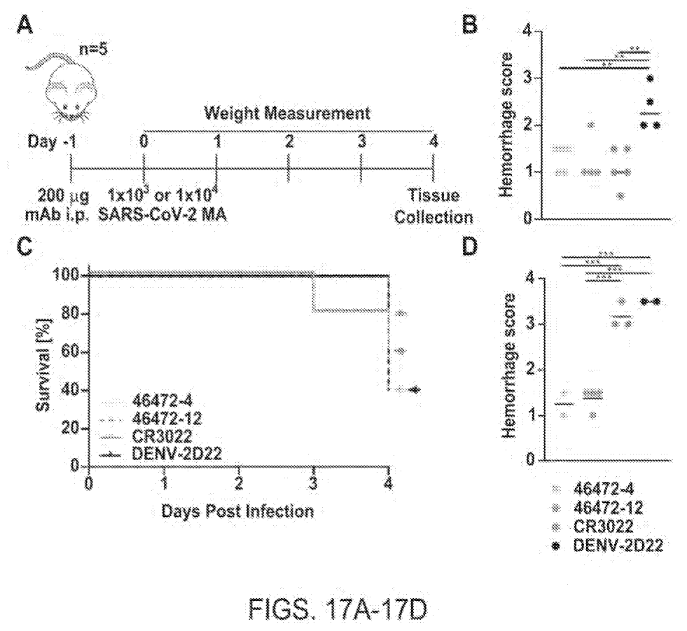

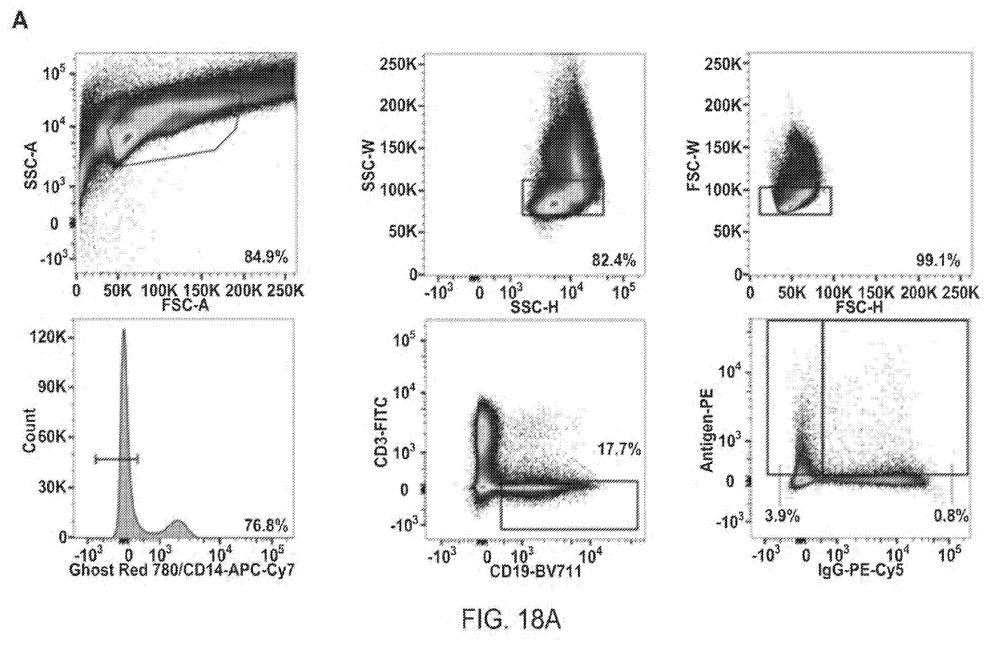

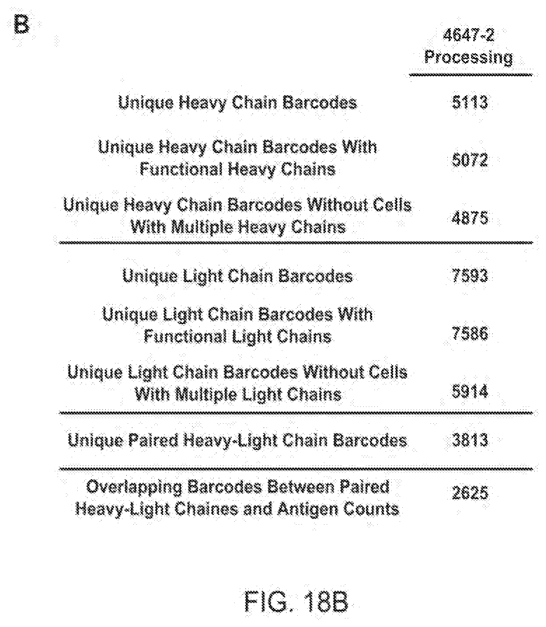

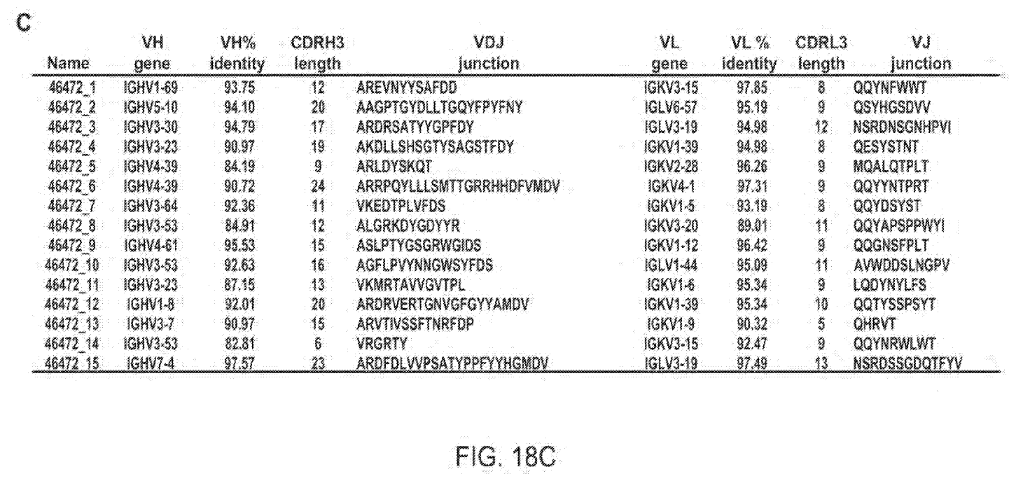

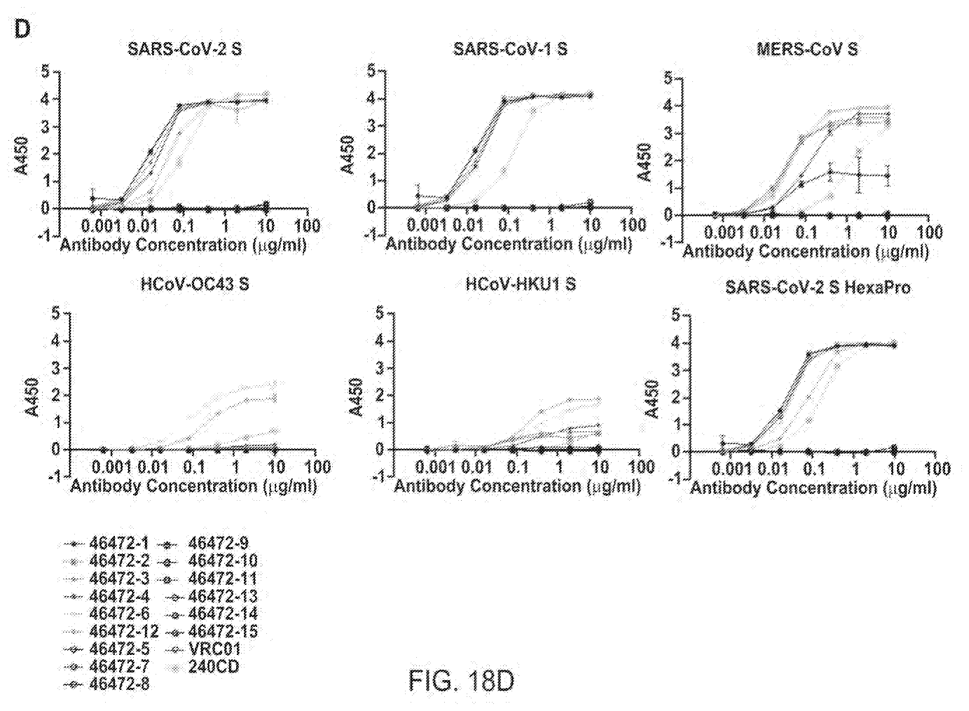

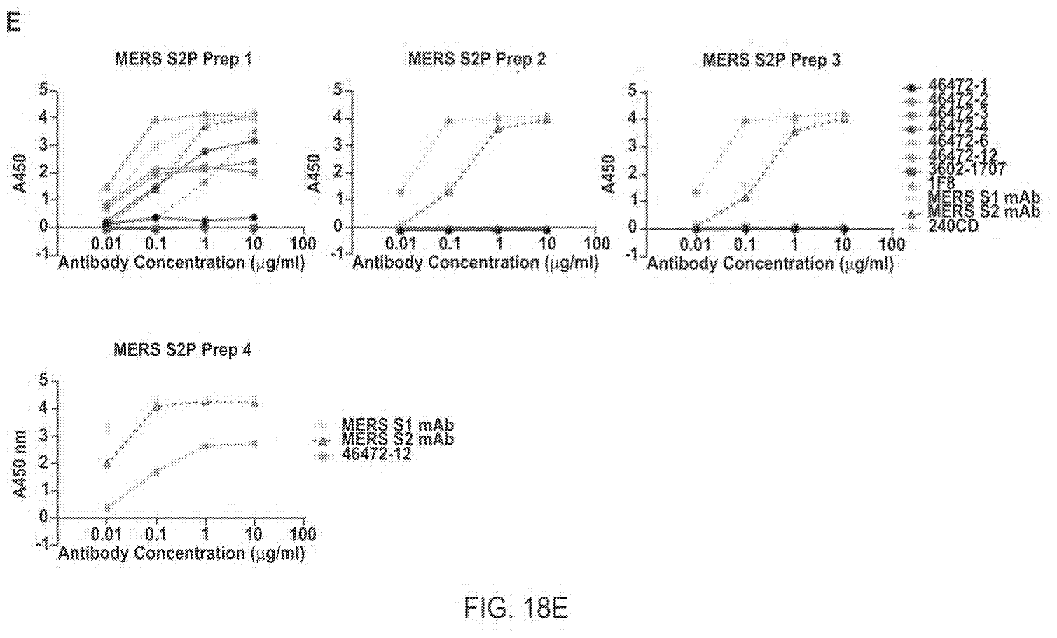

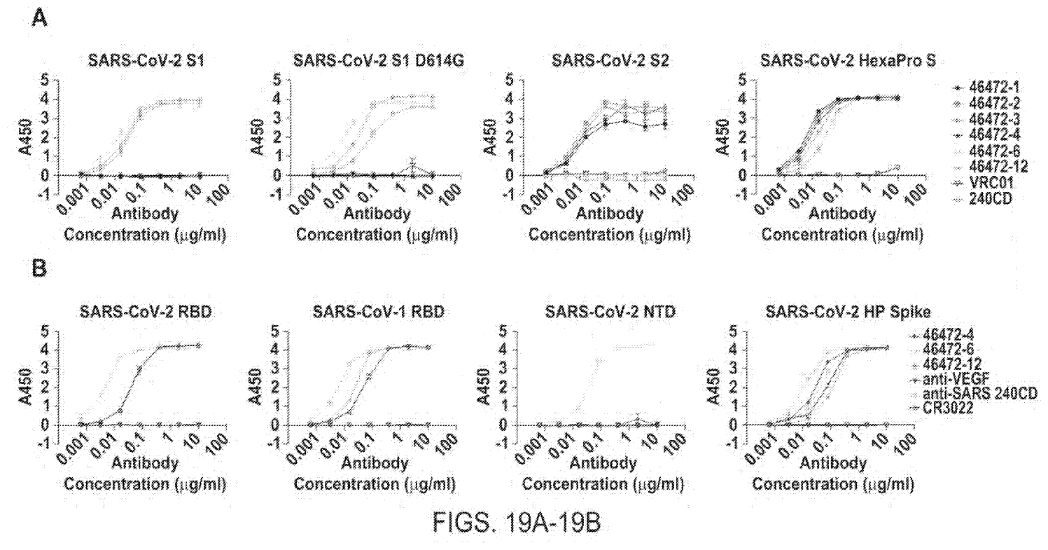

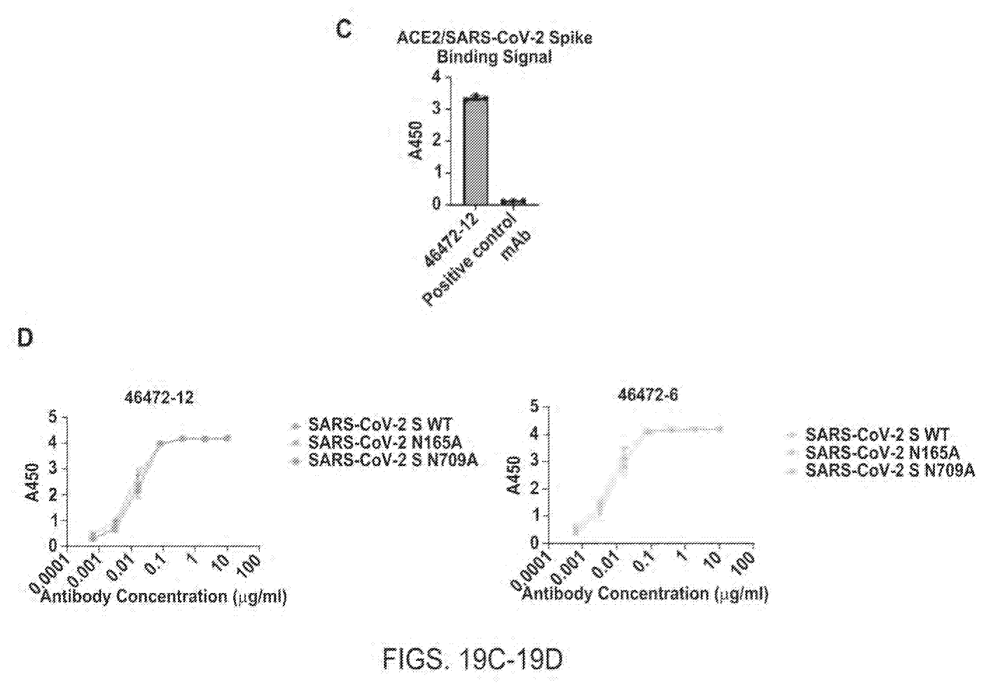

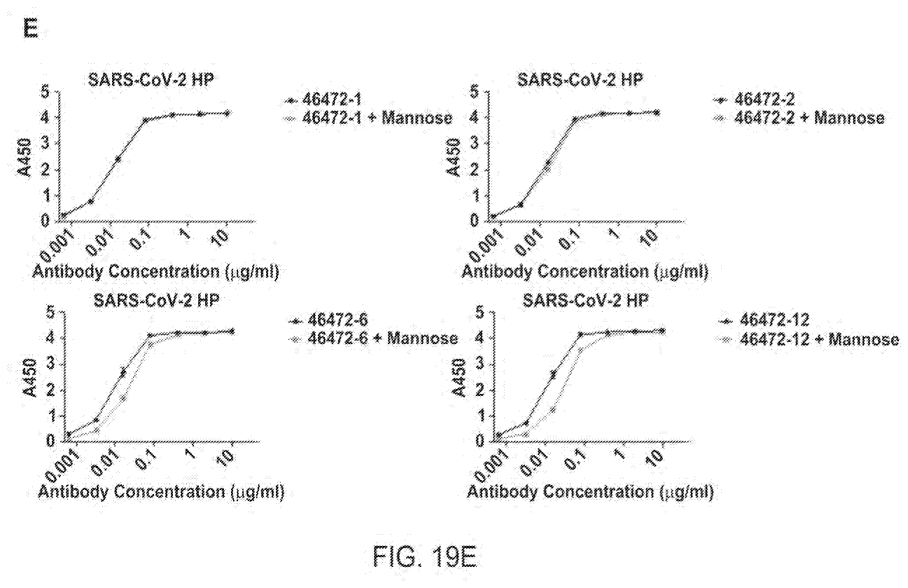

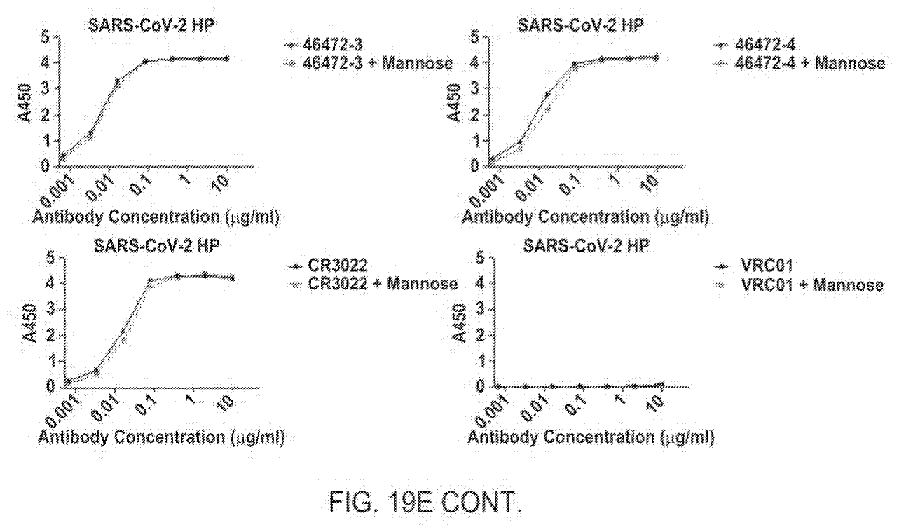

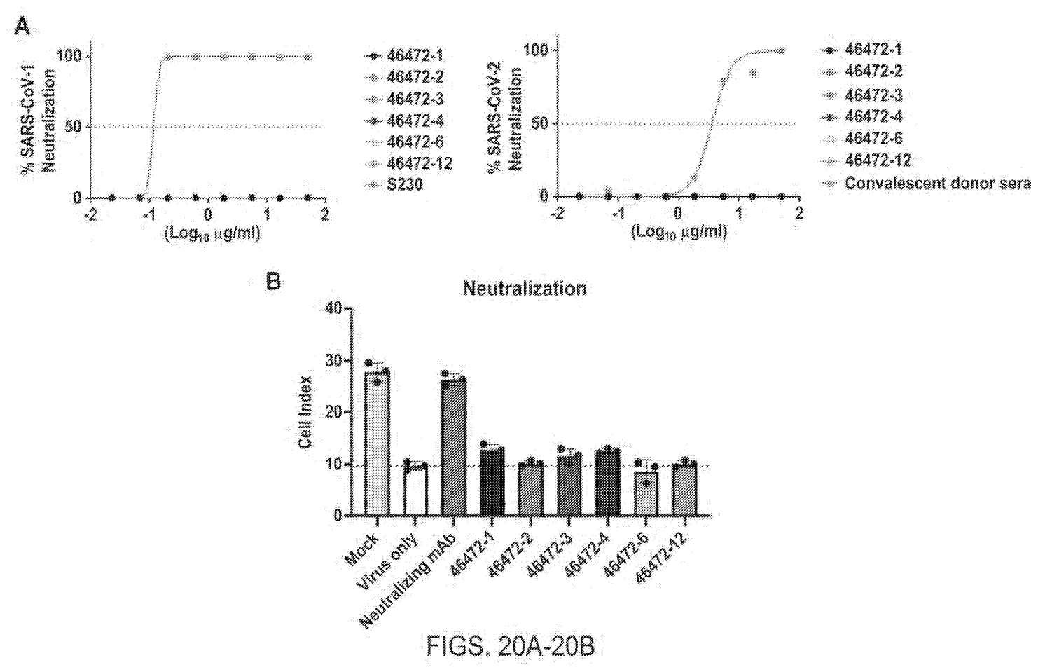

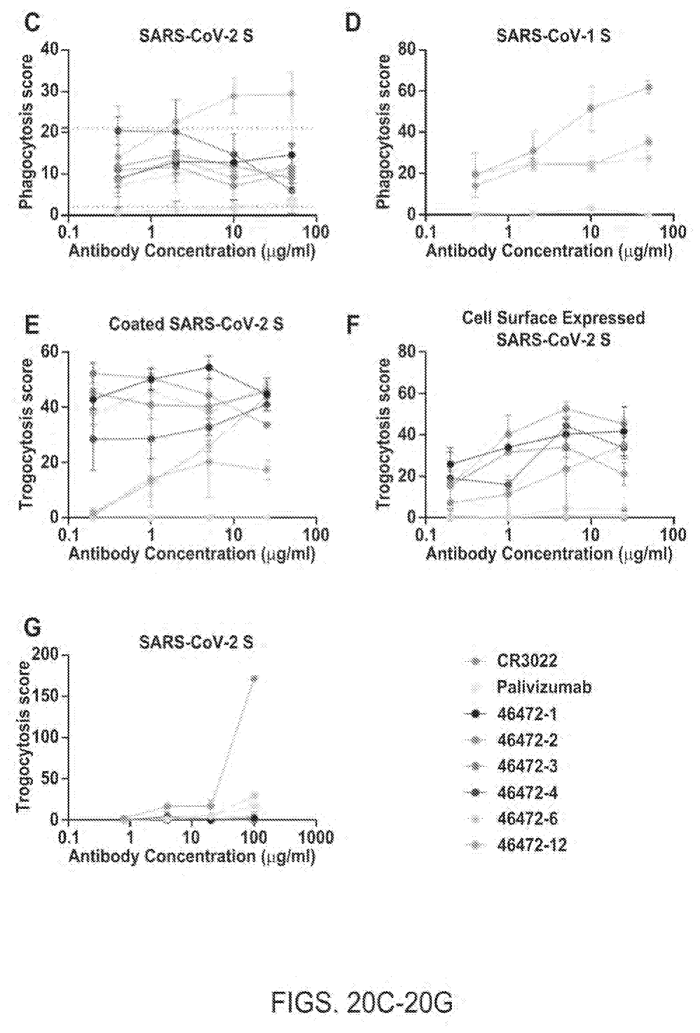

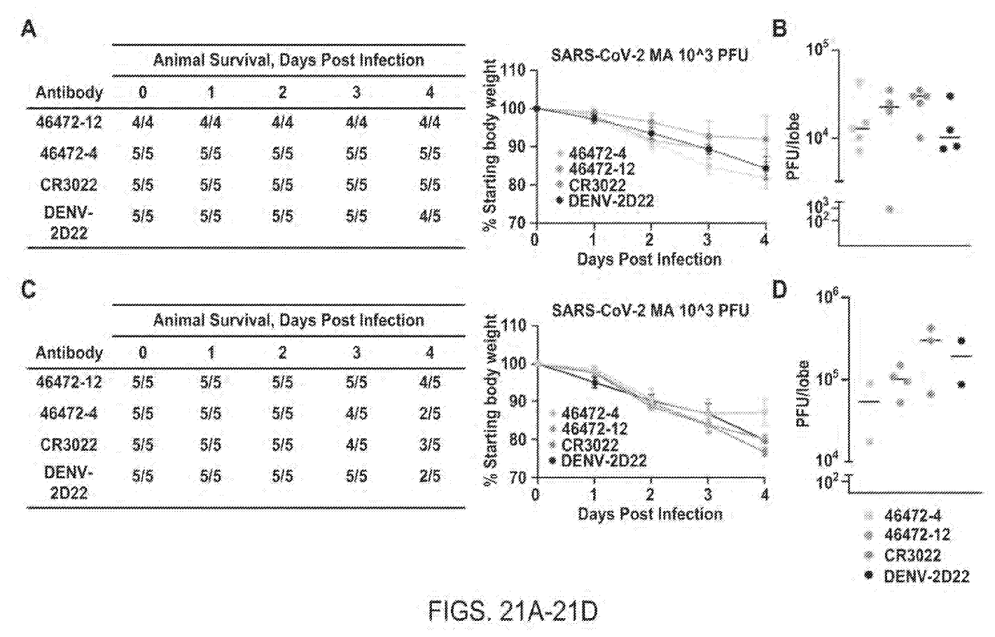

The data (FIGS. 5-17) show that application of the LIBRA-seq method to a SARS convalescent donor PBMC sample using an eight-antigen screening library led to the rapid identification of multiple, genetically distinct antibodies that are cross-reactive to spike trimers from three pandemic strains (SARS-CoV-2, SARS-CoV-1, MERS) and reactive to spike proteins from endemic strains (OC43, HKU1) in some cases.

These antibodies target a variety of epitopes on the spike glycoprotein and can mediate Fc effector functions. Elucidation of cross-reactive CoV epitopes informs rational vaccine design strategies, both for the current and potential future CoV pandemics.

### Example 8. Cross-Reactive Coronavirus Antibodies with Diverse Epitope Specificities and Extra-Neutralization Functions

The emergence of a novel coronavirus (CoV) SARS-CoV-2, the causative agent of COVID-19, has resulted in a worldwide pandemic, threatening the lives of billions and imposing an immense burden on healthcare systems and the global economy. SARS-CoV-2, the seventh coronavirus known to infect humans, is a member of the Betacoronavirus genus which includes the highly pathogenic SARS-CoV-1 and MERS-CoV, as well as endemic variants OC43-CoV and HKU1-CoV. Recent coronavirus outbreaks and the threat of future emerging zoonotic strains highlight the need for broadly applicable coronavirus therapeutic interventions and vaccine design.

Coronaviruses utilize the homotrimeric Spike (S) protein to engage with cell-surface receptors and gain entry into host cells. S consists of two functional subunits: S1 and S2. S1 facilitates attachment to target cells and is composed of the N-terminal domain (NTD) and the receptor-binding domain (RBD), whereas S2, which encodes the fusion peptide and heptad repeats, promotes viral fusion. To facilitate cell entry, human coronaviruses employ different host factors; however, SARS-CoV-1 and SARS-CoV-2 both utilize the cell-surface receptor, angiotensin converting enzyme 2 (ACE2). Additionally, SARS-CoV-2 S shares 76% amino acid identity with SARS-CoV-1 S. Furthermore, S serves as a dominant antibody target and is a focus of countermeasures for the treatment and prevention of COVID-19 infection. S proteins from the Betacoronavirus genus share multiple regions of structural homology and thus can serve as targets for a cross-reactive antibody response. Identifying cross-reactive antibody epitopes can inform rational design strategies for vaccines and therapies that target multiple highly pathogenic coronaviruses, which can be of value both for the current and potential future outbreaks.

Numerous potent neutralizing antibodies against SARS-CoV-2 have been discovered, including multiple candidates currently in clinical trials for prophylactic and acute treatment of COVID-19. Investigation of SARS-CoV-2/SARS-CoV-1 cross-reactive antibodies has focused primarily on the RBD epitope. This has resulted in the identification of a number of SARS-CoV-2/SARS-CoV-1 cross-reactive antibody candidates. However, for cross-reactive antibodies, the diversity of epitopes and functions other than virus neutralization have not been extensively explored. Evidence of Fc effector function eliciting protection in vivo against SARS-CoV-1 and SARS-CoV-2 indicates the role of antibodies beyond neutralization (“extra-neutralization” functions) can be a crucial component of protection and an important consideration in vaccine design strategies for coronaviruses. Defining the genetic features, epitope targets, and Fc effector functions of cross-reactive antibodies can provide insights into current therapeutic strategies and can provide alternative approaches for the prevention and treatment of coronavirus infection.

In this study, antibody cross-reactivity across the Betacoronavirus genus at monoclonal resolution was investigated. To do this, LIBRA-seq (Linking B Cell receptor to antigen specificity through sequencing) was applied, a recently developed high-throughput antibody screening technology that allows for determination of B cell receptor sequence and antigen reactivity simultaneously for many single B cells. From a convalescent SARS-CoV-1 donor sample, SARS-CoV-2/SARS-CoV-1 cross-reactive human antibodies were identified and characterized that target multiple, distinct structural domains of S, mediate Fc effector functions, and reduce pathological burden in vivo. A better understanding of the epitope specificities and functional characteristics of cross-reactive coronavirus antibodies can translate into strategies for current vaccine design efforts and additional measures to counteract potential future pandemic variants.

LIBRA-seq applied to a SARS-CoV-1 convalescent donor. To identify cross-reactive antibodies to multiple coronavirus antigens, LIBRA-seq was applied to a PBMC sample from a donor previously infected with SARS-CoV-1 twelve years prior to sample collection. The antigen screening library consisted of eight oligo-tagged recombinant soluble antigens: six coronavirus trimer antigens (SARS-CoV-2 S, SARS-CoV-1 S, MERS-CoV S, MERS-CoV S1 (with foldon domain), OC43-CoV S, HKU1-CoV S) and two HIV trimer antigens from strains ZM197 and CZA97 as negative controls (FIG. 14A). After the antigen screening library was mixed with donor PBMCs, antigen positive B cells were enriched by fluorescence activated cell sorting and processed for single-cell sequencing (FIG. 18A). After bioinformatic processing, 2625 cells with paired heavy/light chain sequences and antigen reactivity information were recovered (FIG. 18B). Overall, LIBRA-seq enabled rapid screening of PBMCs from a patient sample, with recovery of paired heavy/light chain sequences and antigen reactivity for thousands of single B cells.

Identification of SARS-CoV-2 and SARS-CoV-1 cross-reactive antibodies. To identify antibodies that were cross-reactive to multiple coronavirus S proteins, antibodies were prioritized based on their sequence features and LIBRA-seq scores. 15 antibodies that exhibited diverse sequence features were selected and a number of different variable genes were utilized for expression and characterization (FIG. 14B, FIG. 18C). These antibodies displayed a broad range of somatic hypermutation levels (83-98%) and a variety of CDRH3 and CDRL3 lengths (6-24 and 5-12 amino acids, respectively) (FIG. 18C). Antibodies 46472-1, 46472-2, 46472-3, 46472-4, 46472-6, and 46472-12 showed binding to SARS-CoV-1 S and SARS-CoV-2 S by ELISA (FIGS. 14C-14D, FIG. 18D). Further, antibodies 46472-6 and 46472-12 bound to S proteins from endemic OC43-CoV and HKU1-CoV, albeit generally at lower levels (FIGS. 14C-14D, FIG. 18D). Although the six monoclonal antibodies showed reactivity by ELISA to the MERS antigen probe used in the LIBRA-seq screening library, antibody binding to other independent preparations of this protein was inconsistent, so MERS S reactivity cannot be definitively confirmed (FIG. 18E). Overall, the application of the LIBRA-seq technology enabled the identification of a panel of cross-reactive coronavirus antibodies that recognize the S antigen from multiple coronaviruses.

Cross-reactive coronavirus antibodies target diverse epitopes on S. To elucidate the epitopes targeted by the cross-reactive antibodies, binding assays to various structural domains of S as well as binding-competition experiments were performed. First, antibody binding to the S1 and S2 subdomains of SARS-CoV-2 was assessed. Antibodies 46472-1, 46472-2, 46472-3, and 46472-4 bound to the S2 domain, whereas 46472-6 and 46472-12 recognized the S1 domain but targeted different epitopes, the NTD and RBD, respectively (FIGS. 15A-15C, FIGS. 19A-19B). Although 46472-12 bound to the RBD, it did not compete with ACE2 for binding to SARS-CoV-2 S (FIG. 19C). To determine whether the antibodies targeted overlapping or distinct epitopes, competition ELISA experiments were performed and it was found that the S2-directed antibodies 46472-1, 46472-2, and 46472-4 competed for binding to S (FIG. 19D). This pattern was observed for both SARS-CoV-2 and SARS-CoV-1 S. Of note, this competition group did not include S2-directed antibody 46472-3, revealing the identification of multiple cross-reactive epitope targets on S2 (FIG. 15D). Further, binding assays with glycan knockout mutants and mannose competition experiments revealed no notable glycan dependence for antibody reactivity to S (FIG. 15D-15E). Lastly, antibody autoreactivity was measured, and it was found that with the exception of 46472-6 binding to Jo-1, none of the antibodies showed autoreactivity against the tested antigens (FIG. 15E). Together, these data show that these cross-reactive antibodies are coronavirus-specific and target multiple, diverse epitopes on the S protein (FIG. 15F).

Functional Characterization of Cross-reactive Coronavirus Antibodies. Next, the cross-reactive antibody panel for functional activity was characterized. Although none of the antibodies neutralized live SARS-CoV-1 or SARS-CoV-2 (FIGS. 20A-20B), all six antibodies demonstrated a range of Fc effector functions. Notably, all antibodies showed antibody-dependent cellular phagocytosis (ADCP) in vitro for SARS-CoV-2 S (FIG. 16A). In particular, antibody 46472-12 that targets RBD showed greater ADCP activity compared to the other cross-reactive antibodies and the RBD antibody control, CR3022 (FIG. 16A, FIG. 20C). Further, ADCP activity against SARS-CoV-1 for two antibodies, 46472-4 and 46472-12, was tested and confirmed, illustrating that these antibodies can mediate antiviral function against multiple coronaviruses (FIG. 16B, FIG. 20D). In a coated SARS-CoV-2 S assay (see Methods), the cross-reactive antibodies also mediated trogocytosis, an Fc-mediated immune function defined by the removal of cell membrane proteins from S-coated and opsonized cells to effector cells, which results in rapid cell death and antigen transfer (FIG. 16C, FIG. 20E). Only the S2-targeting antibodies in the panel (46472-1, 46472-2, 46472-3, and 46472-4) mediated trogocytosis for cell-surface expressed SARS-CoV-2 S (FIG. 16D, FIG. 20F). Lastly, none of the antibodies promoted complement deposition (ADCD) (FIG. 16E, FIG. 20G). Together, these results revealed that although this cross-reactive antibody panel is non-neutralizing, the six antibodies are capable of mediating a spectrum of Fc effector functions.

Since non-neutralizing SARS-CoV-2 antibodies with Fc effector function activity have not been extensively characterized in vivo, antibodies 46472-4 and 46472-12 for prophylaxis in a murine infection model were tested using a mouse-adapted virus strain (SARS-CoV-2 MA)(FIG. 17A). Although there were no differences in survival and viral load between experimental and control groups, the hemorrhage scores (see Methods) for 46472-4 and 46472-12 were similar to positive control CR3022, and all three groups were significantly lower than the scores for isotype control 2D22 (p<0.01, ordinary one-way ANOVA with multiple comparisons), suggesting a reduction in pathological burden (FIG. 17B, FIGS. 21A-21B). To evaluate the in vivo effect of these antibodies in a more stringent challenge model in mice, the viral dose was increased from 1×10³ to 1×10⁴. In this experiment, mice that received antibody 46472-12 exhibited the best survival rate (4/5 at day 4), compared to all other treatment groups (including CR3022 as a positive control and DENV-2D22 as a negative control), although statistical significance was not achieved (FIG. 17C, FIG. 21C). There were no significant differences in viral load between groups; however, the surviving animals from the 46472-4 and 46472-12 groups showed statistically significant lower hemorrhagic pathology scores in harvested mouse lungs compared to the negative control treatment group (p<0.001, ordinary one-way ANOVA with multiple comparisons) (FIG. 17D, FIG. 21D). Notably, animals treated with the positive control, CR3022, had significantly higher hemorrhage scores than animals treated with 46472-4 and 46472-12 (p<0.001, ordinary one-way ANOVA with multiple comparisons) (FIG. 17D). Together, the in vivo experiments show these cross-reactive antibodies can contribute to counteracting coronavirus infection in prophylaxis.

Here, a set of cross-reactive Betacoronavirus antibodies isolated from a convalescent SARS-CoV-1 donor were described. The antibodies targeted diverse epitopes on S, including the S2 subdomain as well as the RBD and NTD on S1, and were shown to be functional in vitro. Additionally, two of these antibodies were tested and demonstrated activity in vivo. While the precise in vivo effects of these antibodies have not been elucidated, the Fc effector function profiles in the absence of neutralization points to a role for extra-neutralization activity to reduce pathological burden, as illustrated for other SARS-CoV-2 antibodies. Evidence of protection associated with Fc effector function in SARS-CoV-1, and other infectious diseases including influenza, Ebola, and HIV, motivates further investigation into its contribution for the treatment of COVID-19.

Given the ongoing SARS-CoV-2 pandemic and for future zoonotic coronavirus pathogens to emerge, coronavirus vaccine and therapeutic development is of paramount importance. Antibodies that can cross-react with multiple coronavirus variants are primary targets as broadly reactive therapies. Such antibodies can further reveal cross-reactive epitopes that can serve as templates for the development of broadly protective vaccines. Understanding the spectrum of cross-reactive epitopes targeted by human antibodies, as well as the functional role that such antibodies have in preventing and treating coronavirus infection, are therefore critical for medical countermeasure development. In particular, the identification of functional cross-reactive antibodies that target diverse epitopes on S can present a viable avenue for pan-coronavirus vaccine design strategies.

Methods.

Donor Information. The donor had prior SARS-CoV-1 infection during the SARS-CoV-1 outbreak in 2004, and the PBMC sample was collected ˜12 years post infection (20 million PBMCs).

Antigen Purification. A variety of recombinant soluble protein antigens were used in the LIBRA-seq experiment and other experimental assays. Plasmids encoding residues 1-1208 of the SARS-CoV-2 spike with a mutated S1/S2 cleavage site, proline substitutions at positions 986 and 987, and a C-terminal T4-fibritin trimerization motif, an 8× HisTag, and a TwinStrepTag (SARS-CoV-2 S-2P); residues 1-1190 of the SARS-CoV-1 spike with proline substitutions at positions 968 and 969, and a C-terminal T4-fibritin trimerization motif, an 8× HisTag, and a TwinStrepTag (SARS-CoV-1 S-2P); residues 1-1291 of the MERS-CoV spike with a mutated S1/S2 cleavage site, proline substitutions at positions 1060 and 1061, and a C-terminal T4-fibritin trimerization motif, an AviTag, an 8× HisTag, and a TwinStrepTag (MERS-CoV S-2P Avi); residues 1-751 of the MERS-CoV spike with a C-terminal 8× HisTag, and a TwinStrepTag (MERS-CoV 51); residues 1-1277 of the HCoV-HKU1 spike with a mutated S1/S2 cleavage site, proline substitutions at positions 1067 and 1068, and a C-terminal T4-fibritin trimerization motif, an 8×HisTag, and a TwinStrepTag (HCoV-HKU1 S-2P); residues 1-1278 of the HCoV-OC43 spike with proline substitutions at positions 1070 and 1071, and a C-terminal T4-fibritin trimerization motif, an 8× HisTag, and a TwinStrepTag (HCoV-OC43 S-2P); or residues 319-591 of SARS-CoV-2 S with a C-terminal monomeric human IgG Fc-tag and an 8× HisTag (SARS-CoV-2 RBD-SD1) were transiently transfected into FreeStyle293F cells (Thermo Fisher) using polyethylenimine. The coronavirus trimer spike antigens were in a prefusion-stabilized (S-2P) conformation that better represents neutralization-sensitive epitopes in comparison to their wild-type forms. Two hours post-transfection, cells were treated with kifunensine to ensure uniform glycosylation. Transfected supernatants were harvested after 6 days of expression. SARS-CoV-2 RBD-SD1 was purified using Protein A resin (Pierce), SARS-CoV-2 S-2P, SARS-CoV-1 S-2P, MERS-CoV S-2P Avi, MERS-CoV S1, HCoV-HKU1 S-2P and HCoV-OC43 S-2P were purified using StrepTactin resin (IBA). Affinity-purified SARS-CoV-2 RBD-SD1 was further purified over a Superdex75 column (GE Life Sciences). MERS-CoV S1 was purified over a Superdex200 Increase column (GE Life Sciences). SARS-CoV-2 S-2P, SARS-CoV-1 S-2P, MERS-CoV S-2P Avi, HCoV-HKU1 S-2P and HCoV-OC43 S-2P were purified over a Superose6 Increase column (GE Life Sciences).

For the HIV-1 gp140 SOSIP variant from strain ZM197 (clade C) and CZA97 (clade C), recombinant, soluble antigens contained an AviTag and were expressed in Expi293F cells using polyethylenimine (PEI) transfection reagent and cultured. FreeStyle F17 expression medium supplemented with pluronic acid and glutamine was used. The cells were cultured at 37° C. with 8% CO₂ saturation and shaking. After 5-7 days, cultures were centrifuged and supernatant was filtered and run over an affinity column of agarose bound Galanthus nivalis lectin. The column was washed with PBS and antigens were eluted with 30 mL of 1M methyl-a-D-mannopyranoside. Protein elutions were buffer exchanged into PBS, concentrated, and run on a Superdex 200 Increase 10/300 GL Sizing column on the AKTA FPLC system. Fractions corresponding to correctly folded protein were collected, analyzed by SDS-PAGE and antigenicity was characterized by ELISA using known monoclonal antibodies specific to each antigen. Avitagged antigens were biotinylated using BirA biotin ligase (Avidity LLC).

For binding studies, SARS-CoV-2 HexaPro S, SARS-CoV-1 S, SARS-CoV-2 RBD, SARS-CoV-1 RBD, and MERS-CoV RBD constructs were expressed in the transient expression system previously mentioned. S proteins were purified using StrepTrap HP columns and RBD constructs were purified over protein A resin, respectively. Each resulting protein was further purified to homogeneity by size-exclusion chromatography on a Superose 6 10/300 GL column.

SARS-CoV-2 S1, S1 D614G, S2, NTD truncated proteins were purchased from commercial vendor Sino Biological.

DNA-barcoding of Antigens. Oligos that possess 15 bp antigen barcode were used, a sequence capable of annealing to the template switch oligo that is part of the 10× bead-delivered oligos, and contain truncated TruSeq small RNA read 1 sequences in the following structure: 5′-CCTTGGCACCCGAGAATTCCA CCCATATAAGA*A*A-3′ (SEQ ID NO: 26455), where Ns represent the antigen barcode, and * represents phosphiorate bond modification that can increase oligonucleotide stability. The following antigen barcodes were used: GCTCCTTTACACGTA (SEQ ID NO: 26447) (SARS-CoV-2 S), TGACCTTCCTCTCCT (SEQ ID NO: 26448) (SARS-CoV-1 S), ACAATTTGTCTGCGA (SEQ ID NO: 26449) (MERS-CoV S), TCCTTTCCTGATAGG (SEQ ID NO: 26450) (MERS-CoV 51), CAGGTCCCTTATTTC (SEQ ID NO: 26451) (HKU1-CoV S), TAACTCAGGGCCTAT (SEQ ID NO: 26452) (OC43-CoV S), CAGCCCACTGCAATA (SEQ ID NO: 26453) (CZA97), and ATCGTCGAGAGCTAG (SEQ ID NO: 26454) (ZM197). Oligos were ordered from Sigma-Aldrich and IDT with a 5′ amino modification and HPLC purified.

For each antigen, a unique DNA barcode was directly conjugated to the antigen itself. In particular, 5′amino-oligonucleotides were conjugated directly to each antigen using the Solulink Protein-Oligonucleotide Conjugation Kit (TriLink cat no. S-9011) according to manufacturer's instructions. Briefly, the oligo and protein were desalted, and then the amino-oligo was modified with the 4FB crosslinker, and the biotinylated antigen protein was modified with S-HyNic. Then, the 4FB-oligo and the HyNic-antigen were mixed together. This causes a stable bond to form between the protein and the oligonucleotide. The concentration of the antigen-oligo conjugates was determined by a BCA assay, and the HyNic molar substitution ratio of the antigen-oligo conjugates was analyzed using the NanoDrop according to the Solulink protocol guidelines. AKTA FPLC was used to remove excess oligonucleotide from the protein-oligo conjugates, which were also verified using SDS-PAGE with a silver stain. Antigen-oligo conjugates were also used in flow cytometry titration experiments.

Antigen specific B cell sorting. Cells were stained and mixed with DNA-barcoded antigens and other antibodies, and then sorted using fluorescence activated cell sorting (FACS). First, cells were counted and viability was assessed using Trypan Blue. Then, cells were washed 3× with DPBS supplemented with 0.1% Bovine serum albumin (BSA). Cells were resuspended in DPBS-BSA and stained with cell markers including viability dye (Ghost Red 780), CD14-APCCy7, CD3-FITC, CD19-BV711, and IgG-PECy5. Additionally, antigen-oligo conjugates were added to the stain. After staining in the dark for 30 minutes at room temperature, cells were washed 3 times with PBS-BSA at 300 g for 5 minutes. Cells were then incubated for 15 minutes at room temperature with Streptavidin-PE to label cells with bound antigen. Cells were washed with DPBS-BSA, resuspended in DPBS, and sorted by FACS. Antigen positive cells were bulk sorted and delivered to the Vanderbilt Technologies for Advanced Genomics (VANTAGE) sequencing core at an appropriate target concentration for 10× Genomics library preparation and subsequent sequencing. FACS data were analyzed using FlowJo.

Sample preparation, library preparation, and sequencing. Single-cell suspensions were loaded onto the Chromium Controller microfluidics device (10× Genomics) and processed using the B-cell Single Cell V(D)J solution according to manufacturer's suggestions for a target capture of 10,000 B cells per ⅛ 10× cassette, with minor modifications in order to intercept, amplify and purify the antigen barcode libraries as previously described.

Sequence processing and bioinformatic analysis. The previously described pipeline was utilized and modified to use paired-end FASTQ files of oligo libraries as input, processes and annotates reads for cell barcode, UMI, and antigen barcode, and generates a cell barcode-antigen barcode UMI count matrix. BCR contigs were processed using Cell Ranger (10× Genomics) using GRCh38 as reference. Antigen barcode libraries were also processed using Cell Ranger (10×Genomics). The overlapping cell barcodes between the two libraries were used as the basis of the subsequent analysis. Cell barcodes that had only non-functional heavy chain sequences as well as cells with multiple functional heavy chain sequences and/or multiple functional light chain sequences were removed, reasoning that these can be multiplets. Additionally, the BCR contigs (filtered_contigs.fasta file output by Cell Ranger, 10× Genomics) was aligned to IMGT reference genes using HighV-Quest. The output of HighV-Quest was parsed using ChangeO, and merged with an antigen barcode UMI count matrix. Finally, it was determined the LIBRA-seq score for each antigen in the library for every cell.

Antibody Expression and Purification. For each antibody, variable genes were inserted into custom plasmids encoding the constant region for the IgG1 heavy chain as well as respective lambda and kappa light chains (pTwist CMV BetaGlobin WPRE Neo vector, Twist Bioscience). mAbs were expressed in Expi293F mammalian cells (ThermoFisher) by co-transfecting heavy chain and light chain expressing plasmids using PEI transfection reagent and cultured for 5-7 days. Cells were maintained in FreeStyle F17 expression medium supplemented at final concentrations of 0.1% Pluronic Acid F-68 and 20% 4 mM L-Glutamine. These cells were cultured at 37° C. with 8% CO₂ saturation and shaking. After transfection and 5-7 days of culture, cell cultures were centrifuged and supernatant was 0.45 μm filtered with Nalgene Rapid Flow Disposable Filter Units with PES membrane. Filtered supernatant was run over a column containing Protein A agarose resin equilibrated with PBS. The column was washed with PBS, and then antibodies were eluted with 100 mM Glycine HCl at 2.7 pH directly into a 1:10 volume of 1M Tris-HCl pH 8.0. Eluted antibodies were buffer exchanged into PBS 3 times using Amicon Ultra centrifugal filter units and concentrated. Antibodies were analyzed by SDS-PAGE. Additionally, antibodies 46472-1, 46472-2, 46472-3, 46472-4, 46472-6 and 46472-12 were assessed by size exclusion chromatography on a Superdex 200 Increase 10/300 GL Sizing column with the AKTA FPLC system.

ELISA. To assess antibody binding, soluble protein was plated at 2 μg/ml overnight at 4° C. The next day, plates were washed three times with PBS supplemented with 0.05% Tween-20 (PBS-T) and coated with 5% milk powder in PBS-T. Plates were incubated for one hour at room temperature and then washed three times with PBS-T. Primary antibodies were diluted in 1% milk in PBS-T, starting at 10 μg/ml with a serial 1:5 dilution and then added to the plate. The plates were incubated at room temperature for one hour and then washed three times in PBS-T. The secondary antibody, goat anti-human IgG conjugated to peroxidase, was added at 1:10,000 dilution in 1% milk in PBS-T to the plates, which were incubated for one hour at room temperature. Goat anti-mouse secondary was used for SARS-CoV-1 specific control antibody 240CD (BEI Resources). Plates were washed three times with PBS-T and then developed by adding TMB substrate to each well. The plates were incubated at room temperature for ten minutes, and then IN sulfuric acid was added to stop the reaction. Plates were read at 450 nm.

Data are represented as mean±SEM for one ELISA experiment. ELISAs were repeated 2 or more times. The area under the curve (AUC) was calculated using GraphPad Prism 8.0.0. For antibody 240CD, the following reagent was obtained through BEI Resources, NIAID, NIH: Monoclonal Anti-SARS-CoV S Protein (Similar to 240C), NR-616.

Competition ELISA. Competition ELISAs were performed as described above, with some modifications. After coating with antigen and blocking, 25 μl of non-biotinylated competitor antibody was added to each well at 10 μg/ml and incubated at 37° C. for 10 minutes. Then, without washing, 75 μl biotinylated antibody (final concentration of 1 μg/ml) was added and incubated at 37° C. for 1 hour. After washing three times with PBS-T, streptavidin-HRP was added at 1:10,000 dilution in 1% milk in PBS-T and incubated for 1 hour at room temperature. Plates were washed and substrate and sulfuric acid were added as described above. ELISAs were repeated at least 2 times. Data is shown as the % decrease in binding.

Autoreactivity. Monoclonal antibody reactivity to nine autoantigens (SSA/Ro, SS-B/La, Sm, ribonucleoprotein (RNP), Scl 70, Jo-1, dsDNA, centromere B, and histone) was measured using the AtheNA Multi-Lyte® ANA-II Plus test kit (Zeus scientific, Inc, #A21101). Antibodies were incubated with AtheNA beads for 30 min at concentrations of 50, 25, 12.5 and 6.25 μg/mL. Beads were washed, incubated with secondary and read on the Luminex platform as specified in the kit protocol. Data were analyzed using AtheNA software. Positive (+) specimens received a score >120, and negative (−) specimens received a score <100. Samples between 100-120 were considered indeterminate.

Mannose competition. Mannose competition ELISAs were performed as described above with minor modifications. After antigen coating and washing, nonspecific binding was blocked by incubation with 5% FBS diluted in PBS for 1 hour at RT. Primary antibodies were diluted in 5% FBS-PBST+/−1M D-(+)-Mannose starting at 10 μg/ml with a serial 1:5 dilution and then added to the plate for 1 hour at RT. After washing, antibody binding was detected with goat anti-human IgG conjugated to peroxidase and added at 1:10,000 dilution in 5% FBS in PBS-T to the plates. After 1 hour incubation, plates were washed and substrate and sulfuric acid were added as described above. Data shown is representative of three replicates.

Epitope Mapping Visualization. SARS-CoV-2 Spike (PDB-6VSB) was visualized using PyMOL software. Antibody epitopes were visualized on the SARS-CoV-2 spike using a structure of the pre-fusion stabilized SARS-CoV-2 S-2P construct modeled in the molecular graphics software PyMOL (The PyMOL Molecular Graphics System, Version 2.3.5 Schrödinger, LLC.)

Neutralization Assay 1 RTCA. To assess for neutralizing activity against SARS-CoV-2 strain 2019 n-CoV/USA_WA1/2020 (obtained from the Centers for Disease Control and Prevention, a gift from N. Thornburg), the high-throughput RTCA assay and xCelligence RTCA HT Analyzer (ACEA Biosciences) were used. After obtaining a background reading of a 384-well E-plate, 6,000 Vero-furin cells were seeded per well. Sensograms were visualized using RTCA HT software version 1.0.1 (ACEA Biosciences). One day later, equal volumes of virus were added to antibody samples and incubated for 1 h at 37° C. in 5% CO₂. mAbs were tested in triplicate with a single (1:20) dilution. Virus—mAb mixtures were then added to Vero-furin cells in 384-well E-plates. Controls were included that had Vero-furin cells with virus only (no mAb) and media only (no virus or mAb). E-plates were read every 8-12 h for 72 h to monitor virus neutralization. At 32 h after virus-mAb mixtures were added to the E-plates, cell index values of antibody samples were compared to those of virus only and media only to determine presence of neutralization.

Neutralization Assay 2 nano-luciferase reporter system. A full-length SARS-CoV-2 virus based on the Seattle Washington isolate was designed to express luciferase and GFP and was recovered via reverse genetics and described previously. The virus was titered in Vero E6 USAMRID cells to obtain a relative light units (RLU) signal of at least 10× the cell only control background. Vero E6 USAMRID cells were plated at 20,000 cells per well the day prior in clear bottom black walled 96-well plates (Corning 3904). Neutralizing antibody serum samples were tested at a starting dilution of 1:40 and were serially diluted 4-fold up to eight dilution spots. Antibody-virus complexes were incubated at 37C with 5% CO₂ for 1 hour. Following incubation, growth media was removed and virus-antibody dilution complexes were added to the cells in duplicate. Virus-only controls and cell-only controls were included in each neutralization assay plate. Following infection, plates were incubated at 37C with 5% CO₂ for 48 hours. After the 48 hour incubation, cells were lysed and luciferase activity was measured via Nano-Glo Luciferase Assay System (Promega) according to the manufacturer specifications. SARS-CoV-2 neutralization titers were defined as the sample dilution at which a 50% reduction in RLU was observed relative to the average of the virus control wells.

SPR. His-tagged SARS-CoV RBD-SD1 was immobilized to a NiNTA sensorchip to a level of ˜150 RUs using a Biacore X100. Serial dilutions of purified Fab 12 were evaluated for binding, ranging in concentration from 1 to 0.25 μM. The resulting data were fit to a 1:1 binding model using Biacore Evaluation Software.

Fc Effector Function Assays

Antibody-dependent Cellular Phagocytosis (ADCP). Antibody-dependent cellular phagocytosis (ADCP) was performed using biotinylated SARS-CoV-2 or SARS-CoV-1 S ConC coated fluorescent neutravidin beads. Briefly, beads were incubated for two hours with antibodies at a starting concentration of 50 μg/ml and titrated five fold. CR3022 was used as a positive control while Palivizumab was used as a negative control. Antibodies and beads were incubated with THP-1 cells overnight, fixed and interrogated on the FACSAria II. Phagocytosis score was calculated as the percentage of THP-1 cells that engulfed fluorescent beads multiplied by the geometric mean fluorescence intensity of the population in the FITC channel less the no antibody control.

Antibody-dependent Cellular Trogocytosis (ADCT). ADCT was performed as described in and modified from a previously described study. HEK293T-ACE2 expressing cells were pulsed with SARS-CoV-2 S protein (10 μg/ml) for 75 minutes or HEK293T cells transfected with a SARS-CoV-2 spike pcDNA vector were surface biotinylated with EZ-Link Sulfo-NHS-LC-Biotin as recommended by the manufacturer. Fifty-thousand cells per well were incubated with antibody for 30 minutes starting at 25 μg/ml and titrated 5 fold. CR3022 was used as a positive control with Palivizumab as a negative. Following a RPMI media wash, these were then incubated with carboxyfluorescein succinimidyl ester (CFSE) stained THP-1 cells (5×10⁴ cells per well) for 1 hour and washed with 15 mM EDTA/PBS followed by PBS. Cells were then stained for biotin using Streptavidin-PE and read on a FACSAria II. Trogocytosis score was determined as the proportion of CFSE positive THP-1 cells also positive for streptavidin-PE less the no antibody control.

Antibody-dependent Complement Deposition (ADCD). Antibody-dependent complement deposition was performed. Briefly biotinylated SARS-Cov-2 S protein was coated 1:1 onto fluorescent neutravidin beads for 2 hours at 37 degrees. These beads were incubated with 100 ug/ml of antibody for 1 hour and incubated with guinea pig complement diluted 1 in 50 with gelatin/veronal buffer for 15 minutes at 37 degrees. Beads were washed at 2000 g twice in PBS and stained with guinea pig C3b-FITC, fixed and interrogated on a FACSAria II. Complement deposition score was calculated as the percentage of C3b-FITC positive beads multiplied by the geometric mean fluorescent intensity of FITC in this population less the no antibody or heat inactivated controls

Antibody Prophylaxis—Murine Model of Infection. 12 month old BALB/c mice were treated with 200 ug of mAb administered by intraperitoneal injection 12 hours prior to virus inoculation. The next day, mice were administered 1×10⁴ PFU SARS-CoV-2 MA10. Weights were taken each day, and after four days one lung lobe was taken for pathological analysis and the other lobe was processed for qPCR and viral load determination.

For viral titer and hermorrhage score comparisons, an ordinary one-way ANOVA test with multiple comparisons was performed using Prism software, GraphPad Prism version 8.0.

ACE2 Binding Inhibition Assay. Wells of 384-well microtiter plates were coated with purified recombinant SARS-CoV-2 S-2P ectoprotein at 4° C. overnight. Plates were blocked with 2% non-fat dry milk and 2% normal goat serum in DPBS-T for 1 hr. Purified mAbs from microscale expression were diluted two-fold in blocking buffer starting from 10 μg/mL in triplicate, added to the wells (20 μL/well), and incubated at ambient temperature. Recombinant human ACE2 with a C-terminal FLAG tag protein was added to wells at 2 μg/mL in a 5 μL/well volume (final 0.4 μg/mL concentration of ACE2) without washing of antibody and then incubated for 40 min at ambient temperature. Plates were washed, and bound ACE2 was detected using HRP-conjugated anti-FLAG antibody and TMB substrate. ACE2 binding without antibody served as a control. The signal obtained for binding of the ACE2 in the presence of each dilution of tested antibody was expressed as a percentage of the ACE2 binding without antibody after subtracting the background signal.

Computational Identification of Cross-reactive Epitopes. Cross-reactive epitopes were identified based on sequence and structural homology. Reference sequences for each Coronavirus S used in the LIBRA-seq run were obtained either from NCBI for SARS-CoV-2 (YP_009724390.1) and MERS-CoV (YP_009047204.1) or from Uniprot for SARS-CoV-1 (P59594), HKU1-CoV (Q5MQD0), and OC43-CoV (P36334). A multiple sequence alignment of all 5 spikes was then obtained using MUSCLE and the amino acid similarity to SARS-CoV-2 at each residue position was calculated using the BLOSUM-62 scoring matrix. These scores were then used to color each residue position on the SARS-CoV-2 S structure (PDB ID: 6VSB) in PyMOL (Schrodinger, version 2.3.5) in order to visualize surface patches and linear epitopes with structural homology. These conserved regions were then visualized on the other human coronavirus spike structures by retrieving them from the Protein Databank (SARS-CoV-1: 5X5B, MERS-CoV: 5W9I, OC43-CoV: 6OHW, HKU1-CoV: 5I08) and aligning them to the SARS-CoV-2 S structure. Finally, residues at prominent positions of conserved surface patches were chosen to be mutated and tested for altered binding with antibodies.

Quantification and Statistical Analysis. ELISA error bars (standard error of the mean) were calculated using GraphPad Prism version 8.0.0. ANOVA analysis was performed on viral load titers and hemorrhage scores from animal experiments using GraphPad Prism version 8.0.0.

In the examples above, large numbers of antibody sequences were determined (see sequences provided below). The following paired heavy chain and light chain sequences are used herein for methods of treating, preventing, or detecting coronavirus infections.

TABLE 1
Paired heavy and light chains and the CDRs thereof

| | Cell Barcode | | | | | Cell Barcode | | | | |
|---|---|---|---|---|---|---|---|---|---|---|
| Antibody | and Heavy | V-D-J | | | | and Light | V-J | | | |
| Name | Chain (HC) | (HC) | CDRH1 | CDRH2 | CDRH3 | Chain (LC) | (LC) | CDRL1 | CDRL2 | CDRL3 |
| 46472-12 | AATCCAGGTGT | SEQ ID | SEQ ID | SEQ ID | SEQ ID | AATCCAGGTGTT | SEQ ID | SEQ ID | SEQ ID | SEQ ID |
| | TTGGT (SEQ ID | NO: | NO: | NO: | NO: | TGGT(SEQ ID | NO: | NO: | NO: | NO: |
| | NO: 1).HC | 37 | 73 | 91 | 109 | NO: 2).LC | 55 | 127 | 145 | 163 |
| 46472-8 | ACAGCCGCAG | SEQ ID | SEQ ID | SEQ ID | SEQ ID | ACAGCCGCAGT | SEQ ID | SEQ ID | SEQ ID | SEQ ID |
| | TGACAG(SEQ | NO: | NO: | NO: | NO: | GACAG(SEQ ID | NO: | NO: | NO: | NO: |
| | ID NO: 3).HC | 38 | 74 | 92 | 110 | NO: 4).LC | 56 | 128 | 146 | 164 |
| 46472-5 | ACGCCAGCATC | SEQ ID | SEQ ID | SEQ ID | SEQ ID | ACGCCAGCATC | SEQ ID | SEQ ID | SEQ ID | SEQ ID |
| | ACGAT(SEQ ID | NO: | NO: | NO: | NO: | ACGAT(SEQ ID | NO: | NO: | NO: | NO: |
| | NO: 5).HC | 39 | 75 | 93 | 111 | NO: 6).LC | 57 | 129 | 147 | 165 |
| 46472-13 | ACTGAGTCAA | SEQ ID | SEQ ID | SEQ ID | SEQ ID | ACTGAGTCAAG | SEQ ID | SEQ ID | SEQ ID | SEQ ID |
| | GTTGTC(SEQ | NO: | NO: | NO: | NO: | TTGTC(SEQ ID | NO: | NO: | NO: | NO: |
| | ID NO: 7).HC | 40 | 76 | 94 | 112 | NO: 8).LC | 58 | 130 | 148 | 166 |
| 46472-15 | AGTGGGACAC | SEQ ID | SEQ ID | SEQ ID | SEQ ID | AGTGGGACACG | SEQ ID | SEQ ID | SEQ ID | SEQ ID |
| | GTAAGG(SEQ | NO: | NO: | NO: | NO: | TAAGG(SEQ ID | NO: | NO: | NO: | NO: |
| | ID NO: 9).HC | 41 | 77 | 95 | 113 | NO: 10).LC | 59 | 131 | 149 | 167 |
| 46472-2 | CATTCGCAGTC | SEQ ID | SEQ ID | SEQ ID | SEQ ID | CATTCGCAGTCT | SEQ ID | SEQ ID | SEQ ID | SEQ ID |
| | TCGGC(SEQ ID | NO: | NO: | NO: | NO: | CGGC(SEQ ID | NO: | NO: | NO: | NO: |
| | NO: 11).HC | 42 | 78 | 96 | 114 | NO: 12).LC | 60 | 132 | 150 | 168 |
| 46472-14 | CGAGCACGTCT | SEQ ID | SEQ ID | SEQ ID | SEQ ID | CGAGCACGTCTT | SEQ ID | SEQ ID | SEQ ID | SEQ ID |
| | TCTCG(SEQ ID | NO: | NO: | NO: | NO: | CTCG(SEQ ID | NO: | NO: | NO: | NO: |
| | NO: 13).HC | 43 | 79 | 97 | 115 | NO: 14).LC | 61 | 133 | 151 | 169 |
| 46472-9 | CTGATCCCATT | SEQ ID | SEQ ID | SEQ ID | SEQ ID | CTGATCCCATTC | SEQ ID | SEQ ID | SEQ ID | SEQ ID |
| | CTCAT(SEQ ID | NO: | NO: | NO: | NO: | TCAT(SEQ ID | NO: | NO: | NO: | NO: |
| | NO: 15).HC | 44 | 80 | 98 | 116 | NO: 16).LC | 62 | 134 | 152 | 170 |
| 46472-3 | GACTACAAGG | SEQ ID | SEQ ID | SEQ ID | SEQ ID | GACTACAAGGG | SEQ ID | SEQ ID | SEQ ID | SEQ ID |
| | GCATGT(SEQ | NO: | NO: | NO: | NO: | CATGT(SEQ ID | NO: | NO: | NO: | NO: |
| | ID NO: 17).HC | 45 | 81 | 99 | 117 | NO: 18).LC | 63 | 135 | 153 | 171 |
| 46472-10 | GTACTTTGTGA | SEQ ID | SEQ ID | SEQ ID | SEQ ID | GTACTTTGTGAT | SEQ ID | SEQ ID | SEQ ID | SEQ ID |
| | TAAAC(SEQ ID | NO: | NO: | NO: | NO: | AAAC(SEQ ID | NO: | NO: | NO: | NO: |
| | NO: 19).HC | 46 | 82 | 100 | 118 | NO: 20).LC | 64 | 136 | 154 | 172 |
| 46472-11 | TAGGCATGTA | SEQ ID | SEQ ID | SEQ ID | SEQ ID | TAGGCATGTAA | SEQ ID | SEQ ID | SEQ ID | SEQ ID |
| | AACCTC(SEQ | NO: | NO: | NO: | NO: | ACCTC(SEQ ID | NO: | NO: | NO: | NO: |
| | ID NO: 21).HC | 47 | 83 | 101 | 119 | NO: 22).LC | 65 | 137 | 155 | 173 |
| 46472-6 | TCAACGACAG | SEQ ID | SEQ ID | SEQ ID | SEQ ID | TCAACGACAGA | SEQ ID | SEQ ID | SEQ ID | SEQ ID |
| | ACGTAG(SEQ | NO: | NO: | NO: | NO: | CGTAG(SEQ ID | NO: | NO: | NO: | NO: |
| | ID NO: 23).HC | 48 | 84 | 102 | 120 | NO: 24).LC | 66 | 138 | 156 | 174 |
| 46472-1 | TCAGATGGTTA | SEQ ID | SEQ ID | SEQ ID | SEQ ID | TCAGATGGTTAT | SEQ ID | SEQ ID | SEQ ID | SEQ ID |
| | TCGGT(SEQ ID | NO: | NO: | NO: | NO: | CGGT(SEQ ID | NO: | NO: | NO: | NO: |
| | NO: 25).HC | 49 | 85 | 103 | 121 | NO: 26).LC | 67 | 139 | 157 | 175 |
| 46472-7 | TCAGCAATCAT | SEQ ID | SEQ ID | SEQ ID | SEQ ID | TCAGCAATCATC | SEQ ID | SEQ ID | SEQ ID | SEQ ID |
| | CTGTT(SEQ ID | NO: | NO: | NO: | NO: | TGTT(SEQ ID | NO: | NO: | NO: | NO: |
| | NO: 27).HC | 50 | 86 | 104 | 122 | NO: 28).LC | 68 | 140 | 158 | 176 |
| 46472-4 | TTCTCCTTCTAC | SEQ ID | SEQ ID | SEQ ID | SEQ ID | TTCTCCTTCTACT | SEQ ID | SEQ ID | SEQ ID | SEQ ID |
| | TATC(SEQ ID | NO: | NO: | NO: | NO: | ATC(SEQ ID NO: | NO: | NO: | NO: | NO: |
| | NO: 29).HC | 51 | 87 | 105 | 123 | 30).LC | 69 | 141 | 159 | 177 |
| 46472- | TAGGCATGTA | SEQ ID | SEQ ID | SEQ ID | SEQ ID | TAGGCATGTAA | SEQ ID | SEQ ID | SEQ ID | SEQ ID |
| 11lns | AACCTC(SEQ | NO: | NO: | NO: | NO: | ACCTC(SEQ ID | NO: | NO: | NO: | NO: |
| | ID NO: 31)2.HC | 52 | 88 | 106 | 124 | NO: 32)2.LC | 70 | 142 | 160 | 178 |
| 46472-16 | ACGATACCAAT | SEQ ID | SEQ ID | SEQ ID | SEQ ID | ACGATACCAAT | SEQ ID | SEQ ID | SEQ ID | SEQ ID |
| | GCCAT(SEQ ID | NO: | NO: | NO: | NO: | GCCAT(SEQ ID | NO: | NO: | NO: | NO: |
| | NO: 33).HC | 53 | 89 | 107 | 125 | NO: 34).LC | 71 | 143 | 161 | 179 |
| 46472-17 | TGCCAAAAGC | SEQ ID | SEQ ID | SEQ ID | SEQ ID | TGCCAAAAGCG | SEQ ID | SEQ ID | SEQ ID | SEQ ID |
| | GGATCA(SEQ | NO: | NO: | NO: | NO: | GATCA(SEQ ID | NO: | NO: | NO: | NO: |
| | ID NO: 35).HC | 54 | 90 | 108 | 126 | NO: 36).LC | 72 | 144 | 162 | 180 |

TABLE 2
Additional paired heavy and light chains and the CDRs thereof

| SEQ ID NO | | | | | SEQ ID NO | | | | |
|---|---|---|---|---|---|---|---|---|---|
| of Cell | | | | | of Cell | | | | |
| Barcode | | | | | Barcode | | | | |
| and Heavy | | | | | and Light | | | | |
| Chain (HC) | VH | CDRH1 | CDRH2 | CDRH3 | Chain (LC) | VL | CDRL1 | CDRL2 | CDRL3 |
| designation | SEQ ID NO | SEQ ID NO | SEQ ID NO | SEQ ID NO | designation | SEQ ID NO | SEQ ID NO | SEQ ID NO | SEQ ID NO |
| 181 | 5437 | 10693 | 13312 | 15935 | 182 | 8065 | 18563 | 21191 | 23819 |
| 183 | 5438 | 10694 | 13313 | 15936 | 184 | 8066 | 18564 | 21192 | 23820 |
| 185 | 5439 | 10695 | 13314 | 15937 | 186 | 8067 | 18565 | 21193 | 23821 |
| 187 | 5440 | 10696 | 13315 | 15938 | 188 | 8068 | 18566 | 21194 | 23822 |
| 189 | 5441 | 10697 | 13316 | 15939 | 190 | 8069 | 18567 | 21195 | 23823 |
| 191 | 5442 | 10698 | 13317 | 15940 | 192 | 8070 | 18568 | 21196 | 23824 |
| 193 | 5443 | 10699 | 13318 | 15941 | 194 | 8071 | 18569 | 21197 | 23825 |
| 195 | 5444 | 10700 | 13319 | 15942 | 196 | 8072 | 18570 | 21198 | 23826 |
| 197 | 5445 | 10701 | 13320 | 15943 | 198 | 8073 | 18571 | 21199 | 23827 |
| 199 | 5446 | 10702 | 13321 | 15944 | 200 | 8074 | 18572 | 21200 | 23828 |
| 201 | 5447 | 10703 | 13322 | 15945 | 202 | 8075 | 18573 | 21201 | 23829 |
| 203 | 5448 | 10704 | 13323 | 15946 | 204 | 8076 | 18574 | 21202 | 23830 |
| 205 | 5449 | 10705 | 13324 | 15947 | 206 | 8077 | 18575 | 21203 | 23831 |
| 207 | 5450 | 10706 | 13325 | 15948 | 208 | 8078 | 18576 | 21204 | 23832 |
| 209 | 5451 | 10707 | 13326 | 15949 | 210 | 8079 | 18577 | 21205 | 23833 |
| 211 | 5452 | 10708 | 13327 | 15950 | 212 | 8080 | 18578 | 21206 | 23834 |
| 213 | 5453 | 10709 | 13328 | 15951 | 214 | 8081 | 18579 | 21207 | 23835 |
| 215 | 5454 | 10710 | 13329 | 15952 | 216 | 8082 | 18580 | 21208 | 23836 |
| 217 | 5455 | 10711 | 13330 | 15953 | 218 | 8083 | 18581 | 21209 | 23837 |
| 219 | 5456 | 10712 | 13331 | 15954 | 220 | 8084 | 18582 | 21210 | 23838 |
| 221 | 5457 | 10713 | 13332 | 15955 | 222 | 8085 | 18583 | 21211 | 23839 |
| 223 | 5458 | 10714 | 13333 | 15956 | 224 | 8086 | 18584 | 21212 | 23840 |
| 225 | 5459 | 10715 | 13334 | 15957 | 226 | 8087 | 18585 | 21213 | 23841 |
| 227 | 5460 | 10716 | 13335 | 15958 | 228 | 8088 | 18586 | 21214 | 23842 |
| 229 | 5461 | 10717 | 13336 | 15959 | 230 | 8089 | 18587 | 21215 | 23843 |
| 231 | 5462 | 10718 | 13337 | 15960 | 232 | 8090 | 18588 | 21216 | 23844 |
| 233 | 5463 | 10719 | 13338 | 15961 | 234 | 8091 | 18589 | 21217 | 23845 |
| 235 | 5464 | 10720 | 13339 | 15962 | 236 | 8092 | 18590 | 21218 | 23846 |
| 237 | 5465 | 10721 | 13340 | 15963 | 238 | 8093 | 18591 | 21219 | 23847 |
| 239 | 5466 | 10722 | 13341 | 15964 | 240 | 8094 | 18592 | 21220 | 23848 |
| 241 | 5467 | 10723 | 13342 | 15965 | 242 | 8095 | 18593 | 21221 | 23849 |
| 243 | 5468 | 10724 | 13343 | 15966 | 244 | 8096 | 18594 | 21222 | 23850 |
| 245 | 5469 | 10725 | 13344 | 15967 | 246 | 8097 | 18595 | 21223 | 23851 |
| 247 | 5470 | 10726 | 13345 | 15968 | 248 | 8098 | 18596 | 21224 | 23852 |
| 249 | 5471 | 10727 | 13346 | 15969 | 250 | 8099 | 18597 | 21225 | 23853 |
| 251 | 5472 | 10728 | 13347 | 15970 | 252 | 8100 | 18598 | 21226 | 23854 |
| 253 | 5473 | 10729 | 13348 | 15971 | 254 | 8101 | 18599 | 21227 | 23855 |
| 255 | 5474 | 10730 | 13349 | 15972 | 256 | 8102 | 18600 | 21228 | 23856 |
| 257 | 5475 | 10731 | 13350 | 15973 | 258 | 8103 | 18601 | 21229 | 23857 |
| 259 | 5476 | 10732 | 13351 | 15974 | 260 | 8104 | 18602 | 21230 | 23858 |
| 261 | 5477 | 10733 | 13352 | 15975 | 262 | 8105 | 18603 | 21231 | 23859 |
| 263 | 5478 | 10734 | 13353 | 15976 | 264 | 8106 | 18604 | 21232 | 23860 |
| 265 | 5479 | 10735 | 13354 | 15977 | 266 | 8107 | 18605 | 21233 | 23861 |
| 267 | 5480 | 10736 | 13355 | 15978 | 268 | 8108 | 18606 | 21234 | 23862 |
| 269 | 5481 | 10737 | 13356 | 15979 | 270 | 8109 | 18607 | 21235 | 23863 |
| 271 | 5482 | 10738 | 13357 | 15980 | 272 | 8110 | 18608 | 21236 | 23864 |
| 273 | 5483 | 10739 | 13358 | 15981 | 274 | 8111 | 18609 | 21237 | 23865 |
| 275 | 5484 | 10740 | 13359 | 15982 | 276 | 8112 | 18610 | 21238 | 23866 |
| 277 | 5485 | 10741 | 13360 | 15983 | 278 | 8113 | 18611 | 21239 | 23867 |
| 279 | 5486 | 10742 | 13361 | 15984 | 280 | 8114 | 18612 | 21240 | 23868 |
| 281 | 5487 | 10743 | 13362 | 15985 | 282 | 8115 | 18613 | 21241 | 23869 |
| 283 | 5488 | 10744 | 13363 | 15986 | 284 | 8116 | 18614 | 21242 | 23870 |
| 285 | 5489 | 10745 | 13364 | 15987 | 286 | 8117 | 18615 | 21243 | 23871 |
| 287 | 5490 | 10746 | 13365 | 15988 | 288 | 8118 | 18616 | 21244 | 23872 |
| 289 | 5491 | 10747 | 13366 | 15989 | 290 | 8119 | 18617 | 21245 | 23873 |
| 291 | 5492 | 10748 | 13367 | 15990 | 292 | 8120 | 18618 | 21246 | 23874 |
| 293 | 5493 | 10749 | 13368 | 15991 | 294 | 8121 | 18619 | 21247 | 23875 |
| 295 | 5494 | 10750 | 13369 | 15992 | 296 | 8122 | 18620 | 21248 | 23876 |
| 297 | 5495 | 10751 | 13370 | 15993 | 298 | 8123 | 18621 | 21249 | 23877 |
| 299 | 5496 | 10752 | 13371 | 15994 | 300 | 8124 | 18622 | 21250 | 23878 |
| 301 | 5497 | 10753 | 13372 | 15995 | 302 | 8125 | 18623 | 21251 | 23879 |
| 303 | 5498 | 10754 | 13373 | 15996 | 304 | 8126 | 18624 | 21252 | 23880 |
| 305 | 5499 | 10755 | 13374 | 15997 | 306 | 8127 | 18625 | 21253 | 23881 |
| 307 | 5500 | 10756 | 13375 | 15998 | 308 | 8128 | 18626 | 21254 | 23882 |
| 309 | 5501 | 10757 | 13376 | 15999 | 310 | 8129 | 18627 | 21255 | 23883 |
| 311 | 5502 | 10758 | 13377 | 16000 | 312 | 8130 | 18628 | 21256 | 23884 |
| 313 | 5503 | 10759 | 13378 | 16001 | 314 | 8131 | 18629 | 21257 | 23885 |
| 315 | 5504 | 10760 | 13379 | 16002 | 316 | 8132 | 18630 | 21258 | 23886 |
| 317 | 5505 | 10761 | 13380 | 16003 | 318 | 8133 | 18631 | 21259 | 23887 |
| 319 | 5506 | 10762 | 13381 | 16004 | 320 | 8134 | 18632 | 21260 | 23888 |
| 321 | 5507 | 10763 | 13382 | 16005 | 322 | 8135 | 18633 | 21261 | 23889 |
| 323 | 5508 | 10764 | 13383 | 16006 | 324 | 8136 | 18634 | 21262 | 23890 |
| 325 | 5509 | 10765 | 13384 | 16007 | 326 | 8137 | 18635 | 21263 | 23891 |
| 327 | 5510 | 10766 | 13385 | 16008 | 328 | 8138 | 18636 | 21264 | 23892 |
| 329 | 5511 | 10767 | 13386 | 16009 | 330 | 8139 | 18637 | 21265 | 23893 |
| 331 | 5512 | 10768 | 13387 | 16010 | 332 | 8140 | 18638 | 21266 | 23894 |
| 333 | 5513 | 10769 | 13388 | 16011 | 334 | 8141 | 18639 | 21267 | 23895 |
| 335 | 5514 | 10770 | 13389 | 16012 | 336 | 8142 | 18640 | 21268 | 23896 |
| 337 | 5515 | 10771 | 13390 | 16013 | 338 | 8143 | 18641 | 21269 | 23897 |
| 339 | 5516 | 10772 | 13391 | 16014 | 340 | 8144 | 18642 | 21270 | 23898 |
| 341 | 5517 | 10773 | 13392 | 16015 | 342 | 8145 | 18643 | 21271 | 23899 |
| 343 | 5518 | 10774 | 13393 | 16016 | 344 | 8146 | 18644 | 21272 | 23900 |
| 345 | 5519 | 10775 | 13394 | 16017 | 346 | 8147 | 18645 | 21273 | 23901 |
| 347 | 5520 | 10776 | 13395 | 16018 | 348 | 8148 | 18646 | 21274 | 23902 |
| 349 | 5521 | 10777 | 13396 | 16019 | 350 | 8149 | 18647 | 21275 | 23903 |
| 351 | 5522 | 10778 | 13397 | 16020 | 352 | 8150 | 18648 | 21276 | 23904 |
| 353 | 5523 | 10779 | 13398 | 16021 | 354 | 8151 | 18649 | 21277 | 23905 |
| 355 | 5524 | 10780 | 13399 | 16022 | 356 | 8152 | 18650 | 21278 | 23906 |
| 357 | 5525 | 10781 | 13400 | 16023 | 358 | 8153 | 18651 | 21279 | 23907 |
| 359 | 5526 | 10782 | 13401 | 16024 | 360 | 8154 | 18652 | 21280 | 23908 |
| 361 | 5527 | 10783 | 13402 | 16025 | 362 | 8155 | 18653 | 21281 | 23909 |
| 363 | 5528 | 10784 | 13403 | 16026 | 364 | 8156 | 18654 | 21282 | 23910 |
| 365 | 5529 | 10785 | 13404 | 16027 | 366 | 8157 | 18655 | 21283 | 23911 |
| 367 | 5530 | 10786 | 13405 | 16028 | 368 | 8158 | 18656 | 21284 | 23912 |
| 369 | 5531 | 10787 | 13406 | 16029 | 370 | 8159 | 18657 | 21285 | 23913 |
| 371 | 5532 | 10788 | 13407 | 16030 | 372 | 8160 | 18658 | 21286 | 23914 |
| 373 | 5533 | 10789 | 13408 | 16031 | 374 | 8161 | 18659 | 21287 | 23915 |
| 375 | 5534 | 10790 | 13409 | 16032 | 376 | 8162 | 18660 | 21288 | 23916 |
| 377 | 5535 | 10791 | 13410 | 16033 | 378 | 8163 | 18661 | 21289 | 23917 |
| 379 | 5536 | 10792 | 13411 | 16034 | 380 | 8164 | 18662 | 21290 | 23918 |
| 381 | 5537 | 10793 | 13412 | 16035 | 382 | 8165 | 18663 | 21291 | 23919 |
| 383 | 5538 | 10794 | 13413 | 16036 | 384 | 8166 | 18664 | 21292 | 23920 |
| 385 | 5539 | 10795 | 13414 | 16037 | 386 | 8167 | 18665 | 21293 | 23921 |
| 387 | 5540 | 10796 | 13415 | 16038 | 388 | 8168 | 18666 | 21294 | 23922 |
| 389 | 5541 | 10797 | 13416 | 16039 | 390 | 8169 | 18667 | 21295 | 23923 |
| 391 | 5542 | 10798 | 13417 | 16040 | 392 | 8170 | 18668 | 21296 | 23924 |
| 393 | 5543 | 10799 | 13418 | 16041 | 394 | 8171 | 18669 | 21297 | 23925 |
| 395 | 5544 | 10800 | 13419 | 16042 | 396 | 8172 | 18670 | 21298 | 23926 |
| 397 | 5545 | 10801 | 13420 | 16043 | 398 | 8173 | 18671 | 21299 | 23927 |
| 399 | 5546 | 10802 | 13421 | 16044 | 400 | 8174 | 18672 | 21300 | 23928 |
| 401 | 5547 | 10803 | 13422 | 16045 | 402 | 8175 | 18673 | 21301 | 23929 |
| 403 | 5548 | 10804 | 13423 | 16046 | 404 | 8176 | 18674 | 21302 | 23930 |
| 405 | 5549 | 10805 | 13424 | 16047 | 406 | 8177 | 18675 | 21303 | 23931 |
| 407 | 5550 | 10806 | 13425 | 16048 | 408 | 8178 | 18676 | 21304 | 23932 |
| 409 | 5551 | 10807 | 13426 | 16049 | 410 | 8179 | 18677 | 21305 | 23933 |
| 411 | 5552 | 10808 | 13427 | 16050 | 412 | 8180 | 18678 | 21306 | 23934 |
| 413 | 5553 | 10809 | 13428 | 16051 | 414 | 8181 | 18679 | 21307 | 23935 |
| 415 | 5554 | 10810 | 13429 | 16052 | 416 | 8182 | 18680 | 21308 | 23936 |
| 417 | 5555 | 10811 | 13430 | 16053 | 418 | 8183 | 18681 | 21309 | 23937 |
| 419 | 5556 | 10812 | 13431 | 16054 | 420 | 8184 | 18682 | 21310 | 23938 |
| 421 | 5557 | 10813 | 13432 | 16055 | 422 | 8185 | 18683 | 21311 | 23939 |
| 423 | 5558 | 10814 | 13433 | 16056 | 424 | 8186 | 18684 | 21312 | 23940 |
| 425 | 5559 | 10815 | 13434 | 16057 | 426 | 8187 | 18685 | 21313 | 23941 |
| 427 | 5560 | 10816 | 13435 | 16058 | 428 | 8188 | 18686 | 21314 | 23942 |
| 429 | 5561 | 10817 | 13436 | 16059 | 430 | 8189 | 18687 | 21315 | 23943 |
| 431 | 5562 | 10818 | 13437 | 16060 | 432 | 8190 | 18688 | 21316 | 23944 |
| 433 | 5563 | 10819 | 13438 | 16061 | 434 | 8191 | 18689 | 21317 | 23945 |
| 435 | 5564 | 10820 | 13439 | 16062 | 436 | 8192 | 18690 | 21318 | 23946 |
| 437 | 5565 | 10821 | 13440 | 16063 | 438 | 8193 | 18691 | 21319 | 23947 |
| 439 | 5566 | 10822 | 13441 | 16064 | 440 | 8194 | 18692 | 21320 | 23948 |
| 441 | 5567 | 10823 | 13442 | 16065 | 442 | 8195 | 18693 | 21321 | 23949 |
| 443 | 5568 | 10824 | 13443 | 16066 | 444 | 8196 | 18694 | 21322 | 23950 |
| 445 | 5569 | 10825 | 13444 | 16067 | 446 | 8197 | 18695 | 21323 | 23951 |
| 447 | 5570 | 10826 | 13445 | 16068 | 448 | 8198 | 18696 | 21324 | 23952 |
| 449 | 5571 | 10827 | 13446 | 16069 | 450 | 8199 | 18697 | 21325 | 23953 |
| 451 | 5572 | 10828 | 13447 | 16070 | 452 | 8200 | 18698 | 21326 | 23954 |
| 453 | 5573 | 10829 | 13448 | 16071 | 454 | 8201 | 18699 | 21327 | 23955 |
| 455 | 5574 | 10830 | 13449 | 16072 | 456 | 8202 | 18700 | 21328 | 23956 |
| 457 | 5575 | 10831 | 13450 | 16073 | 458 | 8203 | 18701 | 21329 | 23957 |
| 459 | 5576 | 10832 | 13451 | 16074 | 460 | 8204 | 18702 | 21330 | 23958 |
| 461 | 5577 | 10833 | 13452 | 16075 | 462 | 8205 | 18703 | 21331 | 23959 |
| 463 | 5578 | 10834 | 13453 | 16076 | 464 | 8206 | 18704 | 21332 | 23960 |
| 465 | 5579 | 10835 | 13454 | 16077 | 466 | 8207 | 18705 | 21333 | 23961 |
| 467 | 5580 | 10836 | 13455 | 16078 | 468 | 8208 | 18706 | 21334 | 23962 |
| 469 | 5581 | 10837 | 13456 | 16079 | 470 | 8209 | 18707 | 21335 | 23963 |
| 471 | 5582 | 10838 | 13457 | 16080 | 472 | 8210 | 18708 | 21336 | 23964 |
| 473 | 5583 | 10839 | 13458 | 16081 | 474 | 8211 | 18709 | 21337 | 23965 |
| 475 | 5584 | 10840 | 13459 | 16082 | 476 | 8212 | 18710 | 21338 | 23966 |
| 477 | 5585 | 10841 | 13460 | 16083 | 478 | 8213 | 18711 | 21339 | 23967 |
| 479 | 5586 | 10842 | 13461 | 16084 | 480 | 8214 | 18712 | 21340 | 23968 |
| 481 | 5587 | 10843 | 13462 | 16085 | 482 | 8215 | 18713 | 21341 | 23969 |
| 483 | 5588 | 10844 | 13463 | 16086 | 484 | 8216 | 18714 | 21342 | 23970 |
| 485 | 5589 | 10845 | 13464 | 16087 | 486 | 8217 | 18715 | 21343 | 23971 |
| 487 | 5590 | 10846 | 13465 | 16088 | 488 | 8218 | 18716 | 21344 | 23972 |
| 489 | 5591 | 10847 | 13466 | 16089 | 490 | 8219 | 18717 | 21345 | 23973 |
| 491 | 5592 | 10848 | 13467 | 16090 | 492 | 8220 | 18718 | 21346 | 23974 |
| 493 | 5593 | 10849 | 13468 | 16091 | 494 | 8221 | 18719 | 21347 | 23975 |
| 495 | 5594 | 10850 | 13469 | 16092 | 496 | 8222 | 18720 | 21348 | 23976 |
| 497 | 5595 | 10851 | 13470 | 16093 | 498 | 8223 | 18721 | 21349 | 23977 |
| 499 | 5596 | 10852 | 13471 | 16094 | 500 | 8224 | 18722 | 21350 | 23978 |
| 501 | 5597 | 10853 | 13472 | 16095 | 502 | 8225 | 18723 | 21351 | 23979 |
| 503 | 5598 | 10854 | 13473 | 16096 | 504 | 8226 | 18724 | 21352 | 23980 |
| 505 | 5599 | 10855 | 13474 | 16097 | 506 | 8227 | 18725 | 21353 | 23981 |
| 507 | 5600 | 10856 | 13475 | 16098 | 508 | 8228 | 18726 | 21354 | 23982 |
| 509 | 5601 | 10857 | 13476 | 16099 | 510 | 8229 | 18727 | 21355 | 23983 |
| 511 | 5602 | 10858 | 13477 | 16100 | 512 | 8230 | 18728 | 21356 | 23984 |
| 513 | 5603 | 10859 | 13478 | 16101 | 514 | 8231 | 18729 | 21357 | 23985 |
| 515 | 5604 | 10860 | 13479 | 16102 | 516 | 8232 | 18730 | 21358 | 23986 |
| 517 | 5605 | 10861 | 13480 | 16103 | 518 | 8233 | 18731 | 21359 | 23987 |
| 519 | 5606 | 10862 | 13481 | 16104 | 520 | 8234 | 18732 | 21360 | 23988 |
| 521 | 5607 | 10863 | 13482 | 16105 | 522 | 8235 | 18733 | 21361 | 23989 |
| 523 | 5608 | 10864 | 13483 | 16106 | 524 | 8236 | 18734 | 21362 | 23990 |
| 525 | 5609 | 10865 | 13484 | 16107 | 526 | 8237 | 18735 | 21363 | 23991 |
| 527 | 5610 | 10866 | 13485 | 16108 | 528 | 8238 | 18736 | 21364 | 23992 |
| 529 | 5611 | 10867 | 13486 | 16109 | 530 | 8239 | 18737 | 21365 | 23993 |
| 531 | 5612 | | 13487 | 16110 | 532 | 8240 | 18738 | 21366 | 23994 |
| 533 | 5613 | 10868 | 13488 | 16111 | 534 | 8241 | 18739 | 21367 | 23995 |
| 535 | 5614 | 10869 | 13489 | 16112 | 536 | 8242 | 18740 | 21368 | 23996 |
| 537 | 5615 | 10870 | 13490 | 16113 | 538 | 8243 | 18741 | 21369 | 23997 |
| 539 | 5616 | 10871 | 13491 | 16114 | 540 | 8244 | 18742 | 21370 | 23998 |
| 541 | 5617 | 10872 | 13492 | 16115 | 542 | 8245 | 18743 | 21371 | 23999 |
| 543 | 5618 | 10873 | 13493 | 16116 | 544 | 8246 | 18744 | 21372 | 24000 |
| 545 | 5619 | 10874 | 13494 | 16117 | 546 | 8247 | 18745 | 21373 | 24001 |
| 547 | 5620 | 10875 | 13495 | 16118 | 548 | 8248 | 18746 | 21374 | 24002 |
| 549 | 5621 | 10876 | 13496 | 16119 | 550 | 8249 | 18747 | 21375 | 24003 |
| 551 | 5622 | 10877 | 13497 | 16120 | 552 | 8250 | 18748 | 21376 | 24004 |
| 553 | 5623 | 10878 | 13498 | 16121 | 554 | 8251 | 18749 | 21377 | 24005 |
| 555 | 5624 | 10879 | 13499 | 16122 | 556 | 8252 | 18750 | 21378 | 24006 |
| 557 | 5625 | 10880 | 13500 | 16123 | 558 | 8253 | 18751 | 21379 | 24007 |
| 559 | 5626 | 10881 | 13501 | 16124 | 560 | 8254 | 18752 | 21380 | 24008 |
| 561 | 5627 | 10882 | 13502 | 16125 | 562 | 8255 | 18753 | 21381 | 24009 |
| 563 | 5628 | 10883 | 13503 | 16126 | 564 | 8256 | 18754 | 21382 | 24010 |
| 565 | 5629 | 10884 | 13504 | 16127 | 566 | 8257 | 18755 | 21383 | 24011 |
| 567 | 5630 | 10885 | 13505 | 16128 | 568 | 8258 | 18756 | 21384 | 24012 |
| 569 | 5631 | 10886 | 13506 | 16129 | 570 | 8259 | 18757 | 21385 | 24013 |
| 571 | 5632 | 10887 | 13507 | 16130 | 572 | 8260 | 18758 | 21386 | 24014 |
| 573 | 5633 | 10888 | 13508 | 16131 | 574 | 8261 | 18759 | 21387 | 24015 |
| 575 | 5634 | 10889 | 13509 | 16132 | 576 | 8262 | 18760 | 21388 | 24016 |
| 577 | 5635 | 10890 | 13510 | 16133 | 578 | 8263 | 18761 | 21389 | 24017 |
| 579 | 5636 | 10891 | 13511 | 16134 | 580 | 8264 | 18762 | 21390 | 24018 |
| 581 | 5637 | 10892 | 13512 | 16135 | 582 | 8265 | 18763 | 21391 | 24019 |
| 583 | 5638 | 10893 | 13513 | 16136 | 584 | 8266 | 18764 | 21392 | 24020 |
| 585 | 5639 | 10894 | 13514 | 16137 | 586 | 8267 | 18765 | 21393 | 24021 |
| 587 | 5640 | 10895 | 13515 | 16138 | 588 | 8268 | 18766 | 21394 | 24022 |
| 589 | 5641 | 10896 | 13516 | 16139 | 590 | 8269 | 18767 | 21395 | 24023 |
| 591 | 5642 | 10897 | 13517 | 16140 | 592 | 8270 | 18768 | 21396 | 24024 |
| 593 | 5643 | 10898 | 13518 | 16141 | 594 | 8271 | 18769 | 21397 | 24025 |
| 595 | 5644 | 10899 | 13519 | 16142 | 596 | 8272 | 18770 | 21398 | 24026 |
| 597 | 5645 | 10900 | 13520 | 16143 | 598 | 8273 | 18771 | 21399 | 24027 |
| 599 | 5646 | 10901 | 13521 | 16144 | 600 | 8274 | 18772 | 21400 | 24028 |
| 601 | 5647 | 10902 | 13522 | 16145 | 602 | 8275 | 18773 | 21401 | 24029 |
| 603 | 5648 | 10903 | 13523 | 16146 | 604 | 8276 | 18774 | 21402 | 24030 |
| 605 | 5649 | 10904 | 13524 | 16147 | 606 | 8277 | 18775 | 21403 | 24031 |
| 607 | 5650 | 10905 | 13525 | 16148 | 608 | 8278 | 18776 | 21404 | 24032 |
| 609 | 5651 | 10906 | 13526 | 16149 | 610 | 8279 | 18777 | 21405 | 24033 |
| 611 | 5652 | 10907 | 13527 | 16150 | 612 | 8280 | 18778 | 21406 | 24034 |
| 613 | 5653 | 10908 | 13528 | 16151 | 614 | 8281 | 18779 | 21407 | 24035 |
| 615 | 5654 | 10909 | 13529 | 16152 | 616 | 8282 | 18780 | 21408 | 24036 |
| 617 | 5655 | 10910 | 13530 | 16153 | 618 | 8283 | 18781 | 21409 | 24037 |
| 619 | 5656 | 10911 | 13531 | 16154 | 620 | 8284 | 18782 | 21410 | 24038 |
| 621 | 5657 | 10912 | 13532 | 16155 | 622 | 8285 | 18783 | 21411 | 24039 |
| 623 | 5658 | 10913 | 13533 | 16156 | 624 | 8286 | 18784 | 21412 | 24040 |
| 625 | 5659 | 10914 | 13534 | 16157 | 626 | 8287 | 18785 | 21413 | 24041 |
| 627 | 5660 | 10915 | 13535 | 16158 | 628 | 8288 | 18786 | 21414 | 24042 |
| 629 | 5661 | | | 16159 | 630 | 8289 | 18787 | 21415 | 24043 |
| 631 | 5662 | 10916 | 13536 | 16160 | 632 | 8290 | 18788 | 21416 | 24044 |
| 633 | 5663 | 10917 | 13537 | 16161 | 634 | 8291 | 18789 | 21417 | 24045 |
| 635 | 5664 | 10918 | 13538 | 16162 | 636 | 8292 | 18790 | 21418 | 24046 |
| 637 | 5665 | 10919 | 13539 | 16163 | 638 | 8293 | 18791 | 21419 | 24047 |
| 639 | 5666 | 10920 | 13540 | 16164 | 640 | 8294 | 18792 | 21420 | 24048 |
| 641 | 5667 | 10921 | 13541 | 16165 | 642 | 8295 | 18793 | 21421 | 24049 |
| 643 | 5668 | 10922 | 13542 | 16166 | 644 | 8296 | 18794 | 21422 | 24050 |
| 645 | 5669 | 10923 | 13543 | 16167 | 646 | 8297 | 18795 | 21423 | 24051 |
| 647 | 5670 | 10924 | 13544 | 16168 | 648 | 8298 | 18796 | 21424 | 24052 |
| 649 | 5671 | 10925 | 13545 | 16169 | 650 | 8299 | 18797 | 21425 | 24053 |
| 651 | 5672 | 10926 | 13546 | 16170 | 652 | 8300 | 18798 | 21426 | 24054 |
| 653 | 5673 | 10927 | 13547 | 16171 | 654 | 8301 | 18799 | 21427 | 24055 |
| 655 | 5674 | 10928 | 13548 | 16172 | 656 | 8302 | 18800 | 21428 | 24056 |
| 657 | 5675 | 10929 | 13549 | 16173 | 658 | 8303 | 18801 | 21429 | 24057 |
| 659 | 5676 | 10930 | 13550 | 16174 | 660 | 8304 | 18802 | 21430 | 24058 |
| 661 | 5677 | 10931 | 13551 | 16175 | 662 | 8305 | 18803 | 21431 | 24059 |
| 663 | 5678 | 10932 | 13552 | 16176 | 664 | 8306 | 18804 | 21432 | 24060 |
| 665 | 5679 | 10933 | 13553 | 16177 | 666 | 8307 | 18805 | 21433 | 24061 |
| 667 | 5680 | 10934 | 13554 | 16178 | 668 | 8308 | 18806 | 21434 | 24062 |
| 669 | 5681 | 10935 | 13555 | 16179 | 670 | 8309 | 18807 | 21435 | 24063 |
| 671 | 5682 | 10936 | 13556 | 16180 | 672 | 8310 | 18808 | 21436 | 24064 |
| 673 | 5683 | 10937 | 13557 | 16181 | 674 | 8311 | 18809 | 21437 | 24065 |
| 675 | 5684 | 10938 | 13558 | 16182 | 676 | 8312 | 18810 | 21438 | 24066 |
| 677 | 5685 | 10939 | 13559 | 16183 | 678 | 8313 | 18811 | 21439 | 24067 |
| 679 | 5686 | 10940 | 13560 | 16184 | 680 | 8314 | 18812 | 21440 | 24068 |
| 681 | 5687 | 10941 | 13561 | 16185 | 682 | 8315 | 18813 | 21441 | 24069 |
| 683 | 5688 | 10942 | 13562 | 16186 | 684 | 8316 | 18814 | 21442 | 24070 |
| 685 | 5689 | 10943 | 13563 | 16187 | 686 | 8317 | 18815 | 21443 | 24071 |
| 687 | 5690 | 10944 | 13564 | 16188 | 688 | 8318 | 18816 | 21444 | 24072 |
| 689 | 5691 | 10945 | 13565 | 16189 | 690 | 8319 | 18817 | 21445 | 24073 |
| 691 | 5692 | 10946 | 13566 | 16190 | 692 | 8320 | 18818 | 21446 | 24074 |
| 693 | 5693 | 10947 | 13567 | 16191 | 694 | 8321 | 18819 | 21447 | 24075 |
| 695 | 5694 | 10948 | 13568 | 16192 | 696 | 8322 | 18820 | 21448 | 24076 |
| 697 | 5695 | 10949 | 13569 | 16193 | 698 | 8323 | 18821 | 21449 | 24077 |
| 699 | 5696 | 10950 | 13570 | 16194 | 700 | 8324 | 18822 | 21450 | 24078 |
| 701 | 5697 | 10951 | 13571 | 16195 | 702 | 8325 | 18823 | 21451 | 24079 |
| 703 | 5698 | 10952 | 13572 | 16196 | 704 | 8326 | 18824 | 21452 | 24080 |
| 705 | 5699 | 10953 | 13573 | 16197 | 706 | 8327 | 18825 | 21453 | 24081 |
| 707 | 5700 | 10954 | 13574 | 16198 | 708 | 8328 | 18826 | 21454 | 24082 |
| 709 | 5701 | 10955 | 13575 | 16199 | 710 | 8329 | 18827 | 21455 | 24083 |
| 711 | 5702 | 10956 | 13576 | 16200 | 712 | 8330 | 18828 | 21456 | 24084 |
| 713 | 5703 | 10957 | 13577 | 16201 | 714 | 8331 | 18829 | 21457 | 24085 |
| 715 | 5704 | 10958 | 13578 | 16202 | 716 | 8332 | 18830 | 21458 | 24086 |
| 717 | 5705 | 10959 | 13579 | 16203 | 718 | 8333 | 18831 | 21459 | 24087 |
| 719 | 5706 | 10960 | 13580 | 16204 | 720 | 8334 | 18832 | 21460 | 24088 |
| 721 | 5707 | 10961 | 13581 | 16205 | 722 | 8335 | 18833 | 21461 | 24089 |
| 723 | 5708 | 10962 | 13582 | 16206 | 724 | 8336 | 18834 | 21462 | 24090 |
| 725 | 5709 | 10963 | 13583 | 16207 | 726 | 8337 | 18835 | 21463 | 24091 |
| 727 | 5710 | 10964 | 13584 | 16208 | 728 | 8338 | 18836 | 21464 | 24092 |
| 729 | 5711 | 10965 | 13585 | 16209 | 730 | 8339 | 18837 | 21465 | 24093 |
| 731 | 5712 | 10966 | 13586 | 16210 | 732 | 8340 | 18838 | 21466 | 24094 |
| 733 | 5713 | 10967 | 13587 | 16211 | 734 | 8341 | 18839 | 21467 | 24095 |
| 735 | 5714 | 10968 | 13588 | 16212 | 736 | 8342 | 18840 | 21468 | 24096 |
| 737 | 5715 | 10969 | 13589 | 16213 | 738 | 8343 | 18841 | 21469 | 24097 |
| 739 | 5716 | 10970 | 13590 | 16214 | 740 | 8344 | 18842 | 21470 | 24098 |
| 741 | 5717 | 10971 | 13591 | 16215 | 742 | 8345 | 18843 | 21471 | 24099 |
| 743 | 5718 | 10972 | 13592 | 16216 | 744 | 8346 | 18844 | 21472 | 24100 |
| 745 | 5719 | 10973 | 13593 | 16217 | 746 | 8347 | 18845 | 21473 | 24101 |
| 747 | 5720 | 10974 | 13594 | 16218 | 748 | 8348 | 18846 | 21474 | 24102 |
| 749 | 5721 | 10975 | 13595 | 16219 | 750 | 8349 | 18847 | 21475 | 24103 |
| 751 | 5722 | 10976 | 13596 | 16220 | 752 | 8350 | 18848 | 21476 | 24104 |
| 753 | 5723 | 10977 | 13597 | 16221 | 754 | 8351 | 18849 | 21477 | 24105 |
| 755 | 5724 | 10978 | 13598 | 16222 | 756 | 8352 | 18850 | 21478 | 24106 |
| 757 | 5725 | 10979 | 13599 | 16223 | 758 | 8353 | 18851 | 21479 | 24107 |
| 759 | 5726 | 10980 | 13600 | 16224 | 760 | 8354 | 18852 | 21480 | 24108 |
| 761 | 5727 | 10981 | 13601 | 16225 | 762 | 8355 | 18853 | 21481 | 24109 |
| 763 | 5728 | 10982 | 13602 | 16226 | 764 | 8356 | 18854 | 21482 | 24110 |
| 765 | 5729 | 10983 | 13603 | 16227 | 766 | 8357 | 18855 | 21483 | 24111 |
| 767 | 5730 | 10984 | 13604 | 16228 | 768 | 8358 | 18856 | 21484 | 24112 |
| 769 | 5731 | 10985 | 13605 | 16229 | 770 | 8359 | 18857 | 21485 | 24113 |
| 771 | 5732 | 10986 | 13606 | 16230 | 772 | 8360 | 18858 | 21486 | 24114 |
| 773 | 5733 | 10987 | 13607 | 16231 | 774 | 8361 | 18859 | 21487 | 24115 |
| 775 | 5734 | 10988 | 13608 | 16232 | 776 | 8362 | 18860 | 21488 | 24116 |
| 777 | 5735 | 10989 | 13609 | 16233 | 778 | 8363 | 18861 | 21489 | 24117 |
| 779 | 5736 | 10990 | 13610 | 16234 | 780 | 8364 | 18862 | 21490 | 24118 |
| 781 | 5737 | 10991 | 13611 | 16235 | 782 | 8365 | 18863 | 21491 | 24119 |
| 783 | 5738 | 10992 | 13612 | 16236 | 784 | 8366 | 18864 | 21492 | 24120 |
| 785 | 5739 | 10993 | 13613 | 16237 | 786 | 8367 | 18865 | 21493 | 24121 |
| 787 | 5740 | 10994 | 13614 | 16238 | 788 | 8368 | 18866 | 21494 | 24122 |
| 789 | 5741 | 10995 | 13615 | 16239 | 790 | 8369 | 18867 | 21495 | 24123 |
| 791 | 5742 | 10996 | 13616 | 16240 | 792 | 8370 | 18868 | 21496 | 24124 |
| 793 | 5743 | 10997 | 13617 | 16241 | 794 | 8371 | 18869 | 21497 | 24125 |
| 795 | 5744 | 10998 | 13618 | 16242 | 796 | 8372 | 18870 | 21498 | 24126 |
| 797 | 5745 | 10999 | 13619 | 16243 | 798 | 8373 | 18871 | 21499 | 24127 |
| 799 | 5746 | 11000 | 13620 | 16244 | 800 | 8374 | 18872 | 21500 | 24128 |
| 801 | 5747 | 11001 | 13621 | 16245 | 802 | 8375 | 18873 | 21501 | 24129 |
| 803 | 5748 | 11002 | 13622 | 16246 | 804 | 8376 | 18874 | 21502 | 24130 |
| 805 | 5749 | 11003 | 13623 | 16247 | 806 | 8377 | 18875 | 21503 | 24131 |
| 807 | 5750 | 11004 | 13624 | 16248 | 808 | 8378 | 18876 | 21504 | 24132 |
| 809 | 5751 | 11005 | 13625 | 16249 | 810 | 8379 | 18877 | 21505 | 24133 |
| 811 | 5752 | 11006 | 13626 | 16250 | 812 | 8380 | 18878 | 21506 | 24134 |
| 813 | 5753 | 11007 | 13627 | 16251 | 814 | 8381 | 18879 | 21507 | 24135 |
| 815 | 5754 | 11008 | 13628 | 16252 | 816 | 8382 | 18880 | 21508 | 24136 |
| 817 | 5755 | 11009 | 13629 | 16253 | 818 | 8383 | 18881 | 21509 | 24137 |
| 819 | 5756 | 11010 | 13630 | 16254 | 820 | 8384 | 18882 | 21510 | 24138 |
| 821 | 5757 | 11011 | 13631 | 16255 | 822 | 8385 | 18883 | 21511 | 24139 |
| 823 | 5758 | 11012 | 13632 | 16256 | 824 | 8386 | 18884 | 21512 | 24140 |
| 825 | 5759 | 11013 | 13633 | 16257 | 826 | 8387 | 18885 | 21513 | 24141 |
| 827 | 5760 | 11014 | 13634 | 16258 | 828 | 8388 | 18886 | 21514 | 24142 |
| 829 | 5761 | 11015 | 13635 | 16259 | 830 | 8389 | 18887 | 21515 | 24143 |
| 831 | 5762 | 11016 | 13636 | 16260 | 832 | 8390 | 18888 | 21516 | 24144 |
| 833 | 5763 | 11017 | 13637 | 16261 | 834 | 8391 | 18889 | 21517 | 24145 |
| 835 | 5764 | 11018 | 13638 | 16262 | 836 | 8392 | 18890 | 21518 | 24146 |
| 837 | 5765 | 11019 | 13639 | 16263 | 838 | 8393 | 18891 | 21519 | 24147 |
| 839 | 5766 | 11020 | 13640 | 16264 | 840 | 8394 | 18892 | 21520 | 24148 |
| 841 | 5767 | 11021 | 13641 | 16265 | 842 | 8395 | 18893 | 21521 | 24149 |
| 843 | 5768 | 11022 | 13642 | 16266 | 844 | 8396 | 18894 | 21522 | 24150 |
| 845 | 5769 | 11023 | 13643 | 16267 | 846 | 8397 | 18895 | 21523 | 24151 |
| 847 | 5770 | 11024 | 13644 | 16268 | 848 | 8398 | 18896 | 21524 | 24152 |
| 849 | 5771 | 11025 | 13645 | 16269 | 850 | 8399 | 18897 | 21525 | 24153 |
| 851 | 5772 | 11026 | 13646 | 16270 | 852 | 8400 | 18898 | 21526 | 24154 |
| 853 | 5773 | 11027 | 13647 | 16271 | 854 | 8401 | 18899 | 21527 | 24155 |
| 855 | 5774 | 11028 | 13648 | 16272 | 856 | 8402 | 18900 | 21528 | 24156 |
| 857 | 5775 | 11029 | 13649 | 16273 | 858 | 8403 | 18901 | 21529 | 24157 |
| 859 | 5776 | 11030 | 13650 | 16274 | 860 | 8404 | 18902 | 21530 | 24158 |
| 861 | 5777 | 11031 | 13651 | 16275 | 862 | 8405 | 18903 | 21531 | 24159 |
| 863 | 5778 | 11032 | 13652 | 16276 | 864 | 8406 | 18904 | 21532 | 24160 |
| 865 | 5779 | 11033 | 13653 | 16277 | 866 | 8407 | 18905 | 21533 | 24161 |
| 867 | 5780 | 11034 | 13654 | 16278 | 868 | 8408 | 18906 | 21534 | 24162 |
| 869 | 5781 | 11035 | 13655 | 16279 | 870 | 8409 | 18907 | 21535 | 24163 |
| 871 | 5782 | 11036 | 13656 | 16280 | 872 | 8410 | 18908 | 21536 | 24164 |
| 873 | 5783 | 11037 | 13657 | 16281 | 874 | 8411 | 18909 | 21537 | 24165 |
| 875 | 5784 | 11038 | 13658 | 16282 | 876 | 8412 | 18910 | 21538 | 24166 |
| 877 | 5785 | 11039 | 13659 | 16283 | 878 | 8413 | 18911 | 21539 | 24167 |
| 879 | 5786 | 11040 | 13660 | 16284 | 880 | 8414 | 18912 | 21540 | 24168 |
| 881 | 5787 | 11041 | 13661 | 16285 | 882 | 8415 | 18913 | 21541 | 24169 |
| 883 | 5788 | 11042 | 13662 | 16286 | 884 | 8416 | 18914 | 21542 | 24170 |
| 885 | 5789 | 11043 | 13663 | 16287 | 886 | 8417 | 18915 | 21543 | 24171 |
| 887 | 5790 | 11044 | 13664 | 16288 | 888 | 8418 | 18916 | 21544 | 24172 |
| 889 | 5791 | 11045 | 13665 | 16289 | 890 | 8419 | 18917 | 21545 | 24173 |
| 891 | 5792 | 11046 | 13666 | 16290 | 892 | 8420 | 18918 | 21546 | 24174 |
| 893 | 5793 | 11047 | 13667 | 16291 | 894 | 8421 | 18919 | 21547 | 24175 |
| 895 | 5794 | 11048 | 13668 | 16292 | 896 | 8422 | 18920 | 21548 | 24176 |
| 897 | 5795 | 11049 | 13669 | 16293 | 898 | 8423 | 18921 | 21549 | 24177 |
| 899 | 5796 | 11050 | 13670 | 16294 | 900 | 8424 | 18922 | 21550 | 24178 |
| 901 | 5797 | 11051 | 13671 | 16295 | 902 | 8425 | 18923 | 21551 | 24179 |
| 903 | 5798 | 11052 | 13672 | 16296 | 904 | 8426 | 18924 | 21552 | 24180 |
| 905 | 5799 | 11053 | 13673 | 16297 | 906 | 8427 | 18925 | 21553 | 24181 |
| 907 | 5800 | 11054 | 13674 | 16298 | 908 | 8428 | 18926 | 21554 | 24182 |
| 909 | 5801 | 11055 | 13675 | 16299 | 910 | 8429 | 18927 | 21555 | 24183 |
| 911 | 5802 | | 13676 | 16300 | 912 | 8430 | 18928 | 21556 | 24184 |
| 913 | 5803 | 11056 | 13677 | 16301 | 914 | 8431 | 18929 | 21557 | 24185 |
| 915 | 5804 | 11057 | 13678 | 16302 | 916 | 8432 | 18930 | 21558 | 24186 |
| 917 | 5805 | 11058 | 13679 | 16303 | 918 | 8433 | 18931 | 21559 | 24187 |
| 919 | 5806 | 11059 | 13680 | 16304 | 920 | 8434 | 18932 | 21560 | 24188 |
| 921 | 5807 | 11060 | 13681 | 16305 | 922 | 8435 | 18933 | 21561 | 24189 |
| 923 | 5808 | 11061 | 13682 | 16306 | 924 | 8436 | 18934 | 21562 | 24190 |
| 925 | 5809 | 11062 | 13683 | 16307 | 926 | 8437 | 18935 | 21563 | 24191 |
| 927 | 5810 | 11063 | 13684 | 16308 | 928 | 8438 | 18936 | 21564 | 24192 |
| 929 | 5811 | 11064 | 13685 | 16309 | 930 | 8439 | 18937 | 21565 | 24193 |
| 931 | 5812 | 11065 | 13686 | 16310 | 932 | 8440 | 18938 | 21566 | 24194 |
| 933 | 5813 | 11066 | 13687 | 16311 | 934 | 8441 | 18939 | 21567 | 24195 |
| 935 | 5814 | 11067 | 13688 | 16312 | 936 | 8442 | 18940 | 21568 | 24196 |
| 937 | 5815 | 11068 | 13689 | 16313 | 938 | 8443 | 18941 | 21569 | 24197 |
| 939 | 5816 | 11069 | 13690 | 16314 | 940 | 8444 | 18942 | 21570 | 24198 |
| 941 | 5817 | 11070 | 13691 | 16315 | 942 | 8445 | 18943 | 21571 | 24199 |
| 943 | 5818 | 11071 | 13692 | 16316 | 944 | 8446 | 18944 | 21572 | 24200 |
| 945 | 5819 | 11072 | 13693 | 16317 | 946 | 8447 | 18945 | 21573 | 24201 |
| 947 | 5820 | 11073 | 13694 | 16318 | 948 | 8448 | 18946 | 21574 | 24202 |
| 949 | 5821 | 11074 | 13695 | 16319 | 950 | 8449 | 18947 | 21575 | 24203 |
| 951 | 5822 | 11075 | 13696 | 16320 | 952 | 8450 | 18948 | 21576 | 24204 |
| 953 | 5823 | 11076 | 13697 | 16321 | 954 | 8451 | 18949 | 21577 | 24205 |
| 955 | 5824 | 11077 | 13698 | 16322 | 956 | 8452 | 18950 | 21578 | 24206 |
| 957 | 5825 | 11078 | 13699 | 16323 | 958 | 8453 | 18951 | 21579 | 24207 |
| 959 | 5826 | 11079 | 13700 | 16324 | 960 | 8454 | 18952 | 21580 | 24208 |
| 961 | 5827 | 11080 | 13701 | 16325 | 962 | 8455 | 18953 | 21581 | 24209 |
| 963 | 5828 | 11081 | 13702 | 16326 | 964 | 8456 | 18954 | 21582 | 24210 |
| 965 | 5829 | 11082 | 13703 | 16327 | 966 | 8457 | 18955 | 21583 | 24211 |
| 967 | 5830 | 11083 | 13704 | 16328 | 968 | 8458 | 18956 | 21584 | 24212 |
| 969 | 5831 | 11084 | 13705 | 16329 | 970 | 8459 | 18957 | 21585 | 24213 |
| 971 | 5832 | 11085 | 13706 | 16330 | 972 | 8460 | 18958 | 21586 | 24214 |
| 973 | 5833 | 11086 | 13707 | 16331 | 974 | 8461 | 18959 | 21587 | 24215 |
| 975 | 5834 | 11087 | 13708 | 16332 | 976 | 8462 | 18960 | 21588 | 24216 |
| 977 | 5835 | 11088 | 13709 | 16333 | 978 | 8463 | 18961 | 21589 | 24217 |
| 979 | 5836 | 11089 | 13710 | 16334 | 980 | 8464 | 18962 | 21590 | 24218 |
| 981 | 5837 | 11090 | 13711 | 16335 | 982 | 8465 | 18963 | 21591 | 24219 |
| 983 | 5838 | 11091 | 13712 | 16336 | 984 | 8466 | 18964 | 21592 | 24220 |
| 985 | 5839 | 11092 | 13713 | 16337 | 986 | 8467 | 18965 | 21593 | 24221 |
| 987 | 5840 | 11093 | 13714 | 16338 | 988 | 8468 | 18966 | 21594 | 24222 |
| 989 | 5841 | 11094 | 13715 | 16339 | 990 | 8469 | 18967 | 21595 | 24223 |
| 991 | 5842 | 11095 | 13716 | 16340 | 992 | 8470 | 18968 | 21596 | 24224 |
| 993 | 5843 | 11096 | 13717 | 16341 | 994 | 8471 | 18969 | 21597 | 24225 |
| 995 | 5844 | 11097 | 13718 | 16342 | 996 | 8472 | 18970 | 21598 | 24226 |
| 997 | 5845 | 11098 | 13719 | 16343 | 998 | 8473 | 18971 | 21599 | 24227 |
| 999 | 5846 | | | 16344 | 1000 | 8474 | 18972 | 21600 | 24228 |
| 1001 | 5847 | 11099 | 13720 | 16345 | 1002 | 8475 | 18973 | 21601 | 24229 |
| 1003 | 5848 | 11100 | 13721 | 16346 | 1004 | 8476 | 18974 | 21602 | 24230 |
| 1005 | 5849 | 11101 | 13722 | 16347 | 1006 | 8477 | 18975 | 21603 | 24231 |
| 1007 | 5850 | 11102 | 13723 | 16348 | 1008 | 8478 | 18976 | 21604 | 24232 |
| 1009 | 5851 | 11103 | 13724 | 16349 | 1010 | 8479 | 18977 | 21605 | 24233 |
| 1011 | 5852 | | 13725 | 16350 | 1012 | 8480 | 18978 | 21606 | 24234 |
| 1013 | 5853 | 11104 | 13726 | 16351 | 1014 | 8481 | 18979 | 21607 | 24235 |
| 1015 | 5854 | 11105 | 13727 | 16352 | 1016 | 8482 | 18980 | 21608 | 24236 |
| 1017 | 5855 | 11106 | 13728 | 16353 | 1018 | 8483 | 18981 | 21609 | 24237 |
| 1019 | 5856 | 11107 | 13729 | 16354 | 1020 | 8484 | 18982 | 21610 | 24238 |
| 1021 | 5857 | 11108 | 13730 | 16355 | 1022 | 8485 | 18983 | 21611 | 24239 |
| 1023 | 5858 | 11109 | 13731 | 16356 | 1024 | 8486 | 18984 | 21612 | 24240 |
| 1025 | 5859 | 11110 | 13732 | 16357 | 1026 | 8487 | 18985 | 21613 | 24241 |
| 1027 | 5860 | 11111 | 13733 | 16358 | 1028 | 8488 | 18986 | 21614 | 24242 |
| 1029 | 5861 | 11112 | 13734 | 16359 | 1030 | 8489 | 18987 | 21615 | 24243 |
| 1031 | 5862 | 11113 | 13735 | 16360 | 1032 | 8490 | 18988 | 21616 | 24244 |
| 1033 | 5863 | 11114 | 13736 | 16361 | 1034 | 8491 | 18989 | 21617 | 24245 |
| 1035 | 5864 | 11115 | 13737 | 16362 | 1036 | 8492 | 18990 | 21618 | 24246 |
| 1037 | 5865 | 11116 | 13738 | 16363 | 1038 | 8493 | 18991 | 21619 | 24247 |
| 1039 | 5866 | 11117 | 13739 | 16364 | 1040 | 8494 | 18992 | 21620 | 24248 |
| 1041 | 5867 | 11118 | 13740 | 16365 | 1042 | 8495 | 18993 | 21621 | 24249 |
| 1043 | 5868 | 11119 | 13741 | 16366 | 1044 | 8496 | 18994 | 21622 | 24250 |
| 1045 | 5869 | 11120 | 13742 | 16367 | 1046 | 8497 | 18995 | 21623 | 24251 |
| 1047 | 5870 | 11121 | 13743 | 16368 | 1048 | 8498 | 18996 | 21624 | 24252 |
| 1049 | 5871 | 11122 | 13744 | 16369 | 1050 | 8499 | 18997 | 21625 | 24253 |
| 1051 | 5872 | 11123 | 13745 | 16370 | 1052 | 8500 | 18998 | 21626 | 24254 |
| 1053 | 5873 | 11124 | 13746 | 16371 | 1054 | 8501 | 18999 | 21627 | 24255 |
| 1055 | 5874 | 11125 | 13747 | 16372 | 1056 | 8502 | 19000 | 21628 | 24256 |
| 1057 | 5875 | 11126 | 13748 | 16373 | 1058 | 8503 | 19001 | 21629 | 24257 |
| 1059 | 5876 | 11127 | 13749 | 16374 | 1060 | 8504 | 19002 | 21630 | 24258 |
| 1061 | 5877 | 11128 | 13750 | 16375 | 1062 | 8505 | 19003 | 21631 | 24259 |
| 1063 | 5878 | 11129 | 13751 | 16376 | 1064 | 8506 | 19004 | 21632 | 24260 |
| 1065 | 5879 | 11130 | 13752 | 16377 | 1066 | 8507 | 19005 | 21633 | 24261 |
| 1067 | 5880 | 11131 | 13753 | 16378 | 1068 | 8508 | 19006 | 21634 | 24262 |
| 1069 | 5881 | 11132 | 13754 | 16379 | 1070 | 8509 | 19007 | 21635 | 24263 |
| 1071 | 5882 | 11133 | 13755 | 16380 | 1072 | 8510 | 19008 | 21636 | 24264 |
| 1073 | 5883 | 11134 | 13756 | 16381 | 1074 | 8511 | 19009 | 21637 | 24265 |
| 1075 | 5884 | 11135 | 13757 | 16382 | 1076 | 8512 | 19010 | 21638 | 24266 |
| 1077 | 5885 | 11136 | 13758 | 16383 | 1078 | 8513 | 19011 | 21639 | 24267 |
| 1079 | 5886 | 11137 | 13759 | 16384 | 1080 | 8514 | 19012 | 21640 | 24268 |
| 1081 | 5887 | 11138 | 13760 | 16385 | 1082 | 8515 | 19013 | 21641 | 24269 |
| 1083 | 5888 | 11139 | 13761 | 16386 | 1084 | 8516 | 19014 | 21642 | 24270 |
| 1085 | 5889 | 11140 | 13762 | 16387 | 1086 | 8517 | 19015 | 21643 | 24271 |
| 1087 | 5890 | 11141 | 13763 | 16388 | 1088 | 8518 | 19016 | 21644 | 24272 |
| 1089 | 5891 | 11142 | 13764 | 16389 | 1090 | 8519 | 19017 | 21645 | 24273 |
| 1091 | 5892 | 11143 | 13765 | 16390 | 1092 | 8520 | 19018 | 21646 | 24274 |
| 1093 | 5893 | 11144 | 13766 | 16391 | 1094 | 8521 | 19019 | 21647 | 24275 |
| 1095 | 5894 | 11145 | 13767 | 16392 | 1096 | 8522 | 19020 | 21648 | 24276 |
| 1097 | 5895 | 11146 | 13768 | 16393 | 1098 | 8523 | 19021 | 21649 | 24277 |
| 1099 | 5896 | 11147 | 13769 | 16394 | 1100 | 8524 | 19022 | 21650 | 24278 |
| 1101 | 5897 | 11148 | 13770 | 16395 | 1102 | 8525 | 19023 | 21651 | 24279 |
| 1103 | 5898 | 11149 | 13771 | 16396 | 1104 | 8526 | 19024 | 21652 | 24280 |
| 1105 | 5899 | 11150 | 13772 | 16397 | 1106 | 8527 | 19025 | 21653 | 24281 |
| 1107 | 5900 | 11151 | 13773 | 16398 | 1108 | 8528 | 19026 | 21654 | 24282 |
| 1109 | 5901 | 11152 | 13774 | 16399 | 1110 | 8529 | 19027 | 21655 | 24283 |
| 1111 | 5902 | 11153 | 13775 | 16400 | 1112 | 8530 | 19028 | 21656 | 24284 |
| 1113 | 5903 | 11154 | 13776 | 16401 | 1114 | 8531 | 19029 | 21657 | 24285 |
| 1115 | 5904 | 11155 | 13777 | 16402 | 1116 | 8532 | 19030 | 21658 | 24286 |
| 1117 | 5905 | 11156 | 13778 | 16403 | 1118 | 8533 | 19031 | 21659 | 24287 |
| 1119 | 5906 | 11157 | 13779 | 16404 | 1120 | 8534 | 19032 | 21660 | 24288 |
| 1121 | 5907 | 11158 | 13780 | 16405 | 1122 | 8535 | 19033 | 21661 | 24289 |
| 1123 | 5908 | 11159 | 13781 | 16406 | 1124 | 8536 | 19034 | 21662 | 24290 |
| 1125 | 5909 | 11160 | 13782 | 16407 | 1126 | 8537 | 19035 | 21663 | 24291 |
| 1127 | 5910 | 11161 | 13783 | 16408 | 1128 | 8538 | 19036 | 21664 | 24292 |
| 1129 | 5911 | 11162 | 13784 | 16409 | 1130 | 8539 | 19037 | 21665 | 24293 |
| 1131 | 5912 | 11163 | 13785 | 16410 | 1132 | 8540 | 19038 | 21666 | 24294 |
| 1133 | 5913 | 11164 | 13786 | 16411 | 1134 | 8541 | 19039 | 21667 | 24295 |
| 1135 | 5914 | 11165 | 13787 | 16412 | 1136 | 8542 | 19040 | 21668 | 24296 |
| 1137 | 5915 | 11166 | 13788 | 16413 | 1138 | 8543 | 19041 | 21669 | 24297 |
| 1139 | 5916 | 11167 | 13789 | 16414 | 1140 | 8544 | 19042 | 21670 | 24298 |
| 1141 | 5917 | 11168 | 13790 | 16415 | 1142 | 8545 | 19043 | 21671 | 24299 |
| 1143 | 5918 | 11169 | 13791 | 16416 | 1144 | 8546 | 19044 | 21672 | 24300 |
| 1145 | 5919 | 11170 | 13792 | 16417 | 1146 | 8547 | 19045 | 21673 | 24301 |
| 1147 | 5920 | 11171 | 13793 | 16418 | 1148 | 8548 | 19046 | 21674 | 24302 |
| 1149 | 5921 | 11172 | 13794 | 16419 | 1150 | 8549 | 19047 | 21675 | 24303 |
| 1151 | 5922 | 11173 | 13795 | 16420 | 1152 | 8550 | 19048 | 21676 | 24304 |
| 1153 | 5923 | 11174 | 13796 | 16421 | 1154 | 8551 | 19049 | 21677 | 24305 |
| 1155 | 5924 | 11175 | 13797 | 16422 | 1156 | 8552 | 19050 | 21678 | 24306 |
| 1157 | 5925 | 11176 | 13798 | 16423 | 1158 | 8553 | 19051 | 21679 | 24307 |
| 1159 | 5926 | 11177 | 13799 | 16424 | 1160 | 8554 | 19052 | 21680 | 24308 |
| 1161 | 5927 | 11178 | 13800 | 16425 | 1162 | 8555 | 19053 | 21681 | 24309 |
| 1163 | 5928 | 11179 | 13801 | 16426 | 1164 | 8556 | 19054 | 21682 | 24310 |
| 1165 | 5929 | 11180 | 13802 | 16427 | 1166 | 8557 | 19055 | 21683 | 24311 |
| 1167 | 5930 | 11181 | 13803 | 16428 | 1168 | 8558 | 19056 | 21684 | 24312 |
| 1169 | 5931 | 11182 | 13804 | 16429 | 1170 | 8559 | 19057 | 21685 | 24313 |
| 1171 | 5932 | 11183 | 13805 | 16430 | 1172 | 8560 | 19058 | 21686 | 24314 |
| 1173 | 5933 | 11184 | 13806 | 16431 | 1174 | 8561 | 19059 | 21687 | 24315 |
| 1175 | 5934 | 11185 | 13807 | 16432 | 1176 | 8562 | 19060 | 21688 | 24316 |
| 1177 | 5935 | 11186 | 13808 | 16433 | 1178 | 8563 | 19061 | 21689 | 24317 |
| 1179 | 5936 | 11187 | 13809 | 16434 | 1180 | 8564 | 19062 | 21690 | 24318 |
| 1181 | 5937 | 11188 | 13810 | 16435 | 1182 | 8565 | 19063 | 21691 | 24319 |
| 1183 | 5938 | 11189 | 13811 | 16436 | 1184 | 8566 | 19064 | 21692 | 24320 |
| 1185 | 5939 | 11190 | 13812 | 16437 | 1186 | 8567 | 19065 | 21693 | 24321 |
| 1187 | 5940 | 11191 | 13813 | 16438 | 1188 | 8568 | 19066 | 21694 | 24322 |
| 1189 | 5941 | 11192 | 13814 | 16439 | 1190 | 8569 | 19067 | 21695 | 24323 |
| 1191 | 5942 | 11193 | 13815 | 16440 | 1192 | 8570 | 19068 | 21696 | 24324 |
| 1193 | 5943 | 11194 | 13816 | 16441 | 1194 | 8571 | 19069 | 21697 | 24325 |
| 1195 | 5944 | 11195 | 13817 | 16442 | 1196 | 8572 | 19070 | 21698 | 24326 |
| 1197 | 5945 | 11196 | 13818 | 16443 | 1198 | 8573 | 19071 | 21699 | 24327 |
| 1199 | 5946 | 11197 | 13819 | 16444 | 1200 | 8574 | 19072 | 21700 | 24328 |
| 1201 | 5947 | 11198 | 13820 | 16445 | 1202 | 8575 | 19073 | 21701 | 24329 |
| 1203 | 5948 | 11199 | 13821 | 16446 | 1204 | 8576 | 19074 | 21702 | 24330 |
| 1205 | 5949 | 11200 | 13822 | 16447 | 1206 | 8577 | 19075 | 21703 | 24331 |
| 1207 | 5950 | 11201 | 13823 | 16448 | 1208 | 8578 | 19076 | 21704 | 24332 |
| 1209 | 5951 | 11202 | 13824 | 16449 | 1210 | 8579 | 19077 | 21705 | 24333 |
| 1211 | 5952 | 11203 | 13825 | 16450 | 1212 | 8580 | 19078 | 21706 | 24334 |
| 1213 | 5953 | 11204 | 13826 | 16451 | 1214 | 8581 | 19079 | 21707 | 24335 |
| 1215 | 5954 | 11205 | 13827 | 16452 | 1216 | 8582 | 19080 | 21708 | 24336 |
| 1217 | 5955 | 11206 | 13828 | 16453 | 1218 | 8583 | 19081 | 21709 | 24337 |
| 1219 | 5956 | 11207 | 13829 | 16454 | 1220 | 8584 | 19082 | 21710 | 24338 |
| 1221 | 5957 | 11208 | 13830 | 16455 | 1222 | 8585 | 19083 | 21711 | 24339 |
| 1223 | 5958 | 11209 | 13831 | 16456 | 1224 | 8586 | 19084 | 21712 | 24340 |
| 1225 | 5959 | 11210 | 13832 | 16457 | 1226 | 8587 | 19085 | 21713 | 24341 |
| 1227 | 5960 | 11211 | 13833 | 16458 | 1228 | 8588 | 19086 | 21714 | 24342 |
| 1229 | 5961 | 11212 | 13834 | 16459 | 1230 | 8589 | 19087 | 21715 | 24343 |
| 1231 | 5962 | 11213 | 13835 | 16460 | 1232 | 8590 | 19088 | 21716 | 24344 |
| 1233 | 5963 | 11214 | 13836 | 16461 | 1234 | 8591 | 19089 | 21717 | 24345 |
| 1235 | 5964 | 11215 | 13837 | 16462 | 1236 | 8592 | 19090 | 21718 | 24346 |
| 1237 | 5965 | 11216 | 13838 | 16463 | 1238 | 8593 | 19091 | 21719 | 24347 |
| 1239 | 5966 | 11217 | 13839 | 16464 | 1240 | 8594 | 19092 | 21720 | 24348 |
| 1241 | 5967 | 11218 | 13840 | 16465 | 1242 | 8595 | 19093 | 21721 | 24349 |
| 1243 | 5968 | 11219 | 13841 | 16466 | 1244 | 8596 | 19094 | 21722 | 24350 |
| 1245 | 5969 | 11220 | 13842 | 16467 | 1246 | 8597 | 19095 | 21723 | 24351 |
| 1247 | 5970 | 11221 | 13843 | 16468 | 1248 | 8598 | 19096 | 21724 | 24352 |
| 1249 | 5971 | 11222 | 13844 | 16469 | 1250 | 8599 | 19097 | 21725 | 24353 |
| 1251 | 5972 | 11223 | 13845 | 16470 | 1252 | 8600 | 19098 | 21726 | 24354 |
| 1253 | 5973 | 11224 | 13846 | 16471 | 1254 | 8601 | 19099 | 21727 | 24355 |
| 1255 | 5974 | 11225 | 13847 | 16472 | 1256 | 8602 | 19100 | 21728 | 24356 |
| 1257 | 5975 | 11226 | 13848 | 16473 | 1258 | 8603 | 19101 | 21729 | 24357 |
| 1259 | 5976 | 11227 | 13849 | 16474 | 1260 | 8604 | 19102 | 21730 | 24358 |
| 1261 | 5977 | 11228 | 13850 | 16475 | 1262 | 8605 | 19103 | 21731 | 24359 |
| 1263 | 5978 | 11229 | 13851 | 16476 | 1264 | 8606 | 19104 | 21732 | 24360 |
| 1265 | 5979 | 11230 | 13852 | 16477 | 1266 | 8607 | 19105 | 21733 | 24361 |
| 1267 | 5980 | 11231 | 13853 | 16478 | 1268 | 8608 | 19106 | 21734 | 24362 |
| 1269 | 5981 | 11232 | 13854 | 16479 | 1270 | 8609 | 19107 | 21735 | 24363 |
| 1271 | 5982 | 11233 | 13855 | 16480 | 1272 | 8610 | 19108 | 21736 | 24364 |
| 1273 | 5983 | 11234 | 13856 | 16481 | 1274 | 8611 | 19109 | 21737 | 24365 |
| 1275 | 5984 | 11235 | 13857 | 16482 | 1276 | 8612 | 19110 | 21738 | 24366 |
| 1277 | 5985 | 11236 | 13858 | 16483 | 1278 | 8613 | 19111 | 21739 | 24367 |
| 1279 | 5986 | 11237 | 13859 | 16484 | 1280 | 8614 | 19112 | 21740 | 24368 |
| 1281 | 5987 | 11238 | 13860 | 16485 | 1282 | 8615 | 19113 | 21741 | 24369 |
| 1283 | 5988 | 11239 | 13861 | 16486 | 1284 | 8616 | 19114 | 21742 | 24370 |
| 1285 | 5989 | 11240 | 13862 | 16487 | 1286 | 8617 | 19115 | 21743 | 24371 |
| 1287 | 5990 | 11241 | 13863 | 16488 | 1288 | 8618 | 19116 | 21744 | 24372 |
| 1289 | 5991 | 11242 | 13864 | 16489 | 1290 | 8619 | 19117 | 21745 | 24373 |
| 1291 | 5992 | 11243 | 13865 | 16490 | 1292 | 8620 | 19118 | 21746 | 24374 |
| 1293 | 5993 | 11244 | 13866 | 16491 | 1294 | 8621 | 19119 | 21747 | 24375 |
| 1295 | 5994 | 11245 | 13867 | 16492 | 1296 | 8622 | 19120 | 21748 | 24376 |
| 1297 | 5995 | 11246 | 13868 | 16493 | 1298 | 8623 | 19121 | 21749 | 24377 |
| 1299 | 5996 | 11247 | 13869 | 16494 | 1300 | 8624 | 19122 | 21750 | 24378 |
| 1301 | 5997 | 11248 | 13870 | 16495 | 1302 | 8625 | 19123 | 21751 | 24379 |
| 1303 | 5998 | 11249 | 13871 | 16496 | 1304 | 8626 | 19124 | 21752 | 24380 |
| 1305 | 5999 | 11250 | 13872 | 16497 | 1306 | 8627 | 19125 | 21753 | 24381 |
| 1307 | 6000 | 11251 | 13873 | 16498 | 1308 | 8628 | 19126 | 21754 | 24382 |
| 1309 | 6001 | 11252 | 13874 | 16499 | 1310 | 8629 | 19127 | 21755 | 24383 |
| 1311 | 6002 | 11253 | 13875 | 16500 | 1312 | 8630 | 19128 | 21756 | 24384 |
| 1313 | 6003 | 11254 | 13876 | 16501 | 1314 | 8631 | 19129 | 21757 | 24385 |
| 1315 | 6004 | 11255 | 13877 | 16502 | 1316 | 8632 | 19130 | 21758 | 24386 |
| 1317 | 6005 | 11256 | 13878 | 16503 | 1318 | 8633 | 19131 | 21759 | 24387 |
| 1319 | 6006 | 11257 | 13879 | 16504 | 1320 | 8634 | 19132 | 21760 | 24388 |
| 1321 | 6007 | 11258 | 13880 | 16505 | 1322 | 8635 | 19133 | 21761 | 24389 |
| 1323 | 6008 | 11259 | 13881 | 16506 | 1324 | 8636 | 19134 | 21762 | 24390 |
| 1325 | 6009 | 11260 | 13882 | 16507 | 1326 | 8637 | 19135 | 21763 | 24391 |
| 1327 | 6010 | 11261 | 13883 | 16508 | 1328 | 8638 | 19136 | 21764 | 24392 |
| 1329 | 6011 | 11262 | 13884 | 16509 | 1330 | 8639 | 19137 | 21765 | 24393 |
| 1331 | 6012 | 11263 | 13885 | 16510 | 1332 | 8640 | 19138 | 21766 | 24394 |
| 1333 | 6013 | 11264 | 13886 | 16511 | 1334 | 8641 | 19139 | 21767 | 24395 |
| 1335 | 6014 | 11265 | 13887 | 16512 | 1336 | 8642 | 19140 | 21768 | 24396 |
| 1337 | 6015 | 11266 | 13888 | 16513 | 1338 | 8643 | 19141 | 21769 | 24397 |
| 1339 | 6016 | 11267 | 13889 | 16514 | 1340 | 8644 | 19142 | 21770 | 24398 |
| 1341 | 6017 | 11268 | 13890 | 16515 | 1342 | 8645 | 19143 | 21771 | 24399 |
| 1343 | 6018 | 11269 | 13891 | 16516 | 1344 | 8646 | 19144 | 21772 | 24400 |
| 1345 | 6019 | 11270 | 13892 | 16517 | 1346 | 8647 | 19145 | 21773 | 24401 |
| 1347 | 6020 | 11271 | 13893 | 16518 | 1348 | 8648 | 19146 | 21774 | 24402 |
| 1349 | 6021 | 11272 | 13894 | 16519 | 1350 | 8649 | 19147 | 21775 | 24403 |
| 1351 | 6022 | 11273 | 13895 | 16520 | 1352 | 8650 | 19148 | 21776 | 24404 |
| 1353 | 6023 | 11274 | 13896 | 16521 | 1354 | 8651 | 19149 | 21777 | 24405 |
| 1355 | 6024 | 11275 | 13897 | 16522 | 1356 | 8652 | 19150 | 21778 | 24406 |
| 1357 | 6025 | 11276 | 13898 | 16523 | 1358 | 8653 | 19151 | 21779 | 24407 |
| 1359 | 6026 | 11277 | 13899 | 16524 | 1360 | 8654 | 19152 | 21780 | 24408 |
| 1361 | 6027 | 11278 | 13900 | 16525 | 1362 | 8655 | 19153 | 21781 | 24409 |
| 1363 | 6028 | 11279 | 13901 | 16526 | 1364 | 8656 | 19154 | 21782 | 24410 |
| 1365 | 6029 | 11280 | 13902 | 16527 | 1366 | 8657 | 19155 | 21783 | 24411 |
| 1367 | 6030 | 11281 | 13903 | 16528 | 1368 | 8658 | 19156 | 21784 | 24412 |
| 1369 | 6031 | 11282 | 13904 | 16529 | 1370 | 8659 | 19157 | 21785 | 24413 |
| 1371 | 6032 | 11283 | 13905 | 16530 | 1372 | 8660 | 19158 | 21786 | 24414 |
| 1373 | 6033 | 11284 | 13906 | 16531 | 1374 | 8661 | 19159 | 21787 | 24415 |
| 1375 | 6034 | 11285 | 13907 | 16532 | 1376 | 8662 | 19160 | 21788 | 24416 |
| 1377 | 6035 | 11286 | 13908 | 16533 | 1378 | 8663 | 19161 | 21789 | 24417 |
| 1379 | 6036 | 11287 | 13909 | 16534 | 1380 | 8664 | 19162 | 21790 | 24418 |
| 1381 | 6037 | 11288 | 13910 | 16535 | 1382 | 8665 | 19163 | 21791 | 24419 |
| 1383 | 6038 | 11289 | 13911 | 16536 | 1384 | 8666 | 19164 | 21792 | 24420 |
| 1385 | 6039 | 11290 | 13912 | 16537 | 1386 | 8667 | 19165 | 21793 | 24421 |
| 1387 | 6040 | 11291 | 13913 | 16538 | 1388 | 8668 | 19166 | 21794 | 24422 |
| 1389 | 6041 | 11292 | 13914 | 16539 | 1390 | 8669 | 19167 | 21795 | 24423 |
| 1391 | 6042 | 11293 | 13915 | 16540 | 1392 | 8670 | 19168 | 21796 | 24424 |
| 1393 | 6043 | 11294 | 13916 | 16541 | 1394 | 8671 | 19169 | 21797 | 24425 |
| 1395 | 6044 | 11295 | 13917 | 16542 | 1396 | 8672 | 19170 | 21798 | 24426 |
| 1397 | 6045 | 11296 | 13918 | 16543 | 1398 | 8673 | 19171 | 21799 | 24427 |
| 1399 | 6046 | 11297 | 13919 | 16544 | 1400 | 8674 | 19172 | 21800 | 24428 |
| 1401 | 6047 | 11298 | 13920 | 16545 | 1402 | 8675 | 19173 | 21801 | 24429 |
| 1403 | 6048 | 11299 | 13921 | 16546 | 1404 | 8676 | 19174 | 21802 | 24430 |
| 1405 | 6049 | 11300 | 13922 | 16547 | 1406 | 8677 | 19175 | 21803 | 24431 |
| 1407 | 6050 | 11301 | 13923 | 16548 | 1408 | 8678 | 19176 | 21804 | 24432 |
| 1409 | 6051 | 11302 | 13924 | 16549 | 1410 | 8679 | 19177 | 21805 | 24433 |
| 1411 | 6052 | 11303 | 13925 | 16550 | 1412 | 8680 | 19178 | 21806 | 24434 |
| 1413 | 6053 | 11304 | 13926 | 16551 | 1414 | 8681 | 19179 | 21807 | 24435 |
| 1415 | 6054 | 11305 | 13927 | 16552 | 1416 | 8682 | 19180 | 21808 | 24436 |
| 1417 | 6055 | 11306 | 13928 | 16553 | 1418 | 8683 | 19181 | 21809 | 24437 |
| 1419 | 6056 | 11307 | 13929 | 16554 | 1420 | 8684 | 19182 | 21810 | 24438 |
| 1421 | 6057 | 11308 | 13930 | 16555 | 1422 | 8685 | 19183 | 21811 | 24439 |
| 1423 | 6058 | 11309 | 13931 | 16556 | 1424 | 8686 | 19184 | 21812 | 24440 |
| 1425 | 6059 | 11310 | 13932 | 16557 | 1426 | 8687 | 19185 | 21813 | 24441 |
| 1427 | 6060 | 11311 | 13933 | 16558 | 1428 | 8688 | 19186 | 21814 | 24442 |
| 1429 | 6061 | 11312 | 13934 | 16559 | 1430 | 8689 | 19187 | 21815 | 24443 |
| 1431 | 6062 | 11313 | 13935 | 16560 | 1432 | 8690 | 19188 | 21816 | 24444 |
| 1433 | 6063 | 11314 | 13936 | 16561 | 1434 | 8691 | 19189 | 21817 | 24445 |
| 1435 | 6064 | 11315 | 13937 | 16562 | 1436 | 8692 | 19190 | 21818 | 24446 |
| 1437 | 6065 | 11316 | 13938 | 16563 | 1438 | 8693 | 19191 | 21819 | 24447 |
| 1439 | 6066 | 11317 | 13939 | 16564 | 1440 | 8694 | 19192 | 21820 | 24448 |
| 1441 | 6067 | 11318 | 13940 | 16565 | 1442 | 8695 | 19193 | 21821 | 24449 |
| 1443 | 6068 | 11319 | 13941 | 16566 | 1444 | 8696 | 19194 | 21822 | 24450 |
| 1445 | 6069 | 11320 | 13942 | 16567 | 1446 | 8697 | 19195 | 21823 | 24451 |
| 1447 | 6070 | 11321 | 13943 | 16568 | 1448 | 8698 | 19196 | 21824 | 24452 |
| 1449 | 6071 | 11322 | 13944 | 16569 | 1450 | 8699 | 19197 | 21825 | 24453 |
| 1451 | 6072 | 11323 | 13945 | 16570 | 1452 | 8700 | 19198 | 21826 | 24454 |
| 1453 | 6073 | 11324 | 13946 | 16571 | 1454 | 8701 | 19199 | 21827 | 24455 |
| 1455 | 6074 | 11325 | 13947 | 16572 | 1456 | 8702 | 19200 | 21828 | 24456 |
| 1457 | 6075 | 11326 | 13948 | 16573 | 1458 | 8703 | 19201 | 21829 | 24457 |
| 1459 | 6076 | 11327 | 13949 | 16574 | 1460 | 8704 | 19202 | 21830 | 24458 |
| 1461 | 6077 | 11328 | 13950 | 16575 | 1462 | 8705 | 19203 | 21831 | 24459 |
| 1463 | 6078 | 11329 | 13951 | 16576 | 1464 | 8706 | 19204 | 21832 | 24460 |
| 1465 | 6079 | 11330 | 13952 | 16577 | 1466 | 8707 | 19205 | 21833 | 24461 |
| 1467 | 6080 | 11331 | 13953 | 16578 | 1468 | 8708 | 19206 | 21834 | 24462 |
| 1469 | 6081 | 11332 | 13954 | 16579 | 1470 | 8709 | 19207 | 21835 | 24463 |
| 1471 | 6082 | 11333 | 13955 | 16580 | 1472 | 8710 | 19208 | 21836 | 24464 |
| 1473 | 6083 | 11334 | 13956 | 16581 | 1474 | 8711 | 19209 | 21837 | 24465 |
| 1475 | 6084 | 11335 | 13957 | 16582 | 1476 | 8712 | 19210 | 21838 | 24466 |
| 1477 | 6085 | 11336 | 13958 | 16583 | 1478 | 8713 | 19211 | 21839 | 24467 |
| 1479 | 6086 | 11337 | 13959 | 16584 | 1480 | 8714 | 19212 | 21840 | 24468 |
| 1481 | 6087 | 11338 | 13960 | 16585 | 1482 | 8715 | 19213 | 21841 | 24469 |
| 1483 | 6088 | 11339 | 13961 | 16586 | 1484 | 8716 | 19214 | 21842 | 24470 |
| 1485 | 6089 | 11340 | 13962 | 16587 | 1486 | 8717 | 19215 | 21843 | 24471 |
| 1487 | 6090 | 11341 | 13963 | 16588 | 1488 | 8718 | 19216 | 21844 | 24472 |
| 1489 | 6091 | 11342 | 13964 | 16589 | 1490 | 8719 | 19217 | 21845 | 24473 |
| 1491 | 6092 | 11343 | 13965 | 16590 | 1492 | 8720 | 19218 | 21846 | 24474 |
| 1493 | 6093 | 11344 | 13966 | 16591 | 1494 | 8721 | 19219 | 21847 | 24475 |
| 1495 | 6094 | 11345 | 13967 | 16592 | 1496 | 8722 | 19220 | 21848 | 24476 |
| 1497 | 6095 | 11346 | 13968 | 16593 | 1498 | 8723 | 19221 | 21849 | 24477 |
| 1499 | 6096 | 11347 | 13969 | 16594 | 1500 | 8724 | 19222 | 21850 | 24478 |
| 1501 | 6097 | 11348 | 13970 | 16595 | 1502 | 8725 | 19223 | 21851 | 24479 |
| 1503 | 6098 | 11349 | 13971 | 16596 | 1504 | 8726 | 19224 | 21852 | 24480 |
| 1505 | 6099 | 11350 | 13972 | 16597 | 1506 | 8727 | 19225 | 21853 | 24481 |
| 1507 | 6100 | 11351 | 13973 | 16598 | 1508 | 8728 | 19226 | 21854 | 24482 |
| 1509 | 6101 | 11352 | 13974 | 16599 | 1510 | 8729 | 19227 | 21855 | 24483 |
| 1511 | 6102 | 11353 | 13975 | 16600 | 1512 | 8730 | 19228 | 21856 | 24484 |
| 1513 | 6103 | 11354 | 13976 | 16601 | 1514 | 8731 | 19229 | 21857 | 24485 |
| 1515 | 6104 | 11355 | 13977 | 16602 | 1516 | 8732 | 19230 | 21858 | 24486 |
| 1517 | 6105 | 11356 | 13978 | 16603 | 1518 | 8733 | 19231 | 21859 | 24487 |
| 1519 | 6106 | 11357 | 13979 | 16604 | 1520 | 8734 | 19232 | 21860 | 24488 |
| 1521 | 6107 | 11358 | 13980 | 16605 | 1522 | 8735 | 19233 | 21861 | 24489 |
| 1523 | 6108 | 11359 | 13981 | 16606 | 1524 | 8736 | 19234 | 21862 | 24490 |
| 1525 | 6109 | 11360 | 13982 | 16607 | 1526 | 8737 | 19235 | 21863 | 24491 |
| 1527 | 6110 | 11361 | 13983 | 16608 | 1528 | 8738 | 19236 | 21864 | 24492 |
| 1529 | 6111 | 11362 | 13984 | 16609 | 1530 | 8739 | 19237 | 21865 | 24493 |
| 1531 | 6112 | 11363 | 13985 | 16610 | 1532 | 8740 | 19238 | 21866 | 24494 |
| 1533 | 6113 | 11364 | 13986 | 16611 | 1534 | 8741 | 19239 | 21867 | 24495 |
| 1535 | 6114 | 11365 | 13987 | 16612 | 1536 | 8742 | 19240 | 21868 | 24496 |
| 1537 | 6115 | 11366 | 13988 | 16613 | 1538 | 8743 | 19241 | 21869 | 24497 |
| 1539 | 6116 | 11367 | 13989 | 16614 | 1540 | 8744 | 19242 | 21870 | 24498 |
| 1541 | 6117 | 11368 | 13990 | 16615 | 1542 | 8745 | 19243 | 21871 | 24499 |
| 1543 | 6118 | 11369 | 13991 | 16616 | 1544 | 8746 | 19244 | 21872 | 24500 |
| 1545 | 6119 | 11370 | 13992 | 16617 | 1546 | 8747 | 19245 | 21873 | 24501 |
| 1547 | 6120 | 11371 | 13993 | 16618 | 1548 | 8748 | 19246 | 21874 | 24502 |
| 1549 | 6121 | 11372 | 13994 | 16619 | 1550 | 8749 | 19247 | 21875 | 24503 |
| 1551 | 6122 | 11373 | 13995 | 16620 | 1552 | 8750 | 19248 | 21876 | 24504 |
| 1553 | 6123 | 11374 | 13996 | 16621 | 1554 | 8751 | 19249 | 21877 | 24505 |
| 1555 | 6124 | 11375 | 13997 | 16622 | 1556 | 8752 | 19250 | 21878 | 24506 |
| 1557 | 6125 | 11376 | 13998 | 16623 | 1558 | 8753 | 19251 | 21879 | 24507 |
| 1559 | 6126 | 11377 | 13999 | 16624 | 1560 | 8754 | 19252 | 21880 | 24508 |
| 1561 | 6127 | 11378 | 14000 | 16625 | 1562 | 8755 | 19253 | 21881 | 24509 |
| 1563 | 6128 | 11379 | 14001 | 16626 | 1564 | 8756 | 19254 | 21882 | 24510 |
| 1565 | 6129 | 11380 | 14002 | 16627 | 1566 | 8757 | 19255 | 21883 | 24511 |
| 1567 | 6130 | 11381 | 14003 | 16628 | 1568 | 8758 | 19256 | 21884 | 24512 |
| 1569 | 6131 | 11382 | 14004 | 16629 | 1570 | 8759 | 19257 | 21885 | 24513 |
| 1571 | 6132 | 11383 | 14005 | 16630 | 1572 | 8760 | 19258 | 21886 | 24514 |
| 1573 | 6133 | 11384 | 14006 | 16631 | 1574 | 8761 | 19259 | 21887 | 24515 |
| 1575 | 6134 | 11385 | 14007 | 16632 | 1576 | 8762 | 19260 | 21888 | 24516 |
| 1577 | 6135 | 11386 | 14008 | 16633 | 1578 | 8763 | 19261 | 21889 | 24517 |
| 1579 | 6136 | 11387 | 14009 | 16634 | 1580 | 8764 | 19262 | 21890 | 24518 |
| 1581 | 6137 | 11388 | 14010 | 16635 | 1582 | 8765 | 19263 | 21891 | 24519 |
| 1583 | 6138 | 11389 | 14011 | 16636 | 1584 | 8766 | 19264 | 21892 | 24520 |
| 1585 | 6139 | 11390 | 14012 | 16637 | 1586 | 8767 | 19265 | 21893 | 24521 |
| 1587 | 6140 | 11391 | 14013 | 16638 | 1588 | 8768 | 19266 | 21894 | 24522 |
| 1589 | 6141 | 11392 | 14014 | 16639 | 1590 | 8769 | 19267 | 21895 | 24523 |
| 1591 | 6142 | 11393 | 14015 | 16640 | 1592 | 8770 | 19268 | 21896 | 24524 |
| 1593 | 6143 | 11394 | 14016 | 16641 | 1594 | 8771 | 19269 | 21897 | 24525 |
| 1595 | 6144 | 11395 | 14017 | 16642 | 1596 | 8772 | 19270 | 21898 | 24526 |
| 1597 | 6145 | 11396 | 14018 | 16643 | 1598 | 8773 | 19271 | 21899 | 24527 |
| 1599 | 6146 | 11397 | 14019 | 16644 | 1600 | 8774 | 19272 | 21900 | 24528 |
| 1601 | 6147 | 11398 | 14020 | 16645 | 1602 | 8775 | 19273 | 21901 | 24529 |
| 1603 | 6148 | 11399 | 14021 | 16646 | 1604 | 8776 | 19274 | 21902 | 24530 |
| 1605 | 6149 | 11400 | 14022 | 16647 | 1606 | 8777 | 19275 | 21903 | 24531 |
| 1607 | 6150 | 11401 | 14023 | 16648 | 1608 | 8778 | 19276 | 21904 | 24532 |
| 1609 | 6151 | 11402 | 14024 | 16649 | 1610 | 8779 | 19277 | 21905 | 24533 |
| 1611 | 6152 | 11403 | 14025 | 16650 | 1612 | 8780 | 19278 | 21906 | 24534 |
| 1613 | 6153 | 11404 | 14026 | 16651 | 1614 | 8781 | 19279 | 21907 | 24535 |
| 1615 | 6154 | 11405 | 14027 | 16652 | 1616 | 8782 | 19280 | 21908 | 24536 |
| 1617 | 6155 | 11406 | 14028 | 16653 | 1618 | 8783 | 19281 | 21909 | 24537 |
| 1619 | 6156 | 11407 | 14029 | 16654 | 1620 | 8784 | 19282 | 21910 | 24538 |
| 1621 | 6157 | 11408 | 14030 | 16655 | 1622 | 8785 | 19283 | 21911 | 24539 |
| 1623 | 6158 | 11409 | 14031 | 16656 | 1624 | 8786 | 19284 | 21912 | 24540 |
| 1625 | 6159 | 11410 | 14032 | 16657 | 1626 | 8787 | 19285 | 21913 | 24541 |
| 1627 | 6160 | 11411 | 14033 | 16658 | 1628 | 8788 | 19286 | 21914 | 24542 |
| 1629 | 6161 | 11412 | 14034 | 16659 | 1630 | 8789 | 19287 | 21915 | 24543 |
| 1631 | 6162 | 11413 | 14035 | 16660 | 1632 | 8790 | 19288 | 21916 | 24544 |
| 1633 | 6163 | 11414 | 14036 | 16661 | 1634 | 8791 | 19289 | 21917 | 24545 |
| 1635 | 6164 | 11415 | 14037 | 16662 | 1636 | 8792 | 19290 | 21918 | 24546 |
| 1637 | 6165 | 11416 | 14038 | 16663 | 1638 | 8793 | 19291 | 21919 | 24547 |
| 1639 | 6166 | 11417 | 14039 | 16664 | 1640 | 8794 | 19292 | 21920 | 24548 |
| 1641 | 6167 | 11418 | 14040 | 16665 | 1642 | 8795 | 19293 | 21921 | 24549 |
| 1643 | 6168 | 11419 | 14041 | 16666 | 1644 | 8796 | 19294 | 21922 | 24550 |
| 1645 | 6169 | 11420 | 14042 | 16667 | 1646 | 8797 | 19295 | 21923 | 24551 |
| 1647 | 6170 | 11421 | 14043 | 16668 | 1648 | 8798 | 19296 | 21924 | 24552 |
| 1649 | 6171 | 11422 | 14044 | 16669 | 1650 | 8799 | 19297 | 21925 | 24553 |
| 1651 | 6172 | 11423 | 14045 | 16670 | 1652 | 8800 | 19298 | 21926 | 24554 |
| 1653 | 6173 | 11424 | 14046 | 16671 | 1654 | 8801 | 19299 | 21927 | 24555 |
| 1655 | 6174 | 11425 | 14047 | 16672 | 1656 | 8802 | 19300 | 21928 | 24556 |
| 1657 | 6175 | 11426 | 14048 | 16673 | 1658 | 8803 | 19301 | 21929 | 24557 |
| 1659 | 6176 | 11427 | 14049 | 16674 | 1660 | 8804 | 19302 | 21930 | 24558 |
| 1661 | 6177 | 11428 | 14050 | 16675 | 1662 | 8805 | 19303 | 21931 | 24559 |
| 1663 | 6178 | 11429 | 14051 | 16676 | 1664 | 8806 | 19304 | 21932 | 24560 |
| 1665 | 6179 | 11430 | 14052 | 16677 | 1666 | 8807 | 19305 | 21933 | 24561 |
| 1667 | 6180 | 11431 | 14053 | 16678 | 1668 | 8808 | 19306 | 21934 | 24562 |
| 1669 | 6181 | 11432 | 14054 | 16679 | 1670 | 8809 | 19307 | 21935 | 24563 |
| 1671 | 6182 | 11433 | 14055 | 16680 | 1672 | 8810 | 19308 | 21936 | 24564 |
| 1673 | 6183 | 11434 | 14056 | 16681 | 1674 | 8811 | 19309 | 21937 | 24565 |
| 1675 | 6184 | 11435 | 14057 | 16682 | 1676 | 8812 | 19310 | 21938 | 24566 |
| 1677 | 6185 | 11436 | 14058 | 16683 | 1678 | 8813 | 19311 | 21939 | 24567 |
| 1679 | 6186 | 11437 | 14059 | 16684 | 1680 | 8814 | 19312 | 21940 | 24568 |
| 1681 | 6187 | 11438 | 14060 | 16685 | 1682 | 8815 | 19313 | 21941 | 24569 |
| 1683 | 6188 | 11439 | 14061 | 16686 | 1684 | 8816 | 19314 | 21942 | 24570 |
| 1685 | 6189 | 11440 | 14062 | 16687 | 1686 | 8817 | 19315 | 21943 | 24571 |
| 1687 | 6190 | 11441 | 14063 | 16688 | 1688 | 8818 | 19316 | 21944 | 24572 |
| 1689 | 6191 | 11442 | 14064 | 16689 | 1690 | 8819 | 19317 | 21945 | 24573 |
| 1691 | 6192 | 11443 | 14065 | 16690 | 1692 | 8820 | 19318 | 21946 | 24574 |
| 1693 | 6193 | 11444 | 14066 | 16691 | 1694 | 8821 | 19319 | 21947 | 24575 |
| 1695 | 6194 | 11445 | 14067 | 16692 | 1696 | 8822 | 19320 | 21948 | 24576 |
| 1697 | 6195 | 11446 | 14068 | 16693 | 1698 | 8823 | 19321 | 21949 | 24577 |
| 1699 | 6196 | 11447 | 14069 | 16694 | 1700 | 8824 | 19322 | 21950 | 24578 |
| 1701 | 6197 | 11448 | 14070 | 16695 | 1702 | 8825 | 19323 | 21951 | 24579 |
| 1703 | 6198 | 11449 | 14071 | 16696 | 1704 | 8826 | 19324 | 21952 | 24580 |
| 1705 | 6199 | 11450 | 14072 | 16697 | 1706 | 8827 | 19325 | 21953 | 24581 |
| 1707 | 6200 | 11451 | 14073 | 16698 | 1708 | 8828 | 19326 | 21954 | 24582 |
| 1709 | 6201 | 11452 | 14074 | 16699 | 1710 | 8829 | 19327 | 21955 | 24583 |
| 1711 | 6202 | 11453 | 14075 | 16700 | 1712 | 8830 | 19328 | 21956 | 24584 |
| 1713 | 6203 | 11454 | 14076 | 16701 | 1714 | 8831 | 19329 | 21957 | 24585 |
| 1715 | 6204 | 11455 | 14077 | 16702 | 1716 | 8832 | 19330 | 21958 | 24586 |
| 1717 | 6205 | 11456 | 14078 | 16703 | 1718 | 8833 | 19331 | 21959 | 24587 |
| 1719 | 6206 | 11457 | 14079 | 16704 | 1720 | 8834 | 19332 | 21960 | 24588 |
| 1721 | 6207 | 11458 | 14080 | 16705 | 1722 | 8835 | 19333 | 21961 | 24589 |
| 1723 | 6208 | 11459 | 14081 | 16706 | 1724 | 8836 | 19334 | 21962 | 24590 |
| 1725 | 6209 | 11460 | 14082 | 16707 | 1726 | 8837 | 19335 | 21963 | 24591 |
| 1727 | 6210 | 11461 | 14083 | 16708 | 1728 | 8838 | 19336 | 21964 | 24592 |
| 1729 | 6211 | 11462 | 14084 | 16709 | 1730 | 8839 | 19337 | 21965 | 24593 |
| 1731 | 6212 | 11463 | 14085 | 16710 | 1732 | 8840 | 19338 | 21966 | 24594 |
| 1733 | 6213 | 11464 | 14086 | 16711 | 1734 | 8841 | 19339 | 21967 | 24595 |
| 1735 | 6214 | 11465 | 14087 | 16712 | 1736 | 8842 | 19340 | 21968 | 24596 |
| 1737 | 6215 | 11466 | 14088 | 16713 | 1738 | 8843 | 19341 | 21969 | 24597 |
| 1739 | 6216 | 11467 | 14089 | 16714 | 1740 | 8844 | 19342 | 21970 | 24598 |
| 1741 | 6217 | 11468 | 14090 | 16715 | 1742 | 8845 | 19343 | 21971 | 24599 |
| 1743 | 6218 | 11469 | 14091 | 16716 | 1744 | 8846 | 19344 | 21972 | 24600 |
| 1745 | 6219 | 11470 | 14092 | 16717 | 1746 | 8847 | 19345 | 21973 | 24601 |
| 1747 | 6220 | 11471 | 14093 | 16718 | 1748 | 8848 | 19346 | 21974 | 24602 |
| 1749 | 6221 | 11472 | 14094 | 16719 | 1750 | 8849 | 19347 | 21975 | 24603 |
| 1751 | 6222 | 11473 | 14095 | 16720 | 1752 | 8850 | 19348 | 21976 | 24604 |
| 1753 | 6223 | 11474 | 14096 | 16721 | 1754 | 8851 | 19349 | 21977 | 24605 |
| 1755 | 6224 | 11475 | 14097 | 16722 | 1756 | 8852 | 19350 | 21978 | 24606 |
| 1757 | 6225 | 11476 | 14098 | 16723 | 1758 | 8853 | 19351 | 21979 | 24607 |
| 1759 | 6226 | 11477 | 14099 | 16724 | 1760 | 8854 | 19352 | 21980 | 24608 |
| 1761 | 6227 | 11478 | 14100 | 16725 | 1762 | 8855 | 19353 | 21981 | 24609 |
| 1763 | 6228 | 11479 | 14101 | 16726 | 1764 | 8856 | 19354 | 21982 | 24610 |
| 1765 | 6229 | 11480 | 14102 | 16727 | 1766 | 8857 | 19355 | 21983 | 24611 |
| 1767 | 6230 | 11481 | 14103 | 16728 | 1768 | 8858 | 19356 | 21984 | 24612 |
| 1769 | 6231 | 11482 | 14104 | 16729 | 1770 | 8859 | 19357 | 21985 | 24613 |
| 1771 | 6232 | 11483 | 14105 | 16730 | 1772 | 8860 | 19358 | 21986 | 24614 |
| 1773 | 6233 | 11484 | 14106 | 16731 | 1774 | 8861 | 19359 | 21987 | 24615 |
| 1775 | 6234 | 11485 | 14107 | 16732 | 1776 | 8862 | 19360 | 21988 | 24616 |
| 1777 | 6235 | 11486 | 14108 | 16733 | 1778 | 8863 | 19361 | 21989 | 24617 |
| 1779 | 6236 | 11487 | 14109 | 16734 | 1780 | 8864 | 19362 | 21990 | 24618 |
| 1781 | 6237 | 11488 | 14110 | 16735 | 1782 | 8865 | 19363 | 21991 | 24619 |
| 1783 | 6238 | 11489 | 14111 | 16736 | 1784 | 8866 | 19364 | 21992 | 24620 |
| 1785 | 6239 | 11490 | 14112 | 16737 | 1786 | 8867 | 19365 | 21993 | 24621 |
| 1787 | 6240 | 11491 | 14113 | 16738 | 1788 | 8868 | 19366 | 21994 | 24622 |
| 1789 | 6241 | 11492 | 14114 | 16739 | 1790 | 8869 | 19367 | 21995 | 24623 |
| 1791 | 6242 | 11493 | 14115 | 16740 | 1792 | 8870 | 19368 | 21996 | 24624 |
| 1793 | 6243 | 11494 | 14116 | 16741 | 1794 | 8871 | 19369 | 21997 | 24625 |
| 1795 | 6244 | 11495 | 14117 | 16742 | 1796 | 8872 | 19370 | 21998 | 24626 |
| 1797 | 6245 | 11496 | 14118 | 16743 | 1798 | 8873 | 19371 | 21999 | 24627 |
| 1799 | 6246 | 11497 | 14119 | 16744 | 1800 | 8874 | 19372 | 22000 | 24628 |
| 1801 | 6247 | 11498 | 14120 | 16745 | 1802 | 8875 | 19373 | 22001 | 24629 |
| 1803 | 6248 | 11499 | 14121 | 16746 | 1804 | 8876 | 19374 | 22002 | 24630 |
| 1805 | 6249 | 11500 | 14122 | 16747 | 1806 | 8877 | 19375 | 22003 | 24631 |
| 1807 | 6250 | 11501 | 14123 | 16748 | 1808 | 8878 | 19376 | 22004 | 24632 |
| 1809 | 6251 | 11502 | 14124 | 16749 | 1810 | 8879 | 19377 | 22005 | 24633 |
| 1811 | 6252 | 11503 | 14125 | 16750 | 1812 | 8880 | 19378 | 22006 | 24634 |
| 1813 | 6253 | 11504 | 14126 | 16751 | 1814 | 8881 | 19379 | 22007 | 24635 |
| 1815 | 6254 | 11505 | 14127 | 16752 | 1816 | 8882 | 19380 | 22008 | 24636 |
| 1817 | 6255 | 11506 | 14128 | 16753 | 1818 | 8883 | 19381 | 22009 | 24637 |
| 1819 | 6256 | 11507 | 14129 | 16754 | 1820 | 8884 | 19382 | 22010 | 24638 |
| 1821 | 6257 | 11508 | 14130 | 16755 | 1822 | 8885 | 19383 | 22011 | 24639 |
| 1823 | 6258 | 11509 | 14131 | 16756 | 1824 | 8886 | 19384 | 22012 | 24640 |
| 1825 | 6259 | 11510 | 14132 | 16757 | 1826 | 8887 | 19385 | 22013 | 24641 |
| 1827 | 6260 | 11511 | 14133 | 16758 | 1828 | 8888 | 19386 | 22014 | 24642 |
| 1829 | 6261 | 11512 | 14134 | 16759 | 1830 | 8889 | 19387 | 22015 | 24643 |
| 1831 | 6262 | 11513 | 14135 | 16760 | 1832 | 8890 | 19388 | 22016 | 24644 |
| 1833 | 6263 | 11514 | 14136 | 16761 | 1834 | 8891 | 19389 | 22017 | 24645 |
| 1835 | 6264 | 11515 | 14137 | 16762 | 1836 | 8892 | 19390 | 22018 | 24646 |
| 1837 | 6265 | 11516 | 14138 | 16763 | 1838 | 8893 | 19391 | 22019 | 24647 |
| 1839 | 6266 | 11517 | 14139 | 16764 | 1840 | 8894 | 19392 | 22020 | 24648 |
| 1841 | 6267 | 11518 | 14140 | 16765 | 1842 | 8895 | 19393 | 22021 | 24649 |
| 1843 | 6268 | 11519 | 14141 | 16766 | 1844 | 8896 | 19394 | 22022 | 24650 |
| 1845 | 6269 | 11520 | 14142 | 16767 | 1846 | 8897 | 19395 | 22023 | 24651 |
| 1847 | 6270 | 11521 | 14143 | 16768 | 1848 | 8898 | 19396 | 22024 | 24652 |
| 1849 | 6271 | 11522 | 14144 | 16769 | 1850 | 8899 | 19397 | 22025 | 24653 |
| 1851 | 6272 | 11523 | 14145 | 16770 | 1852 | 8900 | 19398 | 22026 | 24654 |
| 1853 | 6273 | 11524 | 14146 | 16771 | 1854 | 8901 | 19399 | 22027 | 24655 |
| 1855 | 6274 | 11525 | 14147 | 16772 | 1856 | 8902 | 19400 | 22028 | 24656 |
| 1857 | 6275 | 11526 | 14148 | 16773 | 1858 | 8903 | 19401 | 22029 | 24657 |
| 1859 | 6276 | 11527 | 14149 | 16774 | 1860 | 8904 | 19402 | 22030 | 24658 |
| 1861 | 6277 | 11528 | 14150 | 16775 | 1862 | 8905 | 19403 | 22031 | 24659 |
| 1863 | 6278 | 11529 | 14151 | 16776 | 1864 | 8906 | 19404 | 22032 | 24660 |
| 1865 | 6279 | 11530 | 14152 | 16777 | 1866 | 8907 | 19405 | 22033 | 24661 |
| 1867 | 6280 | 11531 | 14153 | 16778 | 1868 | 8908 | 19406 | 22034 | 24662 |
| 1869 | 6281 | 11532 | 14154 | 16779 | 1870 | 8909 | 19407 | 22035 | 24663 |
| 1871 | 6282 | 11533 | 14155 | 16780 | 1872 | 8910 | 19408 | 22036 | 24664 |
| 1873 | 6283 | 11534 | 14156 | 16781 | 1874 | 8911 | 19409 | 22037 | 24665 |
| 1875 | 6284 | 11535 | 14157 | 16782 | 1876 | 8912 | 19410 | 22038 | 24666 |
| 1877 | 6285 | 11536 | 14158 | 16783 | 1878 | 8913 | 19411 | 22039 | 24667 |
| 1879 | 6286 | 11537 | 14159 | 16784 | 1880 | 8914 | 19412 | 22040 | 24668 |
| 1881 | 6287 | 11538 | 14160 | 16785 | 1882 | 8915 | 19413 | 22041 | 24669 |
| 1883 | 6288 | 11539 | 14161 | 16786 | 1884 | 8916 | 19414 | 22042 | 24670 |
| 1885 | 6289 | 11540 | 14162 | 16787 | 1886 | 8917 | 19415 | 22043 | 24671 |
| 1887 | 6290 | 11541 | 14163 | 16788 | 1888 | 8918 | 19416 | 22044 | 24672 |
| 1889 | 6291 | 11542 | 14164 | 16789 | 1890 | 8919 | 19417 | 22045 | 24673 |
| 1891 | 6292 | 11543 | 14165 | 16790 | 1892 | 8920 | 19418 | 22046 | 24674 |
| 1893 | 6293 | 11544 | 14166 | 16791 | 1894 | 8921 | 19419 | 22047 | 24675 |
| 1895 | 6294 | 11545 | 14167 | 16792 | 1896 | 8922 | 19420 | 22048 | 24676 |
| 1897 | 6295 | 11546 | 14168 | 16793 | 1898 | 8923 | 19421 | 22049 | 24677 |
| 1899 | 6296 | 11547 | 14169 | 16794 | 1900 | 8924 | 19422 | 22050 | 24678 |
| 1901 | 6297 | 11548 | 14170 | 16795 | 1902 | 8925 | 19423 | 22051 | 24679 |
| 1903 | 6298 | 11549 | 14171 | 16796 | 1904 | 8926 | 19424 | 22052 | 24680 |
| 1905 | 6299 | 11550 | 14172 | 16797 | 1906 | 8927 | 19425 | 22053 | 24681 |
| 1907 | 6300 | 11551 | 14173 | 16798 | 1908 | 8928 | 19426 | 22054 | 24682 |
| 1909 | 6301 | 11552 | 14174 | 16799 | 1910 | 8929 | 19427 | 22055 | 24683 |
| 1911 | 6302 | 11553 | 14175 | 16800 | 1912 | 8930 | 19428 | 22056 | 24684 |
| 1913 | 6303 | 11554 | 14176 | 16801 | 1914 | 8931 | 19429 | 22057 | 24685 |
| 1915 | 6304 | 11555 | 14177 | 16802 | 1916 | 8932 | 19430 | 22058 | 24686 |
| 1917 | 6305 | 11556 | 14178 | 16803 | 1918 | 8933 | 19431 | 22059 | 24687 |
| 1919 | 6306 | 11557 | 14179 | 16804 | 1920 | 8934 | 19432 | 22060 | 24688 |
| 1921 | 6307 | 11558 | 14180 | 16805 | 1922 | 8935 | 19433 | 22061 | 24689 |
| 1923 | 6308 | 11559 | 14181 | 16806 | 1924 | 8936 | 19434 | 22062 | 24690 |
| 1925 | 6309 | 11560 | 14182 | 16807 | 1926 | 8937 | 19435 | 22063 | 24691 |
| 1927 | 6310 | 11561 | 14183 | 16808 | 1928 | 8938 | 19436 | 22064 | 24692 |
| 1929 | 6311 | 11562 | 14184 | 16809 | 1930 | 8939 | 19437 | 22065 | 24693 |
| 1931 | 6312 | 11563 | 14185 | 16810 | 1932 | 8940 | 19438 | 22066 | 24694 |
| 1933 | 6313 | 11564 | 14186 | 16811 | 1934 | 8941 | 19439 | 22067 | 24695 |
| 1935 | 6314 | 11565 | 14187 | 16812 | 1936 | 8942 | 19440 | 22068 | 24696 |
| 1937 | 6315 | 11566 | 14188 | 16813 | 1938 | 8943 | 19441 | 22069 | 24697 |
| 1939 | 6316 | 11567 | 14189 | 16814 | 1940 | 8944 | 19442 | 22070 | 24698 |
| 1941 | 6317 | 11568 | 14190 | 16815 | 1942 | 8945 | 19443 | 22071 | 24699 |
| 1943 | 6318 | 11569 | 14191 | 16816 | 1944 | 8946 | 19444 | 22072 | 24700 |
| 1945 | 6319 | 11570 | 14192 | 16817 | 1946 | 8947 | 19445 | 22073 | 24701 |
| 1947 | 6320 | 11571 | 14193 | 16818 | 1948 | 8948 | 19446 | 22074 | 24702 |
| 1949 | 6321 | 11572 | 14194 | 16819 | 1950 | 8949 | 19447 | 22075 | 24703 |
| 1951 | 6322 | 11573 | 14195 | 16820 | 1952 | 8950 | 19448 | 22076 | 24704 |
| 1953 | 6323 | 11574 | 14196 | 16821 | 1954 | 8951 | 19449 | 22077 | 24705 |
| 1955 | 6324 | 11575 | 14197 | 16822 | 1956 | 8952 | 19450 | 22078 | 24706 |
| 1957 | 6325 | 11576 | 14198 | 16823 | 1958 | 8953 | 19451 | 22079 | 24707 |
| 1959 | 6326 | 11577 | 14199 | 16824 | 1960 | 8954 | 19452 | 22080 | 24708 |
| 1961 | 6327 | 11578 | 14200 | 16825 | 1962 | 8955 | 19453 | 22081 | 24709 |
| 1963 | 6328 | 11579 | 14201 | 16826 | 1964 | 8956 | 19454 | 22082 | 24710 |
| 1965 | 6329 | 11580 | 14202 | 16827 | 1966 | 8957 | 19455 | 22083 | 24711 |
| 1967 | 6330 | 11581 | 14203 | 16828 | 1968 | 8958 | 19456 | 22084 | 24712 |
| 1969 | 6331 | 11582 | 14204 | 16829 | 1970 | 8959 | 19457 | 22085 | 24713 |
| 1971 | 6332 | 11583 | 14205 | 16830 | 1972 | 8960 | 19458 | 22086 | 24714 |
| 1973 | 6333 | 11584 | 14206 | 16831 | 1974 | 8961 | 19459 | 22087 | 24715 |
| 1975 | 6334 | 11585 | 14207 | 16832 | 1976 | 8962 | 19460 | 22088 | 24716 |
| 1977 | 6335 | 11586 | 14208 | 16833 | 1978 | 8963 | 19461 | 22089 | 24717 |
| 1979 | 6336 | 11587 | 14209 | 16834 | 1980 | 8964 | 19462 | 22090 | 24718 |
| 1981 | 6337 | 11588 | 14210 | 16835 | 1982 | 8965 | 19463 | 22091 | 24719 |
| 1983 | 6338 | 11589 | 14211 | 16836 | 1984 | 8966 | 19464 | 22092 | 24720 |
| 1985 | 6339 | 11590 | 14212 | 16837 | 1986 | 8967 | 19465 | 22093 | 24721 |
| 1987 | 6340 | 11591 | 14213 | 16838 | 1988 | 8968 | 19466 | 22094 | 24722 |
| 1989 | 6341 | 11592 | 14214 | 16839 | 1990 | 8969 | 19467 | 22095 | 24723 |
| 1991 | 6342 | 11593 | 14215 | 16840 | 1992 | 8970 | 19468 | 22096 | 24724 |
| 1993 | 6343 | 11594 | 14216 | 16841 | 1994 | 8971 | 19469 | 22097 | 24725 |
| 1995 | 6344 | 11595 | 14217 | 16842 | 1996 | 8972 | 19470 | 22098 | 24726 |
| 1997 | 6345 | 11596 | 14218 | 16843 | 1998 | 8973 | 19471 | 22099 | 24727 |
| 1999 | 6346 | 11597 | 14219 | 16844 | 2000 | 8974 | 19472 | 22100 | 24728 |
| 2001 | 6347 | 11598 | 14220 | 16845 | 2002 | 8975 | 19473 | 22101 | 24729 |
| 2003 | 6348 | 11599 | 14221 | 16846 | 2004 | 8976 | 19474 | 22102 | 24730 |
| 2005 | 6349 | 11600 | 14222 | 16847 | 2006 | 8977 | 19475 | 22103 | 24731 |
| 2007 | 6350 | 11601 | 14223 | 16848 | 2008 | 8978 | 19476 | 22104 | 24732 |
| 2009 | 6351 | 11602 | 14224 | 16849 | 2010 | 8979 | 19477 | 22105 | 24733 |
| 2011 | 6352 | 11603 | 14225 | 16850 | 2012 | 8980 | 19478 | 22106 | 24734 |
| 2013 | 6353 | 11604 | 14226 | 16851 | 2014 | 8981 | 19479 | 22107 | 24735 |
| 2015 | 6354 | 11605 | 14227 | 16852 | 2016 | 8982 | 19480 | 22108 | 24736 |
| 2017 | 6355 | 11606 | 14228 | 16853 | 2018 | 8983 | 19481 | 22109 | 24737 |
| 2019 | 6356 | 11607 | 14229 | 16854 | 2020 | 8984 | 19482 | 22110 | 24738 |
| 2021 | 6357 | 11608 | 14230 | 16855 | 2022 | 8985 | 19483 | 22111 | 24739 |
| 2023 | 6358 | 11609 | 14231 | 16856 | 2024 | 8986 | 19484 | 22112 | 24740 |
| 2025 | 6359 | 11610 | 14232 | 16857 | 2026 | 8987 | 19485 | 22113 | 24741 |
| 2027 | 6360 | 11611 | 14233 | 16858 | 2028 | 8988 | 19486 | 22114 | 24742 |
| 2029 | 6361 | 11612 | 14234 | 16859 | 2030 | 8989 | 19487 | 22115 | 24743 |
| 2031 | 6362 | 11613 | 14235 | 16860 | 2032 | 8990 | 19488 | 22116 | 24744 |
| 2033 | 6363 | 11614 | 14236 | 16861 | 2034 | 8991 | 19489 | 22117 | 24745 |
| 2035 | 6364 | 11615 | 14237 | 16862 | 2036 | 8992 | 19490 | 22118 | 24746 |
| 2037 | 6365 | 11616 | 14238 | 16863 | 2038 | 8993 | 19491 | 22119 | 24747 |
| 2039 | 6366 | 11617 | 14239 | 16864 | 2040 | 8994 | 19492 | 22120 | 24748 |
| 2041 | 6367 | 11618 | 14240 | 16865 | 2042 | 8995 | 19493 | 22121 | 24749 |
| 2043 | 6368 | 11619 | 14241 | 16866 | 2044 | 8996 | 19494 | 22122 | 24750 |
| 2045 | 6369 | 11620 | 14242 | 16867 | 2046 | 8997 | 19495 | 22123 | 24751 |
| 2047 | 6370 | 11621 | 14243 | 16868 | 2048 | 8998 | 19496 | 22124 | 24752 |
| 2049 | 6371 | 11622 | 14244 | 16869 | 2050 | 8999 | 19497 | 22125 | 24753 |
| 2051 | 6372 | 11623 | 14245 | 16870 | 2052 | 9000 | 19498 | 22126 | 24754 |
| 2053 | 6373 | 11624 | 14246 | 16871 | 2054 | 9001 | 19499 | 22127 | 24755 |
| 2055 | 6374 | 11625 | 14247 | 16872 | 2056 | 9002 | 19500 | 22128 | 24756 |
| 2057 | 6375 | 11626 | 14248 | 16873 | 2058 | 9003 | 19501 | 22129 | 24757 |
| 2059 | 6376 | 11627 | 14249 | 16874 | 2060 | 9004 | 19502 | 22130 | 24758 |
| 2061 | 6377 | 11628 | 14250 | 16875 | 2062 | 9005 | 19503 | 22131 | 24759 |
| 2063 | 6378 | 11629 | 14251 | 16876 | 2064 | 9006 | 19504 | 22132 | 24760 |
| 2065 | 6379 | 11630 | 14252 | 16877 | 2066 | 9007 | 19505 | 22133 | 24761 |
| 2067 | 6380 | 11631 | 14253 | 16878 | 2068 | 9008 | 19506 | 22134 | 24762 |
| 2069 | 6381 | 11632 | 14254 | 16879 | 2070 | 9009 | 19507 | 22135 | 24763 |
| 2071 | 6382 | 11633 | 14255 | 16880 | 2072 | 9010 | 19508 | 22136 | 24764 |
| 2073 | 6383 | 11634 | 14256 | 16881 | 2074 | 9011 | 19509 | 22137 | 24765 |
| 2075 | 6384 | 11635 | 14257 | 16882 | 2076 | 9012 | 19510 | 22138 | 24766 |
| 2077 | 6385 | 11636 | 14258 | 16883 | 2078 | 9013 | 19511 | 22139 | 24767 |
| 2079 | 6386 | 11637 | 14259 | 16884 | 2080 | 9014 | 19512 | 22140 | 24768 |
| 2081 | 6387 | 11638 | 14260 | 16885 | 2082 | 9015 | 19513 | 22141 | 24769 |
| 2083 | 6388 | 11639 | 14261 | 16886 | 2084 | 9016 | 19514 | 22142 | 24770 |
| 2085 | 6389 | 11640 | 14262 | 16887 | 2086 | 9017 | 19515 | 22143 | 24771 |
| 2087 | 6390 | 11641 | 14263 | 16888 | 2088 | 9018 | 19516 | 22144 | 24772 |
| 2089 | 6391 | 11642 | 14264 | 16889 | 2090 | 9019 | 19517 | 22145 | 24773 |
| 2091 | 6392 | 11643 | 14265 | 16890 | 2092 | 9020 | 19518 | 22146 | 24774 |
| 2093 | 6393 | 11644 | 14266 | 16891 | 2094 | 9021 | 19519 | 22147 | 24775 |
| 2095 | 6394 | 11645 | 14267 | 16892 | 2096 | 9022 | 19520 | 22148 | 24776 |
| 2097 | 6395 | 11646 | 14268 | 16893 | 2098 | 9023 | 19521 | 22149 | 24777 |
| 2099 | 6396 | 11647 | 14269 | 16894 | 2100 | 9024 | 19522 | 22150 | 24778 |
| 2101 | 6397 | 11648 | 14270 | 16895 | 2102 | 9025 | 19523 | 22151 | 24779 |
| 2103 | 6398 | 11649 | 14271 | 16896 | 2104 | 9026 | 19524 | 22152 | 24780 |
| 2105 | 6399 | 11650 | 14272 | 16897 | 2106 | 9027 | 19525 | 22153 | 24781 |
| 2107 | 6400 | 11651 | 14273 | 16898 | 2108 | 9028 | 19526 | 22154 | 24782 |
| 2109 | 6401 | 11652 | 14274 | 16899 | 2110 | 9029 | 19527 | 22155 | 24783 |
| 2111 | 6402 | 11653 | 14275 | 16900 | 2112 | 9030 | 19528 | 22156 | 24784 |
| 2113 | 6403 | 11654 | 14276 | 16901 | 2114 | 9031 | 19529 | 22157 | 24785 |
| 2115 | 6404 | 11655 | 14277 | 16902 | 2116 | 9032 | 19530 | 22158 | 24786 |
| 2117 | 6405 | 11656 | 14278 | 16903 | 2118 | 9033 | 19531 | 22159 | 24787 |
| 2119 | 6406 | 11657 | 14279 | 16904 | 2120 | 9034 | 19532 | 22160 | 24788 |
| 2121 | 6407 | 11658 | 14280 | 16905 | 2122 | 9035 | 19533 | 22161 | 24789 |
| 2123 | 6408 | 11659 | 14281 | 16906 | 2124 | 9036 | 19534 | 22162 | 24790 |
| 2125 | 6409 | 11660 | 14282 | 16907 | 2126 | 9037 | 19535 | 22163 | 24791 |
| 2127 | 6410 | 11661 | 14283 | 16908 | 2128 | 9038 | 19536 | 22164 | 24792 |
| 2129 | 6411 | 11662 | 14284 | 16909 | 2130 | 9039 | 19537 | 22165 | 24793 |
| 2131 | 6412 | 11663 | 14285 | 16910 | 2132 | 9040 | 19538 | 22166 | 24794 |
| 2133 | 6413 | 11664 | 14286 | 16911 | 2134 | 9041 | 19539 | 22167 | 24795 |
| 2135 | 6414 | 11665 | 14287 | 16912 | 2136 | 9042 | 19540 | 22168 | 24796 |
| 2137 | 6415 | 11666 | 14288 | 16913 | 2138 | 9043 | 19541 | 22169 | 24797 |
| 2139 | 6416 | 11667 | 14289 | 16914 | 2140 | 9044 | 19542 | 22170 | 24798 |
| 2141 | 6417 | 11668 | 14290 | 16915 | 2142 | 9045 | 19543 | 22171 | 24799 |
| 2143 | 6418 | 11669 | 14291 | 16916 | 2144 | 9046 | 19544 | 22172 | 24800 |
| 2145 | 6419 | 11670 | 14292 | 16917 | 2146 | 9047 | 19545 | 22173 | 24801 |
| 2147 | 6420 | 11671 | 14293 | 16918 | 2148 | 9048 | 19546 | 22174 | 24802 |
| 2149 | 6421 | 11672 | 14294 | 16919 | 2150 | 9049 | 19547 | 22175 | 24803 |
| 2151 | 6422 | 11673 | 14295 | 16920 | 2152 | 9050 | 19548 | 22176 | 24804 |
| 2153 | 6423 | 11674 | 14296 | 16921 | 2154 | 9051 | 19549 | 22177 | 24805 |
| 2155 | 6424 | 11675 | 14297 | 16922 | 2156 | 9052 | 19550 | 22178 | 24806 |
| 2157 | 6425 | 11676 | 14298 | 16923 | 2158 | 9053 | 19551 | 22179 | 24807 |
| 2159 | 6426 | 11677 | 14299 | 16924 | 2160 | 9054 | 19552 | 22180 | 24808 |
| 2161 | 6427 | 11678 | 14300 | 16925 | 2162 | 9055 | 19553 | 22181 | 24809 |
| 2163 | 6428 | 11679 | 14301 | 16926 | 2164 | 9056 | 19554 | 22182 | 24810 |
| 2165 | 6429 | 11680 | 14302 | 16927 | 2166 | 9057 | 19555 | 22183 | 24811 |
| 2167 | 6430 | 11681 | 14303 | 16928 | 2168 | 9058 | 19556 | 22184 | 24812 |
| 2169 | 6431 | 11682 | 14304 | 16929 | 2170 | 9059 | 19557 | 22185 | 24813 |
| 2171 | 6432 | 11683 | 14305 | 16930 | 2172 | 9060 | 19558 | 22186 | 24814 |
| 2173 | 6433 | 11684 | 14306 | 16931 | 2174 | 9061 | 19559 | 22187 | 24815 |
| 2175 | 6434 | 11685 | 14307 | 16932 | 2176 | 9062 | 19560 | 22188 | 24816 |
| 2177 | 6435 | 11686 | 14308 | 16933 | 2178 | 9063 | 19561 | 22189 | 24817 |
| 2179 | 6436 | 11687 | 14309 | 16934 | 2180 | 9064 | 19562 | 22190 | 24818 |
| 2181 | 6437 | 11688 | 14310 | 16935 | 2182 | 9065 | 19563 | 22191 | 24819 |
| 2183 | 6438 | 11689 | 14311 | 16936 | 2184 | 9066 | 19564 | 22192 | 24820 |
| 2185 | 6439 | 11690 | 14312 | 16937 | 2186 | 9067 | 19565 | 22193 | 24821 |
| 2187 | 6440 | 11691 | 14313 | 16938 | 2188 | 9068 | 19566 | 22194 | 24822 |
| 2189 | 6441 | 11692 | 14314 | 16939 | 2190 | 9069 | 19567 | 22195 | 24823 |
| 2191 | 6442 | 11693 | 14315 | 16940 | 2192 | 9070 | 19568 | 22196 | 24824 |
| 2193 | 6443 | 11694 | 14316 | 16941 | 2194 | 9071 | 19569 | 22197 | 24825 |
| 2195 | 6444 | 11695 | 14317 | 16942 | 2196 | 9072 | 19570 | 22198 | 24826 |
| 2197 | 6445 | 11696 | 14318 | 16943 | 2198 | 9073 | 19571 | 22199 | 24827 |
| 2199 | 6446 | 11697 | 14319 | 16944 | 2200 | 9074 | 19572 | 22200 | 24828 |
| 2201 | 6447 | 11698 | 14320 | 16945 | 2202 | 9075 | 19573 | 22201 | 24829 |
| 2203 | 6448 | 11699 | 14321 | 16946 | 2204 | 9076 | 19574 | 22202 | 24830 |
| 2205 | 6449 | 11700 | 14322 | 16947 | 2206 | 9077 | 19575 | 22203 | 24831 |
| 2207 | 6450 | 11701 | 14323 | 16948 | 2208 | 9078 | 19576 | 22204 | 24832 |
| 2209 | 6451 | 11702 | 14324 | 16949 | 2210 | 9079 | 19577 | 22205 | 24833 |
| 2211 | 6452 | 11703 | 14325 | 16950 | 2212 | 9080 | 19578 | 22206 | 24834 |
| 2213 | 6453 | 11704 | 14326 | 16951 | 2214 | 9081 | 19579 | 22207 | 24835 |
| 2215 | 6454 | 11705 | 14327 | 16952 | 2216 | 9082 | 19580 | 22208 | 24836 |
| 2217 | 6455 | 11706 | 14328 | 16953 | 2218 | 9083 | 19581 | 22209 | 24837 |
| 2219 | 6456 | 11707 | 14329 | 16954 | 2220 | 9084 | 19582 | 22210 | 24838 |
| 2221 | 6457 | 11708 | 14330 | 16955 | 2222 | 9085 | 19583 | 22211 | 24839 |
| 2223 | 6458 | 11709 | 14331 | 16956 | 2224 | 9086 | 19584 | 22212 | 24840 |
| 2225 | 6459 | 11710 | 14332 | 16957 | 2226 | 9087 | 19585 | 22213 | 24841 |
| 2227 | 6460 | 11711 | 14333 | 16958 | 2228 | 9088 | 19586 | 22214 | 24842 |
| 2229 | 6461 | 11712 | 14334 | 16959 | 2230 | 9089 | 19587 | 22215 | 24843 |
| 2231 | 6462 | 11713 | 14335 | 16960 | 2232 | 9090 | 19588 | 22216 | 24844 |
| 2233 | 6463 | 11714 | 14336 | 16961 | 2234 | 9091 | 19589 | 22217 | 24845 |
| 2235 | 6464 | 11715 | 14337 | 16962 | 2236 | 9092 | 19590 | 22218 | 24846 |
| 2237 | 6465 | 11716 | 14338 | 16963 | 2238 | 9093 | 19591 | 22219 | 24847 |
| 2239 | 6466 | 11717 | 14339 | 16964 | 2240 | 9094 | 19592 | 22220 | 24848 |
| 2241 | 6467 | 11718 | 14340 | 16965 | 2242 | 9095 | 19593 | 22221 | 24849 |
| 2243 | 6468 | 11719 | 14341 | 16966 | 2244 | 9096 | 19594 | 22222 | 24850 |
| 2245 | 6469 | 11720 | 14342 | 16967 | 2246 | 9097 | 19595 | 22223 | 24851 |
| 2247 | 6470 | 11721 | 14343 | 16968 | 2248 | 9098 | 19596 | 22224 | 24852 |
| 2249 | 6471 | 11722 | 14344 | 16969 | 2250 | 9099 | 19597 | 22225 | 24853 |
| 2251 | 6472 | 11723 | 14345 | 16970 | 2252 | 9100 | 19598 | 22226 | 24854 |
| 2253 | 6473 | 11724 | 14346 | 16971 | 2254 | 9101 | 19599 | 22227 | 24855 |
| 2255 | 6474 | 11725 | 14347 | 16972 | 2256 | 9102 | 19600 | 22228 | 24856 |
| 2257 | 6475 | 11726 | 14348 | 16973 | 2258 | 9103 | 19601 | 22229 | 24857 |
| 2259 | 6476 | 11727 | 14349 | 16974 | 2260 | 9104 | 19602 | 22230 | 24858 |
| 2261 | 6477 | 11728 | 14350 | 16975 | 2262 | 9105 | 19603 | 22231 | 24859 |
| 2263 | 6478 | 11729 | 14351 | 16976 | 2264 | 9106 | 19604 | 22232 | 24860 |
| 2265 | 6479 | 11730 | 14352 | 16977 | 2266 | 9107 | 19605 | 22233 | 24861 |
| 2267 | 6480 | 11731 | 14353 | 16978 | 2268 | 9108 | 19606 | 22234 | 24862 |
| 2269 | 6481 | 11732 | 14354 | 16979 | 2270 | 9109 | 19607 | 22235 | 24863 |
| 2271 | 6482 | 11733 | 14355 | 16980 | 2272 | 9110 | 19608 | 22236 | 24864 |
| 2273 | 6483 | 11734 | 14356 | 16981 | 2274 | 9111 | 19609 | 22237 | 24865 |
| 2275 | 6484 | 11735 | 14357 | 16982 | 2276 | 9112 | 19610 | 22238 | 24866 |
| 2277 | 6485 | 11736 | 14358 | 16983 | 2278 | 9113 | 19611 | 22239 | 24867 |
| 2279 | 6486 | 11737 | 14359 | 16984 | 2280 | 9114 | 19612 | 22240 | 24868 |
| 2281 | 6487 | 11738 | 14360 | 16985 | 2282 | 9115 | 19613 | 22241 | 24869 |
| 2283 | 6488 | 11739 | 14361 | 16986 | 2284 | 9116 | 19614 | 22242 | 24870 |
| 2285 | 6489 | 11740 | 14362 | 16987 | 2286 | 9117 | 19615 | 22243 | 24871 |
| 2287 | 6490 | 11741 | 14363 | 16988 | 2288 | 9118 | 19616 | 22244 | 24872 |
| 2289 | 6491 | 11742 | 14364 | 16989 | 2290 | 9119 | 19617 | 22245 | 24873 |
| 2291 | 6492 | 11743 | 14365 | 16990 | 2292 | 9120 | 19618 | 22246 | 24874 |
| 2293 | 6493 | 11744 | 14366 | 16991 | 2294 | 9121 | 19619 | 22247 | 24875 |
| 2295 | 6494 | 11745 | 14367 | 16992 | 2296 | 9122 | 19620 | 22248 | 24876 |
| 2297 | 6495 | 11746 | 14368 | 16993 | 2298 | 9123 | 19621 | 22249 | 24877 |
| 2299 | 6496 | 11747 | 14369 | 16994 | 2300 | 9124 | 19622 | 22250 | 24878 |
| 2301 | 6497 | 11748 | 14370 | 16995 | 2302 | 9125 | 19623 | 22251 | 24879 |
| 2303 | 6498 | 11749 | 14371 | 16996 | 2304 | 9126 | 19624 | 22252 | 24880 |
| 2305 | 6499 | 11750 | 14372 | 16997 | 2306 | 9127 | 19625 | 22253 | 24881 |
| 2307 | 6500 | 11751 | 14373 | 16998 | 2308 | 9128 | 19626 | 22254 | 24882 |
| 2309 | 6501 | 11752 | 14374 | 16999 | 2310 | 9129 | 19627 | 22255 | 24883 |
| 2311 | 6502 | 11753 | 14375 | 17000 | 2312 | 9130 | 19628 | 22256 | 24884 |
| 2313 | 6503 | 11754 | 14376 | 17001 | 2314 | 9131 | 19629 | 22257 | 24885 |
| 2315 | 6504 | 11755 | 14377 | 17002 | 2316 | 9132 | 19630 | 22258 | 24886 |
| 2317 | 6505 | 11756 | 14378 | 17003 | 2318 | 9133 | 19631 | 22259 | 24887 |
| 2319 | 6506 | 11757 | 14379 | 17004 | 2320 | 9134 | 19632 | 22260 | 24888 |
| 2321 | 6507 | 11758 | 14380 | 17005 | 2322 | 9135 | 19633 | 22261 | 24889 |
| 2323 | 6508 | 11759 | 14381 | 17006 | 2324 | 9136 | 19634 | 22262 | 24890 |
| 2325 | 6509 | 11760 | 14382 | 17007 | 2326 | 9137 | 19635 | 22263 | 24891 |
| 2327 | 6510 | 11761 | 14383 | 17008 | 2328 | 9138 | 19636 | 22264 | 24892 |
| 2329 | 6511 | 11762 | 14384 | 17009 | 2330 | 9139 | 19637 | 22265 | 24893 |
| 2331 | 6512 | 11763 | 14385 | 17010 | 2332 | 9140 | 19638 | 22266 | 24894 |
| 2333 | 6513 | 11764 | 14386 | 17011 | 2334 | 9141 | 19639 | 22267 | 24895 |
| 2335 | 6514 | 11765 | 14387 | 17012 | 2336 | 9142 | 19640 | 22268 | 24896 |
| 2337 | 6515 | 11766 | 14388 | 17013 | 2338 | 9143 | 19641 | 22269 | 24897 |
| 2339 | 6516 | 11767 | 14389 | 17014 | 2340 | 9144 | 19642 | 22270 | 24898 |
| 2341 | 6517 | 11768 | 14390 | 17015 | 2342 | 9145 | 19643 | 22271 | 24899 |
| 2343 | 6518 | 11769 | 14391 | 17016 | 2344 | 9146 | 19644 | 22272 | 24900 |
| 2345 | 6519 | 11770 | 14392 | 17017 | 2346 | 9147 | 19645 | 22273 | 24901 |
| 2347 | 6520 | 11771 | 14393 | 17018 | 2348 | 9148 | 19646 | 22274 | 24902 |
| 2349 | 6521 | 11772 | 14394 | 17019 | 2350 | 9149 | 19647 | 22275 | 24903 |
| 2351 | 6522 | 11773 | 14395 | 17020 | 2352 | 9150 | 19648 | 22276 | 24904 |
| 2353 | 6523 | 11774 | 14396 | 17021 | 2354 | 9151 | 19649 | 22277 | 24905 |
| 2355 | 6524 | 11775 | 14397 | 17022 | 2356 | 9152 | 19650 | 22278 | 24906 |
| 2357 | 6525 | 11776 | 14398 | 17023 | 2358 | 9153 | 19651 | 22279 | 24907 |
| 2359 | 6526 | 11777 | 14399 | 17024 | 2360 | 9154 | 19652 | 22280 | 24908 |
| 2361 | 6527 | 11778 | 14400 | 17025 | 2362 | 9155 | 19653 | 22281 | 24909 |
| 2363 | 6528 | 11779 | 14401 | 17026 | 2364 | 9156 | 19654 | 22282 | 24910 |
| 2365 | 6529 | 11780 | 14402 | 17027 | 2366 | 9157 | 19655 | 22283 | 24911 |
| 2367 | 6530 | 11781 | 14403 | 17028 | 2368 | 9158 | 19656 | 22284 | 24912 |
| 2369 | 6531 | 11782 | 14404 | 17029 | 2370 | 9159 | 19657 | 22285 | 24913 |
| 2371 | 6532 | 11783 | 14405 | 17030 | 2372 | 9160 | 19658 | 22286 | 24914 |
| 2373 | 6533 | 11784 | 14406 | 17031 | 2374 | 9161 | 19659 | 22287 | 24915 |
| 2375 | 6534 | 11785 | 14407 | 17032 | 2376 | 9162 | 19660 | 22288 | 24916 |
| 2377 | 6535 | 11786 | 14408 | 17033 | 2378 | 9163 | 19661 | 22289 | 24917 |
| 2379 | 6536 | 11787 | 14409 | 17034 | 2380 | 9164 | 19662 | 22290 | 24918 |
| 2381 | 6537 | 11788 | 14410 | 17035 | 2382 | 9165 | 19663 | 22291 | 24919 |
| 2383 | 6538 | 11789 | 14411 | 17036 | 2384 | 9166 | 19664 | 22292 | 24920 |
| 2385 | 6539 | 11790 | 14412 | 17037 | 2386 | 9167 | 19665 | 22293 | 24921 |
| 2387 | 6540 | 11791 | 14413 | 17038 | 2388 | 9168 | 19666 | 22294 | 24922 |
| 2389 | 6541 | 11792 | 14414 | 17039 | 2390 | 9169 | 19667 | 22295 | 24923 |
| 2391 | 6542 | 11793 | 14415 | 17040 | 2392 | 9170 | 19668 | 22296 | 24924 |
| 2393 | 6543 | 11794 | 14416 | 17041 | 2394 | 9171 | 19669 | 22297 | 24925 |
| 2395 | 6544 | 11795 | 14417 | 17042 | 2396 | 9172 | 19670 | 22298 | 24926 |
| 2397 | 6545 | 11796 | 14418 | 17043 | 2398 | 9173 | 19671 | 22299 | 24927 |
| 2399 | 6546 | 11797 | 14419 | 17044 | 2400 | 9174 | 19672 | 22300 | 24928 |
| 2401 | 6547 | 11798 | 14420 | 17045 | 2402 | 9175 | 19673 | 22301 | 24929 |
| 2403 | 6548 | 11799 | 14421 | 17046 | 2404 | 9176 | 19674 | 22302 | 24930 |
| 2405 | 6549 | 11800 | 14422 | 17047 | 2406 | 9177 | 19675 | 22303 | 24931 |
| 2407 | 6550 | 11801 | 14423 | 17048 | 2408 | 9178 | 19676 | 22304 | 24932 |
| 2409 | 6551 | 11802 | 14424 | 17049 | 2410 | 9179 | 19677 | 22305 | 24933 |
| 2411 | 6552 | 11803 | 14425 | 17050 | 2412 | 9180 | 19678 | 22306 | 24934 |
| 2413 | 6553 | 11804 | 14426 | 17051 | 2414 | 9181 | 19679 | 22307 | 24935 |
| 2415 | 6554 | 11805 | 14427 | 17052 | 2416 | 9182 | 19680 | 22308 | 24936 |
| 2417 | 6555 | 11806 | 14428 | 17053 | 2418 | 9183 | 19681 | 22309 | 24937 |
| 2419 | 6556 | 11807 | 14429 | 17054 | 2420 | 9184 | 19682 | 22310 | 24938 |
| 2421 | 6557 | 11808 | 14430 | 17055 | 2422 | 9185 | 19683 | 22311 | 24939 |
| 2423 | 6558 | 11809 | 14431 | 17056 | 2424 | 9186 | 19684 | 22312 | 24940 |
| 2425 | 6559 | 11810 | 14432 | 17057 | 2426 | 9187 | 19685 | 22313 | 24941 |
| 2427 | 6560 | 11811 | 14433 | 17058 | 2428 | 9188 | 19686 | 22314 | 24942 |
| 2429 | 6561 | 11812 | 14434 | 17059 | 2430 | 9189 | 19687 | 22315 | 24943 |
| 2431 | 6562 | 11813 | 14435 | 17060 | 2432 | 9190 | 19688 | 22316 | 24944 |
| 2433 | 6563 | 11814 | 14436 | 17061 | 2434 | 9191 | 19689 | 22317 | 24945 |
| 2435 | 6564 | 11815 | 14437 | 17062 | 2436 | 9192 | 19690 | 22318 | 24946 |
| 2437 | 6565 | 11816 | 14438 | 17063 | 2438 | 9193 | 19691 | 22319 | 24947 |
| 2439 | 6566 | 11817 | 14439 | 17064 | 2440 | 9194 | 19692 | 22320 | 24948 |
| 2441 | 6567 | 11818 | 14440 | 17065 | 2442 | 9195 | 19693 | 22321 | 24949 |
| 2443 | 6568 | 11819 | 14441 | 17066 | 2444 | 9196 | 19694 | 22322 | 24950 |
| 2445 | 6569 | 11820 | 14442 | 17067 | 2446 | 9197 | 19695 | 22323 | 24951 |
| 2447 | 6570 | 11821 | 14443 | 17068 | 2448 | 9198 | 19696 | 22324 | 24952 |
| 2449 | 6571 | 11822 | 14444 | 17069 | 2450 | 9199 | 19697 | 22325 | 24953 |
| 2451 | 6572 | 11823 | 14445 | 17070 | 2452 | 9200 | 19698 | 22326 | 24954 |
| 2453 | 6573 | 11824 | 14446 | 17071 | 2454 | 9201 | 19699 | 22327 | 24955 |
| 2455 | 6574 | 11825 | 14447 | 17072 | 2456 | 9202 | 19700 | 22328 | 24956 |
| 2457 | 6575 | 11826 | 14448 | 17073 | 2458 | 9203 | 19701 | 22329 | 24957 |
| 2459 | 6576 | 11827 | 14449 | 17074 | 2460 | 9204 | 19702 | 22330 | 24958 |
| 2461 | 6577 | 11828 | 14450 | 17075 | 2462 | 9205 | 19703 | 22331 | 24959 |
| 2463 | 6578 | 11829 | 14451 | 17076 | 2464 | 9206 | 19704 | 22332 | 24960 |
| 2465 | 6579 | 11830 | 14452 | 17077 | 2466 | 9207 | 19705 | 22333 | 24961 |
| 2467 | 6580 | 11831 | 14453 | 17078 | 2468 | 9208 | 19706 | 22334 | 24962 |
| 2469 | 6581 | 11832 | 14454 | 17079 | 2470 | 9209 | 19707 | 22335 | 24963 |
| 2471 | 6582 | 11833 | 14455 | 17080 | 2472 | 9210 | 19708 | 22336 | 24964 |
| 2473 | 6583 | 11834 | 14456 | 17081 | 2474 | 9211 | 19709 | 22337 | 24965 |
| 2475 | 6584 | 11835 | 14457 | 17082 | 2476 | 9212 | 19710 | 22338 | 24966 |
| 2477 | 6585 | 11836 | 14458 | 17083 | 2478 | 9213 | 19711 | 22339 | 24967 |
| 2479 | 6586 | 11837 | 14459 | 17084 | 2480 | 9214 | 19712 | 22340 | 24968 |
| 2481 | 6587 | 11838 | 14460 | 17085 | 2482 | 9215 | 19713 | 22341 | 24969 |
| 2483 | 6588 | 11839 | 14461 | 17086 | 2484 | 9216 | 19714 | 22342 | 24970 |
| 2485 | 6589 | 11840 | 14462 | 17087 | 2486 | 9217 | 19715 | 22343 | 24971 |
| 2487 | 6590 | 11841 | 14463 | 17088 | 2488 | 9218 | 19716 | 22344 | 24972 |
| 2489 | 6591 | 11842 | 14464 | 17089 | 2490 | 9219 | 19717 | 22345 | 24973 |
| 2491 | 6592 | 11843 | 14465 | 17090 | 2492 | 9220 | 19718 | 22346 | 24974 |
| 2493 | 6593 | 11844 | 14466 | 17091 | 2494 | 9221 | 19719 | 22347 | 24975 |
| 2495 | 6594 | 11845 | 14467 | 17092 | 2496 | 9222 | 19720 | 22348 | 24976 |
| 2497 | 6595 | 11846 | 14468 | 17093 | 2498 | 9223 | 19721 | 22349 | 24977 |
| 2499 | 6596 | 11847 | 14469 | 17094 | 2500 | 9224 | 19722 | 22350 | 24978 |
| 2501 | 6597 | 11848 | 14470 | 17095 | 2502 | 9225 | 19723 | 22351 | 24979 |
| 2503 | 6598 | 11849 | 14471 | 17096 | 2504 | 9226 | 19724 | 22352 | 24980 |
| 2505 | 6599 | 11850 | 14472 | 17097 | 2506 | 9227 | 19725 | 22353 | 24981 |
| 2507 | 6600 | 11851 | 14473 | 17098 | 2508 | 9228 | 19726 | 22354 | 24982 |
| 2509 | 6601 | 11852 | 14474 | 17099 | 2510 | 9229 | 19727 | 22355 | 24983 |
| 2511 | 6602 | 11853 | 14475 | 17100 | 2512 | 9230 | 19728 | 22356 | 24984 |
| 2513 | 6603 | 11854 | 14476 | 17101 | 2514 | 9231 | 19729 | 22357 | 24985 |
| 2515 | 6604 | | 14477 | 17102 | 2516 | 9232 | 19730 | 22358 | 24986 |
| 2517 | 6605 | 11855 | 14478 | 17103 | 2518 | 9233 | 19731 | 22359 | 24987 |
| 2519 | 6606 | 11856 | 14479 | 17104 | 2520 | 9234 | 19732 | 22360 | 24988 |
| 2521 | 6607 | | | 17105 | 2522 | 9235 | 19733 | 22361 | 24989 |
| 2523 | 6608 | 11857 | 14480 | 17106 | 2524 | 9236 | 19734 | 22362 | 24990 |
| 2525 | 6609 | 11858 | 14481 | 17107 | 2526 | 9237 | 19735 | 22363 | 24991 |
| 2527 | 6610 | 11859 | 14482 | 17108 | 2528 | 9238 | 19736 | 22364 | 24992 |
| 2529 | 6611 | 11860 | 14483 | 17109 | 2530 | 9239 | 19737 | 22365 | 24993 |
| 2531 | 6612 | 11861 | 14484 | 17110 | 2532 | 9240 | 19738 | 22366 | 24994 |
| 2533 | 6613 | 11862 | 14485 | 17111 | 2534 | 9241 | 19739 | 22367 | 24995 |
| 2535 | 6614 | 11863 | 14486 | 17112 | 2536 | 9242 | 19740 | 22368 | 24996 |
| 2537 | 6615 | 11864 | 14487 | 17113 | 2538 | 9243 | 19741 | 22369 | 24997 |
| 2539 | 6616 | 11865 | 14488 | 17114 | 2540 | 9244 | 19742 | 22370 | 24998 |
| 2541 | 6617 | 11866 | 14489 | 17115 | 2542 | 9245 | 19743 | 22371 | 24999 |
| 2543 | 6618 | 11867 | 14490 | 17116 | 2544 | 9246 | 19744 | 22372 | 25000 |
| 2545 | 6619 | 11868 | 14491 | 17117 | 2546 | 9247 | 19745 | 22373 | 25001 |
| 2547 | 6620 | 11869 | 14492 | 17118 | 2548 | 9248 | 19746 | 22374 | 25002 |
| 2549 | 6621 | 11870 | 14493 | 17119 | 2550 | 9249 | 19747 | 22375 | 25003 |
| 2551 | 6622 | 11871 | 14494 | 17120 | 2552 | 9250 | 19748 | 22376 | 25004 |
| 2553 | 6623 | 11872 | 14495 | 17121 | 2554 | 9251 | 19749 | 22377 | 25005 |
| 2555 | 6624 | 11873 | 14496 | 17122 | 2556 | 9252 | 19750 | 22378 | 25006 |
| 2557 | 6625 | 11874 | 14497 | 17123 | 2558 | 9253 | 19751 | 22379 | 25007 |
| 2559 | 6626 | 11875 | 14498 | 17124 | 2560 | 9254 | 19752 | 22380 | 25008 |
| 2561 | 6627 | 11876 | 14499 | 17125 | 2562 | 9255 | 19753 | 22381 | 25009 |
| 2563 | 6628 | 11877 | 14500 | 17126 | 2564 | 9256 | 19754 | 22382 | 25010 |
| 2565 | 6629 | 11878 | 14501 | 17127 | 2566 | 9257 | 19755 | 22383 | 25011 |
| 2567 | 6630 | 11879 | 14502 | 17128 | 2568 | 9258 | 19756 | 22384 | 25012 |
| 2569 | 6631 | 11880 | 14503 | 17129 | 2570 | 9259 | 19757 | 22385 | 25013 |
| 2571 | 6632 | 11881 | 14504 | 17130 | 2572 | 9260 | 19758 | 22386 | 25014 |
| 2573 | 6633 | 11882 | 14505 | 17131 | 2574 | 9261 | 19759 | 22387 | 25015 |
| 2575 | 6634 | 11883 | 14506 | 17132 | 2576 | 9262 | 19760 | 22388 | 25016 |
| 2577 | 6635 | 11884 | 14507 | 17133 | 2578 | 9263 | 19761 | 22389 | 25017 |
| 2579 | 6636 | 11885 | 14508 | 17134 | 2580 | 9264 | 19762 | 22390 | 25018 |
| 2581 | 6637 | 11886 | 14509 | 17135 | 2582 | 9265 | 19763 | 22391 | 25019 |
| 2583 | 6638 | 11887 | 14510 | 17136 | 2584 | 9266 | 19764 | 22392 | 25020 |
| 2585 | 6639 | 11888 | 14511 | 17137 | 2586 | 9267 | 19765 | 22393 | 25021 |
| 2587 | 6640 | 11889 | 14512 | 17138 | 2588 | 9268 | 19766 | 22394 | 25022 |
| 2589 | 6641 | 11890 | 14513 | 17139 | 2590 | 9269 | 19767 | 22395 | 25023 |
| 2591 | 6642 | 11891 | 14514 | 17140 | 2592 | 9270 | 19768 | 22396 | 25024 |
| 2593 | 6643 | 11892 | 14515 | 17141 | 2594 | 9271 | 19769 | 22397 | 25025 |
| 2595 | 6644 | 11893 | 14516 | 17142 | 2596 | 9272 | 19770 | 22398 | 25026 |
| 2597 | 6645 | 11894 | 14517 | 17143 | 2598 | 9273 | 19771 | 22399 | 25027 |
| 2599 | 6646 | 11895 | 14518 | 17144 | 2600 | 9274 | 19772 | 22400 | 25028 |
| 2601 | 6647 | 11896 | 14519 | 17145 | 2602 | 9275 | 19773 | 22401 | 25029 |
| 2603 | 6648 | 11897 | 14520 | 17146 | 2604 | 9276 | 19774 | 22402 | 25030 |
| 2605 | 6649 | 11898 | 14521 | 17147 | 2606 | 9277 | 19775 | 22403 | 25031 |
| 2607 | 6650 | 11899 | 14522 | 17148 | 2608 | 9278 | 19776 | 22404 | 25032 |
| 2609 | 6651 | 11900 | 14523 | 17149 | 2610 | 9279 | 19777 | 22405 | 25033 |
| 2611 | 6652 | 11901 | 14524 | 17150 | 2612 | 9280 | 19778 | 22406 | 25034 |
| 2613 | 6653 | 11902 | 14525 | 17151 | 2614 | 9281 | 19779 | 22407 | 25035 |
| 2615 | 6654 | 11903 | 14526 | 17152 | 2616 | 9282 | 19780 | 22408 | 25036 |
| 2617 | 6655 | 11904 | 14527 | 17153 | 2618 | 9283 | 19781 | 22409 | 25037 |
| 2619 | 6656 | 11905 | 14528 | 17154 | 2620 | 9284 | 19782 | 22410 | 25038 |
| 2621 | 6657 | 11906 | 14529 | 17155 | 2622 | 9285 | 19783 | 22411 | 25039 |
| 2623 | 6658 | 11907 | 14530 | 17156 | 2624 | 9286 | 19784 | 22412 | 25040 |
| 2625 | 6659 | 11908 | 14531 | 17157 | 2626 | 9287 | 19785 | 22413 | 25041 |
| 2627 | 6660 | 11909 | 14532 | 17158 | 2628 | 9288 | 19786 | 22414 | 25042 |
| 2629 | 6661 | 11910 | 14533 | 17159 | 2630 | 9289 | 19787 | 22415 | 25043 |
| 2631 | 6662 | 11911 | 14534 | 17160 | 2632 | 9290 | 19788 | 22416 | 25044 |
| 2633 | 6663 | 11912 | 14535 | 17161 | 2634 | 9291 | 19789 | 22417 | 25045 |
| 2635 | 6664 | 11913 | 14536 | 17162 | 2636 | 9292 | 19790 | 22418 | 25046 |
| 2637 | 6665 | 11914 | 14537 | 17163 | 2638 | 9293 | 19791 | 22419 | 25047 |
| 2639 | 6666 | 11915 | 14538 | 17164 | 2640 | 9294 | 19792 | 22420 | 25048 |
| 2641 | 6667 | 11916 | 14539 | 17165 | 2642 | 9295 | 19793 | 22421 | 25049 |
| 2643 | 6668 | 11917 | 14540 | 17166 | 2644 | 9296 | 19794 | 22422 | 25050 |
| 2645 | 6669 | 11918 | 14541 | 17167 | 2646 | 9297 | 19795 | 22423 | 25051 |
| 2647 | 6670 | 11919 | 14542 | 17168 | 2648 | 9298 | 19796 | 22424 | 25052 |
| 2649 | 6671 | 11920 | 14543 | 17169 | 2650 | 9299 | 19797 | 22425 | 25053 |
| 2651 | 6672 | 11921 | 14544 | 17170 | 2652 | 9300 | 19798 | 22426 | 25054 |
| 2653 | 6673 | 11922 | 14545 | 17171 | 2654 | 9301 | 19799 | 22427 | 25055 |
| 2655 | 6674 | 11923 | 14546 | 17172 | 2656 | 9302 | 19800 | 22428 | 25056 |
| 2657 | 6675 | 11924 | 14547 | 17173 | 2658 | 9303 | 19801 | 22429 | 25057 |
| 2659 | 6676 | 11925 | 14548 | 17174 | 2660 | 9304 | 19802 | 22430 | 25058 |
| 2661 | 6677 | 11926 | 14549 | 17175 | 2662 | 9305 | 19803 | 22431 | 25059 |
| 2663 | 6678 | 11927 | 14550 | 17176 | 2664 | 9306 | 19804 | 22432 | 25060 |
| 2665 | 6679 | 11928 | 14551 | 17177 | 2666 | 9307 | 19805 | 22433 | 25061 |
| 2667 | 6680 | 11929 | 14552 | 17178 | 2668 | 9308 | 19806 | 22434 | 25062 |
| 2669 | 6681 | 11930 | 14553 | 17179 | 2670 | 9309 | 19807 | 22435 | 25063 |
| 2671 | 6682 | 11931 | 14554 | 17180 | 2672 | 9310 | 19808 | 22436 | 25064 |
| 2673 | 6683 | 11932 | 14555 | 17181 | 2674 | 9311 | 19809 | 22437 | 25065 |
| 2675 | 6684 | 11933 | 14556 | 17182 | 2676 | 9312 | 19810 | 22438 | 25066 |
| 2677 | 6685 | 11934 | 14557 | 17183 | 2678 | 9313 | 19811 | 22439 | 25067 |
| 2679 | 6686 | 11935 | 14558 | 17184 | 2680 | 9314 | 19812 | 22440 | 25068 |
| 2681 | 6687 | 11936 | 14559 | 17185 | 2682 | 9315 | 19813 | 22441 | 25069 |
| 2683 | 6688 | 11937 | 14560 | 17186 | 2684 | 9316 | 19814 | 22442 | 25070 |
| 2685 | 6689 | 11938 | 14561 | 17187 | 2686 | 9317 | 19815 | 22443 | 25071 |
| 2687 | 6690 | 11939 | 14562 | 17188 | 2688 | 9318 | 19816 | 22444 | 25072 |
| 2689 | 6691 | 11940 | 14563 | 17189 | 2690 | 9319 | 19817 | 22445 | 25073 |
| 2691 | 6692 | 11941 | 14564 | 17190 | 2692 | 9320 | 19818 | 22446 | 25074 |
| 2693 | 6693 | 11942 | 14565 | 17191 | 2694 | 9321 | 19819 | 22447 | 25075 |
| 2695 | 6694 | 11943 | 14566 | 17192 | 2696 | 9322 | 19820 | 22448 | 25076 |
| 2697 | 6695 | 11944 | 14567 | 17193 | 2698 | 9323 | 19821 | 22449 | 25077 |
| 2699 | 6696 | 11945 | 14568 | 17194 | 2700 | 9324 | 19822 | 22450 | 25078 |
| 2701 | 6697 | 11946 | 14569 | 17195 | 2702 | 9325 | 19823 | 22451 | 25079 |
| 2703 | 6698 | 11947 | 14570 | 17196 | 2704 | 9326 | 19824 | 22452 | 25080 |
| 2705 | 6699 | 11948 | 14571 | 17197 | 2706 | 9327 | 19825 | 22453 | 25081 |
| 2707 | 6700 | 11949 | 14572 | 17198 | 2708 | 9328 | 19826 | 22454 | 25082 |
| 2709 | 6701 | 11950 | 14573 | 17199 | 2710 | 9329 | 19827 | 22455 | 25083 |
| 2711 | 6702 | 11951 | 14574 | 17200 | 2712 | 9330 | 19828 | 22456 | 25084 |
| 2713 | 6703 | 11952 | 14575 | 17201 | 2714 | 9331 | 19829 | 22457 | 25085 |
| 2715 | 6704 | 11953 | 14576 | 17202 | 2716 | 9332 | 19830 | 22458 | 25086 |
| 2717 | 6705 | 11954 | 14577 | 17203 | 2718 | 9333 | 19831 | 22459 | 25087 |
| 2719 | 6706 | 11955 | 14578 | 17204 | 2720 | 9334 | 19832 | 22460 | 25088 |
| 2721 | 6707 | 11956 | 14579 | 17205 | 2722 | 9335 | 19833 | 22461 | 25089 |
| 2723 | 6708 | 11957 | 14580 | 17206 | 2724 | 9336 | 19834 | 22462 | 25090 |
| 2725 | 6709 | 11958 | 14581 | 17207 | 2726 | 9337 | 19835 | 22463 | 25091 |
| 2727 | 6710 | 11959 | 14582 | 17208 | 2728 | 9338 | 19836 | 22464 | 25092 |
| 2729 | 6711 | 11960 | 14583 | 17209 | 2730 | 9339 | 19837 | 22465 | 25093 |
| 2731 | 6712 | 11961 | 14584 | 17210 | 2732 | 9340 | 19838 | 22466 | 25094 |
| 2733 | 6713 | 11962 | 14585 | 17211 | 2734 | 9341 | 19839 | 22467 | 25095 |
| 2735 | 6714 | 11963 | 14586 | 17212 | 2736 | 9342 | 19840 | 22468 | 25096 |
| 2737 | 6715 | 11964 | 14587 | 17213 | 2738 | 9343 | 19841 | 22469 | 25097 |
| 2739 | 6716 | 11965 | 14588 | 17214 | 2740 | 9344 | 19842 | 22470 | 25098 |
| 2741 | 6717 | 11966 | 14589 | 17215 | 2742 | 9345 | 19843 | 22471 | 25099 |
| 2743 | 6718 | 11967 | 14590 | 17216 | 2744 | 9346 | 19844 | 22472 | 25100 |
| 2745 | 6719 | 11968 | 14591 | 17217 | 2746 | 9347 | 19845 | 22473 | 25101 |
| 2747 | 6720 | 11969 | 14592 | 17218 | 2748 | 9348 | 19846 | 22474 | 25102 |
| 2749 | 6721 | 11970 | 14593 | 17219 | 2750 | 9349 | 19847 | 22475 | 25103 |
| 2751 | 6722 | 11971 | 14594 | 17220 | 2752 | 9350 | 19848 | 22476 | 25104 |
| 2753 | 6723 | 11972 | 14595 | 17221 | 2754 | 9351 | 19849 | 22477 | 25105 |
| 2755 | 6724 | 11973 | 14596 | 17222 | 2756 | 9352 | 19850 | 22478 | 25106 |
| 2757 | 6725 | 11974 | 14597 | 17223 | 2758 | 9353 | 19851 | 22479 | 25107 |
| 2759 | 6726 | 11975 | 14598 | 17224 | 2760 | 9354 | 19852 | 22480 | 25108 |
| 2761 | 6727 | 11976 | 14599 | 17225 | 2762 | 9355 | 19853 | 22481 | 25109 |
| 2763 | 6728 | 11977 | 14600 | 17226 | 2764 | 9356 | 19854 | 22482 | 25110 |
| 2765 | 6729 | 11978 | 14601 | 17227 | 2766 | 9357 | 19855 | 22483 | 25111 |
| 2767 | 6730 | 11979 | 14602 | 17228 | 2768 | 9358 | 19856 | 22484 | 25112 |
| 2769 | 6731 | 11980 | 14603 | 17229 | 2770 | 9359 | 19857 | 22485 | 25113 |
| 2771 | 6732 | 11981 | 14604 | 17230 | 2772 | 9360 | 19858 | 22486 | 25114 |
| 2773 | 6733 | 11982 | 14605 | 17231 | 2774 | 9361 | 19859 | 22487 | 25115 |
| 2775 | 6734 | 11983 | 14606 | 17232 | 2776 | 9362 | 19860 | 22488 | 25116 |
| 2777 | 6735 | 11984 | 14607 | 17233 | 2778 | 9363 | 19861 | 22489 | 25117 |
| 2779 | 6736 | 11985 | 14608 | 17234 | 2780 | 9364 | 19862 | 22490 | 25118 |
| 2781 | 6737 | 11986 | 14609 | 17235 | 2782 | 9365 | 19863 | 22491 | 25119 |
| 2783 | 6738 | 11987 | 14610 | 17236 | 2784 | 9366 | 19864 | 22492 | 25120 |
| 2785 | 6739 | 11988 | 14611 | 17237 | 2786 | 9367 | 19865 | 22493 | 25121 |
| 2787 | 6740 | 11989 | 14612 | 17238 | 2788 | 9368 | 19866 | 22494 | 25122 |
| 2789 | 6741 | 11990 | 14613 | 17239 | 2790 | 9369 | 19867 | 22495 | 25123 |
| 2791 | 6742 | 11991 | 14614 | 17240 | 2792 | 9370 | 19868 | 22496 | 25124 |
| 2793 | 6743 | 11992 | 14615 | 17241 | 2794 | 9371 | 19869 | 22497 | 25125 |
| 2795 | 6744 | 11993 | 14616 | 17242 | 2796 | 9372 | 19870 | 22498 | 25126 |
| 2797 | 6745 | 11994 | 14617 | 17243 | 2798 | 9373 | 19871 | 22499 | 25127 |
| 2799 | 6746 | 11995 | 14618 | 17244 | 2800 | 9374 | 19872 | 22500 | 25128 |
| 2801 | 6747 | 11996 | 14619 | 17245 | 2802 | 9375 | 19873 | 22501 | 25129 |
| 2803 | 6748 | 11997 | 14620 | 17246 | 2804 | 9376 | 19874 | 22502 | 25130 |
| 2805 | 6749 | 11998 | 14621 | 17247 | 2806 | 9377 | 19875 | 22503 | 25131 |
| 2807 | 6750 | 11999 | 14622 | 17248 | 2808 | 9378 | 19876 | 22504 | 25132 |
| 2809 | 6751 | 12000 | 14623 | 17249 | 2810 | 9379 | 19877 | 22505 | 25133 |
| 2811 | 6752 | 12001 | 14624 | 17250 | 2812 | 9380 | 19878 | 22506 | 25134 |
| 2813 | 6753 | 12002 | 14625 | 17251 | 2814 | 9381 | 19879 | 22507 | 25135 |
| 2815 | 6754 | 12003 | 14626 | 17252 | 2816 | 9382 | 19880 | 22508 | 25136 |
| 2817 | 6755 | 12004 | 14627 | 17253 | 2818 | 9383 | 19881 | 22509 | 25137 |
| 2819 | 6756 | 12005 | 14628 | 17254 | 2820 | 9384 | 19882 | 22510 | 25138 |
| 2821 | 6757 | 12006 | 14629 | 17255 | 2822 | 9385 | 19883 | 22511 | 25139 |
| 2823 | 6758 | 12007 | 14630 | 17256 | 2824 | 9386 | 19884 | 22512 | 25140 |
| 2825 | 6759 | 12008 | 14631 | 17257 | 2826 | 9387 | 19885 | 22513 | 25141 |
| 2827 | 6760 | 12009 | 14632 | 17258 | 2828 | 9388 | 19886 | 22514 | 25142 |
| 2829 | 6761 | 12010 | 14633 | 17259 | 2830 | 9389 | 19887 | 22515 | 25143 |
| 2831 | 6762 | 12011 | 14634 | 17260 | 2832 | 9390 | 19888 | 22516 | 25144 |
| 2833 | 6763 | 12012 | 14635 | 17261 | 2834 | 9391 | 19889 | 22517 | 25145 |
| 2835 | 6764 | 12013 | 14636 | 17262 | 2836 | 9392 | 19890 | 22518 | 25146 |
| 2837 | 6765 | 12014 | 14637 | 17263 | 2838 | 9393 | 19891 | 22519 | 25147 |
| 2839 | 6766 | 12015 | 14638 | 17264 | 2840 | 9394 | 19892 | 22520 | 25148 |
| 2841 | 6767 | 12016 | 14639 | 17265 | 2842 | 9395 | 19893 | 22521 | 25149 |
| 2843 | 6768 | 12017 | 14640 | 17266 | 2844 | 9396 | 19894 | 22522 | 25150 |
| 2845 | 6769 | 12018 | 14641 | 17267 | 2846 | 9397 | 19895 | 22523 | 25151 |
| 2847 | 6770 | 12019 | 14642 | 17268 | 2848 | 9398 | 19896 | 22524 | 25152 |
| 2849 | 6771 | 12020 | 14643 | 17269 | 2850 | 9399 | 19897 | 22525 | 25153 |
| 2851 | 6772 | 12021 | 14644 | 17270 | 2852 | 9400 | 19898 | 22526 | 25154 |
| 2853 | 6773 | 12022 | 14645 | 17271 | 2854 | 9401 | 19899 | 22527 | 25155 |
| 2855 | 6774 | 12023 | 14646 | 17272 | 2856 | 9402 | 19900 | 22528 | 25156 |
| 2857 | 6775 | 12024 | 14647 | 17273 | 2858 | 9403 | 19901 | 22529 | 25157 |
| 2859 | 6776 | 12025 | 14648 | 17274 | 2860 | 9404 | 19902 | 22530 | 25158 |
| 2861 | 6777 | 12026 | 14649 | 17275 | 2862 | 9405 | 19903 | 22531 | 25159 |
| 2863 | 6778 | 12027 | 14650 | 17276 | 2864 | 9406 | 19904 | 22532 | 25160 |
| 2865 | 6779 | 12028 | 14651 | 17277 | 2866 | 9407 | 19905 | 22533 | 25161 |
| 2867 | 6780 | 12029 | 14652 | 17278 | 2868 | 9408 | 19906 | 22534 | 25162 |
| 2869 | 6781 | 12030 | 14653 | 17279 | 2870 | 9409 | 19907 | 22535 | 25163 |
| 2871 | 6782 | 12031 | 14654 | 17280 | 2872 | 9410 | 19908 | 22536 | 25164 |
| 2873 | 6783 | 12032 | 14655 | 17281 | 2874 | 9411 | 19909 | 22537 | 25165 |
| 2875 | 6784 | 12033 | 14656 | 17282 | 2876 | 9412 | 19910 | 22538 | 25166 |
| 2877 | 6785 | 12034 | 14657 | 17283 | 2878 | 9413 | 19911 | 22539 | 25167 |
| 2879 | 6786 | 12035 | 14658 | 17284 | 2880 | 9414 | 19912 | 22540 | 25168 |
| 2881 | 6787 | 12036 | 14659 | 17285 | 2882 | 9415 | 19913 | 22541 | 25169 |
| 2883 | 6788 | 12037 | 14660 | 17286 | 2884 | 9416 | 19914 | 22542 | 25170 |
| 2885 | 6789 | 12038 | 14661 | 17287 | 2886 | 9417 | 19915 | 22543 | 25171 |
| 2887 | 6790 | 12039 | 14662 | 17288 | 2888 | 9418 | 19916 | 22544 | 25172 |
| 2889 | 6791 | 12040 | 14663 | 17289 | 2890 | 9419 | 19917 | 22545 | 25173 |
| 2891 | 6792 | 12041 | 14664 | 17290 | 2892 | 9420 | 19918 | 22546 | 25174 |
| 2893 | 6793 | 12042 | 14665 | 17291 | 2894 | 9421 | 19919 | 22547 | 25175 |
| 2895 | 6794 | 12043 | 14666 | 17292 | 2896 | 9422 | 19920 | 22548 | 25176 |
| 2897 | 6795 | 12044 | 14667 | 17293 | 2898 | 9423 | 19921 | 22549 | 25177 |
| 2899 | 6796 | 12045 | 14668 | 17294 | 2900 | 9424 | 19922 | 22550 | 25178 |
| 2901 | 6797 | 12046 | 14669 | 17295 | 2902 | 9425 | 19923 | 22551 | 25179 |
| 2903 | 6798 | 12047 | 14670 | 17296 | 2904 | 9426 | 19924 | 22552 | 25180 |
| 2905 | 6799 | 12048 | 14671 | 17297 | 2906 | 9427 | 19925 | 22553 | 25181 |
| 2907 | 6800 | 12049 | 14672 | 17298 | 2908 | 9428 | 19926 | 22554 | 25182 |
| 2909 | 6801 | 12050 | 14673 | 17299 | 2910 | 9429 | 19927 | 22555 | 25183 |
| 2911 | 6802 | 12051 | 14674 | 17300 | 2912 | 9430 | 19928 | 22556 | 25184 |
| 2913 | 6803 | 12052 | 14675 | 17301 | 2914 | 9431 | 19929 | 22557 | 25185 |
| 2915 | 6804 | 12053 | 14676 | 17302 | 2916 | 9432 | 19930 | 22558 | 25186 |
| 2917 | 6805 | 12054 | 14677 | 17303 | 2918 | 9433 | 19931 | 22559 | 25187 |
| 2919 | 6806 | 12055 | 14678 | 17304 | 2920 | 9434 | 19932 | 22560 | 25188 |
| 2921 | 6807 | 12056 | 14679 | 17305 | 2922 | 9435 | 19933 | 22561 | 25189 |
| 2923 | 6808 | 12057 | 14680 | 17306 | 2924 | 9436 | 19934 | 22562 | 25190 |
| 2925 | 6809 | 12058 | 14681 | 17307 | 2926 | 9437 | 19935 | 22563 | 25191 |
| 2927 | 6810 | 12059 | 14682 | 17308 | 2928 | 9438 | 19936 | 22564 | 25192 |
| 2929 | 6811 | 12060 | 14683 | 17309 | 2930 | 9439 | 19937 | 22565 | 25193 |
| 2931 | 6812 | 12061 | 14684 | 17310 | 2932 | 9440 | 19938 | 22566 | 25194 |
| 2933 | 6813 | 12062 | 14685 | 17311 | 2934 | 9441 | 19939 | 22567 | 25195 |
| 2935 | 6814 | 12063 | 14686 | 17312 | 2936 | 9442 | 19940 | 22568 | 25196 |
| 2937 | 6815 | 12064 | 14687 | 17313 | 2938 | 9443 | 19941 | 22569 | 25197 |
| 2939 | 6816 | 12065 | 14688 | 17314 | 2940 | 9444 | 19942 | 22570 | 25198 |
| 2941 | 6817 | 12066 | 14689 | 17315 | 2942 | 9445 | 19943 | 22571 | 25199 |
| 2943 | 6818 | 12067 | 14690 | 17316 | 2944 | 9446 | 19944 | 22572 | 25200 |
| 2945 | 6819 | 12068 | 14691 | 17317 | 2946 | 9447 | 19945 | 22573 | 25201 |
| 2947 | 6820 | 12069 | 14692 | 17318 | 2948 | 9448 | 19946 | 22574 | 25202 |
| 2949 | 6821 | 12070 | 14693 | 17319 | 2950 | 9449 | 19947 | 22575 | 25203 |
| 2951 | 6822 | 12071 | 14694 | 17320 | 2952 | 9450 | 19948 | 22576 | 25204 |
| 2953 | 6823 | 12072 | 14695 | 17321 | 2954 | 9451 | 19949 | 22577 | 25205 |
| 2955 | 6824 | 12073 | 14696 | 17322 | 2956 | 9452 | 19950 | 22578 | 25206 |
| 2957 | 6825 | 12074 | 14697 | 17323 | 2958 | 9453 | 19951 | 22579 | 25207 |
| 2959 | 6826 | 12075 | 14698 | 17324 | 2960 | 9454 | 19952 | 22580 | 25208 |
| 2961 | 6827 | 12076 | 14699 | 17325 | 2962 | 9455 | 19953 | 22581 | 25209 |
| 2963 | 6828 | 12077 | 14700 | 17326 | 2964 | 9456 | 19954 | 22582 | 25210 |
| 2965 | 6829 | 12078 | 14701 | 17327 | 2966 | 9457 | 19955 | 22583 | 25211 |
| 2967 | 6830 | 12079 | 14702 | 17328 | 2968 | 9458 | 19956 | 22584 | 25212 |
| 2969 | 6831 | 12080 | 14703 | 17329 | 2970 | 9459 | 19957 | 22585 | 25213 |
| 2971 | 6832 | 12081 | 14704 | 17330 | 2972 | 9460 | 19958 | 22586 | 25214 |
| 2973 | 6833 | 12082 | 14705 | 17331 | 2974 | 9461 | 19959 | 22587 | 25215 |
| 2975 | 6834 | 12083 | 14706 | 17332 | 2976 | 9462 | 19960 | 22588 | 25216 |
| 2977 | 6835 | 12084 | 14707 | 17333 | 2978 | 9463 | 19961 | 22589 | 25217 |
| 2979 | 6836 | 12085 | 14708 | 17334 | 2980 | 9464 | 19962 | 22590 | 25218 |
| 2981 | 6837 | 12086 | 14709 | 17335 | 2982 | 9465 | 19963 | 22591 | 25219 |
| 2983 | 6838 | 12087 | 14710 | 17336 | 2984 | 9466 | 19964 | 22592 | 25220 |
| 2985 | 6839 | 12088 | 14711 | 17337 | 2986 | 9467 | 19965 | 22593 | 25221 |
| 2987 | 6840 | 12089 | 14712 | 17338 | 2988 | 9468 | 19966 | 22594 | 25222 |
| 2989 | 6841 | 12090 | 14713 | 17339 | 2990 | 9469 | 19967 | 22595 | 25223 |
| 2991 | 6842 | 12091 | 14714 | 17340 | 2992 | 9470 | 19968 | 22596 | 25224 |
| 2993 | 6843 | 12092 | 14715 | 17341 | 2994 | 9471 | 19969 | 22597 | 25225 |
| 2995 | 6844 | 12093 | 14716 | 17342 | 2996 | 9472 | 19970 | 22598 | 25226 |
| 2997 | 6845 | 12094 | 14717 | 17343 | 2998 | 9473 | 19971 | 22599 | 25227 |
| 2999 | 6846 | 12095 | 14718 | 17344 | 3000 | 9474 | 19972 | 22600 | 25228 |
| 3001 | 6847 | 12096 | 14719 | 17345 | 3002 | 9475 | 19973 | 22601 | 25229 |
| 3003 | 6848 | 12097 | 14720 | 17346 | 3004 | 9476 | 19974 | 22602 | 25230 |
| 3005 | 6849 | 12098 | 14721 | 17347 | 3006 | 9477 | 19975 | 22603 | 25231 |
| 3007 | 6850 | 12099 | 14722 | 17348 | 3008 | 9478 | 19976 | 22604 | 25232 |
| 3009 | 6851 | 12100 | 14723 | 17349 | 3010 | 9479 | 19977 | 22605 | 25233 |
| 3011 | 6852 | 12101 | 14724 | 17350 | 3012 | 9480 | 19978 | 22606 | 25234 |
| 3013 | 6853 | 12102 | 14725 | 17351 | 3014 | 9481 | 19979 | 22607 | 25235 |
| 3015 | 6854 | 12103 | 14726 | 17352 | 3016 | 9482 | 19980 | 22608 | 25236 |
| 3017 | 6855 | 12104 | 14727 | 17353 | 3018 | 9483 | 19981 | 22609 | 25237 |
| 3019 | 6856 | 12105 | 14728 | 17354 | 3020 | 9484 | 19982 | 22610 | 25238 |
| 3021 | 6857 | 12106 | 14729 | 17355 | 3022 | 9485 | 19983 | 22611 | 25239 |
| 3023 | 6858 | 12107 | 14730 | 17356 | 3024 | 9486 | 19984 | 22612 | 25240 |
| 3025 | 6859 | 12108 | 14731 | 17357 | 3026 | 9487 | 19985 | 22613 | 25241 |
| 3027 | 6860 | 12109 | 14732 | 17358 | 3028 | 9488 | 19986 | 22614 | 25242 |
| 3029 | 6861 | 12110 | 14733 | 17359 | 3030 | 9489 | 19987 | 22615 | 25243 |
| 3031 | 6862 | 12111 | 14734 | 17360 | 3032 | 9490 | 19988 | 22616 | 25244 |
| 3033 | 6863 | 12112 | 14735 | 17361 | 3034 | 9491 | 19989 | 22617 | 25245 |
| 3035 | 6864 | 12113 | 14736 | 17362 | 3036 | 9492 | 19990 | 22618 | 25246 |
| 3037 | 6865 | 12114 | 14737 | 17363 | 3038 | 9493 | 19991 | 22619 | 25247 |
| 3039 | 6866 | 12115 | 14738 | 17364 | 3040 | 9494 | 19992 | 22620 | 25248 |
| 3041 | 6867 | 12116 | 14739 | 17365 | 3042 | 9495 | 19993 | 22621 | 25249 |
| 3043 | 6868 | 12117 | 14740 | 17366 | 3044 | 9496 | 19994 | 22622 | 25250 |
| 3045 | 6869 | 12118 | 14741 | 17367 | 3046 | 9497 | 19995 | 22623 | 25251 |
| 3047 | 6870 | 12119 | 14742 | 17368 | 3048 | 9498 | 19996 | 22624 | 25252 |
| 3049 | 6871 | 12120 | 14743 | 17369 | 3050 | 9499 | 19997 | 22625 | 25253 |
| 3051 | 6872 | 12121 | 14744 | 17370 | 3052 | 9500 | 19998 | 22626 | 25254 |
| 3053 | 6873 | 12122 | 14745 | 17371 | 3054 | 9501 | 19999 | 22627 | 25255 |
| 3055 | 6874 | 12123 | 14746 | 17372 | 3056 | 9502 | 20000 | 22628 | 25256 |
| 3057 | 6875 | 12124 | 14747 | 17373 | 3058 | 9503 | 20001 | 22629 | 25257 |
| 3059 | 6876 | 12125 | 14748 | 17374 | 3060 | 9504 | 20002 | 22630 | 25258 |
| 3061 | 6877 | 12126 | 14749 | 17375 | 3062 | 9505 | 20003 | 22631 | 25259 |
| 3063 | 6878 | 12127 | 14750 | 17376 | 3064 | 9506 | 20004 | 22632 | 25260 |
| 3065 | 6879 | 12128 | 14751 | 17377 | 3066 | 9507 | 20005 | 22633 | 25261 |
| 3067 | 6880 | 12129 | 14752 | 17378 | 3068 | 9508 | 20006 | 22634 | 25262 |
| 3069 | 6881 | 12130 | 14753 | 17379 | 3070 | 9509 | 20007 | 22635 | 25263 |
| 3071 | 6882 | 12131 | 14754 | 17380 | 3072 | 9510 | 20008 | 22636 | 25264 |
| 3073 | 6883 | 12132 | 14755 | 17381 | 3074 | 9511 | 20009 | 22637 | 25265 |
| 3075 | 6884 | 12133 | 14756 | 17382 | 3076 | 9512 | 20010 | 22638 | 25266 |
| 3077 | 6885 | 12134 | 14757 | 17383 | 3078 | 9513 | 20011 | 22639 | 25267 |
| 3079 | 6886 | 12135 | 14758 | 17384 | 3080 | 9514 | 20012 | 22640 | 25268 |
| 3081 | 6887 | 12136 | 14759 | 17385 | 3082 | 9515 | 20013 | 22641 | 25269 |
| 3083 | 6888 | 12137 | 14760 | 17386 | 3084 | 9516 | 20014 | 22642 | 25270 |
| 3085 | 6889 | 12138 | 14761 | 17387 | 3086 | 9517 | 20015 | 22643 | 25271 |
| 3087 | 6890 | 12139 | 14762 | 17388 | 3088 | 9518 | 20016 | 22644 | 25272 |
| 3089 | 6891 | 12140 | 14763 | 17389 | 3090 | 9519 | 20017 | 22645 | 25273 |
| 3091 | 6892 | 12141 | 14764 | 17390 | 3092 | 9520 | 20018 | 22646 | 25274 |
| 3093 | 6893 | 12142 | 14765 | 17391 | 3094 | 9521 | 20019 | 22647 | 25275 |
| 3095 | 6894 | 12143 | 14766 | 17392 | 3096 | 9522 | 20020 | 22648 | 25276 |
| 3097 | 6895 | 12144 | 14767 | 17393 | 3098 | 9523 | 20021 | 22649 | 25277 |
| 3099 | 6896 | 12145 | 14768 | 17394 | 3100 | 9524 | 20022 | 22650 | 25278 |
| 3101 | 6897 | 12146 | 14769 | 17395 | 3102 | 9525 | 20023 | 22651 | 25279 |
| 3103 | 6898 | 12147 | 14770 | 17396 | 3104 | 9526 | 20024 | 22652 | 25280 |
| 3105 | 6899 | 12148 | 14771 | 17397 | 3106 | 9527 | 20025 | 22653 | 25281 |
| 3107 | 6900 | 12149 | 14772 | 17398 | 3108 | 9528 | 20026 | 22654 | 25282 |
| 3109 | 6901 | 12150 | 14773 | 17399 | 3110 | 9529 | 20027 | 22655 | 25283 |
| 3111 | 6902 | 12151 | 14774 | 17400 | 3112 | 9530 | 20028 | 22656 | 25284 |
| 3113 | 6903 | 12152 | 14775 | 17401 | 3114 | 9531 | 20029 | 22657 | 25285 |
| 3115 | 6904 | 12153 | 14776 | 17402 | 3116 | 9532 | 20030 | 22658 | 25286 |
| 3117 | 6905 | 12154 | 14777 | 17403 | 3118 | 9533 | 20031 | 22659 | 25287 |
| 3119 | 6906 | 12155 | 14778 | 17404 | 3120 | 9534 | 20032 | 22660 | 25288 |
| 3121 | 6907 | 12156 | 14779 | 17405 | 3122 | 9535 | 20033 | 22661 | 25289 |
| 3123 | 6908 | 12157 | 14780 | 17406 | 3124 | 9536 | 20034 | 22662 | 25290 |
| 3125 | 6909 | 12158 | 14781 | 17407 | 3126 | 9537 | 20035 | 22663 | 25291 |
| 3127 | 6910 | 12159 | 14782 | 17408 | 3128 | 9538 | 20036 | 22664 | 25292 |
| 3129 | 6911 | 12160 | 14783 | 17409 | 3130 | 9539 | 20037 | 22665 | 25293 |
| 3131 | 6912 | 12161 | 14784 | 17410 | 3132 | 9540 | 20038 | 22666 | 25294 |
| 3133 | 6913 | 12162 | 14785 | 17411 | 3134 | 9541 | 20039 | 22667 | 25295 |
| 3135 | 6914 | 12163 | 14786 | 17412 | 3136 | 9542 | 20040 | 22668 | 25296 |
| 3137 | 6915 | 12164 | 14787 | 17413 | 3138 | 9543 | 20041 | 22669 | 25297 |
| 3139 | 6916 | 12165 | 14788 | 17414 | 3140 | 9544 | 20042 | 22670 | 25298 |
| 3141 | 6917 | 12166 | 14789 | 17415 | 3142 | 9545 | 20043 | 22671 | 25299 |
| 3143 | 6918 | 12167 | 14790 | 17416 | 3144 | 9546 | 20044 | 22672 | 25300 |
| 3145 | 6919 | 12168 | 14791 | 17417 | 3146 | 9547 | 20045 | 22673 | 25301 |
| 3147 | 6920 | 12169 | 14792 | 17418 | 3148 | 9548 | 20046 | 22674 | 25302 |
| 3149 | 6921 | 12170 | 14793 | 17419 | 3150 | 9549 | 20047 | 22675 | 25303 |
| 3151 | 6922 | 12171 | 14794 | 17420 | 3152 | 9550 | 20048 | 22676 | 25304 |
| 3153 | 6923 | 12172 | 14795 | 17421 | 3154 | 9551 | 20049 | 22677 | 25305 |
| 3155 | 6924 | 12173 | 14796 | 17422 | 3156 | 9552 | 20050 | 22678 | 25306 |
| 3157 | 6925 | 12174 | 14797 | 17423 | 3158 | 9553 | 20051 | 22679 | 25307 |
| 3159 | 6926 | 12175 | 14798 | 17424 | 3160 | 9554 | 20052 | 22680 | 25308 |
| 3161 | 6927 | 12176 | 14799 | 17425 | 3162 | 9555 | 20053 | 22681 | 25309 |
| 3163 | 6928 | 12177 | 14800 | 17426 | 3164 | 9556 | 20054 | 22682 | 25310 |
| 3165 | 6929 | 12178 | 14801 | 17427 | 3166 | 9557 | 20055 | 22683 | 25311 |
| 3167 | 6930 | 12179 | 14802 | 17428 | 3168 | 9558 | 20056 | 22684 | 25312 |
| 3169 | 6931 | 12180 | 14803 | 17429 | 3170 | 9559 | 20057 | 22685 | 25313 |
| 3171 | 6932 | 12181 | 14804 | 17430 | 3172 | 9560 | 20058 | 22686 | 25314 |
| 3173 | 6933 | 12182 | 14805 | 17431 | 3174 | 9561 | 20059 | 22687 | 25315 |
| 3175 | 6934 | 12183 | 14806 | 17432 | 3176 | 9562 | 20060 | 22688 | 25316 |
| 3177 | 6935 | 12184 | 14807 | 17433 | 3178 | 9563 | 20061 | 22689 | 25317 |
| 3179 | 6936 | 12185 | 14808 | 17434 | 3180 | 9564 | 20062 | 22690 | 25318 |
| 3181 | 6937 | 12186 | 14809 | 17435 | 3182 | 9565 | 20063 | 22691 | 25319 |
| 3183 | 6938 | 12187 | 14810 | 17436 | 3184 | 9566 | 20064 | 22692 | 25320 |
| 3185 | 6939 | 12188 | 14811 | 17437 | 3186 | 9567 | 20065 | 22693 | 25321 |
| 3187 | 6940 | 12189 | 14812 | 17438 | 3188 | 9568 | 20066 | 22694 | 25322 |
| 3189 | 6941 | 12190 | 14813 | 17439 | 3190 | 9569 | 20067 | 22695 | 25323 |
| 3191 | 6942 | 12191 | 14814 | 17440 | 3192 | 9570 | 20068 | 22696 | 25324 |
| 3193 | 6943 | 12192 | 14815 | 17441 | 3194 | 9571 | 20069 | 22697 | 25325 |
| 3195 | 6944 | 12193 | 14816 | 17442 | 3196 | 9572 | 20070 | 22698 | 25326 |
| 3197 | 6945 | 12194 | 14817 | 17443 | 3198 | 9573 | 20071 | 22699 | 25327 |
| 3199 | 6946 | 12195 | 14818 | 17444 | 3200 | 9574 | 20072 | 22700 | 25328 |
| 3201 | 6947 | 12196 | 14819 | 17445 | 3202 | 9575 | 20073 | 22701 | 25329 |
| 3203 | 6948 | 12197 | 14820 | 17446 | 3204 | 9576 | 20074 | 22702 | 25330 |
| 3205 | 6949 | 12198 | 14821 | 17447 | 3206 | 9577 | 20075 | 22703 | 25331 |
| 3207 | 6950 | 12199 | 14822 | 17448 | 3208 | 9578 | 20076 | 22704 | 25332 |
| 3209 | 6951 | 12200 | 14823 | 17449 | 3210 | 9579 | 20077 | 22705 | 25333 |
| 3211 | 6952 | 12201 | 14824 | 17450 | 3212 | 9580 | 20078 | 22706 | 25334 |
| 3213 | 6953 | 12202 | 14825 | 17451 | 3214 | 9581 | 20079 | 22707 | 25335 |
| 3215 | 6954 | 12203 | 14826 | 17452 | 3216 | 9582 | 20080 | 22708 | 25336 |
| 3217 | 6955 | 12204 | 14827 | 17453 | 3218 | 9583 | 20081 | 22709 | 25337 |
| 3219 | 6956 | 12205 | 14828 | 17454 | 3220 | 9584 | 20082 | 22710 | 25338 |
| 3221 | 6957 | 12206 | 14829 | 17455 | 3222 | 9585 | 20083 | 22711 | 25339 |
| 3223 | 6958 | 12207 | 14830 | 17456 | 3224 | 9586 | 20084 | 22712 | 25340 |
| 3225 | 6959 | 12208 | 14831 | 17457 | 3226 | 9587 | 20085 | 22713 | 25341 |
| 3227 | 6960 | 12209 | 14832 | 17458 | 3228 | 9588 | 20086 | 22714 | 25342 |
| 3229 | 6961 | 12210 | 14833 | 17459 | 3230 | 9589 | 20087 | 22715 | 25343 |
| 3231 | 6962 | 12211 | 14834 | 17460 | 3232 | 9590 | 20088 | 22716 | 25344 |
| 3233 | 6963 | 12212 | 14835 | 17461 | 3234 | 9591 | 20089 | 22717 | 25345 |
| 3235 | 6964 | 12213 | 14836 | 17462 | 3236 | 9592 | 20090 | 22718 | 25346 |
| 3237 | 6965 | 12214 | 14837 | 17463 | 3238 | 9593 | 20091 | 22719 | 25347 |
| 3239 | 6966 | 12215 | 14838 | 17464 | 3240 | 9594 | 20092 | 22720 | 25348 |
| 3241 | 6967 | 12216 | 14839 | 17465 | 3242 | 9595 | 20093 | 22721 | 25349 |
| 3243 | 6968 | 12217 | 14840 | 17466 | 3244 | 9596 | 20094 | 22722 | 25350 |
| 3245 | 6969 | 12218 | 14841 | 17467 | 3246 | 9597 | 20095 | 22723 | 25351 |
| 3247 | 6970 | 12219 | 14842 | 17468 | 3248 | 9598 | 20096 | 22724 | 25352 |
| 3249 | 6971 | 12220 | 14843 | 17469 | 3250 | 9599 | 20097 | 22725 | 25353 |
| 3251 | 6972 | 12221 | 14844 | 17470 | 3252 | 9600 | 20098 | 22726 | 25354 |
| 3253 | 6973 | 12222 | 14845 | 17471 | 3254 | 9601 | 20099 | 22727 | 25355 |
| 3255 | 6974 | 12223 | 14846 | 17472 | 3256 | 9602 | 20100 | 22728 | 25356 |
| 3257 | 6975 | 12224 | 14847 | 17473 | 3258 | 9603 | 20101 | 22729 | 25357 |
| 3259 | 6976 | 12225 | 14848 | 17474 | 3260 | 9604 | 20102 | 22730 | 25358 |
| 3261 | 6977 | 12226 | 14849 | 17475 | 3262 | 9605 | 20103 | 22731 | 25359 |
| 3263 | 6978 | 12227 | 14850 | 17476 | 3264 | 9606 | 20104 | 22732 | 25360 |
| 3265 | 6979 | 12228 | 14851 | 17477 | 3266 | 9607 | 20105 | 22733 | 25361 |
| 3267 | 6980 | 12229 | 14852 | 17478 | 3268 | 9608 | 20106 | 22734 | 25362 |
| 3269 | 6981 | 12230 | 14853 | 17479 | 3270 | 9609 | 20107 | 22735 | 25363 |
| 3271 | 6982 | 12231 | 14854 | 17480 | 3272 | 9610 | 20108 | 22736 | 25364 |
| 3273 | 6983 | 12232 | 14855 | 17481 | 3274 | 9611 | 20109 | 22737 | 25365 |
| 3275 | 6984 | 12233 | 14856 | 17482 | 3276 | 9612 | 20110 | 22738 | 25366 |
| 3277 | 6985 | 12234 | 14857 | 17483 | 3278 | 9613 | 20111 | 22739 | 25367 |
| 3279 | 6986 | 12235 | 14858 | 17484 | 3280 | 9614 | 20112 | 22740 | 25368 |
| 3281 | 6987 | 12236 | 14859 | 17485 | 3282 | 9615 | 20113 | 22741 | 25369 |
| 3283 | 6988 | 12237 | 14860 | 17486 | 3284 | 9616 | 20114 | 22742 | 25370 |
| 3285 | 6989 | 12238 | 14861 | 17487 | 3286 | 9617 | 20115 | 22743 | 25371 |
| 3287 | 6990 | 12239 | 14862 | 17488 | 3288 | 9618 | 20116 | 22744 | 25372 |
| 3289 | 6991 | 12240 | 14863 | 17489 | 3290 | 9619 | 20117 | 22745 | 25373 |
| 3291 | 6992 | 12241 | 14864 | 17490 | 3292 | 9620 | 20118 | 22746 | 25374 |
| 3293 | 6993 | 12242 | 14865 | 17491 | 3294 | 9621 | 20119 | 22747 | 25375 |
| 3295 | 6994 | 12243 | 14866 | 17492 | 3296 | 9622 | 20120 | 22748 | 25376 |
| 3297 | 6995 | 12244 | 14867 | 17493 | 3298 | 9623 | 20121 | 22749 | 25377 |
| 3299 | 6996 | 12245 | 14868 | 17494 | 3300 | 9624 | 20122 | 22750 | 25378 |
| 3301 | 6997 | 12246 | 14869 | 17495 | 3302 | 9625 | 20123 | 22751 | 25379 |
| 3303 | 6998 | 12247 | 14870 | 17496 | 3304 | 9626 | 20124 | 22752 | 25380 |
| 3305 | 6999 | 12248 | 14871 | 17497 | 3306 | 9627 | 20125 | 22753 | 25381 |
| 3307 | 7000 | 12249 | 14872 | 17498 | 3308 | 9628 | 20126 | 22754 | 25382 |
| 3309 | 7001 | 12250 | 14873 | 17499 | 3310 | 9629 | 20127 | 22755 | 25383 |
| 3311 | 7002 | 12251 | 14874 | 17500 | 3312 | 9630 | 20128 | 22756 | 25384 |
| 3313 | 7003 | 12252 | 14875 | 17501 | 3314 | 9631 | 20129 | 22757 | 25385 |
| 3315 | 7004 | 12253 | 14876 | 17502 | 3316 | 9632 | 20130 | 22758 | 25386 |
| 3317 | 7005 | 12254 | 14877 | 17503 | 3318 | 9633 | 20131 | 22759 | 25387 |
| 3319 | 7006 | 12255 | 14878 | 17504 | 3320 | 9634 | 20132 | 22760 | 25388 |
| 3321 | 7007 | 12256 | 14879 | 17505 | 3322 | 9635 | 20133 | 22761 | 25389 |
| 3323 | 7008 | 12257 | 14880 | 17506 | 3324 | 9636 | 20134 | 22762 | 25390 |
| 3325 | 7009 | 12258 | 14881 | 17507 | 3326 | 9637 | 20135 | 22763 | 25391 |
| 3327 | 7010 | 12259 | 14882 | 17508 | 3328 | 9638 | 20136 | 22764 | 25392 |
| 3329 | 7011 | 12260 | 14883 | 17509 | 3330 | 9639 | 20137 | 22765 | 25393 |
| 3331 | 7012 | 12261 | 14884 | 17510 | 3332 | 9640 | 20138 | 22766 | 25394 |
| 3333 | 7013 | 12262 | 14885 | 17511 | 3334 | 9641 | 20139 | 22767 | 25395 |
| 3335 | 7014 | 12263 | 14886 | 17512 | 3336 | 9642 | 20140 | 22768 | 25396 |
| 3337 | 7015 | 12264 | 14887 | 17513 | 3338 | 9643 | 20141 | 22769 | 25397 |
| 3339 | 7016 | 12265 | 14888 | 17514 | 3340 | 9644 | 20142 | 22770 | 25398 |
| 3341 | 7017 | 12266 | 14889 | 17515 | 3342 | 9645 | 20143 | 22771 | 25399 |
| 3343 | 7018 | 12267 | 14890 | 17516 | 3344 | 9646 | 20144 | 22772 | 25400 |
| 3345 | 7019 | 12268 | 14891 | 17517 | 3346 | 9647 | 20145 | 22773 | 25401 |
| 3347 | 7020 | 12269 | 14892 | 17518 | 3348 | 9648 | 20146 | 22774 | 25402 |
| 3349 | 7021 | 12270 | 14893 | 17519 | 3350 | 9649 | 20147 | 22775 | 25403 |
| 3351 | 7022 | 12271 | 14894 | 17520 | 3352 | 9650 | 20148 | 22776 | 25404 |
| 3353 | 7023 | 12272 | 14895 | 17521 | 3354 | 9651 | 20149 | 22777 | 25405 |
| 3355 | 7024 | 12273 | 14896 | 17522 | 3356 | 9652 | 20150 | 22778 | 25406 |
| 3357 | 7025 | 12274 | 14897 | 17523 | 3358 | 9653 | 20151 | 22779 | 25407 |
| 3359 | 7026 | 12275 | 14898 | 17524 | 3360 | 9654 | 20152 | 22780 | 25408 |
| 3361 | 7027 | 12276 | 14899 | 17525 | 3362 | 9655 | 20153 | 22781 | 25409 |
| 3363 | 7028 | 12277 | 14900 | 17526 | 3364 | 9656 | 20154 | 22782 | 25410 |
| 3365 | 7029 | 12278 | 14901 | 17527 | 3366 | 9657 | 20155 | 22783 | 25411 |
| 3367 | 7030 | 12279 | 14902 | 17528 | 3368 | 9658 | 20156 | 22784 | 25412 |
| 3369 | 7031 | 12280 | 14903 | 17529 | 3370 | 9659 | 20157 | 22785 | 25413 |
| 3371 | 7032 | 12281 | 14904 | 17530 | 3372 | 9660 | 20158 | 22786 | 25414 |
| 3373 | 7033 | 12282 | 14905 | 17531 | 3374 | 9661 | 20159 | 22787 | 25415 |
| 3375 | 7034 | 12283 | 14906 | 17532 | 3376 | 9662 | 20160 | 22788 | 25416 |
| 3377 | 7035 | 12284 | 14907 | 17533 | 3378 | 9663 | 20161 | 22789 | 25417 |
| 3379 | 7036 | 12285 | 14908 | 17534 | 3380 | 9664 | 20162 | 22790 | 25418 |
| 3381 | 7037 | 12286 | 14909 | 17535 | 3382 | 9665 | 20163 | 22791 | 25419 |
| 3383 | 7038 | 12287 | 14910 | 17536 | 3384 | 9666 | 20164 | 22792 | 25420 |
| 3385 | 7039 | 12288 | 14911 | 17537 | 3386 | 9667 | 20165 | 22793 | 25421 |
| 3387 | 7040 | 12289 | 14912 | 17538 | 3388 | 9668 | 20166 | 22794 | 25422 |
| 3389 | 7041 | 12290 | 14913 | 17539 | 3390 | 9669 | 20167 | 22795 | 25423 |
| 3391 | 7042 | 12291 | 14914 | 17540 | 3392 | 9670 | 20168 | 22796 | 25424 |
| 3393 | 7043 | 12292 | 14915 | 17541 | 3394 | 9671 | 20169 | 22797 | 25425 |
| 3395 | 7044 | 12293 | 14916 | 17542 | 3396 | 9672 | 20170 | 22798 | 25426 |
| 3397 | 7045 | 12294 | 14917 | 17543 | 3398 | 9673 | 20171 | 22799 | 25427 |
| 3399 | 7046 | 12295 | 14918 | 17544 | 3400 | 9674 | 20172 | 22800 | 25428 |
| 3401 | 7047 | 12296 | 14919 | 17545 | 3402 | 9675 | 20173 | 22801 | 25429 |
| 3403 | 7048 | 12297 | 14920 | 17546 | 3404 | 9676 | 20174 | 22802 | 25430 |
| 3405 | 7049 | 12298 | 14921 | 17547 | 3406 | 9677 | 20175 | 22803 | 25431 |
| 3407 | 7050 | 12299 | 14922 | 17548 | 3408 | 9678 | 20176 | 22804 | 25432 |
| 3409 | 7051 | 12300 | 14923 | 17549 | 3410 | 9679 | 20177 | 22805 | 25433 |
| 3411 | 7052 | | | 17550 | 3412 | 9680 | 20178 | 22806 | 25434 |
| 3413 | 7053 | 12301 | 14924 | 17551 | 3414 | 9681 | 20179 | 22807 | 25435 |
| 3415 | 7054 | 12302 | 14925 | 17552 | 3416 | 9682 | 20180 | 22808 | 25436 |
| 3417 | 7055 | 12303 | 14926 | 17553 | 3418 | 9683 | 20181 | 22809 | 25437 |
| 3419 | 7056 | 12304 | 14927 | 17554 | 3420 | 9684 | 20182 | 22810 | 25438 |
| 3421 | 7057 | 12305 | 14928 | 17555 | 3422 | 9685 | 20183 | 22811 | 25439 |
| 3423 | 7058 | 12306 | 14929 | 17556 | 3424 | 9686 | 20184 | 22812 | 25440 |
| 3425 | 7059 | 12307 | 14930 | 17557 | 3426 | 9687 | 20185 | 22813 | 25441 |
| 3427 | 7060 | 12308 | 14931 | 17558 | 3428 | 9688 | 20186 | 22814 | 25442 |
| 3429 | 7061 | 12309 | 14932 | 17559 | 3430 | 9689 | 20187 | 22815 | 25443 |
| 3431 | 7062 | 12310 | 14933 | 17560 | 3432 | 9690 | 20188 | 22816 | 25444 |
| 3433 | 7063 | 12311 | 14934 | 17561 | 3434 | 9691 | 20189 | 22817 | 25445 |
| 3435 | 7064 | 12312 | 14935 | 17562 | 3436 | 9692 | 20190 | 22818 | 25446 |
| 3437 | 7065 | 12313 | 14936 | 17563 | 3438 | 9693 | 20191 | 22819 | 25447 |
| 3439 | 7066 | 12314 | 14937 | 17564 | 3440 | 9694 | 20192 | 22820 | 25448 |
| 3441 | 7067 | 12315 | 14938 | 17565 | 3442 | 9695 | 20193 | 22821 | 25449 |
| 3443 | 7068 | 12316 | 14939 | 17566 | 3444 | 9696 | 20194 | 22822 | 25450 |
| 3445 | 7069 | 12317 | 14940 | 17567 | 3446 | 9697 | 20195 | 22823 | 25451 |
| 3447 | 7070 | 12318 | 14941 | 17568 | 3448 | 9698 | 20196 | 22824 | 25452 |
| 3449 | 7071 | 12319 | 14942 | 17569 | 3450 | 9699 | 20197 | 22825 | 25453 |
| 3451 | 7072 | 12320 | 14943 | 17570 | 3452 | 9700 | 20198 | 22826 | 25454 |
| 3453 | 7073 | 12321 | 14944 | 17571 | 3454 | 9701 | 20199 | 22827 | 25455 |
| 3455 | 7074 | 12322 | 14945 | 17572 | 3456 | 9702 | 20200 | 22828 | 25456 |
| 3457 | 7075 | 12323 | 14946 | 17573 | 3458 | 9703 | 20201 | 22829 | 25457 |
| 3459 | 7076 | 12324 | 14947 | 17574 | 3460 | 9704 | 20202 | 22830 | 25458 |
| 3461 | 7077 | 12325 | 14948 | 17575 | 3462 | 9705 | 20203 | 22831 | 25459 |
| 3463 | 7078 | 12326 | 14949 | 17576 | 3464 | 9706 | 20204 | 22832 | 25460 |
| 3465 | 7079 | 12327 | 14950 | 17577 | 3466 | 9707 | 20205 | 22833 | 25461 |
| 3467 | 7080 | 12328 | 14951 | 17578 | 3468 | 9708 | 20206 | 22834 | 25462 |
| 3469 | 7081 | 12329 | 14952 | 17579 | 3470 | 9709 | 20207 | 22835 | 25463 |
| 3471 | 7082 | 12330 | 14953 | 17580 | 3472 | 9710 | 20208 | 22836 | 25464 |
| 3473 | 7083 | 12331 | 14954 | 17581 | 3474 | 9711 | 20209 | 22837 | 25465 |
| 3475 | 7084 | 12332 | 14955 | 17582 | 3476 | 9712 | 20210 | 22838 | 25466 |
| 3477 | 7085 | 12333 | 14956 | 17583 | 3478 | 9713 | 20211 | 22839 | 25467 |
| 3479 | 7086 | 12334 | 14957 | 17584 | 3480 | 9714 | 20212 | 22840 | 25468 |
| 3481 | 7087 | 12335 | 14958 | 17585 | 3482 | 9715 | 20213 | 22841 | 25469 |
| 3483 | 7088 | 12336 | 14959 | 17586 | 3484 | 9716 | 20214 | 22842 | 25470 |
| 3485 | 7089 | 12337 | 14960 | 17587 | 3486 | 9717 | 20215 | 22843 | 25471 |
| 3487 | 7090 | 12338 | 14961 | 17588 | 3488 | 9718 | 20216 | 22844 | 25472 |
| 3489 | 7091 | 12339 | 14962 | 17589 | 3490 | 9719 | 20217 | 22845 | 25473 |
| 3491 | 7092 | 12340 | 14963 | 17590 | 3492 | 9720 | 20218 | 22846 | 25474 |
| 3493 | 7093 | 12341 | 14964 | 17591 | 3494 | 9721 | 20219 | 22847 | 25475 |
| 3495 | 7094 | 12342 | 14965 | 17592 | 3496 | 9722 | 20220 | 22848 | 25476 |
| 3497 | 7095 | 12343 | 14966 | 17593 | 3498 | 9723 | 20221 | 22849 | 25477 |
| 3499 | 7096 | 12344 | 14967 | 17594 | 3500 | 9724 | 20222 | 22850 | 25478 |
| 3501 | 7097 | 12345 | 14968 | 17595 | 3502 | 9725 | 20223 | 22851 | 25479 |
| 3503 | 7098 | 12346 | 14969 | 17596 | 3504 | 9726 | 20224 | 22852 | 25480 |
| 3505 | 7099 | 12347 | 14970 | 17597 | 3506 | 9727 | 20225 | 22853 | 25481 |
| 3507 | 7100 | 12348 | 14971 | 17598 | 3508 | 9728 | 20226 | 22854 | 25482 |
| 3509 | 7101 | 12349 | 14972 | 17599 | 3510 | 9729 | 20227 | 22855 | 25483 |
| 3511 | 7102 | 12350 | 14973 | 17600 | 3512 | 9730 | 20228 | 22856 | 25484 |
| 3513 | 7103 | 12351 | 14974 | 17601 | 3514 | 9731 | 20229 | 22857 | 25485 |
| 3515 | 7104 | 12352 | 14975 | 17602 | 3516 | 9732 | 20230 | 22858 | 25486 |
| 3517 | 7105 | 12353 | 14976 | 17603 | 3518 | 9733 | 20231 | 22859 | 25487 |
| 3519 | 7106 | 12354 | 14977 | 17604 | 3520 | 9734 | 20232 | 22860 | 25488 |
| 3521 | 7107 | 12355 | 14978 | 17605 | 3522 | 9735 | 20233 | 22861 | 25489 |
| 3523 | 7108 | 12356 | 14979 | 17606 | 3524 | 9736 | 20234 | 22862 | 25490 |
| 3525 | 7109 | 12357 | 14980 | 17607 | 3526 | 9737 | 20235 | 22863 | 25491 |
| 3527 | 7110 | 12358 | 14981 | 17608 | 3528 | 9738 | 20236 | 22864 | 25492 |
| 3529 | 7111 | 12359 | 14982 | 17609 | 3530 | 9739 | 20237 | 22865 | 25493 |
| 3531 | 7112 | 12360 | 14983 | 17610 | 3532 | 9740 | 20238 | 22866 | 25494 |
| 3533 | 7113 | 12361 | 14984 | 17611 | 3534 | 9741 | 20239 | 22867 | 25495 |
| 3535 | 7114 | 12362 | 14985 | 17612 | 3536 | 9742 | 20240 | 22868 | 25496 |
| 3537 | 7115 | 12363 | 14986 | 17613 | 3538 | 9743 | 20241 | 22869 | 25497 |
| 3539 | 7116 | 12364 | 14987 | 17614 | 3540 | 9744 | 20242 | 22870 | 25498 |
| 3541 | 7117 | 12365 | 14988 | 17615 | 3542 | 9745 | 20243 | 22871 | 25499 |
| 3543 | 7118 | 12366 | 14989 | 17616 | 3544 | 9746 | 20244 | 22872 | 25500 |
| 3545 | 7119 | 12367 | 14990 | 17617 | 3546 | 9747 | 20245 | 22873 | 25501 |
| 3547 | 7120 | 12368 | 14991 | 17618 | 3548 | 9748 | 20246 | 22874 | 25502 |
| 3549 | 7121 | 12369 | 14992 | 17619 | 3550 | 9749 | 20247 | 22875 | 25503 |
| 3551 | 7122 | 12370 | 14993 | 17620 | 3552 | 9750 | 20248 | 22876 | 25504 |
| 3553 | 7123 | 12371 | 14994 | 17621 | 3554 | 9751 | 20249 | 22877 | 25505 |
| 3555 | 7124 | 12372 | 14995 | 17622 | 3556 | 9752 | 20250 | 22878 | 25506 |
| 3557 | 7125 | 12373 | 14996 | 17623 | 3558 | 9753 | 20251 | 22879 | 25507 |
| 3559 | 7126 | 12374 | 14997 | 17624 | 3560 | 9754 | 20252 | 22880 | 25508 |
| 3561 | 7127 | 12375 | 14998 | 17625 | 3562 | 9755 | 20253 | 22881 | 25509 |
| 3563 | 7128 | 12376 | 14999 | 17626 | 3564 | 9756 | 20254 | 22882 | 25510 |
| 3565 | 7129 | 12377 | 15000 | 17627 | 3566 | 9757 | 20255 | 22883 | 25511 |
| 3567 | 7130 | 12378 | 15001 | 17628 | 3568 | 9758 | 20256 | 22884 | 25512 |
| 3569 | 7131 | 12379 | 15002 | 17629 | 3570 | 9759 | 20257 | 22885 | 25513 |
| 3571 | 7132 | 12380 | 15003 | 17630 | 3572 | 9760 | 20258 | 22886 | 25514 |
| 3573 | 7133 | 12381 | 15004 | 17631 | 3574 | 9761 | 20259 | 22887 | 25515 |
| 3575 | 7134 | 12382 | 15005 | 17632 | 3576 | 9762 | 20260 | 22888 | 25516 |
| 3577 | 7135 | 12383 | 15006 | 17633 | 3578 | 9763 | 20261 | 22889 | 25517 |
| 3579 | 7136 | 12384 | 15007 | 17634 | 3580 | 9764 | 20262 | 22890 | 25518 |
| 3581 | 7137 | 12385 | 15008 | 17635 | 3582 | 9765 | 20263 | 22891 | 25519 |
| 3583 | 7138 | 12386 | 15009 | 17636 | 3584 | 9766 | 20264 | 22892 | 25520 |
| 3585 | 7139 | 12387 | 15010 | 17637 | 3586 | 9767 | 20265 | 22893 | 25521 |
| 3587 | 7140 | 12388 | 15011 | 17638 | 3588 | 9768 | 20266 | 22894 | 25522 |
| 3589 | 7141 | 12389 | 15012 | 17639 | 3590 | 9769 | 20267 | 22895 | 25523 |
| 3591 | 7142 | 12390 | 15013 | 17640 | 3592 | 9770 | 20268 | 22896 | 25524 |
| 3593 | 7143 | 12391 | 15014 | 17641 | 3594 | 9771 | 20269 | 22897 | 25525 |
| 3595 | 7144 | 12392 | 15015 | 17642 | 3596 | 9772 | 20270 | 22898 | 25526 |
| 3597 | 7145 | 12393 | 15016 | 17643 | 3598 | 9773 | 20271 | 22899 | 25527 |
| 3599 | 7146 | 12394 | 15017 | 17644 | 3600 | 9774 | 20272 | 22900 | 25528 |
| 3601 | 7147 | 12395 | 15018 | 17645 | 3602 | 9775 | 20273 | 22901 | 25529 |
| 3603 | 7148 | 12396 | 15019 | 17646 | 3604 | 9776 | 20274 | 22902 | 25530 |
| 3605 | 7149 | 12397 | 15020 | 17647 | 3606 | 9777 | 20275 | 22903 | 25531 |
| 3607 | 7150 | 12398 | 15021 | 17648 | 3608 | 9778 | 20276 | 22904 | 25532 |
| 3609 | 7151 | 12399 | 15022 | 17649 | 3610 | 9779 | 20277 | 22905 | 25533 |
| 3611 | 7152 | 12400 | 15023 | 17650 | 3612 | 9780 | 20278 | 22906 | 25534 |
| 3613 | 7153 | 12401 | 15024 | 17651 | 3614 | 9781 | 20279 | 22907 | 25535 |
| 3615 | 7154 | 12402 | 15025 | 17652 | 3616 | 9782 | 20280 | 22908 | 25536 |
| 3617 | 7155 | 12403 | 15026 | 17653 | 3618 | 9783 | 20281 | 22909 | 25537 |
| 3619 | 7156 | 12404 | 15027 | 17654 | 3620 | 9784 | 20282 | 22910 | 25538 |
| 3621 | 7157 | 12405 | 15028 | 17655 | 3622 | 9785 | 20283 | 22911 | 25539 |
| 3623 | 7158 | 12406 | 15029 | 17656 | 3624 | 9786 | 20284 | 22912 | 25540 |
| 3625 | 7159 | 12407 | 15030 | 17657 | 3626 | 9787 | 20285 | 22913 | 25541 |
| 3627 | 7160 | 12408 | 15031 | 17658 | 3628 | 9788 | 20286 | 22914 | 25542 |
| 3629 | 7161 | 12409 | 15032 | 17659 | 3630 | 9789 | 20287 | 22915 | 25543 |
| 3631 | 7162 | 12410 | 15033 | 17660 | 3632 | 9790 | 20288 | 22916 | 25544 |
| 3633 | 7163 | 12411 | 15034 | 17661 | 3634 | 9791 | 20289 | 22917 | 25545 |
| 3635 | 7164 | 12412 | 15035 | 17662 | 3636 | 9792 | 20290 | 22918 | 25546 |
| 3637 | 7165 | 12413 | 15036 | 17663 | 3638 | 9793 | 20291 | 22919 | 25547 |
| 3639 | 7166 | 12414 | 15037 | 17664 | 3640 | 9794 | 20292 | 22920 | 25548 |
| 3641 | 7167 | 12415 | 15038 | 17665 | 3642 | 9795 | 20293 | 22921 | 25549 |
| 3643 | 7168 | 12416 | 15039 | 17666 | 3644 | 9796 | 20294 | 22922 | 25550 |
| 3645 | 7169 | 12417 | 15040 | 17667 | 3646 | 9797 | 20295 | 22923 | 25551 |
| 3647 | 7170 | 12418 | 15041 | 17668 | 3648 | 9798 | 20296 | 22924 | 25552 |
| 3649 | 7171 | 12419 | 15042 | 17669 | 3650 | 9799 | 20297 | 22925 | 25553 |
| 3651 | 7172 | 12420 | 15043 | 17670 | 3652 | 9800 | 20298 | 22926 | 25554 |
| 3653 | 7173 | 12421 | 15044 | 17671 | 3654 | 9801 | 20299 | 22927 | 25555 |
| 3655 | 7174 | 12422 | 15045 | 17672 | 3656 | 9802 | 20300 | 22928 | 25556 |
| 3657 | 7175 | 12423 | 15046 | 17673 | 3658 | 9803 | 20301 | 22929 | 25557 |
| 3659 | 7176 | 12424 | 15047 | 17674 | 3660 | 9804 | 20302 | 22930 | 25558 |
| 3661 | 7177 | 12425 | 15048 | 17675 | 3662 | 9805 | 20303 | 22931 | 25559 |
| 3663 | 7178 | 12426 | 15049 | 17676 | 3664 | 9806 | 20304 | 22932 | 25560 |
| 3665 | 7179 | 12427 | 15050 | 17677 | 3666 | 9807 | 20305 | 22933 | 25561 |
| 3667 | 7180 | 12428 | 15051 | 17678 | 3668 | 9808 | 20306 | 22934 | 25562 |
| 3669 | 7181 | 12429 | 15052 | 17679 | 3670 | 9809 | 20307 | 22935 | 25563 |
| 3671 | 7182 | 12430 | 15053 | 17680 | 3672 | 9810 | 20308 | 22936 | 25564 |
| 3673 | 7183 | 12431 | 15054 | 17681 | 3674 | 9811 | 20309 | 22937 | 25565 |
| 3675 | 7184 | 12432 | 15055 | 17682 | 3676 | 9812 | 20310 | 22938 | 25566 |
| 3677 | 7185 | 12433 | 15056 | 17683 | 3678 | 9813 | 20311 | 22939 | 25567 |
| 3679 | 7186 | 12434 | 15057 | 17684 | 3680 | 9814 | 20312 | 22940 | 25568 |
| 3681 | 7187 | 12435 | 15058 | 17685 | 3682 | 9815 | 20313 | 22941 | 25569 |
| 3683 | 7188 | 12436 | 15059 | 17686 | 3684 | 9816 | 20314 | 22942 | 25570 |
| 3685 | 7189 | 12437 | 15060 | 17687 | 3686 | 9817 | 20315 | 22943 | 25571 |
| 3687 | 7190 | 12438 | 15061 | 17688 | 3688 | 9818 | 20316 | 22944 | 25572 |
| 3689 | 7191 | 12439 | 15062 | 17689 | 3690 | 9819 | 20317 | 22945 | 25573 |
| 3691 | 7192 | 12440 | 15063 | 17690 | 3692 | 9820 | 20318 | 22946 | 25574 |
| 3693 | 7193 | 12441 | 15064 | 17691 | 3694 | 9821 | 20319 | 22947 | 25575 |
| 3695 | 7194 | 12442 | 15065 | 17692 | 3696 | 9822 | 20320 | 22948 | 25576 |
| 3697 | 7195 | 12443 | 15066 | 17693 | 3698 | 9823 | 20321 | 22949 | 25577 |
| 3699 | 7196 | 12444 | 15067 | 17694 | 3700 | 9824 | 20322 | 22950 | 25578 |
| 3701 | 7197 | 12445 | 15068 | 17695 | 3702 | 9825 | 20323 | 22951 | 25579 |
| 3703 | 7198 | 12446 | 15069 | 17696 | 3704 | 9826 | 20324 | 22952 | 25580 |
| 3705 | 7199 | 12447 | 15070 | 17697 | 3706 | 9827 | 20325 | 22953 | 25581 |
| 3707 | 7200 | 12448 | 15071 | 17698 | 3708 | 9828 | 20326 | 22954 | 25582 |
| 3709 | 7201 | 12449 | 15072 | 17699 | 3710 | 9829 | 20327 | 22955 | 25583 |
| 3711 | 7202 | 12450 | 15073 | 17700 | 3712 | 9830 | 20328 | 22956 | 25584 |
| 3713 | 7203 | 12451 | 15074 | 17701 | 3714 | 9831 | 20329 | 22957 | 25585 |
| 3715 | 7204 | 12452 | 15075 | 17702 | 3716 | 9832 | 20330 | 22958 | 25586 |
| 3717 | 7205 | 12453 | 15076 | 17703 | 3718 | 9833 | 20331 | 22959 | 25587 |
| 3719 | 7206 | 12454 | 15077 | 17704 | 3720 | 9834 | 20332 | 22960 | 25588 |
| 3721 | 7207 | 12455 | 15078 | 17705 | 3722 | 9835 | 20333 | 22961 | 25589 |
| 3723 | 7208 | 12456 | 15079 | 17706 | 3724 | 9836 | 20334 | 22962 | 25590 |
| 3725 | 7209 | 12457 | 15080 | 17707 | 3726 | 9837 | 20335 | 22963 | 25591 |
| 3727 | 7210 | 12458 | 15081 | 17708 | 3728 | 9838 | 20336 | 22964 | 25592 |
| 3729 | 7211 | 12459 | 15082 | 17709 | 3730 | 9839 | 20337 | 22965 | 25593 |
| 3731 | 7212 | 12460 | 15083 | 17710 | 3732 | 9840 | 20338 | 22966 | 25594 |
| 3733 | 7213 | 12461 | 15084 | 17711 | 3734 | 9841 | 20339 | 22967 | 25595 |
| 3735 | 7214 | 12462 | 15085 | 17712 | 3736 | 9842 | 20340 | 22968 | 25596 |
| 3737 | 7215 | 12463 | 15086 | 17713 | 3738 | 9843 | 20341 | 22969 | 25597 |
| 3739 | 7216 | 12464 | 15087 | 17714 | 3740 | 9844 | 20342 | 22970 | 25598 |
| 3741 | 7217 | 12465 | 15088 | 17715 | 3742 | 9845 | 20343 | 22971 | 25599 |
| 3743 | 7218 | 12466 | 15089 | 17716 | 3744 | 9846 | 20344 | 22972 | 25600 |
| 3745 | 7219 | 12467 | 15090 | 17717 | 3746 | 9847 | 20345 | 22973 | 25601 |
| 3747 | 7220 | 12468 | 15091 | 17718 | 3748 | 9848 | 20346 | 22974 | 25602 |
| 3749 | 7221 | 12469 | 15092 | 17719 | 3750 | 9849 | 20347 | 22975 | 25603 |
| 3751 | 7222 | 12470 | 15093 | 17720 | 3752 | 9850 | 20348 | 22976 | 25604 |
| 3753 | 7223 | 12471 | 15094 | 17721 | 3754 | 9851 | 20349 | 22977 | 25605 |
| 3755 | 7224 | 12472 | 15095 | 17722 | 3756 | 9852 | 20350 | 22978 | 25606 |
| 3757 | 7225 | 12473 | 15096 | 17723 | 3758 | 9853 | 20351 | 22979 | 25607 |
| 3759 | 7226 | 12474 | 15097 | 17724 | 3760 | 9854 | 20352 | 22980 | 25608 |
| 3761 | 7227 | 12475 | 15098 | 17725 | 3762 | 9855 | 20353 | 22981 | 25609 |
| 3763 | 7228 | 12476 | 15099 | 17726 | 3764 | 9856 | 20354 | 22982 | 25610 |
| 3765 | 7229 | 12477 | 15100 | 17727 | 3766 | 9857 | 20355 | 22983 | 25611 |
| 3767 | 7230 | 12478 | 15101 | 17728 | 3768 | 9858 | 20356 | 22984 | 25612 |
| 3769 | 7231 | 12479 | 15102 | 17729 | 3770 | 9859 | 20357 | 22985 | 25613 |
| 3771 | 7232 | 12480 | 15103 | 17730 | 3772 | 9860 | 20358 | 22986 | 25614 |
| 3773 | 7233 | 12481 | 15104 | 17731 | 3774 | 9861 | 20359 | 22987 | 25615 |
| 3775 | 7234 | 12482 | 15105 | 17732 | 3776 | 9862 | 20360 | 22988 | 25616 |
| 3777 | 7235 | 12483 | 15106 | 17733 | 3778 | 9863 | 20361 | 22989 | 25617 |
| 3779 | 7236 | 12484 | 15107 | 17734 | 3780 | 9864 | 20362 | 22990 | 25618 |
| 3781 | 7237 | 12485 | 15108 | 17735 | 3782 | 9865 | 20363 | 22991 | 25619 |
| 3783 | 7238 | 12486 | 15109 | 17736 | 3784 | 9866 | 20364 | 22992 | 25620 |
| 3785 | 7239 | 12487 | 15110 | 17737 | 3786 | 9867 | 20365 | 22993 | 25621 |
| 3787 | 7240 | 12488 | 15111 | 17738 | 3788 | 9868 | 20366 | 22994 | 25622 |
| 3789 | 7241 | 12489 | 15112 | 17739 | 3790 | 9869 | 20367 | 22995 | 25623 |
| 3791 | 7242 | 12490 | 15113 | 17740 | 3792 | 9870 | 20368 | 22996 | 25624 |
| 3793 | 7243 | 12491 | 15114 | 17741 | 3794 | 9871 | 20369 | 22997 | 25625 |
| 3795 | 7244 | 12492 | 15115 | 17742 | 3796 | 9872 | 20370 | 22998 | 25626 |
| 3797 | 7245 | 12493 | 15116 | 17743 | 3798 | 9873 | 20371 | 22999 | 25627 |
| 3799 | 7246 | 12494 | 15117 | 17744 | 3800 | 9874 | 20372 | 23000 | 25628 |
| 3801 | 7247 | 12495 | 15118 | 17745 | 3802 | 9875 | 20373 | 23001 | 25629 |
| 3803 | 7248 | 12496 | 15119 | 17746 | 3804 | 9876 | 20374 | 23002 | 25630 |
| 3805 | 7249 | 12497 | 15120 | 17747 | 3806 | 9877 | 20375 | 23003 | 25631 |
| 3807 | 7250 | 12498 | 15121 | 17748 | 3808 | 9878 | 20376 | 23004 | 25632 |
| 3809 | 7251 | 12499 | 15122 | 17749 | 3810 | 9879 | 20377 | 23005 | 25633 |
| 3811 | 7252 | 12500 | 15123 | 17750 | 3812 | 9880 | 20378 | 23006 | 25634 |
| 3813 | 7253 | 12501 | 15124 | 17751 | 3814 | 9881 | 20379 | 23007 | 25635 |
| 3815 | 7254 | 12502 | 15125 | 17752 | 3816 | 9882 | 20380 | 23008 | 25636 |
| 3817 | 7255 | 12503 | 15126 | 17753 | 3818 | 9883 | 20381 | 23009 | 25637 |
| 3819 | 7256 | 12504 | 15127 | 17754 | 3820 | 9884 | 20382 | 23010 | 25638 |
| 3821 | 7257 | 12505 | 15128 | 17755 | 3822 | 9885 | 20383 | 23011 | 25639 |
| 3823 | 7258 | 12506 | 15129 | 17756 | 3824 | 9886 | 20384 | 23012 | 25640 |
| 3825 | 7259 | 12507 | 15130 | 17757 | 3826 | 9887 | 20385 | 23013 | 25641 |
| 3827 | 7260 | 12508 | 15131 | 17758 | 3828 | 9888 | 20386 | 23014 | 25642 |
| 3829 | 7261 | 12509 | 15132 | 17759 | 3830 | 9889 | 20387 | 23015 | 25643 |
| 3831 | 7262 | 12510 | 15133 | 17760 | 3832 | 9890 | 20388 | 23016 | 25644 |
| 3833 | 7263 | 12511 | 15134 | 17761 | 3834 | 9891 | 20389 | 23017 | 25645 |
| 3835 | 7264 | 12512 | 15135 | 17762 | 3836 | 9892 | 20390 | 23018 | 25646 |
| 3837 | 7265 | 12513 | 15136 | 17763 | 3838 | 9893 | 20391 | 23019 | 25647 |
| 3839 | 7266 | 12514 | 15137 | 17764 | 3840 | 9894 | 20392 | 23020 | 25648 |
| 3841 | 7267 | 12515 | 15138 | 17765 | 3842 | 9895 | 20393 | 23021 | 25649 |
| 3843 | 7268 | 12516 | 15139 | 17766 | 3844 | 9896 | 20394 | 23022 | 25650 |
| 3845 | 7269 | 12517 | 15140 | 17767 | 3846 | 9897 | 20395 | 23023 | 25651 |
| 3847 | 7270 | 12518 | 15141 | 17768 | 3848 | 9898 | 20396 | 23024 | 25652 |
| 3849 | 7271 | 12519 | 15142 | 17769 | 3850 | 9899 | 20397 | 23025 | 25653 |
| 3851 | 7272 | 12520 | 15143 | 17770 | 3852 | 9900 | 20398 | 23026 | 25654 |
| 3853 | 7273 | 12521 | 15144 | 17771 | 3854 | 9901 | 20399 | 23027 | 25655 |
| 3855 | 7274 | 12522 | 15145 | 17772 | 3856 | 9902 | 20400 | 23028 | 25656 |
| 3857 | 7275 | 12523 | 15146 | 17773 | 3858 | 9903 | 20401 | 23029 | 25657 |
| 3859 | 7276 | 12524 | 15147 | 17774 | 3860 | 9904 | 20402 | 23030 | 25658 |
| 3861 | 7277 | 12525 | 15148 | 17775 | 3862 | 9905 | 20403 | 23031 | 25659 |
| 3863 | 7278 | 12526 | 15149 | 17776 | 3864 | 9906 | 20404 | 23032 | 25660 |
| 3865 | 7279 | 12527 | 15150 | 17777 | 3866 | 9907 | 20405 | 23033 | 25661 |
| 3867 | 7280 | 12528 | 15151 | 17778 | 3868 | 9908 | 20406 | 23034 | 25662 |
| 3869 | 7281 | 12529 | 15152 | 17779 | 3870 | 9909 | 20407 | 23035 | 25663 |
| 3871 | 7282 | 12530 | 15153 | 17780 | 3872 | 9910 | 20408 | 23036 | 25664 |
| 3873 | 7283 | 12531 | 15154 | 17781 | 3874 | 9911 | 20409 | 23037 | 25665 |
| 3875 | 7284 | 12532 | 15155 | 17782 | 3876 | 9912 | 20410 | 23038 | 25666 |
| 3877 | 7285 | 12533 | 15156 | 17783 | 3878 | 9913 | 20411 | 23039 | 25667 |
| 3879 | 7286 | 12534 | 15157 | 17784 | 3880 | 9914 | 20412 | 23040 | 25668 |
| 3881 | 7287 | 12535 | 15158 | 17785 | 3882 | 9915 | 20413 | 23041 | 25669 |
| 3883 | 7288 | 12536 | 15159 | 17786 | 3884 | 9916 | 20414 | 23042 | 25670 |
| 3885 | 7289 | 12537 | 15160 | 17787 | 3886 | 9917 | 20415 | 23043 | 25671 |
| 3887 | 7290 | 12538 | 15161 | 17788 | 3888 | 9918 | 20416 | 23044 | 25672 |
| 3889 | 7291 | 12539 | 15162 | 17789 | 3890 | 9919 | 20417 | 23045 | 25673 |
| 3891 | 7292 | 12540 | 15163 | 17790 | 3892 | 9920 | 20418 | 23046 | 25674 |
| 3893 | 7293 | 12541 | 15164 | 17791 | 3894 | 9921 | 20419 | 23047 | 25675 |
| 3895 | 7294 | 12542 | 15165 | 17792 | 3896 | 9922 | 20420 | 23048 | 25676 |
| 3897 | 7295 | 12543 | 15166 | 17793 | 3898 | 9923 | 20421 | 23049 | 25677 |
| 3899 | 7296 | 12544 | 15167 | 17794 | 3900 | 9924 | 20422 | 23050 | 25678 |
| 3901 | 7297 | 12545 | 15168 | 17795 | 3902 | 9925 | 20423 | 23051 | 25679 |
| 3903 | 7298 | 12546 | 15169 | 17796 | 3904 | 9926 | 20424 | 23052 | 25680 |
| 3905 | 7299 | 12547 | 15170 | 17797 | 3906 | 9927 | 20425 | 23053 | 25681 |
| 3907 | 7300 | 12548 | 15171 | 17798 | 3908 | 9928 | 20426 | 23054 | 25682 |
| 3909 | 7301 | 12549 | 15172 | 17799 | 3910 | 9929 | 20427 | 23055 | 25683 |
| 3911 | 7302 | 12550 | 15173 | 17800 | 3912 | 9930 | 20428 | 23056 | 25684 |
| 3913 | 7303 | 12551 | 15174 | 17801 | 3914 | 9931 | 20429 | 23057 | 25685 |
| 3915 | 7304 | 12552 | 15175 | 17802 | 3916 | 9932 | 20430 | 23058 | 25686 |
| 3917 | 7305 | 12553 | 15176 | 17803 | 3918 | 9933 | 20431 | 23059 | 25687 |
| 3919 | 7306 | 12554 | 15177 | 17804 | 3920 | 9934 | 20432 | 23060 | 25688 |
| 3921 | 7307 | 12555 | 15178 | 17805 | 3922 | 9935 | 20433 | 23061 | 25689 |
| 3923 | 7308 | 12556 | 15179 | 17806 | 3924 | 9936 | 20434 | 23062 | 25690 |
| 3925 | 7309 | 12557 | 15180 | 17807 | 3926 | 9937 | 20435 | 23063 | 25691 |
| 3927 | 7310 | 12558 | 15181 | 17808 | 3928 | 9938 | 20436 | 23064 | 25692 |
| 3929 | 7311 | 12559 | 15182 | 17809 | 3930 | 9939 | 20437 | 23065 | 25693 |
| 3931 | 7312 | 12560 | 15183 | 17810 | 3932 | 9940 | 20438 | 23066 | 25694 |
| 3933 | 7313 | 12561 | 15184 | 17811 | 3934 | 9941 | 20439 | 23067 | 25695 |
| 3935 | 7314 | 12562 | 15185 | 17812 | 3936 | 9942 | 20440 | 23068 | 25696 |
| 3937 | 7315 | 12563 | 15186 | 17813 | 3938 | 9943 | 20441 | 23069 | 25697 |
| 3939 | 7316 | 12564 | 15187 | 17814 | 3940 | 9944 | 20442 | 23070 | 25698 |
| 3941 | 7317 | 12565 | 15188 | 17815 | 3942 | 9945 | 20443 | 23071 | 25699 |
| 3943 | 7318 | 12566 | 15189 | 17816 | 3944 | 9946 | 20444 | 23072 | 25700 |
| 3945 | 7319 | 12567 | 15190 | 17817 | 3946 | 9947 | 20445 | 23073 | 25701 |
| 3947 | 7320 | 12568 | 15191 | 17818 | 3948 | 9948 | 20446 | 23074 | 25702 |
| 3949 | 7321 | 12569 | 15192 | 17819 | 3950 | 9949 | 20447 | 23075 | 25703 |
| 3951 | 7322 | 12570 | 15193 | 17820 | 3952 | 9950 | 20448 | 23076 | 25704 |
| 3953 | 7323 | 12571 | 15194 | 17821 | 3954 | 9951 | 20449 | 23077 | 25705 |
| 3955 | 7324 | 12572 | 15195 | 17822 | 3956 | 9952 | 20450 | 23078 | 25706 |
| 3957 | 7325 | 12573 | 15196 | 17823 | 3958 | 9953 | 20451 | 23079 | 25707 |
| 3959 | 7326 | 12574 | 15197 | 17824 | 3960 | 9954 | 20452 | 23080 | 25708 |
| 3961 | 7327 | 12575 | 15198 | 17825 | 3962 | 9955 | 20453 | 23081 | 25709 |
| 3963 | 7328 | 12576 | 15199 | 17826 | 3964 | 9956 | 20454 | 23082 | 25710 |
| 3965 | 7329 | 12577 | 15200 | 17827 | 3966 | 9957 | 20455 | 23083 | 25711 |
| 3967 | 7330 | 12578 | 15201 | 17828 | 3968 | 9958 | 20456 | 23084 | 25712 |
| 3969 | 7331 | 12579 | 15202 | 17829 | 3970 | 9959 | 20457 | 23085 | 25713 |
| 3971 | 7332 | 12580 | 15203 | 17830 | 3972 | 9960 | 20458 | 23086 | 25714 |
| 3973 | 7333 | 12581 | 15204 | 17831 | 3974 | 9961 | 20459 | 23087 | 25715 |
| 3975 | 7334 | 12582 | 15205 | 17832 | 3976 | 9962 | 20460 | 23088 | 25716 |
| 3977 | 7335 | 12583 | 15206 | 17833 | 3978 | 9963 | 20461 | 23089 | 25717 |
| 3979 | 7336 | 12584 | 15207 | 17834 | 3980 | 9964 | 20462 | 23090 | 25718 |
| 3981 | 7337 | 12585 | 15208 | 17835 | 3982 | 9965 | 20463 | 23091 | 25719 |
| 3983 | 7338 | 12586 | 15209 | 17836 | 3984 | 9966 | 20464 | 23092 | 25720 |
| 3985 | 7339 | 12587 | 15210 | 17837 | 3986 | 9967 | 20465 | 23093 | 25721 |
| 3987 | 7340 | 12588 | 15211 | 17838 | 3988 | 9968 | 20466 | 23094 | 25722 |
| 3989 | 7341 | 12589 | 15212 | 17839 | 3990 | 9969 | 20467 | 23095 | 25723 |
| 3991 | 7342 | 12590 | 15213 | 17840 | 3992 | 9970 | 20468 | 23096 | 25724 |
| 3993 | 7343 | 12591 | 15214 | 17841 | 3994 | 9971 | 20469 | 23097 | 25725 |
| 3995 | 7344 | 12592 | 15215 | 17842 | 3996 | 9972 | 20470 | 23098 | 25726 |
| 3997 | 7345 | 12593 | 15216 | 17843 | 3998 | 9973 | 20471 | 23099 | 25727 |
| 3999 | 7346 | 12594 | 15217 | 17844 | 4000 | 9974 | 20472 | 23100 | 25728 |
| 4001 | 7347 | 12595 | 15218 | 17845 | 4002 | 9975 | 20473 | 23101 | 25729 |
| 4003 | 7348 | 12596 | 15219 | 17846 | 4004 | 9976 | 20474 | 23102 | 25730 |
| 4005 | 7349 | 12597 | 15220 | 17847 | 4006 | 9977 | 20475 | 23103 | 25731 |
| 4007 | 7350 | 12598 | 15221 | 17848 | 4008 | 9978 | 20476 | 23104 | 25732 |
| 4009 | 7351 | 12599 | 15222 | 17849 | 4010 | 9979 | 20477 | 23105 | 25733 |
| 4011 | 7352 | 12600 | 15223 | 17850 | 4012 | 9980 | 20478 | 23106 | 25734 |
| 4013 | 7353 | 12601 | 15224 | 17851 | 4014 | 9981 | 20479 | 23107 | 25735 |
| 4015 | 7354 | 12602 | 15225 | 17852 | 4016 | 9982 | 20480 | 23108 | 25736 |
| 4017 | 7355 | 12603 | 15226 | 17853 | 4018 | 9983 | 20481 | 23109 | 25737 |
| 4019 | 7356 | 12604 | 15227 | 17854 | 4020 | 9984 | 20482 | 23110 | 25738 |
| 4021 | 7357 | 12605 | 15228 | 17855 | 4022 | 9985 | 20483 | 23111 | 25739 |
| 4023 | 7358 | 12606 | 15229 | 17856 | 4024 | 9986 | 20484 | 23112 | 25740 |
| 4025 | 7359 | 12607 | 15230 | 17857 | 4026 | 9987 | 20485 | 23113 | 25741 |
| 4027 | 7360 | 12608 | 15231 | 17858 | 4028 | 9988 | 20486 | 23114 | 25742 |
| 4029 | 7361 | 12609 | 15232 | 17859 | 4030 | 9989 | 20487 | 23115 | 25743 |
| 4031 | 7362 | 12610 | 15233 | 17860 | 4032 | 9990 | 20488 | 23116 | 25744 |
| 4033 | 7363 | 12611 | 15234 | 17861 | 4034 | 9991 | 20489 | 23117 | 25745 |
| 4035 | 7364 | 12612 | 15235 | 17862 | 4036 | 9992 | 20490 | 23118 | 25746 |
| 4037 | 7365 | 12613 | 15236 | 17863 | 4038 | 9993 | 20491 | 23119 | 25747 |
| 4039 | 7366 | 12614 | 15237 | 17864 | 4040 | 9994 | 20492 | 23120 | 25748 |
| 4041 | 7367 | 12615 | 15238 | 17865 | 4042 | 9995 | 20493 | 23121 | 25749 |
| 4043 | 7368 | 12616 | 15239 | 17866 | 4044 | 9996 | 20494 | 23122 | 25750 |
| 4045 | 7369 | 12617 | 15240 | 17867 | 4046 | 9997 | 20495 | 23123 | 25751 |
| 4047 | 7370 | 12618 | 15241 | 17868 | 4048 | 9998 | 20496 | 23124 | 25752 |
| 4049 | 7371 | 12619 | 15242 | 17869 | 4050 | 9999 | 20497 | 23125 | 25753 |
| 4051 | 7372 | 12620 | 15243 | 17870 | 4052 | 10000 | 20498 | 23126 | 25754 |
| 4053 | 7373 | 12621 | 15244 | 17871 | 4054 | 10001 | 20499 | 23127 | 25755 |
| 4055 | 7374 | 12622 | 15245 | 17872 | 4056 | 10002 | 20500 | 23128 | 25756 |
| 4057 | 7375 | 12623 | 15246 | 17873 | 4058 | 10003 | 20501 | 23129 | 25757 |
| 4059 | 7376 | 12624 | 15247 | 17874 | 4060 | 10004 | 20502 | 23130 | 25758 |
| 4061 | 7377 | 12625 | 15248 | 17875 | 4062 | 10005 | 20503 | 23131 | 25759 |
| 4063 | 7378 | 12626 | 15249 | 17876 | 4064 | 10006 | 20504 | 23132 | 25760 |
| 4065 | 7379 | 12627 | 15250 | 17877 | 4066 | 10007 | 20505 | 23133 | 25761 |
| 4067 | 7380 | 12628 | 15251 | 17878 | 4068 | 10008 | 20506 | 23134 | 25762 |
| 4069 | 7381 | 12629 | 15252 | 17879 | 4070 | 10009 | 20507 | 23135 | 25763 |
| 4071 | 7382 | 12630 | 15253 | 17880 | 4072 | 10010 | 20508 | 23136 | 25764 |
| 4073 | 7383 | 12631 | 15254 | 17881 | 4074 | 10011 | 20509 | 23137 | 25765 |
| 4075 | 7384 | 12632 | 15255 | 17882 | 4076 | 10012 | 20510 | 23138 | 25766 |
| 4077 | 7385 | 12633 | 15256 | 17883 | 4078 | 10013 | 20511 | 23139 | 25767 |
| 4079 | 7386 | 12634 | 15257 | 17884 | 4080 | 10014 | 20512 | 23140 | 25768 |
| 4081 | 7387 | 12635 | 15258 | 17885 | 4082 | 10015 | 20513 | 23141 | 25769 |
| 4083 | 7388 | 12636 | 15259 | 17886 | 4084 | 10016 | 20514 | 23142 | 25770 |
| 4085 | 7389 | 12637 | 15260 | 17887 | 4086 | 10017 | 20515 | 23143 | 25771 |
| 4087 | 7390 | 12638 | 15261 | 17888 | 4088 | 10018 | 20516 | 23144 | 25772 |
| 4089 | 7391 | 12639 | 15262 | 17889 | 4090 | 10019 | 20517 | 23145 | 25773 |
| 4091 | 7392 | 12640 | 15263 | 17890 | 4092 | 10020 | 20518 | 23146 | 25774 |
| 4093 | 7393 | 12641 | 15264 | 17891 | 4094 | 10021 | 20519 | 23147 | 25775 |
| 4095 | 7394 | 12642 | 15265 | 17892 | 4096 | 10022 | 20520 | 23148 | 25776 |
| 4097 | 7395 | 12643 | 15266 | 17893 | 4098 | 10023 | 20521 | 23149 | 25777 |
| 4099 | 7396 | 12644 | 15267 | 17894 | 4100 | 10024 | 20522 | 23150 | 25778 |
| 4101 | 7397 | 12645 | 15268 | 17895 | 4102 | 10025 | 20523 | 23151 | 25779 |
| 4103 | 7398 | 12646 | 15269 | 17896 | 4104 | 10026 | 20524 | 23152 | 25780 |
| 4105 | 7399 | 12647 | 15270 | 17897 | 4106 | 10027 | 20525 | 23153 | 25781 |
| 4107 | 7400 | 12648 | 15271 | 17898 | 4108 | 10028 | 20526 | 23154 | 25782 |
| 4109 | 7401 | 12649 | 15272 | 17899 | 4110 | 10029 | 20527 | 23155 | 25783 |
| 4111 | 7402 | 12650 | 15273 | 17900 | 4112 | 10030 | 20528 | 23156 | 25784 |
| 4113 | 7403 | 12651 | 15274 | 17901 | 4114 | 10031 | 20529 | 23157 | 25785 |
| 4115 | 7404 | 12652 | 15275 | 17902 | 4116 | 10032 | 20530 | 23158 | 25786 |
| 4117 | 7405 | 12653 | 15276 | 17903 | 4118 | 10033 | 20531 | 23159 | 25787 |
| 4119 | 7406 | 12654 | 15277 | 17904 | 4120 | 10034 | 20532 | 23160 | 25788 |
| 4121 | 7407 | 12655 | 15278 | 17905 | 4122 | 10035 | 20533 | 23161 | 25789 |
| 4123 | 7408 | 12656 | 15279 | 17906 | 4124 | 10036 | 20534 | 23162 | 25790 |
| 4125 | 7409 | 12657 | 15280 | 17907 | 4126 | 10037 | 20535 | 23163 | 25791 |
| 4127 | 7410 | 12658 | 15281 | 17908 | 4128 | 10038 | 20536 | 23164 | 25792 |
| 4129 | 7411 | 12659 | 15282 | 17909 | 4130 | 10039 | 20537 | 23165 | 25793 |
| 4131 | 7412 | 12660 | 15283 | 17910 | 4132 | 10040 | 20538 | 23166 | 25794 |
| 4133 | 7413 | 12661 | 15284 | 17911 | 4134 | 10041 | 20539 | 23167 | 25795 |
| 4135 | 7414 | 12662 | 15285 | 17912 | 4136 | 10042 | 20540 | 23168 | 25796 |
| 4137 | 7415 | 12663 | 15286 | 17913 | 4138 | 10043 | 20541 | 23169 | 25797 |
| 4139 | 7416 | 12664 | 15287 | 17914 | 4140 | 10044 | 20542 | 23170 | 25798 |
| 4141 | 7417 | 12665 | 15288 | 17915 | 4142 | 10045 | 20543 | 23171 | 25799 |
| 4143 | 7418 | 12666 | 15289 | 17916 | 4144 | 10046 | 20544 | 23172 | 25800 |
| 4145 | 7419 | 12667 | 15290 | 17917 | 4146 | 10047 | 20545 | 23173 | 25801 |
| 4147 | 7420 | 12668 | 15291 | 17918 | 4148 | 10048 | 20546 | 23174 | 25802 |
| 4149 | 7421 | 12669 | 15292 | 17919 | 4150 | 10049 | 20547 | 23175 | 25803 |
| 4151 | 7422 | 12670 | 15293 | 17920 | 4152 | 10050 | 20548 | 23176 | 25804 |
| 4153 | 7423 | 12671 | 15294 | 17921 | 4154 | 10051 | 20549 | 23177 | 25805 |
| 4155 | 7424 | 12672 | 15295 | 17922 | 4156 | 10052 | 20550 | 23178 | 25806 |
| 4157 | 7425 | 12673 | 15296 | 17923 | 4158 | 10053 | 20551 | 23179 | 25807 |
| 4159 | 7426 | 12674 | 15297 | 17924 | 4160 | 10054 | 20552 | 23180 | 25808 |
| 4161 | 7427 | 12675 | 15298 | 17925 | 4162 | 10055 | 20553 | 23181 | 25809 |
| 4163 | 7428 | 12676 | 15299 | 17926 | 4164 | 10056 | 20554 | 23182 | 25810 |
| 4165 | 7429 | 12677 | 15300 | 17927 | 4166 | 10057 | 20555 | 23183 | 25811 |
| 4167 | 7430 | 12678 | 15301 | 17928 | 4168 | 10058 | 20556 | 23184 | 25812 |
| 4169 | 7431 | 12679 | 15302 | 17929 | 4170 | 10059 | 20557 | 23185 | 25813 |
| 4171 | 7432 | 12680 | 15303 | 17930 | 4172 | 10060 | 20558 | 23186 | 25814 |
| 4173 | 7433 | 12681 | 15304 | 17931 | 4174 | 10061 | 20559 | 23187 | 25815 |
| 4175 | 7434 | 12682 | 15305 | 17932 | 4176 | 10062 | 20560 | 23188 | 25816 |
| 4177 | 7435 | 12683 | 15306 | 17933 | 4178 | 10063 | 20561 | 23189 | 25817 |
| 4179 | 7436 | 12684 | 15307 | 17934 | 4180 | 10064 | 20562 | 23190 | 25818 |
| 4181 | 7437 | 12685 | 15308 | 17935 | 4182 | 10065 | 20563 | 23191 | 25819 |
| 4183 | 7438 | 12686 | 15309 | 17936 | 4184 | 10066 | 20564 | 23192 | 25820 |
| 4185 | 7439 | 12687 | 15310 | 17937 | 4186 | 10067 | 20565 | 23193 | 25821 |
| 4187 | 7440 | 12688 | 15311 | 17938 | 4188 | 10068 | 20566 | 23194 | 25822 |
| 4189 | 7441 | 12689 | 15312 | 17939 | 4190 | 10069 | 20567 | 23195 | 25823 |
| 4191 | 7442 | 12690 | 15313 | 17940 | 4192 | 10070 | 20568 | 23196 | 25824 |
| 4193 | 7443 | 12691 | 15314 | 17941 | 4194 | 10071 | 20569 | 23197 | 25825 |
| 4195 | 7444 | 12692 | 15315 | 17942 | 4196 | 10072 | 20570 | 23198 | 25826 |
| 4197 | 7445 | 12693 | 15316 | 17943 | 4198 | 10073 | 20571 | 23199 | 25827 |
| 4199 | 7446 | 12694 | 15317 | 17944 | 4200 | 10074 | 20572 | 23200 | 25828 |
| 4201 | 7447 | 12695 | 15318 | 17945 | 4202 | 10075 | 20573 | 23201 | 25829 |
| 4203 | 7448 | 12696 | 15319 | 17946 | 4204 | 10076 | 20574 | 23202 | 25830 |
| 4205 | 7449 | 12697 | 15320 | 17947 | 4206 | 10077 | 20575 | 23203 | 25831 |
| 4207 | 7450 | 12698 | 15321 | 17948 | 4208 | 10078 | 20576 | 23204 | 25832 |
| 4209 | 7451 | 12699 | 15322 | 17949 | 4210 | 10079 | 20577 | 23205 | 25833 |
| 4211 | 7452 | 12700 | 15323 | 17950 | 4212 | 10080 | 20578 | 23206 | 25834 |
| 4213 | 7453 | 12701 | 15324 | 17951 | 4214 | 10081 | 20579 | 23207 | 25835 |
| 4215 | 7454 | 12702 | 15325 | 17952 | 4216 | 10082 | 20580 | 23208 | 25836 |
| 4217 | 7455 | 12703 | 15326 | 17953 | 4218 | 10083 | 20581 | 23209 | 25837 |
| 4219 | 7456 | 12704 | 15327 | 17954 | 4220 | 10084 | 20582 | 23210 | 25838 |
| 4221 | 7457 | 12705 | 15328 | 17955 | 4222 | 10085 | 20583 | 23211 | 25839 |
| 4223 | 7458 | 12706 | 15329 | 17956 | 4224 | 10086 | 20584 | 23212 | 25840 |
| 4225 | 7459 | 12707 | 15330 | 17957 | 4226 | 10087 | 20585 | 23213 | 25841 |
| 4227 | 7460 | 12708 | 15331 | 17958 | 4228 | 10088 | 20586 | 23214 | 25842 |
| 4229 | 7461 | 12709 | 15332 | 17959 | 4230 | 10089 | 20587 | 23215 | 25843 |
| 4231 | 7462 | 12710 | 15333 | 17960 | 4232 | 10090 | 20588 | 23216 | 25844 |
| 4233 | 7463 | 12711 | 15334 | 17961 | 4234 | 10091 | 20589 | 23217 | 25845 |
| 4235 | 7464 | 12712 | 15335 | 17962 | 4236 | 10092 | 20590 | 23218 | 25846 |
| 4237 | 7465 | 12713 | 15336 | 17963 | 4238 | 10093 | 20591 | 23219 | 25847 |
| 4239 | 7466 | 12714 | 15337 | 17964 | 4240 | 10094 | 20592 | 23220 | 25848 |
| 4241 | 7467 | 12715 | 15338 | 17965 | 4242 | 10095 | 20593 | 23221 | 25849 |
| 4243 | 7468 | 12716 | 15339 | 17966 | 4244 | 10096 | 20594 | 23222 | 25850 |
| 4245 | 7469 | 12717 | 15340 | 17967 | 4246 | 10097 | 20595 | 23223 | 25851 |
| 4247 | 7470 | 12718 | 15341 | 17968 | 4248 | 10098 | 20596 | 23224 | 25852 |
| 4249 | 7471 | 12719 | 15342 | 17969 | 4250 | 10099 | 20597 | 23225 | 25853 |
| 4251 | 7472 | 12720 | 15343 | 17970 | 4252 | 10100 | 20598 | 23226 | 25854 |
| 4253 | 7473 | 12721 | 15344 | 17971 | 4254 | 10101 | 20599 | 23227 | 25855 |
| 4255 | 7474 | 12722 | 15345 | 17972 | 4256 | 10102 | 20600 | 23228 | 25856 |
| 4257 | 7475 | 12723 | 15346 | 17973 | 4258 | 10103 | 20601 | 23229 | 25857 |
| 4259 | 7476 | 12724 | 15347 | 17974 | 4260 | 10104 | 20602 | 23230 | 25858 |
| 4261 | 7477 | 12725 | 15348 | 17975 | 4262 | 10105 | 20603 | 23231 | 25859 |
| 4263 | 7478 | 12726 | 15349 | 17976 | 4264 | 10106 | 20604 | 23232 | 25860 |
| 4265 | 7479 | 12727 | 15350 | 17977 | 4266 | 10107 | 20605 | 23233 | 25861 |
| 4267 | 7480 | 12728 | 15351 | 17978 | 4268 | 10108 | 20606 | 23234 | 25862 |
| 4269 | 7481 | 12729 | 15352 | 17979 | 4270 | 10109 | 20607 | 23235 | 25863 |
| 4271 | 7482 | 12730 | 15353 | 17980 | 4272 | 10110 | 20608 | 23236 | 25864 |
| 4273 | 7483 | 12731 | 15354 | 17981 | 4274 | 10111 | 20609 | 23237 | 25865 |
| 4275 | 7484 | 12732 | 15355 | 17982 | 4276 | 10112 | 20610 | 23238 | 25866 |
| 4277 | 7485 | 12733 | 15356 | 17983 | 4278 | 10113 | 20611 | 23239 | 25867 |
| 4279 | 7486 | 12734 | 15357 | 17984 | 4280 | 10114 | 20612 | 23240 | 25868 |
| 4281 | 7487 | 12735 | 15358 | 17985 | 4282 | 10115 | 20613 | 23241 | 25869 |
| 4283 | 7488 | 12736 | 15359 | 17986 | 4284 | 10116 | 20614 | 23242 | 25870 |
| 4285 | 7489 | 12737 | 15360 | 17987 | 4286 | 10117 | 20615 | 23243 | 25871 |
| 4287 | 7490 | 12738 | 15361 | 17988 | 4288 | 10118 | 20616 | 23244 | 25872 |
| 4289 | 7491 | 12739 | 15362 | 17989 | 4290 | 10119 | 20617 | 23245 | 25873 |
| 4291 | 7492 | 12740 | 15363 | 17990 | 4292 | 10120 | 20618 | 23246 | 25874 |
| 4293 | 7493 | 12741 | 15364 | 17991 | 4294 | 10121 | 20619 | 23247 | 25875 |
| 4295 | 7494 | 12742 | 15365 | 17992 | 4296 | 10122 | 20620 | 23248 | 25876 |
| 4297 | 7495 | 12743 | 15366 | 17993 | 4298 | 10123 | 20621 | 23249 | 25877 |
| 4299 | 7496 | 12744 | 15367 | 17994 | 4300 | 10124 | 20622 | 23250 | 25878 |
| 4301 | 7497 | 12745 | 15368 | 17995 | 4302 | 10125 | 20623 | 23251 | 25879 |
| 4303 | 7498 | 12746 | 15369 | 17996 | 4304 | 10126 | 20624 | 23252 | 25880 |
| 4305 | 7499 | 12747 | 15370 | 17997 | 4306 | 10127 | 20625 | 23253 | 25881 |
| 4307 | 7500 | 12748 | 15371 | 17998 | 4308 | 10128 | 20626 | 23254 | 25882 |
| 4309 | 7501 | 12749 | 15372 | 17999 | 4310 | 10129 | 20627 | 23255 | 25883 |
| 4311 | 7502 | 12750 | 15373 | 18000 | 4312 | 10130 | 20628 | 23256 | 25884 |
| 4313 | 7503 | 12751 | 15374 | 18001 | 4314 | 10131 | 20629 | 23257 | 25885 |
| 4315 | 7504 | 12752 | 15375 | 18002 | 4316 | 10132 | 20630 | 23258 | 25886 |
| 4317 | 7505 | 12753 | 15376 | 18003 | 4318 | 10133 | 20631 | 23259 | 25887 |
| 4319 | 7506 | 12754 | 15377 | 18004 | 4320 | 10134 | 20632 | 23260 | 25888 |
| 4321 | 7507 | 12755 | 15378 | 18005 | 4322 | 10135 | 20633 | 23261 | 25889 |
| 4323 | 7508 | 12756 | 15379 | 18006 | 4324 | 10136 | 20634 | 23262 | 25890 |
| 4325 | 7509 | 12757 | 15380 | 18007 | 4326 | 10137 | 20635 | 23263 | 25891 |
| 4327 | 7510 | 12758 | 15381 | 18008 | 4328 | 10138 | 20636 | 23264 | 25892 |
| 4329 | 7511 | 12759 | 15382 | 18009 | 4330 | 10139 | 20637 | 23265 | 25893 |
| 4331 | 7512 | 12760 | 15383 | 18010 | 4332 | 10140 | 20638 | 23266 | 25894 |
| 4333 | 7513 | 12761 | 15384 | 18011 | 4334 | 10141 | 20639 | 23267 | 25895 |
| 4335 | 7514 | 12762 | 15385 | 18012 | 4336 | 10142 | 20640 | 23268 | 25896 |
| 4337 | 7515 | 12763 | 15386 | 18013 | 4338 | 10143 | 20641 | 23269 | 25897 |
| 4339 | 7516 | 12764 | 15387 | 18014 | 4340 | 10144 | 20642 | 23270 | 25898 |
| 4341 | 7517 | 12765 | 15388 | 18015 | 4342 | 10145 | 20643 | 23271 | 25899 |
| 4343 | 7518 | 12766 | 15389 | 18016 | 4344 | 10146 | 20644 | 23272 | 25900 |
| 4345 | 7519 | 12767 | 15390 | 18017 | 4346 | 10147 | 20645 | 23273 | 25901 |
| 4347 | 7520 | 12768 | 15391 | 18018 | 4348 | 10148 | 20646 | 23274 | 25902 |
| 4349 | 7521 | 12769 | 15392 | 18019 | 4350 | 10149 | 20647 | 23275 | 25903 |
| 4351 | 7522 | 12770 | 15393 | 18020 | 4352 | 10150 | 20648 | 23276 | 25904 |
| 4353 | 7523 | 12771 | 15394 | 18021 | 4354 | 10151 | 20649 | 23277 | 25905 |
| 4355 | 7524 | 12772 | 15395 | 18022 | 4356 | 10152 | 20650 | 23278 | 25906 |
| 4357 | 7525 | 12773 | 15396 | 18023 | 4358 | 10153 | 20651 | 23279 | 25907 |
| 4359 | 7526 | 12774 | 15397 | 18024 | 4360 | 10154 | 20652 | 23280 | 25908 |
| 4361 | 7527 | 12775 | 15398 | 18025 | 4362 | 10155 | 20653 | 23281 | 25909 |
| 4363 | 7528 | 12776 | 15399 | 18026 | 4364 | 10156 | 20654 | 23282 | 25910 |
| 4365 | 7529 | 12777 | 15400 | 18027 | 4366 | 10157 | 20655 | 23283 | 25911 |
| 4367 | 7530 | 12778 | 15401 | 18028 | 4368 | 10158 | 20656 | 23284 | 25912 |
| 4369 | 7531 | 12779 | 15402 | 18029 | 4370 | 10159 | 20657 | 23285 | 25913 |
| 4371 | 7532 | 12780 | 15403 | 18030 | 4372 | 10160 | 20658 | 23286 | 25914 |
| 4373 | 7533 | 12781 | 15404 | 18031 | 4374 | 10161 | 20659 | 23287 | 25915 |
| 4375 | 7534 | 12782 | 15405 | 18032 | 4376 | 10162 | 20660 | 23288 | 25916 |
| 4377 | 7535 | 12783 | 15406 | 18033 | 4378 | 10163 | 20661 | 23289 | 25917 |
| 4379 | 7536 | 12784 | 15407 | 18034 | 4380 | 10164 | 20662 | 23290 | 25918 |
| 4381 | 7537 | 12785 | 15408 | 18035 | 4382 | 10165 | 20663 | 23291 | 25919 |
| 4383 | 7538 | 12786 | 15409 | 18036 | 4384 | 10166 | 20664 | 23292 | 25920 |
| 4385 | 7539 | 12787 | 15410 | 18037 | 4386 | 10167 | 20665 | 23293 | 25921 |
| 4387 | 7540 | 12788 | 15411 | 18038 | 4388 | 10168 | 20666 | 23294 | 25922 |
| 4389 | 7541 | 12789 | 15412 | 18039 | 4390 | 10169 | 20667 | 23295 | 25923 |
| 4391 | 7542 | 12790 | 15413 | 18040 | 4392 | 10170 | 20668 | 23296 | 25924 |
| 4393 | 7543 | 12791 | 15414 | 18041 | 4394 | 10171 | 20669 | 23297 | 25925 |
| 4395 | 7544 | 12792 | 15415 | 18042 | 4396 | 10172 | 20670 | 23298 | 25926 |
| 4397 | 7545 | 12793 | 15416 | 18043 | 4398 | 10173 | 20671 | 23299 | 25927 |
| 4399 | 7546 | 12794 | 15417 | 18044 | 4400 | 10174 | 20672 | 23300 | 25928 |
| 4401 | 7547 | 12795 | 15418 | 18045 | 4402 | 10175 | 20673 | 23301 | 25929 |
| 4403 | 7548 | 12796 | 15419 | 18046 | 4404 | 10176 | 20674 | 23302 | 25930 |
| 4405 | 7549 | 12797 | 15420 | 18047 | 4406 | 10177 | 20675 | 23303 | 25931 |
| 4407 | 7550 | 12798 | 15421 | 18048 | 4408 | 10178 | 20676 | 23304 | 25932 |
| 4409 | 7551 | 12799 | 15422 | 18049 | 4410 | 10179 | 20677 | 23305 | 25933 |
| 4411 | 7552 | 12800 | 15423 | 18050 | 4412 | 10180 | 20678 | 23306 | 25934 |
| 4413 | 7553 | 12801 | 15424 | 18051 | 4414 | 10181 | 20679 | 23307 | 25935 |
| 4415 | 7554 | 12802 | 15425 | 18052 | 4416 | 10182 | 20680 | 23308 | 25936 |
| 4417 | 7555 | 12803 | 15426 | 18053 | 4418 | 10183 | 20681 | 23309 | 25937 |
| 4419 | 7556 | 12804 | 15427 | 18054 | 4420 | 10184 | 20682 | 23310 | 25938 |
| 4421 | 7557 | 12805 | 15428 | 18055 | 4422 | 10185 | 20683 | 23311 | 25939 |
| 4423 | 7558 | 12806 | 15429 | 18056 | 4424 | 10186 | 20684 | 23312 | 25940 |
| 4425 | 7559 | | | 18057 | 4426 | 10187 | 20685 | 23313 | 25941 |
| 4427 | 7560 | 12807 | 15430 | 18058 | 4428 | 10188 | 20686 | 23314 | 25942 |
| 4429 | 7561 | 12808 | 15431 | 18059 | 4430 | 10189 | 20687 | 23315 | 25943 |
| 4431 | 7562 | 12809 | 15432 | 18060 | 4432 | 10190 | 20688 | 23316 | 25944 |
| 4433 | 7563 | 12810 | 15433 | 18061 | 4434 | 10191 | 20689 | 23317 | 25945 |
| 4435 | 7564 | 12811 | 15434 | 18062 | 4436 | 10192 | 20690 | 23318 | 25946 |
| 4437 | 7565 | 12812 | 15435 | 18063 | 4438 | 10193 | 20691 | 23319 | 25947 |
| 4439 | 7566 | 12813 | 15436 | 18064 | 4440 | 10194 | 20692 | 23320 | 25948 |
| 4441 | 7567 | 12814 | 15437 | 18065 | 4442 | 10195 | 20693 | 23321 | 25949 |
| 4443 | 7568 | 12815 | 15438 | 18066 | 4444 | 10196 | 20694 | 23322 | 25950 |
| 4445 | 7569 | 12816 | 15439 | 18067 | 4446 | 10197 | 20695 | 23323 | 25951 |
| 4447 | 7570 | 12817 | 15440 | 18068 | 4448 | 10198 | 20696 | 23324 | 25952 |
| 4449 | 7571 | 12818 | 15441 | 18069 | 4450 | 10199 | 20697 | 23325 | 25953 |
| 4451 | 7572 | 12819 | 15442 | 18070 | 4452 | 10200 | 20698 | 23326 | 25954 |
| 4453 | 7573 | 12820 | 15443 | 18071 | 4454 | 10201 | 20699 | 23327 | 25955 |
| 4455 | 7574 | 12821 | 15444 | 18072 | 4456 | 10202 | 20700 | 23328 | 25956 |
| 4457 | 7575 | 12822 | 15445 | 18073 | 4458 | 10203 | 20701 | 23329 | 25957 |
| 4459 | 7576 | 12823 | 15446 | 18074 | 4460 | 10204 | 20702 | 23330 | 25958 |
| 4461 | 7577 | 12824 | 15447 | 18075 | 4462 | 10205 | 20703 | 23331 | 25959 |
| 4463 | 7578 | 12825 | 15448 | 18076 | 4464 | 10206 | 20704 | 23332 | 25960 |
| 4465 | 7579 | 12826 | 15449 | 18077 | 4466 | 10207 | 20705 | 23333 | 25961 |
| 4467 | 7580 | 12827 | 15450 | 18078 | 4468 | 10208 | 20706 | 23334 | 25962 |
| 4469 | 7581 | 12828 | 15451 | 18079 | 4470 | 10209 | 20707 | 23335 | 25963 |
| 4471 | 7582 | 12829 | 15452 | 18080 | 4472 | 10210 | 20708 | 23336 | 25964 |
| 4473 | 7583 | 12830 | 15453 | 18081 | 4474 | 10211 | 20709 | 23337 | 25965 |
| 4475 | 7584 | 12831 | 15454 | 18082 | 4476 | 10212 | 20710 | 23338 | 25966 |
| 4477 | 7585 | 12832 | 15455 | 18083 | 4478 | 10213 | 20711 | 23339 | 25967 |
| 4479 | 7586 | 12833 | 15456 | 18084 | 4480 | 10214 | 20712 | 23340 | 25968 |
| 4481 | 7587 | 12834 | 15457 | 18085 | 4482 | 10215 | 20713 | 23341 | 25969 |
| 4483 | 7588 | 12835 | 15458 | 18086 | 4484 | 10216 | 20714 | 23342 | 25970 |
| 4485 | 7589 | 12836 | 15459 | 18087 | 4486 | 10217 | 20715 | 23343 | 25971 |
| 4487 | 7590 | 12837 | 15460 | 18088 | 4488 | 10218 | 20716 | 23344 | 25972 |
| 4489 | 7591 | 12838 | 15461 | 18089 | 4490 | 10219 | 20717 | 23345 | 25973 |
| 4491 | 7592 | 12839 | 15462 | 18090 | 4492 | 10220 | 20718 | 23346 | 25974 |
| 4493 | 7593 | 12840 | 15463 | 18091 | 4494 | 10221 | 20719 | 23347 | 25975 |
| 4495 | 7594 | 12841 | 15464 | 18092 | 4496 | 10222 | 20720 | 23348 | 25976 |
| 4497 | 7595 | 12842 | 15465 | 18093 | 4498 | 10223 | 20721 | 23349 | 25977 |
| 4499 | 7596 | 12843 | 15466 | 18094 | 4500 | 10224 | 20722 | 23350 | 25978 |
| 4501 | 7597 | 12844 | 15467 | 18095 | 4502 | 10225 | 20723 | 23351 | 25979 |
| 4503 | 7598 | 12845 | 15468 | 18096 | 4504 | 10226 | 20724 | 23352 | 25980 |
| 4505 | 7599 | 12846 | 15469 | 18097 | 4506 | 10227 | 20725 | 23353 | 25981 |
| 4507 | 7600 | 12847 | 15470 | 18098 | 4508 | 10228 | 20726 | 23354 | 25982 |
| 4509 | 7601 | 12848 | 15471 | 18099 | 4510 | 10229 | 20727 | 23355 | 25983 |
| 4511 | 7602 | 12849 | 15472 | 18100 | 4512 | 10230 | 20728 | 23356 | 25984 |
| 4513 | 7603 | 12850 | 15473 | 18101 | 4514 | 10231 | 20729 | 23357 | 25985 |
| 4515 | 7604 | 12851 | 15474 | 18102 | 4516 | 10232 | 20730 | 23358 | 25986 |
| 4517 | 7605 | 12852 | 15475 | 18103 | 4518 | 10233 | 20731 | 23359 | 25987 |
| 4519 | 7606 | 12853 | 15476 | 18104 | 4520 | 10234 | 20732 | 23360 | 25988 |
| 4521 | 7607 | 12854 | 15477 | 18105 | 4522 | 10235 | 20733 | 23361 | 25989 |
| 4523 | 7608 | 12855 | 15478 | 18106 | 4524 | 10236 | 20734 | 23362 | 25990 |
| 4525 | 7609 | 12856 | 15479 | 18107 | 4526 | 10237 | 20735 | 23363 | 25991 |
| 4527 | 7610 | 12857 | 15480 | 18108 | 4528 | 10238 | 20736 | 23364 | 25992 |
| 4529 | 7611 | 12858 | 15481 | 18109 | 4530 | 10239 | 20737 | 23365 | 25993 |
| 4531 | 7612 | 12859 | 15482 | 18110 | 4532 | 10240 | 20738 | 23366 | 25994 |
| 4533 | 7613 | 12860 | 15483 | 18111 | 4534 | 10241 | 20739 | 23367 | 25995 |
| 4535 | 7614 | 12861 | 15484 | 18112 | 4536 | 10242 | 20740 | 23368 | 25996 |
| 4537 | 7615 | 12862 | 15485 | 18113 | 4538 | 10243 | 20741 | 23369 | 25997 |
| 4539 | 7616 | 12863 | 15486 | 18114 | 4540 | 10244 | 20742 | 23370 | 25998 |
| 4541 | 7617 | 12864 | 15487 | 18115 | 4542 | 10245 | 20743 | 23371 | 25999 |
| 4543 | 7618 | 12865 | 15488 | 18116 | 4544 | 10246 | 20744 | 23372 | 26000 |
| 4545 | 7619 | 12866 | 15489 | 18117 | 4546 | 10247 | 20745 | 23373 | 26001 |
| 4547 | 7620 | 12867 | 15490 | 18118 | 4548 | 10248 | 20746 | 23374 | 26002 |
| 4549 | 7621 | 12868 | 15491 | 18119 | 4550 | 10249 | 20747 | 23375 | 26003 |
| 4551 | 7622 | 12869 | 15492 | 18120 | 4552 | 10250 | 20748 | 23376 | 26004 |
| 4553 | 7623 | 12870 | 15493 | 18121 | 4554 | 10251 | 20749 | 23377 | 26005 |
| 4555 | 7624 | 12871 | 15494 | 18122 | 4556 | 10252 | 20750 | 23378 | 26006 |
| 4557 | 7625 | 12872 | 15495 | 18123 | 4558 | 10253 | 20751 | 23379 | 26007 |
| 4559 | 7626 | 12873 | 15496 | 18124 | 4560 | 10254 | 20752 | 23380 | 26008 |
| 4561 | 7627 | 12874 | 15497 | 18125 | 4562 | 10255 | 20753 | 23381 | 26009 |
| 4563 | 7628 | 12875 | 15498 | 18126 | 4564 | 10256 | 20754 | 23382 | 26010 |
| 4565 | 7629 | 12876 | 15499 | 18127 | 4566 | 10257 | 20755 | 23383 | 26011 |
| 4567 | 7630 | 12877 | 15500 | 18128 | 4568 | 10258 | 20756 | 23384 | 26012 |
| 4569 | 7631 | 12878 | 15501 | 18129 | 4570 | 10259 | 20757 | 23385 | 26013 |
| 4571 | 7632 | 12879 | 15502 | 18130 | 4572 | 10260 | 20758 | 23386 | 26014 |
| 4573 | 7633 | 12880 | 15503 | 18131 | 4574 | 10261 | 20759 | 23387 | 26015 |
| 4575 | 7634 | 12881 | 15504 | 18132 | 4576 | 10262 | 20760 | 23388 | 26016 |
| 4577 | 7635 | 12882 | 15505 | 18133 | 4578 | 10263 | 20761 | 23389 | 26017 |
| 4579 | 7636 | 12883 | 15506 | 18134 | 4580 | 10264 | 20762 | 23390 | 26018 |
| 4581 | 7637 | 12884 | 15507 | 18135 | 4582 | 10265 | 20763 | 23391 | 26019 |
| 4583 | 7638 | 12885 | 15508 | 18136 | 4584 | 10266 | 20764 | 23392 | 26020 |
| 4585 | 7639 | 12886 | 15509 | 18137 | 4586 | 10267 | 20765 | 23393 | 26021 |
| 4587 | 7640 | 12887 | 15510 | 18138 | 4588 | 10268 | 20766 | 23394 | 26022 |
| 4589 | 7641 | 12888 | 15511 | 18139 | 4590 | 10269 | 20767 | 23395 | 26023 |
| 4591 | 7642 | 12889 | 15512 | 18140 | 4592 | 10270 | 20768 | 23396 | 26024 |
| 4593 | 7643 | 12890 | 15513 | 18141 | 4594 | 10271 | 20769 | 23397 | 26025 |
| 4595 | 7644 | 12891 | 15514 | 18142 | 4596 | 10272 | 20770 | 23398 | 26026 |
| 4597 | 7645 | 12892 | 15515 | 18143 | 4598 | 10273 | 20771 | 23399 | 26027 |
| 4599 | 7646 | 12893 | 15516 | 18144 | 4600 | 10274 | 20772 | 23400 | 26028 |
| 4601 | 7647 | 12894 | 15517 | 18145 | 4602 | 10275 | 20773 | 23401 | 26029 |
| 4603 | 7648 | 12895 | 15518 | 18146 | 4604 | 10276 | 20774 | 23402 | 26030 |
| 4605 | 7649 | 12896 | 15519 | 18147 | 4606 | 10277 | 20775 | 23403 | 26031 |
| 4607 | 7650 | 12897 | 15520 | 18148 | 4608 | 10278 | 20776 | 23404 | 26032 |
| 4609 | 7651 | 12898 | 15521 | 18149 | 4610 | 10279 | 20777 | 23405 | 26033 |
| 4611 | 7652 | 12899 | 15522 | 18150 | 4612 | 10280 | 20778 | 23406 | 26034 |
| 4613 | 7653 | 12900 | 15523 | 18151 | 4614 | 10281 | 20779 | 23407 | 26035 |
| 4615 | 7654 | 12901 | 15524 | 18152 | 4616 | 10282 | 20780 | 23408 | 26036 |
| 4617 | 7655 | 12902 | 15525 | 18153 | 4618 | 10283 | 20781 | 23409 | 26037 |
| 4619 | 7656 | 12903 | 15526 | 18154 | 4620 | 10284 | 20782 | 23410 | 26038 |
| 4621 | 7657 | 12904 | 15527 | 18155 | 4622 | 10285 | 20783 | 23411 | 26039 |
| 4623 | 7658 | 12905 | 15528 | 18156 | 4624 | 10286 | 20784 | 23412 | 26040 |
| 4625 | 7659 | 12906 | 15529 | 18157 | 4626 | 10287 | 20785 | 23413 | 26041 |
| 4627 | 7660 | 12907 | 15530 | 18158 | 4628 | 10288 | 20786 | 23414 | 26042 |
| 4629 | 7661 | 12908 | 15531 | 18159 | 4630 | 10289 | 20787 | 23415 | 26043 |
| 4631 | 7662 | 12909 | 15532 | 18160 | 4632 | 10290 | 20788 | 23416 | 26044 |
| 4633 | 7663 | 12910 | 15533 | 18161 | 4634 | 10291 | 20789 | 23417 | 26045 |
| 4635 | 7664 | 12911 | 15534 | 18162 | 4636 | 10292 | 20790 | 23418 | 26046 |
| 4637 | 7665 | 12912 | 15535 | 18163 | 4638 | 10293 | 20791 | 23419 | 26047 |
| 4639 | 7666 | 12913 | 15536 | 18164 | 4640 | 10294 | 20792 | 23420 | 26048 |
| 4641 | 7667 | 12914 | 15537 | 18165 | 4642 | 10295 | 20793 | 23421 | 26049 |
| 4643 | 7668 | 12915 | 15538 | 18166 | 4644 | 10296 | 20794 | 23422 | 26050 |
| 4645 | 7669 | 12916 | 15539 | 18167 | 4646 | 10297 | 20795 | 23423 | 26051 |
| 4647 | 7670 | 12917 | 15540 | 18168 | 4648 | 10298 | 20796 | 23424 | 26052 |
| 4649 | 7671 | 12918 | 15541 | 18169 | 4650 | 10299 | 20797 | 23425 | 26053 |
| 4651 | 7672 | 12919 | 15542 | 18170 | 4652 | 10300 | 20798 | 23426 | 26054 |
| 4653 | 7673 | 12920 | 15543 | 18171 | 4654 | 10301 | 20799 | 23427 | 26055 |
| 4655 | 7674 | 12921 | 15544 | 18172 | 4656 | 10302 | 20800 | 23428 | 26056 |
| 4657 | 7675 | 12922 | 15545 | 18173 | 4658 | 10303 | 20801 | 23429 | 26057 |
| 4659 | 7676 | 12923 | 15546 | 18174 | 4660 | 10304 | 20802 | 23430 | 26058 |
| 4661 | 7677 | 12924 | 15547 | 18175 | 4662 | 10305 | 20803 | 23431 | 26059 |
| 4663 | 7678 | 12925 | 15548 | 18176 | 4664 | 10306 | 20804 | 23432 | 26060 |
| 4665 | 7679 | 12926 | 15549 | 18177 | 4666 | 10307 | 20805 | 23433 | 26061 |
| 4667 | 7680 | 12927 | 15550 | 18178 | 4668 | 10308 | 20806 | 23434 | 26062 |
| 4669 | 7681 | 12928 | 15551 | 18179 | 4670 | 10309 | 20807 | 23435 | 26063 |
| 4671 | 7682 | 12929 | 15552 | 18180 | 4672 | 10310 | 20808 | 23436 | 26064 |
| 4673 | 7683 | 12930 | 15553 | 18181 | 4674 | 10311 | 20809 | 23437 | 26065 |
| 4675 | 7684 | 12931 | 15554 | 18182 | 4676 | 10312 | 20810 | 23438 | 26066 |
| 4677 | 7685 | 12932 | 15555 | 18183 | 4678 | 10313 | 20811 | 23439 | 26067 |
| 4679 | 7686 | 12933 | 15556 | 18184 | 4680 | 10314 | 20812 | 23440 | 26068 |
| 4681 | 7687 | 12934 | 15557 | 18185 | 4682 | 10315 | 20813 | 23441 | 26069 |
| 4683 | 7688 | 12935 | 15558 | 18186 | 4684 | 10316 | 20814 | 23442 | 26070 |
| 4685 | 7689 | 12936 | 15559 | 18187 | 4686 | 10317 | 20815 | 23443 | 26071 |
| 4687 | 7690 | 12937 | 15560 | 18188 | 4688 | 10318 | 20816 | 23444 | 26072 |
| 4689 | 7691 | 12938 | 15561 | 18189 | 4690 | 10319 | 20817 | 23445 | 26073 |
| 4691 | 7692 | 12939 | 15562 | 18190 | 4692 | 10320 | 20818 | 23446 | 26074 |
| 4693 | 7693 | 12940 | 15563 | 18191 | 4694 | 10321 | 20819 | 23447 | 26075 |
| 4695 | 7694 | 12941 | 15564 | 18192 | 4696 | 10322 | 20820 | 23448 | 26076 |
| 4697 | 7695 | 12942 | 15565 | 18193 | 4698 | 10323 | 20821 | 23449 | 26077 |
| 4699 | 7696 | 12943 | 15566 | 18194 | 4700 | 10324 | 20822 | 23450 | 26078 |
| 4701 | 7697 | 12944 | 15567 | 18195 | 4702 | 10325 | 20823 | 23451 | 26079 |
| 4703 | 7698 | 12945 | 15568 | 18196 | 4704 | 10326 | 20824 | 23452 | 26080 |
| 4705 | 7699 | 12946 | 15569 | 18197 | 4706 | 10327 | 20825 | 23453 | 26081 |
| 4707 | 7700 | 12947 | 15570 | 18198 | 4708 | 10328 | 20826 | 23454 | 26082 |
| 4709 | 7701 | 12948 | 15571 | 18199 | 4710 | 10329 | 20827 | 23455 | 26083 |
| 4711 | 7702 | 12949 | 15572 | 18200 | 4712 | 10330 | 20828 | 23456 | 26084 |
| 4713 | 7703 | 12950 | 15573 | 18201 | 4714 | 10331 | 20829 | 23457 | 26085 |
| 4715 | 7704 | 12951 | 15574 | 18202 | 4716 | 10332 | 20830 | 23458 | 26086 |
| 4717 | 7705 | 12952 | 15575 | 18203 | 4718 | 10333 | 20831 | 23459 | 26087 |
| 4719 | 7706 | 12953 | 15576 | 18204 | 4720 | 10334 | 20832 | 23460 | 26088 |
| 4721 | 7707 | 12954 | 15577 | 18205 | 4722 | 10335 | 20833 | 23461 | 26089 |
| 4723 | 7708 | 12955 | 15578 | 18206 | 4724 | 10336 | 20834 | 23462 | 26090 |
| 4725 | 7709 | 12956 | 15579 | 18207 | 4726 | 10337 | 20835 | 23463 | 26091 |
| 4727 | 7710 | 12957 | 15580 | 18208 | 4728 | 10338 | 20836 | 23464 | 26092 |
| 4729 | 7711 | 12958 | 15581 | 18209 | 4730 | 10339 | 20837 | 23465 | 26093 |
| 4731 | 7712 | 12959 | 15582 | 18210 | 4732 | 10340 | 20838 | 23466 | 26094 |
| 4733 | 7713 | 12960 | 15583 | 18211 | 4734 | 10341 | 20839 | 23467 | 26095 |
| 4735 | 7714 | 12961 | 15584 | 18212 | 4736 | 10342 | 20840 | 23468 | 26096 |
| 4737 | 7715 | 12962 | 15585 | 18213 | 4738 | 10343 | 20841 | 23469 | 26097 |
| 4739 | 7716 | 12963 | 15586 | 18214 | 4740 | 10344 | 20842 | 23470 | 26098 |
| 4741 | 7717 | 12964 | 15587 | 18215 | 4742 | 10345 | 20843 | 23471 | 26099 |
| 4743 | 7718 | 12965 | 15588 | 18216 | 4744 | 10346 | 20844 | 23472 | 26100 |
| 4745 | 7719 | 12966 | 15589 | 18217 | 4746 | 10347 | 20845 | 23473 | 26101 |
| 4747 | 7720 | 12967 | 15590 | 18218 | 4748 | 10348 | 20846 | 23474 | 26102 |
| 4749 | 7721 | 12968 | 15591 | 18219 | 4750 | 10349 | 20847 | 23475 | 26103 |
| 4751 | 7722 | 12969 | 15592 | 18220 | 4752 | 10350 | 20848 | 23476 | 26104 |
| 4753 | 7723 | 12970 | 15593 | 18221 | 4754 | 10351 | 20849 | 23477 | 26105 |
| 4755 | 7724 | 12971 | 15594 | 18222 | 4756 | 10352 | 20850 | 23478 | 26106 |
| 4757 | 7725 | 12972 | 15595 | 18223 | 4758 | 10353 | 20851 | 23479 | 26107 |
| 4759 | 7726 | 12973 | 15596 | 18224 | 4760 | 10354 | 20852 | 23480 | 26108 |
| 4761 | 7727 | 12974 | 15597 | 18225 | 4762 | 10355 | 20853 | 23481 | 26109 |
| 4763 | 7728 | 12975 | 15598 | 18226 | 4764 | 10356 | 20854 | 23482 | 26110 |
| 4765 | 7729 | 12976 | 15599 | 18227 | 4766 | 10357 | 20855 | 23483 | 26111 |
| 4767 | 7730 | 12977 | 15600 | 18228 | 4768 | 10358 | 20856 | 23484 | 26112 |
| 4769 | 7731 | 12978 | 15601 | 18229 | 4770 | 10359 | 20857 | 23485 | 26113 |
| 4771 | 7732 | 12979 | 15602 | 18230 | 4772 | 10360 | 20858 | 23486 | 26114 |
| 4773 | 7733 | 12980 | 15603 | 18231 | 4774 | 10361 | 20859 | 23487 | 26115 |
| 4775 | 7734 | 12981 | 15604 | 18232 | 4776 | 10362 | 20860 | 23488 | 26116 |
| 4777 | 7735 | 12982 | 15605 | 18233 | 4778 | 10363 | 20861 | 23489 | 26117 |
| 4779 | 7736 | 12983 | 15606 | 18234 | 4780 | 10364 | 20862 | 23490 | 26118 |
| 4781 | 7737 | 12984 | 15607 | 18235 | 4782 | 10365 | 20863 | 23491 | 26119 |
| 4783 | 7738 | 12985 | 15608 | 18236 | 4784 | 10366 | 20864 | 23492 | 26120 |
| 4785 | 7739 | 12986 | 15609 | 18237 | 4786 | 10367 | 20865 | 23493 | 26121 |
| 4787 | 7740 | 12987 | 15610 | 18238 | 4788 | 10368 | 20866 | 23494 | 26122 |
| 4789 | 7741 | 12988 | 15611 | 18239 | 4790 | 10369 | 20867 | 23495 | 26123 |
| 4791 | 7742 | 12989 | 15612 | 18240 | 4792 | 10370 | 20868 | 23496 | 26124 |
| 4793 | 7743 | 12990 | 15613 | 18241 | 4794 | 10371 | 20869 | 23497 | 26125 |
| 4795 | 7744 | 12991 | 15614 | 18242 | 4796 | 10372 | 20870 | 23498 | 26126 |
| 4797 | 7745 | 12992 | 15615 | 18243 | 4798 | 10373 | 20871 | 23499 | 26127 |
| 4799 | 7746 | 12993 | 15616 | 18244 | 4800 | 10374 | 20872 | 23500 | 26128 |
| 4801 | 7747 | 12994 | 15617 | 18245 | 4802 | 10375 | 20873 | 23501 | 26129 |
| 4803 | 7748 | 12995 | 15618 | 18246 | 4804 | 10376 | 20874 | 23502 | 26130 |
| 4805 | 7749 | 12996 | 15619 | 18247 | 4806 | 10377 | 20875 | 23503 | 26131 |
| 4807 | 7750 | 12997 | 15620 | 18248 | 4808 | 10378 | 20876 | 23504 | 26132 |
| 4809 | 7751 | 12998 | 15621 | 18249 | 4810 | 10379 | 20877 | 23505 | 26133 |
| 4811 | 7752 | 12999 | 15622 | 18250 | 4812 | 10380 | 20878 | 23506 | 26134 |
| 4813 | 7753 | 13000 | 15623 | 18251 | 4814 | 10381 | 20879 | 23507 | 26135 |
| 4815 | 7754 | 13001 | 15624 | 18252 | 4816 | 10382 | 20880 | 23508 | 26136 |
| 4817 | 7755 | 13002 | 15625 | 18253 | 4818 | 10383 | 20881 | 23509 | 26137 |
| 4819 | 7756 | 13003 | 15626 | 18254 | 4820 | 10384 | 20882 | 23510 | 26138 |
| 4821 | 7757 | 13004 | 15627 | 18255 | 4822 | 10385 | 20883 | 23511 | 26139 |
| 4823 | 7758 | 13005 | 15628 | 18256 | 4824 | 10386 | 20884 | 23512 | 26140 |
| 4825 | 7759 | 13006 | 15629 | 18257 | 4826 | 10387 | 20885 | 23513 | 26141 |
| 4827 | 7760 | 13007 | 15630 | 18258 | 4828 | 10388 | 20886 | 23514 | 26142 |
| 4829 | 7761 | 13008 | 15631 | 18259 | 4830 | 10389 | 20887 | 23515 | 26143 |
| 4831 | 7762 | 13009 | 15632 | 18260 | 4832 | 10390 | 20888 | 23516 | 26144 |
| 4833 | 7763 | 13010 | 15633 | 18261 | 4834 | 10391 | 20889 | 23517 | 26145 |
| 4835 | 7764 | 13011 | 15634 | 18262 | 4836 | 10392 | 20890 | 23518 | 26146 |
| 4837 | 7765 | 13012 | 15635 | 18263 | 4838 | 10393 | 20891 | 23519 | 26147 |
| 4839 | 7766 | 13013 | 15636 | 18264 | 4840 | 10394 | 20892 | 23520 | 26148 |
| 4841 | 7767 | 13014 | 15637 | 18265 | 4842 | 10395 | 20893 | 23521 | 26149 |
| 4843 | 7768 | 13015 | 15638 | 18266 | 4844 | 10396 | 20894 | 23522 | 26150 |
| 4845 | 7769 | 13016 | 15639 | 18267 | 4846 | 10397 | 20895 | 23523 | 26151 |
| 4847 | 7770 | 13017 | 15640 | 18268 | 4848 | 10398 | 20896 | 23524 | 26152 |
| 4849 | 7771 | 13018 | 15641 | 18269 | 4850 | 10399 | 20897 | 23525 | 26153 |
| 4851 | 7772 | 13019 | 15642 | 18270 | 4852 | 10400 | 20898 | 23526 | 26154 |
| 4853 | 7773 | 13020 | 15643 | 18271 | 4854 | 10401 | 20899 | 23527 | 26155 |
| 4855 | 7774 | 13021 | 15644 | 18272 | 4856 | 10402 | 20900 | 23528 | 26156 |
| 4857 | 7775 | 13022 | 15645 | 18273 | 4858 | 10403 | 20901 | 23529 | 26157 |
| 4859 | 7776 | 13023 | 15646 | 18274 | 4860 | 10404 | 20902 | 23530 | 26158 |
| 4861 | 7777 | 13024 | 15647 | 18275 | 4862 | 10405 | 20903 | 23531 | 26159 |
| 4863 | 7778 | 13025 | 15648 | 18276 | 4864 | 10406 | 20904 | 23532 | 26160 |
| 4865 | 7779 | 13026 | 15649 | 18277 | 4866 | 10407 | 20905 | 23533 | 26161 |
| 4867 | 7780 | 13027 | 15650 | 18278 | 4868 | 10408 | 20906 | 23534 | 26162 |
| 4869 | 7781 | 13028 | 15651 | 18279 | 4870 | 10409 | 20907 | 23535 | 26163 |
| 4871 | 7782 | 13029 | 15652 | 18280 | 4872 | 10410 | 20908 | 23536 | 26164 |
| 4873 | 7783 | 13030 | 15653 | 18281 | 4874 | 10411 | 20909 | 23537 | 26165 |
| 4875 | 7784 | 13031 | 15654 | 18282 | 4876 | 10412 | 20910 | 23538 | 26166 |
| 4877 | 7785 | 13032 | 15655 | 18283 | 4878 | 10413 | 20911 | 23539 | 26167 |
| 4879 | 7786 | 13033 | 15656 | 18284 | 4880 | 10414 | 20912 | 23540 | 26168 |
| 4881 | 7787 | 13034 | 15657 | 18285 | 4882 | 10415 | 20913 | 23541 | 26169 |
| 4883 | 7788 | 13035 | 15658 | 18286 | 4884 | 10416 | 20914 | 23542 | 26170 |
| 4885 | 7789 | 13036 | 15659 | 18287 | 4886 | 10417 | 20915 | 23543 | 26171 |
| 4887 | 7790 | 13037 | 15660 | 18288 | 4888 | 10418 | 20916 | 23544 | 26172 |
| 4889 | 7791 | 13038 | 15661 | 18289 | 4890 | 10419 | 20917 | 23545 | 26173 |
| 4891 | 7792 | 13039 | 15662 | 18290 | 4892 | 10420 | 20918 | 23546 | 26174 |
| 4893 | 7793 | 13040 | 15663 | 18291 | 4894 | 10421 | 20919 | 23547 | 26175 |
| 4895 | 7794 | 13041 | 15664 | 18292 | 4896 | 10422 | 20920 | 23548 | 26176 |
| 4897 | 7795 | 13042 | 15665 | 18293 | 4898 | 10423 | 20921 | 23549 | 26177 |
| 4899 | 7796 | 13043 | 15666 | 18294 | 4900 | 10424 | 20922 | 23550 | 26178 |
| 4901 | 7797 | 13044 | 15667 | 18295 | 4902 | 10425 | 20923 | 23551 | 26179 |
| 4903 | 7798 | 13045 | 15668 | 18296 | 4904 | 10426 | 20924 | 23552 | 26180 |
| 4905 | 7799 | 13046 | 15669 | 18297 | 4906 | 10427 | 20925 | 23553 | 26181 |
| 4907 | 7800 | 13047 | 15670 | 18298 | 4908 | 10428 | 20926 | 23554 | 26182 |
| 4909 | 7801 | 13048 | 15671 | 18299 | 4910 | 10429 | 20927 | 23555 | 26183 |
| 4911 | 7802 | 13049 | 15672 | 18300 | 4912 | 10430 | 20928 | 23556 | 26184 |
| 4913 | 7803 | 13050 | 15673 | 18301 | 4914 | 10431 | 20929 | 23557 | 26185 |
| 4915 | 7804 | 13051 | 15674 | 18302 | 4916 | 10432 | 20930 | 23558 | 26186 |
| 4917 | 7805 | 13052 | 15675 | 18303 | 4918 | 10433 | 20931 | 23559 | 26187 |
| 4919 | 7806 | 13053 | 15676 | 18304 | 4920 | 10434 | 20932 | 23560 | 26188 |
| 4921 | 7807 | 13054 | 15677 | 18305 | 4922 | 10435 | 20933 | 23561 | 26189 |
| 4923 | 7808 | 13055 | 15678 | 18306 | 4924 | 10436 | 20934 | 23562 | 26190 |
| 4925 | 7809 | 13056 | 15679 | 18307 | 4926 | 10437 | 20935 | 23563 | 26191 |
| 4927 | 7810 | 13057 | 15680 | 18308 | 4928 | 10438 | 20936 | 23564 | 26192 |
| 4929 | 7811 | 13058 | 15681 | 18309 | 4930 | 10439 | 20937 | 23565 | 26193 |
| 4931 | 7812 | 13059 | 15682 | 18310 | 4932 | 10440 | 20938 | 23566 | 26194 |
| 4933 | 7813 | 13060 | 15683 | 18311 | 4934 | 10441 | 20939 | 23567 | 26195 |
| 4935 | 7814 | 13061 | 15684 | 18312 | 4936 | 10442 | 20940 | 23568 | 26196 |
| 4937 | 7815 | 13062 | 15685 | 18313 | 4938 | 10443 | 20941 | 23569 | 26197 |
| 4939 | 7816 | 13063 | 15686 | 18314 | 4940 | 10444 | 20942 | 23570 | 26198 |
| 4941 | 7817 | 13064 | 15687 | 18315 | 4942 | 10445 | 20943 | 23571 | 26199 |
| 4943 | 7818 | 13065 | 15688 | 18316 | 4944 | 10446 | 20944 | 23572 | 26200 |
| 4945 | 7819 | 13066 | 15689 | 18317 | 4946 | 10447 | 20945 | 23573 | 26201 |
| 4947 | 7820 | 13067 | 15690 | 18318 | 4948 | 10448 | 20946 | 23574 | 26202 |
| 4949 | 7821 | 13068 | 15691 | 18319 | 4950 | 10449 | 20947 | 23575 | 26203 |
| 4951 | 7822 | 13069 | 15692 | 18320 | 4952 | 10450 | 20948 | 23576 | 26204 |
| 4953 | 7823 | 13070 | 15693 | 18321 | 4954 | 10451 | 20949 | 23577 | 26205 |
| 4955 | 7824 | 13071 | 15694 | 18322 | 4956 | 10452 | 20950 | 23578 | 26206 |
| 4957 | 7825 | 13072 | 15695 | 18323 | 4958 | 10453 | 20951 | 23579 | 26207 |
| 4959 | 7826 | 13073 | 15696 | 18324 | 4960 | 10454 | 20952 | 23580 | 26208 |
| 4961 | 7827 | 13074 | 15697 | 18325 | 4962 | 10455 | 20953 | 23581 | 26209 |
| 4963 | 7828 | 13075 | 15698 | 18326 | 4964 | 10456 | 20954 | 23582 | 26210 |
| 4965 | 7829 | 13076 | 15699 | 18327 | 4966 | 10457 | 20955 | 23583 | 26211 |
| 4967 | 7830 | 13077 | 15700 | 18328 | 4968 | 10458 | 20956 | 23584 | 26212 |
| 4969 | 7831 | 13078 | 15701 | 18329 | 4970 | 10459 | 20957 | 23585 | 26213 |
| 4971 | 7832 | 13079 | 15702 | 18330 | 4972 | 10460 | 20958 | 23586 | 26214 |
| 4973 | 7833 | 13080 | 15703 | 18331 | 4974 | 10461 | 20959 | 23587 | 26215 |
| 4975 | 7834 | 13081 | 15704 | 18332 | 4976 | 10462 | 20960 | 23588 | 26216 |
| 4977 | 7835 | 13082 | 15705 | 18333 | 4978 | 10463 | 20961 | 23589 | 26217 |
| 4979 | 7836 | 13083 | 15706 | 18334 | 4980 | 10464 | 20962 | 23590 | 26218 |
| 4981 | 7837 | 13084 | 15707 | 18335 | 4982 | 10465 | 20963 | 23591 | 26219 |
| 4983 | 7838 | 13085 | 15708 | 18336 | 4984 | 10466 | 20964 | 23592 | 26220 |
| 4985 | 7839 | 13086 | 15709 | 18337 | 4986 | 10467 | 20965 | 23593 | 26221 |
| 4987 | 7840 | 13087 | 15710 | 18338 | 4988 | 10468 | 20966 | 23594 | 26222 |
| 4989 | 7841 | 13088 | 15711 | 18339 | 4990 | 10469 | 20967 | 23595 | 26223 |
| 4991 | 7842 | 13089 | 15712 | 18340 | 4992 | 10470 | 20968 | 23596 | 26224 |
| 4993 | 7843 | 13090 | 15713 | 18341 | 4994 | 10471 | 20969 | 23597 | 26225 |
| 4995 | 7844 | 13091 | 15714 | 18342 | 4996 | 10472 | 20970 | 23598 | 26226 |
| 4997 | 7845 | 13092 | 15715 | 18343 | 4998 | 10473 | 20971 | 23599 | 26227 |
| 4999 | 7846 | 13093 | 15716 | 18344 | 5000 | 10474 | 20972 | 23600 | 26228 |
| 5001 | 7847 | 13094 | 15717 | 18345 | 5002 | 10475 | 20973 | 23601 | 26229 |
| 5003 | 7848 | 13095 | 15718 | 18346 | 5004 | 10476 | 20974 | 23602 | 26230 |
| 5005 | 7849 | 13096 | 15719 | 18347 | 5006 | 10477 | 20975 | 23603 | 26231 |
| 5007 | 7850 | 13097 | 15720 | 18348 | 5008 | 10478 | 20976 | 23604 | 26232 |
| 5009 | 7851 | 13098 | 15721 | 18349 | 5010 | 10479 | 20977 | 23605 | 26233 |
| 5011 | 7852 | 13099 | 15722 | 18350 | 5012 | 10480 | 20978 | 23606 | 26234 |
| 5013 | 7853 | 13100 | 15723 | 18351 | 5014 | 10481 | 20979 | 23607 | 26235 |
| 5015 | 7854 | 13101 | 15724 | 18352 | 5016 | 10482 | 20980 | 23608 | 26236 |
| 5017 | 7855 | 13102 | 15725 | 18353 | 5018 | 10483 | 20981 | 23609 | 26237 |
| 5019 | 7856 | 13103 | 15726 | 18354 | 5020 | 10484 | 20982 | 23610 | 26238 |
| 5021 | 7857 | 13104 | 15727 | 18355 | 5022 | 10485 | 20983 | 23611 | 26239 |
| 5023 | 7858 | 13105 | 15728 | 18356 | 5024 | 10486 | 20984 | 23612 | 26240 |
| 5025 | 7859 | 13106 | 15729 | 18357 | 5026 | 10487 | 20985 | 23613 | 26241 |
| 5027 | 7860 | 13107 | 15730 | 18358 | 5028 | 10488 | 20986 | 23614 | 26242 |
| 5029 | 7861 | 13108 | 15731 | 18359 | 5030 | 10489 | 20987 | 23615 | 26243 |
| 5031 | 7862 | 13109 | 15732 | 18360 | 5032 | 10490 | 20988 | 23616 | 26244 |
| 5033 | 7863 | 13110 | 15733 | 18361 | 5034 | 10491 | 20989 | 23617 | 26245 |
| 5035 | 7864 | 13111 | 15734 | 18362 | 5036 | 10492 | 20990 | 23618 | 26246 |
| 5037 | 7865 | 13112 | 15735 | 18363 | 5038 | 10493 | 20991 | 23619 | 26247 |
| 5039 | 7866 | 13113 | 15736 | 18364 | 5040 | 10494 | 20992 | 23620 | 26248 |
| 5041 | 7867 | 13114 | 15737 | 18365 | 5042 | 10495 | 20993 | 23621 | 26249 |
| 5043 | 7868 | 13115 | 15738 | 18366 | 5044 | 10496 | 20994 | 23622 | 26250 |
| 5045 | 7869 | 13116 | 15739 | 18367 | 5046 | 10497 | 20995 | 23623 | 26251 |
| 5047 | 7870 | 13117 | 15740 | 18368 | 5048 | 10498 | 20996 | 23624 | 26252 |
| 5049 | 7871 | 13118 | 15741 | 18369 | 5050 | 10499 | 20997 | 23625 | 26253 |
| 5051 | 7872 | 13119 | 15742 | 18370 | 5052 | 10500 | 20998 | 23626 | 26254 |
| 5053 | 7873 | 13120 | 15743 | 18371 | 5054 | 10501 | 20999 | 23627 | 26255 |
| 5055 | 7874 | 13121 | 15744 | 18372 | 5056 | 10502 | 21000 | 23628 | 26256 |
| 5057 | 7875 | 13122 | 15745 | 18373 | 5058 | 10503 | 21001 | 23629 | 26257 |
| 5059 | 7876 | 13123 | 15746 | 18374 | 5060 | 10504 | 21002 | 23630 | 26258 |
| 5061 | 7877 | 13124 | 15747 | 18375 | 5062 | 10505 | 21003 | 23631 | 26259 |
| 5063 | 7878 | 13125 | 15748 | 18376 | 5064 | 10506 | 21004 | 23632 | 26260 |
| 5065 | 7879 | 13126 | 15749 | 18377 | 5066 | 10507 | 21005 | 23633 | 26261 |
| 5067 | 7880 | 13127 | 15750 | 18378 | 5068 | 10508 | 21006 | 23634 | 26262 |
| 5069 | 7881 | 13128 | 15751 | 18379 | 5070 | 10509 | 21007 | 23635 | 26263 |
| 5071 | 7882 | 13129 | 15752 | 18380 | 5072 | 10510 | 21008 | 23636 | 26264 |
| 5073 | 7883 | 13130 | 15753 | 18381 | 5074 | 10511 | 21009 | 23637 | 26265 |
| 5075 | 7884 | 13131 | 15754 | 18382 | 5076 | 10512 | 21010 | 23638 | 26266 |
| 5077 | 7885 | 13132 | 15755 | 18383 | 5078 | 10513 | 21011 | 23639 | 26267 |
| 5079 | 7886 | 13133 | 15756 | 18384 | 5080 | 10514 | 21012 | 23640 | 26268 |
| 5081 | 7887 | 13134 | 15757 | 18385 | 5082 | 10515 | 21013 | 23641 | 26269 |
| 5083 | 7888 | 13135 | 15758 | 18386 | 5084 | 10516 | 21014 | 23642 | 26270 |
| 5085 | 7889 | 13136 | 15759 | 18387 | 5086 | 10517 | 21015 | 23643 | 26271 |
| 5087 | 7890 | 13137 | 15760 | 18388 | 5088 | 10518 | 21016 | 23644 | 26272 |
| 5089 | 7891 | 13138 | 15761 | 18389 | 5090 | 10519 | 21017 | 23645 | 26273 |
| 5091 | 7892 | 13139 | 15762 | 18390 | 5092 | 10520 | 21018 | 23646 | 26274 |
| 5093 | 7893 | 13140 | 15763 | 18391 | 5094 | 10521 | 21019 | 23647 | 26275 |
| 5095 | 7894 | 13141 | 15764 | 18392 | 5096 | 10522 | 21020 | 23648 | 26276 |
| 5097 | 7895 | 13142 | 15765 | 18393 | 5098 | 10523 | 21021 | 23649 | 26277 |
| 5099 | 7896 | 13143 | 15766 | 18394 | 5100 | 10524 | 21022 | 23650 | 26278 |
| 5101 | 7897 | 13144 | 15767 | 18395 | 5102 | 10525 | 21023 | 23651 | 26279 |
| 5103 | 7898 | 13145 | 15768 | 18396 | 5104 | 10526 | 21024 | 23652 | 26280 |
| 5105 | 7899 | 13146 | 15769 | 18397 | 5106 | 10527 | 21025 | 23653 | 26281 |
| 5107 | 7900 | 13147 | 15770 | 18398 | 5108 | 10528 | 21026 | 23654 | 26282 |
| 5109 | 7901 | 13148 | 15771 | 18399 | 5110 | 10529 | 21027 | 23655 | 26283 |
| 5111 | 7902 | 13149 | 15772 | 18400 | 5112 | 10530 | 21028 | 23656 | 26284 |
| 5113 | 7903 | 13150 | 15773 | 18401 | 5114 | 10531 | 21029 | 23657 | 26285 |
| 5115 | 7904 | 13151 | 15774 | 18402 | 5116 | 10532 | 21030 | 23658 | 26286 |
| 5117 | 7905 | 13152 | 15775 | 18403 | 5118 | 10533 | 21031 | 23659 | 26287 |
| 5119 | 7906 | 13153 | 15776 | 18404 | 5120 | 10534 | 21032 | 23660 | 26288 |
| 5121 | 7907 | 13154 | 15777 | 18405 | 5122 | 10535 | 21033 | 23661 | 26289 |
| 5123 | 7908 | 13155 | 15778 | 18406 | 5124 | 10536 | 21034 | 23662 | 26290 |
| 5125 | 7909 | 13156 | 15779 | 18407 | 5126 | 10537 | 21035 | 23663 | 26291 |
| 5127 | 7910 | 13157 | 15780 | 18408 | 5128 | 10538 | 21036 | 23664 | 26292 |
| 5129 | 7911 | 13158 | 15781 | 18409 | 5130 | 10539 | 21037 | 23665 | 26293 |
| 5131 | 7912 | 13159 | 15782 | 18410 | 5132 | 10540 | 21038 | 23666 | 26294 |
| 5133 | 7913 | 13160 | 15783 | 18411 | 5134 | 10541 | 21039 | 23667 | 26295 |
| 5135 | 7914 | 13161 | 15784 | 18412 | 5136 | 10542 | 21040 | 23668 | 26296 |
| 5137 | 7915 | 13162 | 15785 | 18413 | 5138 | 10543 | 21041 | 23669 | 26297 |
| 5139 | 7916 | 13163 | 15786 | 18414 | 5140 | 10544 | 21042 | 23670 | 26298 |
| 5141 | 7917 | 13164 | 15787 | 18415 | 5142 | 10545 | 21043 | 23671 | 26299 |
| 5143 | 7918 | 13165 | 15788 | 18416 | 5144 | 10546 | 21044 | 23672 | 26300 |
| 5145 | 7919 | 13166 | 15789 | 18417 | 5146 | 10547 | 21045 | 23673 | 26301 |
| 5147 | 7920 | 13167 | 15790 | 18418 | 5148 | 10548 | 21046 | 23674 | 26302 |
| 5149 | 7921 | 13168 | 15791 | 18419 | 5150 | 10549 | 21047 | 23675 | 26303 |
| 5151 | 7922 | 13169 | 15792 | 18420 | 5152 | 10550 | 21048 | 23676 | 26304 |
| 5153 | 7923 | 13170 | 15793 | 18421 | 5154 | 10551 | 21049 | 23677 | 26305 |
| 5155 | 7924 | 13171 | 15794 | 18422 | 5156 | 10552 | 21050 | 23678 | 26306 |
| 5157 | 7925 | 13172 | 15795 | 18423 | 5158 | 10553 | 21051 | 23679 | 26307 |
| 5159 | 7926 | 13173 | 15796 | 18424 | 5160 | 10554 | 21052 | 23680 | 26308 |
| 5161 | 7927 | 13174 | 15797 | 18425 | 5162 | 10555 | 21053 | 23681 | 26309 |
| 5163 | 7928 | 13175 | 15798 | 18426 | 5164 | 10556 | 21054 | 23682 | 26310 |
| 5165 | 7929 | 13176 | 15799 | 18427 | 5166 | 10557 | 21055 | 23683 | 26311 |
| 5167 | 7930 | 13177 | 15800 | 18428 | 5168 | 10558 | 21056 | 23684 | 26312 |
| 5169 | 7931 | 13178 | 15801 | 18429 | 5170 | 10559 | 21057 | 23685 | 26313 |
| 5171 | 7932 | 13179 | 15802 | 18430 | 5172 | 10560 | 21058 | 23686 | 26314 |
| 5173 | 7933 | 13180 | 15803 | 18431 | 5174 | 10561 | 21059 | 23687 | 26315 |
| 5175 | 7934 | 13181 | 15804 | 18432 | 5176 | 10562 | 21060 | 23688 | 26316 |
| 5177 | 7935 | 13182 | 15805 | 18433 | 5178 | 10563 | 21061 | 23689 | 26317 |
| 5179 | 7936 | 13183 | 15806 | 18434 | 5180 | 10564 | 21062 | 23690 | 26318 |
| 5181 | 7937 | 13184 | 15807 | 18435 | 5182 | 10565 | 21063 | 23691 | 26319 |
| 5183 | 7938 | 13185 | 15808 | 18436 | 5184 | 10566 | 21064 | 23692 | 26320 |
| 5185 | 7939 | 13186 | 15809 | 18437 | 5186 | 10567 | 21065 | 23693 | 26321 |
| 5187 | 7940 | 13187 | 15810 | 18438 | 5188 | 10568 | 21066 | 23694 | 26322 |
| 5189 | 7941 | 13188 | 15811 | 18439 | 5190 | 10569 | 21067 | 23695 | 26323 |
| 5191 | 7942 | 13189 | 15812 | 18440 | 5192 | 10570 | 21068 | 23696 | 26324 |
| 5193 | 7943 | 13190 | 15813 | 18441 | 5194 | 10571 | 21069 | 23697 | 26325 |
| 5195 | 7944 | 13191 | 15814 | 18442 | 5196 | 10572 | 21070 | 23698 | 26326 |
| 5197 | 7945 | 13192 | 15815 | 18443 | 5198 | 10573 | 21071 | 23699 | 26327 |
| 5199 | 7946 | 13193 | 15816 | 18444 | 5200 | 10574 | 21072 | 23700 | 26328 |
| 5201 | 7947 | 13194 | 15817 | 18445 | 5202 | 10575 | 21073 | 23701 | 26329 |
| 5203 | 7948 | 13195 | 15818 | 18446 | 5204 | 10576 | 21074 | 23702 | 26330 |
| 5205 | 7949 | 13196 | 15819 | 18447 | 5206 | 10577 | 21075 | 23703 | 26331 |
| 5207 | 7950 | 13197 | 15820 | 18448 | 5208 | 10578 | 21076 | 23704 | 26332 |
| 5209 | 7951 | 13198 | 15821 | 18449 | 5210 | 10579 | 21077 | 23705 | 26333 |
| 5211 | 7952 | 13199 | 15822 | 18450 | 5212 | 10580 | 21078 | 23706 | 26334 |
| 5213 | 7953 | 13200 | 15823 | 18451 | 5214 | 10581 | 21079 | 23707 | 26335 |
| 5215 | 7954 | 13201 | 15824 | 18452 | 5216 | 10582 | 21080 | 23708 | 26336 |
| 5217 | 7955 | 13202 | 15825 | 18453 | 5218 | 10583 | 21081 | 23709 | 26337 |
| 5219 | 7956 | 13203 | 15826 | 18454 | 5220 | 10584 | 21082 | 23710 | 26338 |
| 5221 | 7957 | 13204 | 15827 | 18455 | 5222 | 10585 | 21083 | 23711 | 26339 |
| 5223 | 7958 | 13205 | 15828 | 18456 | 5224 | 10586 | 21084 | 23712 | 26340 |
| 5225 | 7959 | 13206 | 15829 | 18457 | 5226 | 10587 | 21085 | 23713 | 26341 |
| 5227 | 7960 | 13207 | 15830 | 18458 | 5228 | 10588 | 21086 | 23714 | 26342 |
| 5229 | 7961 | 13208 | 15831 | 18459 | 5230 | 10589 | 21087 | 23715 | 26343 |
| 5231 | 7962 | 13209 | 15832 | 18460 | 5232 | 10590 | 21088 | 23716 | 26344 |
| 5233 | 7963 | 13210 | 15833 | 18461 | 5234 | 10591 | 21089 | 23717 | 26345 |
| 5235 | 7964 | 13211 | 15834 | 18462 | 5236 | 10592 | 21090 | 23718 | 26346 |
| 5237 | 7965 | 13212 | 15835 | 18463 | 5238 | 10593 | 21091 | 23719 | 26347 |
| 5239 | 7966 | 13213 | 15836 | 18464 | 5240 | 10594 | 21092 | 23720 | 26348 |
| 5241 | 7967 | 13214 | 15837 | 18465 | 5242 | 10595 | 21093 | 23721 | 26349 |
| 5243 | 7968 | 13215 | 15838 | 18466 | 5244 | 10596 | 21094 | 23722 | 26350 |
| 5245 | 7969 | 13216 | 15839 | 18467 | 5246 | 10597 | 21095 | 23723 | 26351 |
| 5247 | 7970 | 13217 | 15840 | 18468 | 5248 | 10598 | 21096 | 23724 | 26352 |
| 5249 | 7971 | 13218 | 15841 | 18469 | 5250 | 10599 | 21097 | 23725 | 26353 |
| 5251 | 7972 | 13219 | 15842 | 18470 | 5252 | 10600 | 21098 | 23726 | 26354 |
| 5253 | 7973 | 13220 | 15843 | 18471 | 5254 | 10601 | 21099 | 23727 | 26355 |
| 5255 | 7974 | 13221 | 15844 | 18472 | 5256 | 10602 | 21100 | 23728 | 26356 |
| 5257 | 7975 | 13222 | 15845 | 18473 | 5258 | 10603 | 21101 | 23729 | 26357 |
| 5259 | 7976 | 13223 | 15846 | 18474 | 5260 | 10604 | 21102 | 23730 | 26358 |
| 5261 | 7977 | 13224 | 15847 | 18475 | 5262 | 10605 | 21103 | 23731 | 26359 |
| 5263 | 7978 | 13225 | 15848 | 18476 | 5264 | 10606 | 21104 | 23732 | 26360 |
| 5265 | 7979 | 13226 | 15849 | 18477 | 5266 | 10607 | 21105 | 23733 | 26361 |
| 5267 | 7980 | 13227 | 15850 | 18478 | 5268 | 10608 | 21106 | 23734 | 26362 |
| 5269 | 7981 | 13228 | 15851 | 18479 | 5270 | 10609 | 21107 | 23735 | 26363 |
| 5271 | 7982 | 13229 | 15852 | 18480 | 5272 | 10610 | 21108 | 23736 | 26364 |
| 5273 | 7983 | 13230 | 15853 | 18481 | 5274 | 10611 | 21109 | 23737 | 26365 |
| 5275 | 7984 | 13231 | 15854 | 18482 | 5276 | 10612 | 21110 | 23738 | 26366 |
| 5277 | 7985 | 13232 | 15855 | 18483 | 5278 | 10613 | 21111 | 23739 | 26367 |
| 5279 | 7986 | 13233 | 15856 | 18484 | 5280 | 10614 | 21112 | 23740 | 26368 |
| 5281 | 7987 | 13234 | 15857 | 18485 | 5282 | 10615 | 21113 | 23741 | 26369 |
| 5283 | 7988 | 13235 | 15858 | 18486 | 5284 | 10616 | 21114 | 23742 | 26370 |
| 5285 | 7989 | 13236 | 15859 | 18487 | 5286 | 10617 | 21115 | 23743 | 26371 |
| 5287 | 7990 | 13237 | 15860 | 18488 | 5288 | 10618 | 21116 | 23744 | 26372 |
| 5289 | 7991 | 13238 | 15861 | 18489 | 5290 | 10619 | 21117 | 23745 | 26373 |
| 5291 | 7992 | 13239 | 15862 | 18490 | 5292 | 10620 | 21118 | 23746 | 26374 |
| 5293 | 7993 | 13240 | 15863 | 18491 | 5294 | 10621 | 21119 | 23747 | 26375 |
| 5295 | 7994 | 13241 | 15864 | 18492 | 5296 | 10622 | 21120 | 23748 | 26376 |
| 5297 | 7995 | 13242 | 15865 | 18493 | 5298 | 10623 | 21121 | 23749 | 26377 |
| 5299 | 7996 | 13243 | 15866 | 18494 | 5300 | 10624 | 21122 | 23750 | 26378 |
| 5301 | 7997 | 13244 | 15867 | 18495 | 5302 | 10625 | 21123 | 23751 | 26379 |
| 5303 | 7998 | 13245 | 15868 | 18496 | 5304 | 10626 | 21124 | 23752 | 26380 |
| 5305 | 7999 | 13246 | 15869 | 18497 | 5306 | 10627 | 21125 | 23753 | 26381 |
| 5307 | 8000 | 13247 | 15870 | 18498 | 5308 | 10628 | 21126 | 23754 | 26382 |
| 5309 | 8001 | 13248 | 15871 | 18499 | 5310 | 10629 | 21127 | 23755 | 26383 |
| 5311 | 8002 | 13249 | 15872 | 18500 | 5312 | 10630 | 21128 | 23756 | 26384 |
| 5313 | 8003 | 13250 | 15873 | 18501 | 5314 | 10631 | 21129 | 23757 | 26385 |
| 5315 | 8004 | 13251 | 15874 | 18502 | 5316 | 10632 | 21130 | 23758 | 26386 |
| 5317 | 8005 | 13252 | 15875 | 18503 | 5318 | 10633 | 21131 | 23759 | 26387 |
| 5319 | 8006 | 13253 | 15876 | 18504 | 5320 | 10634 | 21132 | 23760 | 26388 |
| 5321 | 8007 | 13254 | 15877 | 18505 | 5322 | 10635 | 21133 | 23761 | 26389 |
| 5323 | 8008 | 13255 | 15878 | 18506 | 5324 | 10636 | 21134 | 23762 | 26390 |
| 5325 | 8009 | 13256 | 15879 | 18507 | 5326 | 10637 | 21135 | 23763 | 26391 |
| 5327 | 8010 | 13257 | 15880 | 18508 | 5328 | 10638 | 21136 | 23764 | 26392 |
| 5329 | 8011 | 13258 | 15881 | 18509 | 5330 | 10639 | 21137 | 23765 | 26393 |
| 5331 | 8012 | 13259 | 15882 | 18510 | 5332 | 10640 | 21138 | 23766 | 26394 |
| 5333 | 8013 | 13260 | 15883 | 18511 | 5334 | 10641 | 21139 | 23767 | 26395 |
| 5335 | 8014 | 13261 | 15884 | 18512 | 5336 | 10642 | 21140 | 23768 | 26396 |
| 5337 | 8015 | 13262 | 15885 | 18513 | 5338 | 10643 | 21141 | 23769 | 26397 |
| 5339 | 8016 | 13263 | 15886 | 18514 | 5340 | 10644 | 21142 | 23770 | 26398 |
| 5341 | 8017 | 13264 | 15887 | 18515 | 5342 | 10645 | 21143 | 23771 | 26399 |
| 5343 | 8018 | 13265 | 15888 | 18516 | 5344 | 10646 | 21144 | 23772 | 26400 |
| 5345 | 8019 | 13266 | 15889 | 18517 | 5346 | 10647 | 21145 | 23773 | 26401 |
| 5347 | 8020 | 13267 | 15890 | 18518 | 5348 | 10648 | 21146 | 23774 | 26402 |
| 5349 | 8021 | 13268 | 15891 | 18519 | 5350 | 10649 | 21147 | 23775 | 26403 |
| 5351 | 8022 | 13269 | 15892 | 18520 | 5352 | 10650 | 21148 | 23776 | 26404 |
| 5353 | 8023 | 13270 | 15893 | 18521 | 5354 | 10651 | 21149 | 23777 | 26405 |
| 5355 | 8024 | 13271 | 15894 | 18522 | 5356 | 10652 | 21150 | 23778 | 26406 |
| 5357 | 8025 | 13272 | 15895 | 18523 | 5358 | 10653 | 21151 | 23779 | 26407 |
| 5359 | 8026 | 13273 | 15896 | 18524 | 5360 | 10654 | 21152 | 23780 | 26408 |
| 5361 | 8027 | 13274 | 15897 | 18525 | 5362 | 10655 | 21153 | 23781 | 26409 |
| 5363 | 8028 | 13275 | 15898 | 18526 | 5364 | 10656 | 21154 | 23782 | 26410 |
| 5365 | 8029 | 13276 | 15899 | 18527 | 5366 | 10657 | 21155 | 23783 | 26411 |
| 5367 | 8030 | 13277 | 15900 | 18528 | 5368 | 10658 | 21156 | 23784 | 26412 |
| 5369 | 8031 | 13278 | 15901 | 18529 | 5370 | 10659 | 21157 | 23785 | 26413 |
| 5371 | 8032 | 13279 | 15902 | 18530 | 5372 | 10660 | 21158 | 23786 | 26414 |
| 5373 | 8033 | 13280 | 15903 | 18531 | 5374 | 10661 | 21159 | 23787 | 26415 |
| 5375 | 8034 | 13281 | 15904 | 18532 | 5376 | 10662 | 21160 | 23788 | 26416 |
| 5377 | 8035 | 13282 | 15905 | 18533 | 5378 | 10663 | 21161 | 23789 | 26417 |
| 5379 | 8036 | 13283 | 15906 | 18534 | 5380 | 10664 | 21162 | 23790 | 26418 |
| 5381 | 8037 | 13284 | 15907 | 18535 | 5382 | 10665 | 21163 | 23791 | 26419 |
| 5383 | 8038 | 13285 | 15908 | 18536 | 5384 | 10666 | 21164 | 23792 | 26420 |
| 5385 | 8039 | 13286 | 15909 | 18537 | 5386 | 10667 | 21165 | 23793 | 26421 |
| 5387 | 8040 | 13287 | 15910 | 18538 | 5388 | 10668 | 21166 | 23794 | 26422 |
| 5389 | 8041 | 13288 | 15911 | 18539 | 5390 | 10669 | 21167 | 23795 | 26423 |
| 5391 | 8042 | 13289 | 15912 | 18540 | 5392 | 10670 | 21168 | 23796 | 26424 |
| 5393 | 8043 | 13290 | 15913 | 18541 | 5394 | 10671 | 21169 | 23797 | 26425 |
| 5395 | 8044 | 13291 | 15914 | 18542 | 5396 | 10672 | 21170 | 23798 | 26426 |
| 5397 | 8045 | 13292 | 15915 | 18543 | 5398 | 10673 | 21171 | 23799 | 26427 |
| 5399 | 8046 | 13293 | 15916 | 18544 | 5400 | 10674 | 21172 | 23800 | 26428 |
| 5401 | 8047 | 13294 | 15917 | 18545 | 5402 | 10675 | 21173 | 2380 | 26429 |
| 5403 | 8048 | 13295 | 15918 | 18546 | 5404 | 10676 | 21174 | 23802 | 26430 |
| 5405 | 8049 | 13296 | 15919 | 18547 | 5406 | 10677 | 21175 | 23803 | 26431 |
| 5407 | 8050 | 13297 | 15920 | 18548 | 5408 | 10678 | 21176 | 23804 | 26432 |
| 5409 | 8051 | 13298 | 15921 | 18549 | 5410 | 10679 | 21177 | 23805 | 26433 |
| 5411 | 8052 | 13299 | 15922 | 18550 | 5412 | 10680 | 21178 | 23806 | 26434 |
| 5413 | 8053 | 13300 | 15923 | 18551 | 5414 | 10681 | 21179 | 23807 | 26435 |
| 5415 | 8054 | 13301 | 15924 | 18552 | 5416 | 10682 | 21180 | 23808 | 26436 |
| 5417 | 8055 | 13302 | 15925 | 18553 | 5418 | 10683 | 21181 | 23809 | 26437 |
| 5419 | 8056 | 13303 | 15926 | 18554 | 5420 | 10684 | 21182 | 23810 | 26438 |
| 5421 | 8057 | 13304 | 15927 | 18555 | 5422 | 10685 | 21183 | 23811 | 26439 |
| 5423 | 8058 | 13305 | 15928 | 18556 | 5424 | 10686 | 21184 | 23812 | 26440 |
| 5425 | 8059 | 13306 | 15929 | 18557 | 5426 | 10687 | 21185 | 23813 | 26441 |
| 5427 | 8060 | 13307 | 15930 | 18558 | 5428 | 10688 | 21186 | 23814 | 26442 |
| 5429 | 8061 | 13308 | 15931 | 18559 | 5430 | 10689 | 21187 | 23815 | 26443 |
| 5431 | 8062 | 13309 | 15932 | 18560 | 5432 | 10690 | 21188 | 23816 | 26444 |
| 5433 | 8063 | 13310 | 15933 | 18561 | 5434 | 10691 | 21189 | 23817 | 26445 |
| 5435 | 8064 | 13311 | 15934 | 18562 | 5436 | 10692 | 21190 | 23818 | 26446 |

1. A recombinant antibody, wherein the antibody comprises a light chain variable region (VL) that comprises a light chain complementarity determining region (CDRL)1, CDRL2, and CDRL3 and/or a heavy chain variable region (VH) that comprises a heavy chain complementarity determining region (CDRH)1, CDRH2, and CDRH3, wherein:
CDRH3 comprises an amino acid sequence at least 60% identical to SEQ ID NOs: 109-126; and
CDRL3 comprises an amino acid sequence at least 60% identical to SEQ ID NOs: 163-180.

2. The recombinant antibody of claim 1, wherein CDRH3 comprises at least one amino acid substitution when compared to SEQ ID NOs: 109-126.

3. The recombinant antibody of claim 1, wherein CDRL3 comprises at least one amino acid substitution when compared to SEQ ID NOs: 163-180.

4. The recombinant antibody of claim 1, wherein:
CDRH1 comprises an amino acid sequence at least 60% identical to SEQ ID NOs: 73-90; and/or
CDRL1 comprises an amino acid sequence at least 60% identical to SEQ ID NOs: 127-144.

5. The recombinant antibody of claim 1, wherein CDRH1 comprises at least one amino acid substitution when compared to SEQ ID NOs: 73-90.

6. The recombinant antibody of claim 1, wherein CDRL1 comprises at least one amino acid substitution when compared to SEQ ID NOs: 127-144.

7. The recombinant antibody of claim 1, wherein:
CDRH2 comprises an amino acid sequence at least 60% identical to SEQ ID NOs: 91-108; and/or
CDRL2 comprises an amino acid sequence at least 60% identical to SEQ ID NOs: 145-162.

8. The recombinant antibody of claim 1, wherein CDRH2 comprises at least one amino acid substitution when compared to SEQ ID NOs: 91-108.

9. The recombinant antibody of claim 1, wherein CDRL2 comprises at least one amino acid substitution when compared to SEQ ID NOs: 145-162.

10. The recombinant antibody of claim 1, wherein VH comprises an amino acid sequence selected from the group consisting of SEQ ID NOs: 37-54.

11. The recombinant antibody of claim 1, wherein VL comprises an amino acid sequence selected from the group consisting of SEQ ID NOs: 55-72.

12. The recombinant antibody of claim 1, wherein the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO:73,
CDRH2 is SEQ ID NO:91,
CDRH3 is SEQ ID NO:109,
CDRL1 is SEQ ID NO:127,
CDRL2 is SEQ ID NO:145, and
CDRL3 is SEQ ID NO:163;
CDRH1 is SEQ ID NO:74,
CDRH2 is SEQ ID NO:92,
CDRH3 is SEQ ID NO:110,
CDRL1 is SEQ ID NO:128,
CDRL2 is SEQ ID NO:146, and
CDRL3 is SEQ ID NO:164;
CDRH1 is SEQ ID NO:75,
CDRH2 is SEQ ID NO:93,
CDRH3 is SEQ ID NO:111,
CDRL1 is SEQ ID NO:129,
CDRL2 is SEQ ID NO:147, and
CDRL3 is SEQ ID NO:165;
CDRH1 is SEQ ID NO:76,
CDRH2 is SEQ ID NO:94,
CDRH3 is SEQ ID NO:112,
CDRL1 is SEQ ID NO:130,
CDRL2 is SEQ ID NO:148, and
CDRL3 is SEQ ID NO:166;
CDRH1 is SEQ ID NO:77,
CDRH2 is SEQ ID NO:95,
CDRH3 is SEQ ID NO:113,
CDRL1 is SEQ ID NO:131,
CDRL2 is SEQ ID NO:149, and
CDRL3 is SEQ ID NO:167;
CDRH1 is SEQ ID NO:78,
CDRH2 is SEQ ID NO:96,
CDRH3 is SEQ ID NO:114,
CDRL1 is SEQ ID NO:132,
CDRL2 is SEQ ID NO:150, and
CDRL3 is SEQ ID NO:168;
CDRH1 is SEQ ID NO:79,
CDRH2 is SEQ ID NO:97,
CDRH3 is SEQ ID NO:115,
CDRL1 is SEQ ID NO:133,
CDRL2 is SEQ ID NO:151, and
CDRL3 is SEQ ID NO:169;
CDRH1 is SEQ ID NO:80,
CDRH2 is SEQ ID NO:98,
CDRH3 is SEQ ID NO:116,
CDRL1 is SEQ ID NO:134,
CDRL2 is SEQ ID NO:152, and
CDRL3 is SEQ ID NO:170;
CDRH1 is SEQ ID NO:81,
CDRH2 is SEQ ID NO:99,
CDRH3 is SEQ ID NO:117,
CDRL1 is SEQ ID NO:135,
CDRL2 is SEQ ID NO:153, and
CDRL3 is SEQ ID NO:171;
CDRH1 is SEQ ID NO:82,
CDRH2 is SEQ ID NO:100,
CDRH3 is SEQ ID NO:118,
CDRL1 is SEQ ID NO:136,
CDRL2 is SEQ ID NO:154, and
CDRL3 is SEQ ID NO:172;
CDRH1 is SEQ ID NO:83,
CDRH2 is SEQ ID NO:101,
CDRH3 is SEQ ID NO:119,
CDRL1 is SEQ ID NO:137,
CDRL2 is SEQ ID NO:155, and
CDRL3 is SEQ ID NO:173;
CDRH1 is SEQ ID NO:84,
CDRH2 is SEQ ID NO:102,
CDRH3 is SEQ ID NO:120,
CDRL1 is SEQ ID NO:138,
CDRL2 is SEQ ID NO:156, and
CDRL3 is SEQ ID NO:174;
CDRH1 is SEQ ID NO:85,
CDRH2 is SEQ ID NO:103,
CDRH3 is SEQ ID NO:121,
CDRL1 is SEQ ID NO:139,
CDRL2 is SEQ ID NO:157, and
CDRL3 is SEQ ID NO:175;
CDRH1 is SEQ ID NO:86,
CDRH2 is SEQ ID NO:104,
CDRH3 is SEQ ID NO:122,
CDRL1 is SEQ ID NO:140,
CDRL2 is SEQ ID NO:158, and
CDRL3 is SEQ ID NO:176;
CDRH1 is SEQ ID NO:87,
CDRH2 is SEQ ID NO:105,
CDRH3 is SEQ ID NO:123,
CDRL1 is SEQ ID NO:141,
CDRL2 is SEQ ID NO:159, and
CDRL3 is SEQ ID NO:177;
CDRH1 is SEQ ID NO:88,
CDRH2 is SEQ ID NO:106,
CDRH3 is SEQ ID NO:124,
CDRL1 is SEQ ID NO:142,
CDRL2 is SEQ ID NO:160, and
CDRL3 is SEQ ID NO:178;
CDRH1 is SEQ ID NO:89,
CDRH2 is SEQ ID NO:107,
CDRH3 is SEQ ID NO:125,
CDRL1 is SEQ ID NO:143,
CDRL2 is SEQ ID NO:161, and
CDRL3 is SEQ ID NO:179;
or
CDRH1 is SEQ ID NO:90,
CDRH2 is SEQ ID NO:108,
CDRH3 is SEQ ID NO:126,
CDRL1 is SEQ ID NO:144,
CDRL2 is SEQ ID NO:162, and
CDRL3 is SEQ ID NO:180.

13.-29. (canceled)

30. A nucleic acid encoding the recombinant antibody of claim 1.

31. A recombinant expression cassette or plasmid comprising a sequence to express a recombinant antibody of claim 1.

32. A host cell comprising the expression cassette or the plasmid of claim 31.

33. A method of producing an antibody, comprising cultivating or maintaining the host cell of claim 32 under conditions to produce the antibody.

34. A method of treating a coronavirus infection in a subject, comprising administering to the subject a therapeutically effective amount of the recombinant antibody of claim 1.

35. The method of claim 34, where the coronavirus is SARS-CoV-2.
